(19) United States
(12) Patent Application Publication
(10) Pub. No.: US 2025/0084094 A1
(43) Pub. Date: Mar. 13, 2025

(54) SPIROCYCLIC INHIBITORS OF APOL1 AND METHODS OF USING SAME

(71) Applicant: Vertex Pharmaceuticals Incorporated, Boston, MA (US)

(72) Inventors: Timothy J. Senter, Arlington, MA (US); Leslie A. Dakin, Lakeville, MA (US); Ming Chen, Medford, MA (US); Steven D. Stone, Quincy, MA (US); Elena Dolgikh, Boston, MA (US); Jessica H. Olsen, Jamaica Plain, MA (US); Haoxuan Wang, Somerville, MA (US)

(21) Appl. No.: 18/714,031

(22) PCT Filed: Nov. 29, 2022
(86) PCT No.: PCT/US2022/051274

§ 371 Date: May 28, 2024

Related U.S. Application Data
(60) Provisional application No. 63/284,195, filed on Nov. 30, 2021.

(51) Int. Cl. C07D 491/107; A61K 31/438; C07D 471/10

(52) U.S. Cl. CPC C07D 491/107; A61K 31/438; C07D 471/10

(57) ABSTRACT

The disclosure provides at least one compound, tautomer, deuterated derivative, or pharmaceutically acceptable salt chosen from compounds of Formula I, tautomers thereof, deuterated derivatives of those compounds or tautomers, and pharmaceutically acceptable salts of any of the foregoing, compositions comprising the same, and methods of using the same, including uses in treating APOL1-mediated diseases, including pancreatic cancer, focal segmental glomerulosclerosis (FSGS), and/or non-diabetic kidney disease (NDKD). (I)

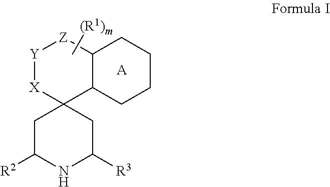

This application claims the benefit of priority of U.S. Provisional Application No. 63/284,195, filed Nov. 30, 2021, the contents of which are incorporated by reference herein in their entirety.

This disclosure provides compounds that may inhibit apolipoprotein L1 (APOL1) and methods of using those compounds to treat APOL1-mediated diseases, such as, e.g., pancreatic cancer, focal segmental glomerulosclerosis (FSGS), and/or non-diabetic kidney disease (NDKD). In some embodiments, the FSGS and/or NDKD is associated with at least one of the 2 common APOL1 genetic variants (GI: S342G:1384M and G2: N388del:Y389del). In some embodiments, the pancreatic cancer is associated with elevated levels of APOL1 (such as, e.g., elevated levels of APOL1 in pancreatic cancer tissues).

FSGS is a rare kidney disease with an estimated global incidence of 0.2 to 1.1/100,000/year. FSGS is a disease of the podocyte (glomerular visceral epithelial cells) responsible for proteinuria and progressive decline in kidney function. NDKD is a kidney disease involving damage to the podocyte or glomerular vascular bed that is not attributable to diabetes. NDKD is a disease characterized by hypertension and progressive decline in kidney function. Human genetics support a causal role for the G1 and G2 APOL1 variants in inducing kidney disease. Individuals with 2 APOL1 alleles are at increased risk of developing end-stage kidney disease (ESKD), including primary (idiopathic) FSGS, human immunodeficiency virus (HIV)_associated FSGS, NDKD, arterionephrosclerosis, lupus nephritis, microalbuminuria, and chronic kidney disease. See, P. Dummer et al., Semin Nephrol. 35(3): 222-236 (2015).

FSGS and NDKD can be divided into different subgroups based on the underlying etiology. One homogeneous subgroup of FSGS is characterized by the presence of independent common sequence variants in the apolipoprotein L1 (APOL1) gene termed GI and G2, which are referred to as the “APOL1 risk alleles.” G1 encodes a correlated pair of non-synonymous amino acid changes (S342G and 1384M), G2 encodes a 2 amino acid deletion (N388del:Y389del) near the C terminus of the protein, and G0 is the ancestral (low risk) allele. A distinct phenotype ofNDKD is found in patients with APOL1 genetic risk variants as well. In both APOL1-mediated FSGS and NDKD, higher levels of proteinuria and a more accelerated loss of kidney function occur in patients with two risk alleles compared to patients with the same disease who have no orjust 1 APOL1 genetic risk variant. Alternatively in AMKD, higher levels of proteinuria and accelerated loss of kidney function can also occur in patients with one risk allele. See, G. Vajgel et al., J. Rheumatol., November 2019, jrheum.190684.

APOL1 is a 44 kDa protein that is only expressed in humans, gorillas, and baboons. The APOL1 gene is expressed in multiple organs in humans, including the liver and kidney. APOL1 is produced mainly by the liver and contains a signal peptide that allows for secretion into the bloodstream, where it circulates bound to a subset of high-density lipoproteins. APOL1 is responsible for protection against the invasive parasite, Trypanosoma brucei brucei (T. b. brucei). APOL1 is endocytosed by T. b. brucei and transported to lysosomes, where it inserts into the lysosomal membrane and forms pores that lead to parasite swelling and death.

While the ability to lyse T. b. brucei is shared by all 3 APOL1 variants (G0, G1, and G2), APOL1 G1 and G2 variants confer additional protection against parasite species that have evolved a serum resistant associated-protein (SRA) which inhibits APOL1 G0; APOL1 G1 and G2 variants confer additional protection against trypanosoma species that cause sleeping sickness. G1 and G2 variants evade inhibition by SRA; G1 confers additional protection against T. b. gambiense (which causes West African sleeping sickness) while G2 confers additional protection against T. b. rhodesiense (which causes East African sleeping sickness).

In the kidney, APOL1 is expressed in podocytes, endothelial cells (including glomerular endothelial cells), and some tubular cells. Podocyte-specific expression of APOL1 G1 or G2 (but not G0) in transgenic mice induces structural and functional changes, including albuminuria, decreased kidney function, podocyte abnormalities, and glomerulosclerosis. Consistent with these data, G1 and G2 variants of APOL1 play a causative role in inducing FSGS and accelerating its progression in humans. Individuals with APOL1 risk alleles (i.e., homozygous or compound heterozygous for the APOL1 G1 or APOL1 G2 alleles) have increased risk of developing FSGS and they are at risk for rapid decline in kidney function if they develop FSGS. Thus, inhibition of APOL1 could have a positive impact in individuals who harbor APOL1 risk alleles.

Although normal plasma concentrations of APOL1 are relatively high and can vary at least 20-fold in humans, circulating APOL1 is not causally associated with kidney disease. However, APOL1 in the kidney is thought to be responsible for the development of kidney diseases, including FSGS and NDKD. Under certain circumstances, APOL1 protein synthesis can be increased by approximately 200-fold by pro-inflammatory cytokines such as interferons or tumor necrosis factor-α. In addition, several studies have shown that APOL1 protein can form pH-gated Na⁺/K⁺ pores in the cell membrane, resulting in a net efflux of intracellular K⁺, ultimately resulting in activation of local and systemic inflammatory responses, cell swelling, and death.

The risk of ESKD is substantially higher in people of recent sub-Saharan African ancestry as compared to those of European ancestry. In the United States, ESKD is responsible for nearly as many lost years of life in women as from breast cancer and more lost years of life in men than from colorectal cancer.

FSGS and NDKD are caused by damage to podocytes, which are part of the glomerular filtration barrier, resulting in proteinuria. Patients with proteinuria are at a higher risk of developing end-stage kidney disease (ESKD) and developing proteinuria-related complications, such as infections or thromboembolic events. There is no standardized treatment regimen nor approved drugs for FSGS or NDKD. Currently, FSGS and NDKD are managed with symptomatic treatment (including blood pressure control using blockers of the renin angiotensin system), and patients with FSGS and heavy proteinuria may be offered high dose steroids. Current therapeutic options for NDKD are anchored on blood pressure control and blockade of the renin angiotensin system.

Corticosteroids, alone or in combination with other immunosuppressants, induce remission in a minority of patients (e.g., remission of proteinuria in a minority of patients) and are associated with numerous side effects. However, remission is frequently indurable even in patients initially responsive to corticosteroid and/or immunosuppressant treatment. As a result, patients, in particular individuals of recent sub-Saharan African ancestry with 2 APOL1 risk alleles, experience rapid disease progression leading to end-stage renal disease (ESRD). Thus, there is an unmet medical need for treatment for FSGS and NDKD. Illustratively, in view of evidence that APOL1 plays a causative role in inducing and accelerating the progression of kidney disease, inhibition of APOL1 should have a positive impact on patients with APOL1 mediated kidney disease, particularly those who carry two APOL1 risk alleles (i.e., are homozygous or compound heterozygous for the G1 or G2 alleles).

Additionally, APOL1 is an aberrantly expressed gene in multiple cancers (Lin et al., Cell Death and Disease (2021), 12:760). Recently, APOL1 was found to be abnormally elevated in human pancreatic cancer tissues compared with adjacent tissues and was associated with poor prognosis in pancreatic cancer patients. In in vivo and in vitro experiments, knockdown of APOL1 significantly inhibited cancer cell proliferation and promoted the apoptosis of pancreatic cancer cells.

One aspect of the disclosure provides at least one compound, tautomer, deuterated derivative, or pharmaceutically acceptable salt chosen from compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, tautomers thereof, deuterated derivatives of those compounds or tautomers, and pharmaceutically acceptable salts of any of the foregoing, which can be employed in the treatment of diseases mediated by APOL1, such as FSGS and NDKD. For example, in some embodiments, the at least one compound is a compound represented by Formula I:

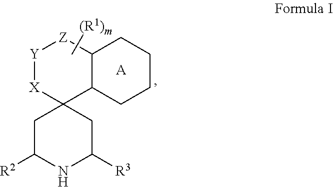

wherein X, Y, Z, R¹, R², R³, Ring A, and m are as defined in an embodiment disclosed herein.

In some embodiments, at least one compound, tautomer, deuterated derivative, or pharmaceutically acceptable salt of the disclosure is a compound represented by the following structural formula:

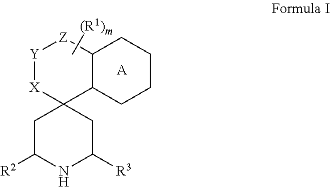

a tautomer thereof, a deuterated derivative of that compound or tautomer, or a pharmaceutically acceptable salt of any of the foregoing, wherein:
   Ring A is chosen from 6-membered aryl and 6-membered heteroaryl groups;
   X is chosen from —CH₂—, —C(O)—, —S(O)₂—, —NH—, and —O—;
   Y is chosen from —CH₂—, —C(O)—, —S(O)₂—, —NH—, and —O—;
   Z is chosen from a bond, —CH₂—, —NH—, —C(O)—, —S(O)₂—, and —O—, wherein:
      at least one of X and Y is chosen from —CH₂— and —C(O)—; and
      for each of X, Y, and Z, a hydrogen atom in each instance of —CH₂— or —NH— is optionally replaced by R¹;
   R¹, for each occurrence, is independently chosen from halogen, —OH, cyano, phenyl, C₁-C₆ alkyl, C₁-C₆ alkoxy, C₃-C₆ carbocyclyl, 4- to 6-membered heterocyclyl, —C(═O)OR^{c}, —C(═O)N(R^{c})₂, and —OS(═O)₂R^{c} groups, wherein:
      R^{c}, for each occurrence, is independently chosen from hydrogen, C₁-C₄ alkyl, and C₁-C₄ haloalkyl groups;
      the 4- to 6-membered heterocyclyl of R¹ comprises one heteroatom chosen from nitrogen and oxygen;
      the C₁-C₆ alkyl of R¹ is optionally substituted with 1 to 3 groups independently chosen from halogen, cyano, —OH, —NH₂, —NH(C₁-C₄ alkyl), —N(C₁-C₄ alkyl)₂, and C₁-C₄ alkoxy groups;
      the C₁-C₆ alkoxy of R¹ is optionally substituted with 1 to 3 groups independently chosen from —OH, cyano, and halogen groups;
      the C₃-C₆ carbocyclyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups; and
      the phenyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups;
   R² is chosen from cyano, C₁-C₆ alkyl, —C(═O)O(C₁-C₄ alkyl), C₂-C₆ alkynyl, and

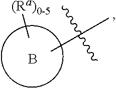

wherein:
   the C₁-C₆ alkyl of R² is optionally substituted with 1 to 3 groups independently chosen from halogen, cyano, —OH, —NH₂, —NH(C₁-C₄ alkyl), —N(C₁-C₄ alkyl)₂, C₁-C₄ alkoxy, —C(═O)NH₂, —C(═O)NH(C₁-C₄ alkyl), —C(═O)N(C₁-C₄ alkyl)₂, C₃-C₆ carbocyclyl, 5- to 10-membered heterocyclyl, C₆ aryl, and 5- to 10-membered heteroaryl groups;
   Ring B is chosen from C₃-C₁₂ carbocyclyl, 3- to 12-membered heterocyclyl, C₆ and C₁₀ aryl, and 5- to 10-membered heteroaryl groups, wherein Ring B is optionally substituted with 1, 2, 3, 4, or 5 Ra groups, wherein:
      R^{a}, for each occurrence, is independently chosen from halogen, cyano, C₁-C₆ alkyl, C₂-C₆ alkenyl, C₁-C₆ alkoxy, C₁-C₆ haloalkyl, C₁-C₆ haloalkenyl, C₁-C₆ haloalkoxy, —C(═O)NR^{h}Rⁱ, —NR^{h}Rⁱ, —NR^{h}C(O)R^{k}, —NR^{h}C(O)OR^{k}, —NR^{h}C(═O)NRⁱR^{j}, —NR^{h}S(═O)ₚR^{k}, —OR^{k}, —OC(═O)R^{k}, —OC(═O)OR^{k}, —OC(═O)NR^{h}Rⁱ, —[O(CH₂)_{q}]ᵣO(C₁-C₆ alkyl), —S(═O)ₚR^{k}, —S(═O)ₚNR^{h}Rⁱ, —C(═O)OR^{k}, C₃-C₁₂ carbocyclyl, 3- to 12-membered heterocyclyl, C₆ and C₁₀ aryl, and 5- to 10-membered heteroaryl groups, wherein:
      the C₁-C₆ alkyl, C₁-C₆ alkoxy, and the C₂-C₆ alkenyl of R^{a} are each optionally substituted with 1 to 3 groups independently chosen from C₆ to C₁₀ aryl (optionally substituted with 1 to 3 R^{m} groups), 5- to 10-membered heterocyclyl (optionally substituted with 1 to 3 R^{m} groups), 5-to 10-membered heteroaryl (optionally substituted with 1 to 3 R^{m} groups), cyano, —C(═O)R^{k}, —C(═O)OR^{k}, —C(═O)NR^{h}Rⁱ, —NR^{h}Rⁱ, —NR^{h}C(═O)R^{k},
      —NR^{h}C(═O)OR^{k}, —NR^{h}C(═O)NRⁱR^{j}, —NR^{h}S(═O)ₚR^{k}, —OR^{k}, —OC(═O)R^{k}, —OC(═O)OR^{k}, —OC(═O)NR^{h}Rⁱ, —S(═O)ₚR^{k}, —S(═O)ₚNR^{h}Rⁱ, —O(C₆ aryl) (optionally substituted with 1 to 3 R^{m} groups), and C₃-C₆ carbocyclyl groups (optionally substituted with 1 to 3 R^{m} groups);
      the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from halogen, cyano, C₁-C₄ alkyl, —NR^{h}Rⁱ, and —OR^{k} groups, wherein:
      R^{h}, Rⁱ, and R^{j}, for each occurrence, are each independently chosen from hydrogen, C₁-C₄ alkyl, C₆-C₁₀ aryl, and C₃-C₆ cycloalkyl groups, wherein:
      the C₁-C₄ alkyl of any one of R^{h}, Rⁱ, and R^{j} is optionally substituted with 1 to 3 groups independently chosen from halogen, cyano, and —OH groups;
      R^{k}, for each occurrence, is independently chosen from hydrogen, C₁-C₄ alkyl, 5- to 10-membered heterocyclyl, and C₃-C₆ carbocyclyl groups, wherein:
      the C₁-C₄ alkyl of any one of R^{k} is optionally substituted with 1 to 3 groups independently chosen from halogen, cyano, and —OH groups;
      R^{m}, for each occurrence, is independently chosen from halogen, cyano, oxo, C₁-C₆ alkyl, C₁-C₆ alkoxy, —S(═O)ₚR^{k}, and —OR^{k} groups, wherein:
      the C₁-C₆ alkyl of R^{m} is optionally substituted with 1 to 3 groups independently chosen from halogen, cyano, —OH, and —O(C₁-C₄ alkyl) groups;
R³ is chosen from C₁-C₆ alkyl, —C(═O)O(C₁-C₄ alkyl), C₃-C₁₂ carbocyclyl, 3- to 12-membered heterocyclyl, C₆ and C₁₀ aryl, and 5- to 10-membered heteroaryl groups, wherein:
   the C₁-C₆ alkyl of R³ is optionally substituted with 1 to 3 groups independently chosen from halogen, cyano, —OH, —NH₂, —NH(C₁-C₄ alkyl), —N(C₁-C₄ alkyl)₂, C₁-C₄ alkoxy, —C(═O)NH₂, —C(═O)NH(C₁-C₄ alkyl), and —C(═O)N(C₁-C₄ alkyl)₂ groups;
   the C₃-C₁₂ carbocyclyl, the 3- to 12-membered heterocyclyl, the C₆ and C₁₀ aryl, and the 5- to 10-membered heteroaryl of R³ are each optionally substituted with 1 to 3 groups independently chosen from halogen, cyano, —OH, —NH₂, —NH(C₁-C₄ alkyl) (optionally substituted with —OH), —N(C₁-C₄ alkyl)₂, C₁-C₅ alkyl (optionally substituted with —OH or —S(═O)₂(C₁-C₄ alkyl)), C₁-C₄ alkoxy, —C(═O)NH₂, —C(═O)NH(C₁-C₄ alkyl), —NHC(═O)(C₁-C₄ alkyl), —C(═O)(C₁-C₄ alkoxy), and —C(═O)N(C₁-C₄ alkyl)₂ groups;
m is an integer chosen from 0, 1, 2, 3, 4, and 5;
p, for each occurrence, is an integer independently chosen from 1 and 2; and
q and r, for each occurrence, are each an integer independently chosen from 1, 2, 3, and 4.

In some embodiments, in a compound, tautomer, deuterated derivative, or pharmaceutically acceptable salt of the disclosure, Ring A is phenyl;
X is chosen from —CH₂—, —C(O)—, —S(O)₂—, —NH—, and —O—;
Y is chosen from —CH₂—, —C(O)—, —S(O)₂—, —NH—, and —O—;
Z is chosen from a bond, —CH₂—, —NH—, —C(O)—, —S(O)₂—, and —O—, wherein:
   at least one of X and Y is chosen from —CH₂— and —C(O)—; and
   for each of X, Y, and Z, a hydrogen atom in each instance of —CH₂— or —NH— is optionally replaced by R¹;
R¹, for each occurrence, is independently chosen from halogen, —OH, cyano, phenyl, C₁-C₆ alkyl, C₁-C₆ alkoxy, —C(═O)OR^{c}, —C(═O)N(R^{c})₂, and —OS(═O)₂R^{c} groups, wherein:
   R^{c}, for each occurrence, is independently chosen from hydrogen, C₁-C₄ alkyl, and C₁-C₄ haloalkyl groups;
   the C₁-C₆ alkyl of R¹ is optionally substituted with 1 to 3 groups independently chosen from halogen, cyano, —OH, —NH₂, —NH(C₁-C₄ alkyl), —N(C₁-C₄ alkyl)₂, and C₁-C₄ alkoxy groups;
   the C₁-C₆ alkoxy of R¹ is optionally substituted with 1 to 3 groups independently chosen from —OH, cyano, and halogen groups;
   the phenyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups;
R² is chosen from C₁-C₆ alkyl and

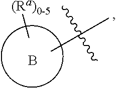

wherein:
   the C₁-C₆ alkyl of R² is optionally substituted with 1 to 3 groups independently chosen from halogen, cyano, —OH, —NH₂, —NH(C₁-C₄ alkyl), —N(C₁-C₄ alkyl)₂, C₁-C₄ alkoxy, —C(═O)NH₂, —C(═O)NH(C₁-C₄ alkyl), —C(═O)N(C₁-C₄ alkyl)₂, C₃-C₆ carbocyclyl, 5- to 10-membered heterocyclyl, C₆ aryl, and 5- to 10-membered heteroaryl groups;
   Ring B is chosen from 3- to 12-membered heterocyclyl, C₆ aryl, and 5- to 10-membered heteroaryl groups, wherein Ring B is optionally substituted with 1, 2, 3, 4, or 5 R^{a} groups, wherein:
      R^{a}, for each occurrence, is independently chosen from halogen, cyano, C₁-C₆ alkyl, C₂-C₆ alkenyl, C₁-C₆ alkoxy, C₁-C₆ haloalkyl, C₁-C₆ haloalkenyl, C₁-C₆ haloalkoxy, —C(═O)NR^{h}Rⁱ, —NR^{h}Rⁱ, —NR^{h}C(═O)R^{k}, —NR^{h}C(═O)OR^{k}, —NR^{h}C(═O)NRⁱR^{j}, —NR^{h}S(═O)ₚR^{k}, —OR^{k}, —OC(═O)R^{k}, —OC(═O)OR^{k}, —OC(═O)NR^{h}Rⁱ, —[O(CH₂)_{q}]ᵣO(C₁-C₆ alkyl), —S(═O)ₚR^{k}, —S(═O)ₚNR^{h}Rⁱ, —C(═O)OR^{k}, C₃-C₁₂ carbocyclyl, 3- to 12-membered heterocyclyl, C₆ and C₁₀ aryl, and 5- to 10-membered heteroaryl groups, wherein:
         the C₁-C₆ alkyl, C₁-C₆ alkoxy, and the C₂-C₆ alkenyl of R^{a} are each optionally substituted with 1 to 3 groups independently chosen from C₆ to C₁₀ aryl (optionally substituted with 1 to 3 R^{m} groups), 5- to 10-membered heterocyclyl (optionally substituted with 1 to 3 R^{m} groups), 5-to 10-membered heteroaryl (optionally substituted with 1 to 3 R^{m} groups), cyano, —C(═O)R^{k}, —C(═O)OR^{k}, —C(═O)NR^{h}Rⁱ, —NR^{h}Rⁱ, —NR^{h}C(═O)R^{k},
      —NR^{h}C(═O)OR^{k}, —NR^{h}C(═O)NRⁱR^{j}, —NR^{h}S(═O)ₚR^{k}, —OR^{k}, —OC(═O)R^{k}, —OC(═O)OR^{k}, —OC(═O)NR^{h}Rⁱ, —S(═O)ₚR^{k}, —S(═O)ₚNR^{h}Rⁱ, and C₃-C₆ carbocyclyl groups (optionally substituted with 1 to 3 R^{m} groups);
         the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from halogen, cyano, C₁-C₄ alkyl, —NR^{h}Rⁱ, and —OR^{k} groups, wherein:
         R^{h}, Rⁱ, and R^{j}, for each occurrence, are each independently chosen from hydrogen, C₁-C₄ alkyl, C₆-C₁₀ aryl, and C₃-C₆ cycloalkyl groups, wherein:
         the C₁-C₄ alkyl of any one of R^{h}, Rⁱ, and R^{j} is optionally substituted with 1 to 3 groups independently chosen from halogen, cyano, and —OH groups;
         R^{k}, for each occurrence, is independently chosen from hydrogen, C₁-C₄ alkyl, 5- to 10-membered heterocyclyl, and C₃-C₆ carbocyclyl groups, wherein:
         the C₁-C₄ alkyl of any one of R^{k} is optionally substituted with 1 to 3 groups independently chosen from halogen, cyano, and —OH groups;
         R^{m}, for each occurrence, is independently chosen from halogen, cyano, oxo, C₁-C₆ alkyl, C₁-C₆ alkoxy, —S(═O)ₚR^{k}, and —OR^{k} groups, wherein:
         the C₁-C₆ alkyl of R^{m} is optionally substituted with 1 to 3 groups independently chosen from halogen, cyano, and —OH groups;
R³ is chosen from C₁-C₆ alkyl groups, wherein:
   the C₁-C₆ alkyl of R³ is optionally substituted with 1 to 3 groups independently chosen from halogen, cyano, —OH, —NH₂, —NH(C₁-C₄ alkyl), —N(C₁-C₄ alkyl)₂, C₁-C₄ alkoxy, —C(═O)NH₂, —C(═O)NH(C₁-C₄ alkyl), and —C(═O)N(C₁-C₄ alkyl)₂ groups;
m is an integer chosen from 0, 1, 2, and 3;
p, for each occurrence, is an integer independently chosen from 1 and 2; and
q and r, for each occurrence, are each an integer independently chosen from 1, 2, 3, and 4.

In some embodiments, in a compound, tautomer, deuterated derivative, or pharmaceutically acceptable salt of the disclosure, Ring A is phenyl;
X is chosen from —CH₂—, —C(O)—, —S(O)₂—, —NH—, and —O—;
Y is chosen from —CH₂—, —C(O)—, —S(O)₂—, —NH—, and —O—;
Z is chosen from a bond, —CH₂—, —NH—, —C(O)—, —S(O)₂—, and —O—, wherein:
   at least one of X and Y is chosen from —CH₂— and —C(O)—; and
   for each of X, Y, and Z, a hydrogen atom in each instance of —CH₂— or —NH— is optionally replaced by R¹;
R¹, for each occurrence, is independently chosen from halogen, —OH, cyano, C₁-C₄ alkyl, C₁-C₄ alkoxy, —C(═O)OR^{c}, —C(═O)N(R^{c})₂, and —OS(═O)₂R^{c} groups, wherein:
   R^{c}, for each occurrence, is independently chosen from hydrogen, C₁-C₄ alkyl, and C₁-C₄ haloalkyl groups;
   the C₁-C₆ alkyl of R¹ is optionally substituted with 1 to 3 groups independently chosen from halogen and —OH groups;
   R² is

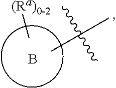

wherein:
Ring B is chosen from 5-membered heterocyclyl and 5-membered heteroaryl groups, wherein Ring B is optionally substituted with 1 or 2 R^{a} groups, wherein:
   R^{a}, for each occurrence, is independently chosen from C₁-C₆ alkyl groups optionally substituted with 1 group independently chosen from —S(═O)ₚR^{k} groups, wherein:
      R^{k}, for each occurrence, is independently chosen from C₁-C₄ alkyl groups;
R³ is chosen from C₁-C₃ alkyl groups;
m is an integer chosen from 0, 1, 2, and 3; and
p, for each occurrence, is an integer independently chosen from 1 and 2.

In some embodiments, in a compound, tautomer, deuterated derivative, or pharmaceutically acceptable salt of the disclosure, Ring A is phenyl;
X is chosen from —CH₂—, —C(O)—, —S(O)₂—, —NH—, and —O—;
Y is chosen from —CH₂—, —C(O)—, —S(O)₂—, —NH—, and —O—;
Z is chosen from a bond, —CH₂—, —NH—, —C(O)—, —S(O)₂—, and —O—, wherein:
   at least one of X and Y is chosen from —CH₂— and —C(O)—; and
   for each of X, Y, and Z, a hydrogen atom in each instance of —CH₂— or —NH— is optionally replaced by R¹;
R¹, for each occurrence, is independently chosen from halogen, —OH, cyano, C₁-C₄ alkyl, C₁-C₄ alkoxy, —C(═O)OR^{c}, —C(═O)N(R^{c})₂, and —OS(═O)₂R^{c} groups, wherein:
   R^{c}, for each occurrence, is independently chosen from hydrogen, C₁-C₄ alkyl, and C₁-C₄ haloalkyl groups;
   the C₁-C₆ alkyl of R¹ is optionally substituted with 1 to 3 groups independently chosen from halogen and —OH groups;
   R² is

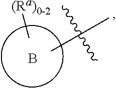

wherein:
Ring B is chosen from pyrazole and triazole groups, wherein Ring B is optionally substituted with 1 or 2 R^{a} groups, wherein:
   R^{a}, for each occurrence, is independently chosen from C₁-C₆ alkyl groups optionally substituted with 1 group independently chosen from —S(═O)ₚR^{k} groups, wherein:
      R^{k}, for each occurrence, is independently chosen from C₁-C₄ alkyl groups;
R³ is methyl;
m is an integer chosen from 0, 1, 2, and 3; and
p, for each occurrence, is an integer independently chosen from 1 and 2.

In one aspect of the disclosure, the compounds of Formula I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC are chosen from Compounds 1 to 42 and Compounds I1 to I36, such that the at least one compound, pharmaceutically acceptable salt, solvate, or deuterated derivative is chosen from Compounds 1 to 42 and Compounds I1 to I36, pharmaceutically acceptable salts of any of those compounds, solvates of any of the foregoing, and deuterated derivatives of any of the foregoing.

In some embodiments, the disclosure provides a pharmaceutical composition comprising at least one compound, tautomer, deuterated derivative, or pharmaceutically acceptable salt chosen from compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, tautomers thereof, deuterated derivatives of those compounds or tautomers, and pharmaceutically acceptable salts of any of the foregoing. In some embodiments, the pharmaceutical composition may comprise at least one compound chosen from Compounds 1 to 42 and Compounds I1 to I36, pharmaceutically acceptable salts of any of those compounds, solvates of any of the foregoing, and deuterated derivatives of any of the foregoing. These compositions may further include at least one additional active pharmaceutical ingredient and/or at least one carrier.

Another aspect of the disclosure provides methods of treating an APOL1-mediated disease comprising administering to a subject in need thereof, at least one compound, tautomer, deuterated derivative, or pharmaceutically acceptable salt chosen from compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods comprise administering at least one compound, tautomer, deuterated derivative, or pharmaceutically acceptable salt chosen from Compounds 1 to 42 and Compounds I1 to 136, tautomers thereof, deuterated derivatives of those compounds or tautomers, and pharmaceutically acceptable salts of any of the foregoing.

Another aspect of the disclosure provides methods of treating an APOL1-mediated cancer (such as, e.g., pancreatic cancer) comprising administering to a subject in need thereof, at least one compound, tautomer, deuterated derivative, or pharmaceutically acceptable salt chosen from compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods comprise administering at least one compound, tautomer, deuterated derivative, or pharmaceutically acceptable salt chosen from Compounds 1 to 42 and Compounds I1 to I36, tautomers thereof, deuterated derivatives of those compounds or tautomers, and pharmaceutically acceptable salts of any of the foregoing.

Another aspect of the disclosure provides methods of treating APOL1-mediated kidney disease (such as, e.g., ESKD, FSGS and/or NDKD) comprising administering to a subject in need thereof, at least one compound, tautomer, deuterated derivative, or pharmaceutically acceptable salt chosen from compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods comprise administering at least one compound, tautomer, deuterated derivative, or pharmaceutically acceptable salt chosen from Compounds 1 to 42 and Compounds I1 to I36, tautomers thereof, deuterated derivatives of those compounds or tautomers, and pharmaceutically acceptable salts of any of the foregoing.

In some embodiments, the methods of treatment include administration of at least one additional active agent to the subject in need thereof, either in the same pharmaceutical composition as the at least one compound, tautomer, deuterated derivative, or pharmaceutically acceptable salt chosen from compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, tautomers thereof, deuterated derivatives of those compounds or tautomers, and pharmaceutically acceptable salts of any of the foregoing, or as separate compositions. In some embodiments, the methods comprise administering at least one compound, tautomer, deuterated derivative, or pharmaceutically acceptable salt chosen from Compounds 1 to 42 and Compounds I1 to I36, tautomers thereof, deuterated derivatives of those compounds or tautomers, and pharmaceutically acceptable salts of any of the foregoing with at least one additional active agent, either in the same pharmaceutical composition or in a separate composition.

Also provided are methods of inhibiting APOL1, comprising administering to a subject in need thereof, at least one compound, tautomer, deuterated derivative, or pharmaceutically acceptable salt chosen from compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods of inhibiting APOL1 comprise administering at least one compound, tautomer, deuterated derivative, or pharmaceutically acceptable salt chosen from Compounds 1 to 42 and Compounds I1 to I36, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt.

### DETAILED DESCRIPTION

### Definitions

The term “APOL1,” as used herein, means apolipoprotein L1 protein and the term “APOL1” means apolipoprotein L1 gene.

The term “APOL1 mediated disease” refers to a disease or condition associated with aberrant APOL1 (e.g., certain APOL1 genetic variants; elevated levels of APOL1). In some embodiments, an APOL1 mediated disease is an APOL1 mediated kidney disease. In some embodiments, an APOL1 mediated disease is associated with patients having two APOL1 risk alleles, e.g., patients who are homozygous or compound heterozygous for the G1 or G2 alleles. In some embodiments, an APOL1 mediated disease is associated with patients having one APOL1 risk allele.

The term “APOL1 mediated kidney disease” refers to a disease or condition that impairs kidney function and can be attributed to APOL1. In some embodiments, APOL1 mediated kidney disease is associated with patients having two APOL1 risk alleles, e.g., patients who are homozygous or compound heterozygous for the G1 or G2 alleles. In some embodiments, the APOL1 mediated kidney disease is chosen from ESKD, NDKD, FSGS, HIV-associated nephropathy, arterionephrosclerosis, lupus nephritis, microalbuminuria, and chronic kidney disease. In some embodiments, the APOL1 mediated kidney disease is chronic kidney disease or proteinuria.

The term “FSGS,” as used herein, means focal segmental glomerulosclerosis, which is a disease of the podocyte (glomerular visceral epithelial cells) responsible for proteinuria and progressive decline in kidney function, and associated with 2 common APOL1 genetic variants (GI: S342G:I384M and G2: N388del:Y389del).

The term “NDKD,” as used herein, means non-diabetic kidney disease, which is characterized by severe hypertension and progressive decline in kidney function, and associated with 2 common APOL1 genetic variants (GI: S342G:I384M and G2: N388del:Y389del).

The terms “ESKD” and “ESRD” are used interchangeably herein to refer to end stage kidney disease or end stage renal disease. ESKD/ESRD is the last stage of kidney disease, i.e., kidney failure, and means that the kidneys have stopped working well enough for the patient to survive without dialysis or a kidney transplant. In some embodiments, ESKD/ESRD is associated with two APOL1 risk alleles.

The term “compound,” when referring to a compound of this disclosure, refers to a collection of molecules having an identical chemical structure unless otherwise indicated as a collection of stereoisomers (for example, a collection of racemates, a collection of cis/trans stereoisomers, or a collection of (E) and (Z) stereoisomers), except that there may be isotopic variation among the constituent atoms of the molecules. Thus, it will be clear to those of skill in the art that a compound represented by a particular chemical structure containing indicated deuterium atoms will also contain lesser amounts of isotopologues having hydrogen atoms at one or more of the designated deuterium positions in that structure. The relative amount of such isotopologues in a compound of this disclosure will depend upon a number of factors including the isotopic purity of reagents used to make the compound and the efficiency of incorporation of isotopes in the various synthesis steps used to prepare the compound. However, as set forth above, the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

As used herein, “optionally substituted” is interchangeable with the phrase “substituted or unsubstituted.” In general, the term “substituted,” whether preceded by the term “optionally” or not, refers to the replacement of hydrogen radicals in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by this disclosure are those that result in the formation of stable or chemically feasible compounds.

The term “isotopologue” refers to a species in which the chemical structure differs from a reference compound only in the isotopic composition thereof. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C or ¹⁴C, are within the scope of this disclosure.

Unless otherwise indicated, structures depicted herein are also meant to include all isomeric forms of the structures, e.g., racemic mixtures, cis/trans isomers, geometric (or conformational) isomers, such as (Z) and (E) double bond isomers, and (Z) and (E) conformational isomers. Therefore, geometric and conformational mixtures of the present compounds are within the scope of the disclosure. Unless otherwise stated, all tautomeric forms of the compounds of the disclosure are within the scope of the disclosure.

The term “tautomer,” as used herein, refers to one of two or more isomers of compound that exist together in equilibrium, and are readily interchanged by migration of an atom, e.g., a hydrogen atom, or group within the molecule.

“Stereoisomer,” as used herein, refers to enantiomers and diastereomers.

As used herein, “deuterated derivative” refers to a compound having the same chemical structure as a reference compound, but with one or more hydrogen atoms replaced by a deuterium atom (“D” or “²H”). It will be recognized that some variation of natural isotopic abundance occurs in a synthesized compound depending on the origin of chemical materials used in the synthesis. The concentration of naturally abundant stable hydrogen isotopes, notwithstanding this variation, is small and immaterial as compared to the degree of stable isotopic substitution of deuterated derivatives described herein. Thus, unless otherwise stated, when a reference is made to a “deuterated derivative” of a compound of the disclosure, at least one hydrogen is replaced with deuterium at well above its natural isotopic abundance (which is typically about 0.015%). In some embodiments, the deuterated derivatives of the disclosure have an isotopic enrichment factor for each deuterium atom, of at least 3500 (52.5% deuterium incorporation at each designated deuterium), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), or at least 6600 (99% deuterium incorporation).

The term “isotopic enrichment factor,” as used herein, means the ratio between the isotopic abundance and the natural abundance of a specified isotope.

The term “alkyl” or “aliphatic,” as used herein, means a straight-chain (i.e., linear or unbranched) or branched, substituted or unsubstituted hydrocarbon chain that is completely saturated. Unless otherwise specified, alkyl groups contain 1 to 20 alkyl carbon atoms. In some embodiments, alkyl groups contain 1 to 10 aliphatic carbon atoms. In some embodiments, alkyl groups contain 1 to 8 aliphatic carbon atoms. In some embodiments, alkyl groups contain 1 to 6 alkyl carbon atoms. In some embodiments, alkyl groups contain 1 to 4 alkyl carbon atoms, in other embodiments, alkyl groups contain 1 to 3 alkyl carbon atoms, and in yet other embodiments, alkyl groups contain 1 or 2 alkyl carbon atoms. In some embodiments, alkyl groups are linear or straight-chain or unbranched. In some embodiments, alkyl groups are branched.

The terms “cycloalkyl” and “cyclic alkyl,” as used herein, refer to a monocyclic C₃₋₈ hydrocarbon or a spirocyclic, fused, or bridged bicyclic or tricyclic C₈₋₁₄ hydrocarbon that is completely saturated, wherein any individual ring in said bicyclic ring system has 3 to 7 members. In some embodiments, the cycloalkyl is a C₃ to C₁₂ cycloalkyl. In some embodiments, the cycloalkyl is a C₃ to C₈ cycloalkyl. In some embodiments, the cycloalkyl is a C₃ to C₆ cycloalkyl. Non-limiting examples of monocyclic cycloalkyls include cyclopropyl, cyclobutyl, cyclopentanyl, and cyclohexyl.

The terms “carbocyclyl” or “cycloaliphatic,” as used herein, encompass the terms “cycloalkyl” or “cyclic alkyl,” and refer to a monocyclic C₃₋₈ hydrocarbon or a spirocyclic, fused, or bridged bicyclic or tricyclic C₈-14 hydrocarbon that is completely saturated, or is partially saturated as in it contains one or more units of unsaturation but is not aromatic, wherein any individual ring in said bicyclic ring system has 3 to 7 members. Bicyclic carbocyclyls include combinations of a monocyclic carbocyclic ring fused to a phenyl. In some embodiments, the carbocyclyl is a C₃ to C₁₂ carbocyclyl. In some embodiments, the carbocyclyl is a C₃ to C₁₀ carbocyclyl. In some embodiments, the carbocyclyl is a C₃ to C₈ carbocyclyl.

The term “heteroalkyl,” or “heteroaliphatic,” as used herein, means an alkyl or aliphatic group as defined above, wherein one or two carbon atoms are independently replaced by one or more of oxygen, sulfur, nitrogen, phosphorus, or silicon.

The term “alkenyl,” as used herein, means a straight-chain (i.e., linear or unbranched) or branched hydrocarbon chain that contains one or more double bonds. In some embodiments, alkenyl groups are straight-chain. In some embodiments, alkenyl groups are branched.

The term “heterocycle,” “heterocyclyl,” “heterocycloaliphatic,” or “heterocyclic,” as used herein, means non-aromatic (i.e., completely saturated or partially saturated as in it contains one or more units of unsaturation but is not aromatic), monocyclic, or spirocyclic, fused, or bridged bicyclic or tricyclic ring systems in which one or more ring members is an independently chosen heteroatom. Bicyclic heterocyclyls include the following combinations of monocyclic rings: a monocyclic heteroaryl fused to a monocyclic heterocyclyl; a monocyclic heterocyclyl fused to another monocyclic heterocyclyl; a monocyclic heterocyclyl fused to phenyl; a monocyclic heterocyclyl fused to a monocyclic carbocyclyl/cycloalkyl; and a monocyclic heteroaryl fused to a monocyclic carbocyclyl/cycloalkyl.

In some embodiments, the heterocycle comprises a ring atom substituted with one or more oxo groups (such as, e.g., a C═O group, a S═O group, or a SO₂ group).

In some embodiments, the “heterocycle,” “heterocyclyl,” “heterocycloaliphatic,” or “heterocyclic” group has 3 to 14 ring members in which one or more ring members is a heteroatom independently chosen from oxygen, sulfur, nitrogen, and phosphorus. In some embodiments, each ring in a bicyclic or tricyclic ring system contains 3 to 7 ring members. In some embodiments, the heterocycle has at least one unsaturated carbon-carbon bond. In some embodiments, the heterocycle has at least one unsaturated carbon-nitrogen bond. In some embodiments, the heterocycle has one heteroatom independently chosen from oxygen, sulfur, nitrogen, and phosphorus. In some embodiments, the heterocycle has one heteroatom that is a nitrogen atom. In some embodiments, the heterocycle has one heteroatom that is an oxygen atom. In some embodiments, the heterocycle has two heteroatoms that are each independently chosen from nitrogen and oxygen. In some embodiments, the heterocycle has three heteroatoms that are each independently chosen from nitrogen and oxygen. In some embodiments, the heterocyclyl is a 3- to 12-membered heterocyclyl. In some embodiments, the heterocyclyl is a 3-to 10-membered heterocyclyl. In some embodiments, the heterocyclyl is a 3- to 8-membered heterocyclyl. In some embodiments, the heterocyclyl is a 5- to 10-membered heterocyclyl. In some embodiments, the heterocyclyl is a 5- to 8-membered heterocyclyl. In some embodiments, the heterocyclyl is a 5- or 6-membered heterocyclyl. Non-limiting examples of monocyclic heterocyclyls include piperidinyl, piperazinyl, tetrahydropyranyl, azetidinyl, tetrahydrothiophenyl 1,1-dioxide, etc.

The term “heteroatom” means one or more of oxygen, sulfur, nitrogen, phosphorus, or silicon (including, e.g., any oxidized form of nitrogen, sulfur, phosphorus, or silicon; the quaternized form of any basic nitrogen or; a substitutable nitrogen of a heterocyclic ring, for example, N (as in 3,4-dihydro-2H-pyrrolyl), NH (as in pyrrolidinyl) or NR (as in N-substituted pyrrolidinyl)).

The term “unsaturated,” as used herein, means that a moiety has one or more units or degrees of unsaturation. Unsaturation is the state in which not all of the available valence bonds in a compound are satisfied by substituents and thus the compound contains double or triple bonds.

The term “alkoxy” or “thioalkyl,” as used herein, refers to an alkyl group, as previously defined, wherein one carbon of the alkyl group is replaced by an oxygen (“alkoxy”) or sulfur (“thioalkyl”) atom, respectively, provided that the oxygen and sulfur atoms are linked between two carbon atoms. A “cyclic alkoxy” refers to a monocyclic, spirocyclic, bicyclic, bridged bicyclic, tricyclic, or bridged tricyclic hydrocarbon that contains at least one alkoxy group, but is not aromatic. Non-limiting examples of cyclic alkoxy groups include tetrahydropyranyl, tetrahydrofuranyl, oxetanyl, 8-oxabicyclo[3.2.1]octanyl, and oxepanyl.

The terms “haloalkyl,” “haloalkenyl,” and “haloalkoxy,” as used herein, mean a linear or branched alkyl, alkenyl, or alkoxy, respectively, which is substituted with one or more halogen atoms. Non-limiting examples of haloalkyl groups include —CHF₂, —CH₂F, —CF₃, —CF₂-, and perhaloalkyls, such as —CF₂CF₃. Non-limiting examples of haloalkoxy groups include —OCHF₂, —OCH₂F, —OCF₃, and —OCF₂.

The term “halogen” includes F, Cl, Br, and I, i.e., fluoro, chloro, bromo, and iodo, respectively.

The term “aminoalkyl” means an alkyl group which is substituted with or contains an amino group.

As used herein, an “amino” refers to a group which is a primary, secondary, or tertiary amine.

As used herein, a “carbonyl” group refers to C═O.

As used herein, a “cyano” or “nitrile” group refer to —C≡N.

As used herein, a “hydroxy” group refers to —OH.

As used herein, a “thiol” group refers to —SH.

As used herein, “tert” and “t-” each refer to tertiary.

As used herein, “aromatic groups” or “aromatic rings” refer to chemical groups that contain conjugated, planar ring systems with delocalized pi electron orbitals comprised of [4n+2] p orbital electrons, wherein n is an integer ranging from 0 to 6. Non-limiting examples of aromatic groups include aryl and heteroaryl groups.

The term “aryl,” used alone or as part of a larger moiety as in “arylalkyl,” “arylalkoxy,” or “aryloxyalkyl,” refers to monocyclic or spirocyclic, fused, or bridged bicyclic or tricyclic ring systems having a total of five to fourteen ring members, wherein every ring in the system is an aromatic ring containing only carbon atoms and wherein each ring in a bicyclic or tricyclic ring system contains 3 to 7 ring members. Non-limiting examples of aryl groups include phenyl (C₆) and naphthyl (C₁₀) rings.

The term “heteroaryl,” used alone or as part of a larger moiety as in “heteroarylalkyl” or “heteroarylalkoxy,” refers to monocyclic or spirocyclic, fused, or bridged bicyclic or tricyclic ring systems having a total of five to fourteen ring members, wherein at least one ring in the system is aromatic, wherein at least one ring in the system contains one or more heteroatoms, and wherein each ring in a bicyclic or tricyclic ring system contains 3 to 7 ring members. Bicyclic heteroaryls include the following combinations of monocyclic rings: a monocyclic heteroaryl fused to another monocyclic heteroaryl; and a monocyclic heteroaryl fused to a phenyl. In some embodiments, heteroaryl groups have one or more heteroatoms chosen from nitrogen, oxygen, and sulfur. In some embodiments, heteroaryl groups have one heteroatom. In some embodiments, heteroaryl groups have two heteroatoms. In some embodiments, heteroaryl groups are monocyclic ring systems having five ring members. In some embodiments, heteroaryl groups are monocyclic ring systems having six ring members. In some embodiments, the heteroaryl is a 3- to 12-membered heteroaryl. In some embodiments, the heteroaryl is a 3- to 10-membered heteroaryl. In some embodiments, the heteroaryl is a 3- to 8-membered heteroaryl. In some embodiments, the heteroaryl is a 5- to 10-membered heteroaryl. In some embodiments, the heteroaryl is a 5- to 8-membered heteroaryl. In some embodiments, the heteroaryl is a 5- or 6-membered heteroaryl. Non-limiting examples of monocyclic heteroaryls are pyridinyl, pyrimidinyl, thiophenyl, thiazolyl, isoxazolyl, etc.

In some embodiments, the heteroaryl comprises a ring atom substituted with one or more oxo groups (such as, e.g., a C═O group, a S═O group, or a SO₂ group). Illustratively, a non-limiting example of a heteroaryl group is a benzo[d]oxazol-2(3H)-one group.

Non-limiting examples of useful protecting groups for nitrogen-containing groups, such as amine groups, include, for example, t-butyl carbamate (Boc), benzyl (Bn), tetrahydropyranyl (THP), 9-fluorenylmethyl carbamate (Fmoc) benzyl carbamate (Cbz), acetamide, trifluoroacetamide, triphenylmethylamine, benzylideneamine, and p-toluenesulfonamide. Methods of adding (a process generally referred to as “protecting”) and removing (process generally referred to as “deprotecting”) such amine protecting groups are well-known in the art and available, for example, in P. J. Kocienski, Protecting Groups, Thieme, 1994, which is hereby incorporated by reference in its entirety and in Greene and Wuts, Protective Groups in Organic Synthesis, 3rd Edition (John Wiley & Sons, New York, 1999) and 4^{th} Edition (John Wiley & Sons, New Jersey, 2014).

Non-limiting examples of suitable solvents that may be used in this disclosure include, but are not limited to, water, methanol (MeOH), ethanol (EtOH), dichloromethane or “methylene chloride” (CH₂Cl₂), toluene, acetonitrile (MeCN), dimethylformamide (DMF), dimethyl sulfoxide (DMSO), methyl acetate (MeOAc), ethyl acetate (EtOAc), heptane, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in this disclosure include, but are not limited to, 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

The disclosure includes pharmaceutically acceptable salts of the disclosed compounds. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response, and the like, and are commensurate with a reasonable benefit/risk ratio. A “pharmaceutically acceptable salt” means any non-toxic salt that, upon administration to a recipient, is capable of providing, either directly or indirectly, a compound of this disclosure. Suitable pharmaceutically acceptable salts are, for example, those disclosed in S. M. Berge, et al. J. Pharmaceutical Sciences, 1977, 66, 1 to 19.

Acids commonly employed to form pharmaceutically acceptable salts include inorganic acids such as hydrogen bisulfide, hydrochloric acid, hydrobromic acid, hydroiodic acid, sulfuric acid, and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, O-hydroxybutyrate, glycolate, maleate, tartrate, methanesulfonate, propanesulfonate, naphthalene-1-sulfonate, naphthalene-2-sulfonate, mandelate, and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄ alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate, and aryl sulfonate. Other suitable, non-limiting examples of pharmaceutically acceptable salts include besylate and glucosamine salts.

The terms “patient” and “subject” are used interchangeably herein and refer to an animal, including a human.

The terms “effective dose” and “effective amount” are used interchangeably herein and refer to that amount of compound that produces a desired effect for which it is administered (e.g., improvement in a symptom of FSGS and/or NDKD, lessening the severity of FSGS and/NDKD or a symptom of FSGS and/or NDKD, and/or reducing progression of FSGS and/or NDKD or a symptom of FSGS and/or NDKD). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the term “treatment” and its cognates refer to slowing or stopping disease progression. “Treatment” and its cognates as used herein, include, but are not limited to, the following: complete or partial remission, lower risk of kidney failure (e.g., ESRD), and disease-related complications (e.g., edema, susceptibility to infections, or thrombo-embolic events). Improvements in or lessening the severity of any of these symptoms can be readily assessed according to methods and techniques known in the art or subsequently developed.

The terms “about” and “approximately,” when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent.

The at least one compound, tautomer, deuterated derivative, or pharmaceutically acceptable salt chosen from compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, a tautomer thereof, a deuterated derivative of that compound or tautomer, or a pharmaceutically acceptable salt of any of the foregoing may be administered once daily, twice daily, or three times daily, for example, for the treatment of FSGS. In some embodiments, the compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC are chosen from Compounds 1 to 42 and Compounds I1 to I36, a tautomer thereof, a deuterated derivative of that compound or tautomer, or a pharmaceutically acceptable salt of any of the foregoing. In some embodiments, at least one compound, tautomer, deuterated derivative, or pharmaceutically acceptable salt chosen from compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, a tautomer thereof, a deuterated derivative of that compound or tautomer, or a pharmaceutically acceptable salt of any of the foregoing is administered once daily. In some embodiments, at least one compound, tautomer, deuterated derivative, or pharmaceutically acceptable salt chosen from Compounds 1 to 42 and Compounds I1 to I36, a tautomer thereof, a deuterated derivative of that compound or tautomer, or a pharmaceutically acceptable salt of any of the foregoing is administered once daily. In some embodiments, at least one compound, tautomer, deuterated derivative, or pharmaceutically acceptable salt chosen from compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, a tautomer thereof, a deuterated derivative of that compound or tautomer, or a pharmaceutically acceptable salt of any of the foregoing is administered twice daily. In some embodiments, at least one compound, tautomer, deuterated derivative, or pharmaceutically acceptable salt chosen from Compounds 1 to 42 and Compounds I1 to I36, a tautomer thereof, a deuterated derivative of that compound or tautomer, or a pharmaceutically acceptable salt of any of the foregoing is administered twice daily. In some embodiments, at least one compound, tautomer, deuterated derivative, or pharmaceutically acceptable salt chosen from compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, a tautomer thereof, a deuterated derivative of that compound or tautomer, or a pharmaceutically acceptable salt of any of the foregoing are administered three times daily. In some embodiments, at least one compound, tautomer, deuterated derivative, or pharmaceutically acceptable salt chosen from Compounds 1 to 42 and Compounds I1 to I36, a tautomer thereof, a deuterated derivative of that compound or tautomer, or a pharmaceutically acceptable salt of any of the foregoing is administered three times daily.

In some embodiments, 2 mg to 1500 mg or 5 mg to 1000 mg of at least one compound, tautomer, deuterated derivative, or pharmaceutically acceptable salt chosen from compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, a tautomer thereof, a deuterated derivative of that compound or tautomer, or a pharmaceutically acceptable salt of any of the foregoing are administered once daily, twice daily, or three times daily. In some embodiments, 2 mg to 1500 mg or 5 mg to 1000 mg of at least one compound, tautomer, deuterated derivative, or pharmaceutically acceptable salt chosen from Compounds 1 to 42 and Compounds I1 to I36, a tautomer thereof, a deuterated derivative of that compound or tautomer, or a pharmaceutically acceptable salt of any of the foregoing is administered once daily, twice daily, or three times daily.

One of ordinary skill in the art would recognize that, when an amount of compound is disclosed, the relevant amount of a pharmaceutically acceptable salt form of the compound is an amount equivalent to the concentration of the free base of the compound. The amounts of the compounds, pharmaceutically acceptable salts, solvates, and deuterated derivatives disclosed herein are based upon the free base form of the reference compound. For example, “1000 mg of at least one compound or pharmaceutically acceptable salt chosen from compounds of Formula I and pharmaceutically acceptable salts thereof” includes 1000 mg of a compound of Formula I and a concentration of a pharmaceutically acceptable salt of compounds of Formula I equivalent to 1000 mg of a compound of Formula I.

As used herein, the term “ambient conditions” means room temperature, open air condition, and uncontrolled humidity condition.

### Compounds and Compositions

In some embodiments, at least one compound, tautomer, deuterated derivative, or pharmaceutically acceptable salt of the disclosure is a compound represented by the following structural formula:

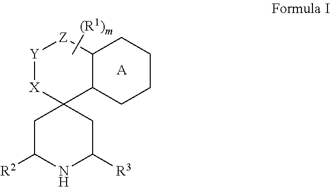

a tautomer thereof, a deuterated derivative of that compound or tautomer, or a pharmaceutically acceptable salt of any of the foregoing, wherein:
   Ring A is chosen from 6-membered aryl and 6-membered heteroaryl groups;
   X is chosen from —CH₂—, —C(O)—, —S(O)₂—, —NH—, and —O—;
   Y is chosen from —CH₂—, —C(O)—, —S(O)₂—, —NH—, and —O—;
   Z is chosen from a bond, —CH₂—, —NH—, —C(O)—, —S(O)₂—, and —O—, wherein:
      at least one of X and Y is chosen from —CH₂— and —C(O)—; and
      for each of X, Y, and Z, a hydrogen atom in each instance of —CH₂— or —NH— is optionally replaced by R¹;
   R¹, for each occurrence, is independently chosen from halogen, —OH, cyano, phenyl, C₁-C₆ alkyl, C₁-C₆ alkoxy, C₃-C₆ carbocyclyl, 4- to 6-membered heterocyclyl, —C(═O)OR^{c}, —C(═O)N(R^{c})₂, and —OS(═O)₂R^{c} groups, wherein:
      R^{c}, for each occurrence, is independently chosen from hydrogen, C₁-C₄ alkyl, and C₁-C₄ haloalkyl groups;
      the 4- to 6-membered heterocyclyl of R¹ comprises one heteroatom chosen from nitrogen and oxygen;
      the C₁-C₆ alkyl of R¹ is optionally substituted with 1 to 3 groups independently chosen from halogen, cyano, —OH, —NH₂, —NH(C₁-C₄ alkyl), —N(C₁-C₄ alkyl)₂, and C₁-C₄ alkoxy groups;
      the C₁-C₆ alkoxy of R¹ is optionally substituted with 1 to 3 groups independently chosen from —OH, cyano, and halogen groups;
      the C₃-C₆ carbocyclyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups; and
      the phenyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups;
   R² is chosen from cyano, C₁-C₆ alkyl, —C(═O)O(C₁-C₄ alkyl), C₂-C₆ alkynyl, and

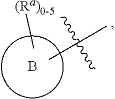

wherein:
   the C₁-C₆ alkyl of R² is optionally substituted with 1 to 3 groups independently chosen from halogen, cyano, —OH, —NH₂, —NH(C₁-C₄ alkyl), —N(C₁-C₄ alkyl)₂, C₁-C₄ alkoxy, —C(═O)NH₂, —C(═O)NH(C₁-C₄ alkyl), —C(═O)N(C₁-C₄ alkyl)₂, C₃-C₆ carbocyclyl, 5- to 10-membered heterocyclyl, C₆ aryl, and 5- to 10-membered heteroaryl groups;
   Ring B is chosen from C₃-C₁₂ carbocyclyl, 3- to 12-membered heterocyclyl, C₆ and C₁₀ aryl, and 5- to 10-membered heteroaryl groups, wherein Ring B is optionally substituted with 1, 2, 3, 4, or 5 R^{a} groups, wherein:
      R^{a}, for each occurrence, is independently chosen from halogen, cyano, C₁-C₆ alkyl, C₂-C₆ alkenyl, C₁-C₆ alkoxy, C₁-C₆ haloalkyl, C₁-C₆ haloalkenyl, C₁-C₆ haloalkoxy, —C(═O)NR^{h}Rⁱ, —NR^{h}i, —NR^{h}C(═O)R^{k}, —NR^{h}C(═O)OR^{k}, —NR^{h}C(═O)NRⁱR^{j}, —NR^{h}S(═O)ₚR^{k}, —OR^{k}, —OC(═O)R^{k}, —OC(═O)OR^{k}, —OC(═O)NR^{h}Rⁱ, —[O(CH₂)_{q}]ᵣO(C₁-C₆ alkyl), —S(═O)ₚR^{k}, —S(═O)ₚNR^{h}Rⁱ, —C(═O)OR^{k}, C₃-C₁₂ carbocyclyl, 3- to 12-membered heterocyclyl, C₆ and C₁₀ aryl, and 5- to 10-membered heteroaryl groups, wherein:
      the C₁-C₆ alkyl, C₁-C₆ alkoxy, and the C₂-C₆ alkenyl of R^{a} are each optionally substituted with 1 to 3 groups independently chosen from C₆ to C₁₀ aryl (optionally substituted with 1 to 3 R^{m} groups), 5- to 10-membered heterocyclyl (optionally substituted with 1 to 3 R^{m} groups), 5-to 10-membered heteroaryl (optionally substituted with 1 to 3 R^{m} groups), cyano, —C(═O)R^{k}, —C(═O)OR^{k}, —C(═O)NR^{h}Rⁱ, —NR^{h}Rⁱ, —NR^{h}C(═O)R^{k},
      —NR^{h}C(═O)OR^{k}, —NR^{h}C(═O)NRⁱR^{j}, —NR^{h}S(═O)ₚR^{k}, —OR^{k}, —OC(═O)R^{k}, —OC(═O)OR^{k}, —OC(═O)NR^{h}Rⁱ, —S(═O)ₚR^{k}, —S(═O)ₚNR^{h}Rⁱ, —O(C₆ aryl) (optionally substituted with 1 to 3 R^{m} groups), and C₃-C₆ carbocyclyl groups (optionally substituted with 1 to 3 R^{m} groups);
      the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from halogen, cyano, C₁-C₄ alkyl, —NR^{h}Rⁱ, and —OR^{k} groups, wherein:
      R^{h}, Rⁱ, and R^{j}, for each occurrence, are each independently chosen from hydrogen, C₁-C₄ alkyl, C₆-C₁₀ aryl, and C₃-C₆ cycloalkyl groups, wherein:
      the C₁-C₄ alkyl of any one of R^{h}, Rⁱ, and R^{j} is optionally substituted with 1 to 3 groups independently chosen from halogen, cyano, and —OH groups;
      R^{k}, for each occurrence, is independently chosen from hydrogen, C₁-C₄ alkyl, 5- to 10-membered heterocyclyl, and C₃-C₆ carbocyclyl groups, wherein:
      the C₁-C₄ alkyl of any one of R^{k} is optionally substituted with 1 to 3 groups independently chosen from halogen, cyano, and —OH groups;
      R^{m}, for each occurrence, is independently chosen from halogen, cyano, oxo, C₁-C₆ alkyl, C₁-C₆ alkoxy, —S(═O)ₚR^{k}, and —OR^{k} groups, wherein:
      the C₁-C₆ alkyl of R^{m} is optionally substituted with 1 to 3 groups independently chosen from halogen, cyano, —OH, and —O(C₁-C₄ alkyl) groups;
R³ is chosen from C₁-C₆ alkyl, —C(═O)O(C₁-C₄ alkyl), C₃-C₁₂ carbocyclyl, 3- to 12-membered heterocyclyl, C₆ and C₁₀ aryl, and 5- to 10-membered heteroaryl groups, wherein:
   the C₁-C₆ alkyl of R³ is optionally substituted with 1 to 3 groups independently chosen from halogen, cyano, —OH, —NH₂, —NH(C₁-C₄ alkyl), —N(C₁-C₄ alkyl)₂, C₁-C₄ alkoxy, —C(═O)NH₂, —C(═O)NH(C₁-C₄ alkyl), and —C(═O)N(C₁-C₄ alkyl)₂ groups;
   the C₃-C₁₂ carbocyclyl, the 3- to 12-membered heterocyclyl, the C₆ and C₁₀ aryl, and the 5- to 10-membered heteroaryl of R³ are each optionally substituted with 1 to 3 groups independently chosen from halogen, cyano, —OH, —NH₂, —NH(C₁-C₄ alkyl) (optionally substituted with —OH), —N(C₁-C₄ alkyl)₂, C₁-C₅ alkyl (optionally substituted with —OH or —S(═O)₂(C₁-C₄ alkyl)), C₁-C₄ alkoxy, —C(═O)NH₂, —C(═O)NH(C₁-C₄ alkyl), —NHC(═O)(C₁-C₄ alkyl), —C(═O)(C₁-C₄ alkoxy), and —C(═O)N(C₁-C₄ alkyl)₂ groups;
m is an integer chosen from 0, 1, 2, 3, 4, and 5;
p, for each occurrence, is an integer independently chosen from 1 and 2; and
q and r, for each occurrence, are each an integer independently chosen from 1, 2, 3, and 4.

In some embodiments, in a compound, tautomer, deuterated derivative, or pharmaceutically acceptable salt of the disclosure, m is an integer chosen from 0, 1, and 2; and all other variables not specifically defined herein are as defined in any one of the foregoing embodiments. In some embodiments, in a compound, tautomer, deuterated derivative, or pharmaceutically acceptable salt of the disclosure, m is 0; and all other variables not specifically defined herein are as defined in any one of the foregoing embodiments. In some embodiments, in a compound, tautomer, deuterated derivative, or pharmaceutically acceptable salt of the disclosure, m is 1; and all other variables not specifically defined herein are as defined in any one of the foregoing embodiments.

In some embodiments, in a compound, tautomer, deuterated derivative, or pharmaceutically acceptable salt of the disclosure, Ring A is phenyl, pyrimidinyl, or pyridinyl; and all other variables not specifically defined herein are as defined in any one of the foregoing embodiments. In some embodiments, in a compound, tautomer, deuterated derivative, or pharmaceutically acceptable salt of the disclosure, Ring A is phenyl; and all other variables not specifically defined herein are as defined in any one of the foregoing embodiments. In some embodiments, in a compound, tautomer, deuterated derivative, or pharmaceutically acceptable salt of the disclosure, Ring A is pyrimidinyl; and all other variables not specifically defined herein are as defined in any one of the foregoing embodiments. In some embodiments, in a compound, tautomer, deuterated derivative, or pharmaceutically acceptable salt of the disclosure, Ring A is pyridinyl; and all other variables not specifically defined herein are as defined in any one of the foregoing embodiments.

In some embodiments, in a compound, tautomer, deuterated derivative, or pharmaceutically acceptable salt of the disclosure, R² is chosen from C₁-C₄ alkyl and

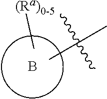

groups, wherein:
the C₁-C₄ alkyl of R² is optionally substituted with 1 to 3 groups independently chosen from halogen, cyano, —OH, —NH₂, —NH(C₁-C₄ alkyl), —N(C₁-C₄ alkyl)₂, C₁-C₂ alkoxy, C₃-C₆ cycloalkyl, 5- to 6-membered heterocyclyl, phenyl, and 5- to 6-membered heteroaryl groups; and all other variables not specifically defined herein are as defined in any one of the foregoing embodiments.

In some embodiments, in a compound, tautomer, deuterated derivative, or pharmaceutically acceptable salt of the disclosure, R² is chosen from C₁-C₂ alkyl and

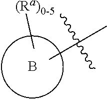

groups, wherein:
the C₁-C₂ alkyl of R² is optionally substituted with 1 to 3 groups independently chosen from halogen, cyano, —OH, and 5- to 6-membered heterocyclyl groups; and all other variables not specifically defined herein are as defined in any one of the foregoing embodiments.

In some embodiments, in a compound, tautomer, deuterated derivative, or pharmaceutically acceptable salt of the disclosure, R² is chosen from R² is chosen from —CH₃ and

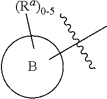

groups; and all other variables not specifically defined herein are as defined in any one of the foregoing embodiments.

In some embodiments, in a compound, tautomer, deuterated derivative, or pharmaceutically acceptable salt of the disclosure, R² is chosen from —CH₃, —CH₂OH, and (tetrahydro-2H-pyran-4-yl)methyl; and all other variables not specifically defined herein are as defined in any one of the foregoing embodiments.

In some embodiments, in a compound, tautomer, deuterated derivative, or pharmaceutically acceptable salt of the disclosure, Ring B is chosen from cyclopropyl, 5- to 10-membered heterocyclyl, phenyl, and 5 to 9-membered heteroaryl groups, each of which is optionally substituted with 1, 2, 3, 4, or 5 R^{a} groups; and all other variables not specifically defined herein are as defined in any one of the foregoing embodiments.

In some embodiments, in a compound, tautomer, deuterated derivative, or pharmaceutically acceptable salt of the disclosure, Ring B is chosen from cyclopropyl, 5- to 10-membered heterocyclyl comprising 1 to 3 heteroatoms chosen from N and O, phenyl, and 5- to 9-membered heteroaryl comprising 1 to 3 heteroatoms chosen from N and O; each of which is optionally substituted with 1, 2, 3, 4, or 5 R^{a} groups; and all other variables not specifically defined herein are as defined in any one of the foregoing embodiments.

In some embodiments, in a compound, tautomer, deuterated derivative, or pharmaceutically acceptable salt of the disclosure, Ring B is chosen from cyclopropyl, 5-membered heterocyclyl comprising 1 to 3 heteroatoms chosen from N and O, 6-membered heterocyclyl comprising 1 to 3 heteroatoms chosen from N and O, 9-membered heterocyclyl comprising 1 to 3 heteroatoms chosen from N and O, 10-membered heterocyclyl comprising 1 to 3 heteroatoms chosen from N and O, phenyl, 5-membered heteroaryl comprising 1 to 3 heteroatoms chosen from N and O, 6-membered heteroaryl comprising 1 to 3 heteroatoms chosen from N and O, and 9-membered heteroaryl comprising 1 to 3 heteroatoms chosen from N and O; each of which is optionally substituted with 1, 2, 3, 4, or 5 R^{a} groups; and all other variables not specifically defined herein are as defined in any one of the foregoing embodiments.

In some embodiments, in a compound, tautomer, deuterated derivative, or pharmaceutically acceptable salt of the disclosure, Ring B is chosen from

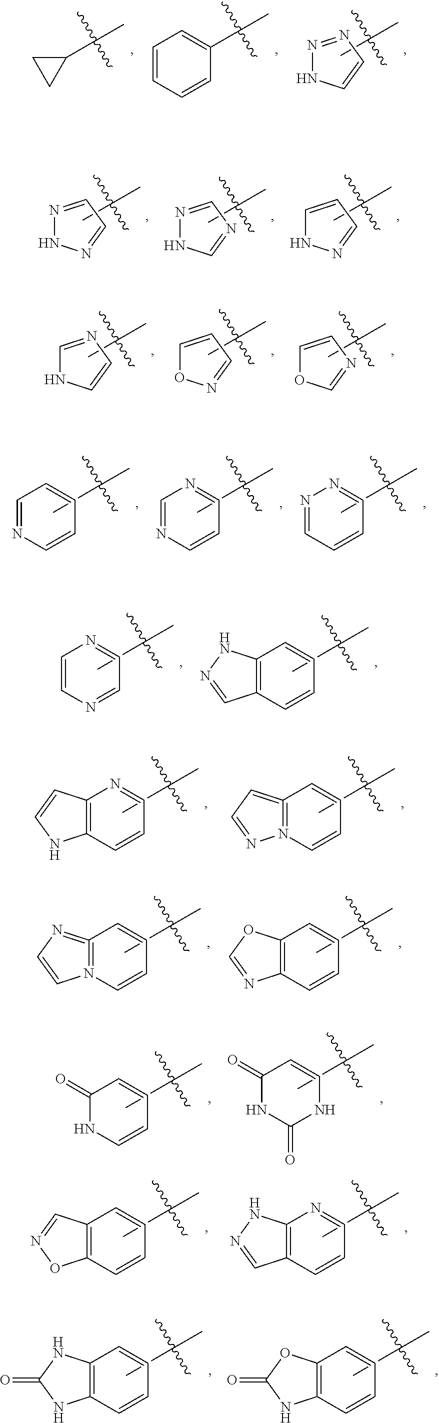

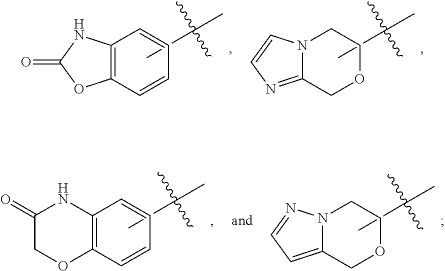

each of which is optionally substituted with 1, 2, 3, 4, or 5 R^{a} groups; and all other variables not specifically defined herein are as defined in any one of the foregoing embodiments.

In some embodiments, in a compound, tautomer, deuterated derivative, or pharmaceutically acceptable salt of the disclosure, Ring B is chosen from

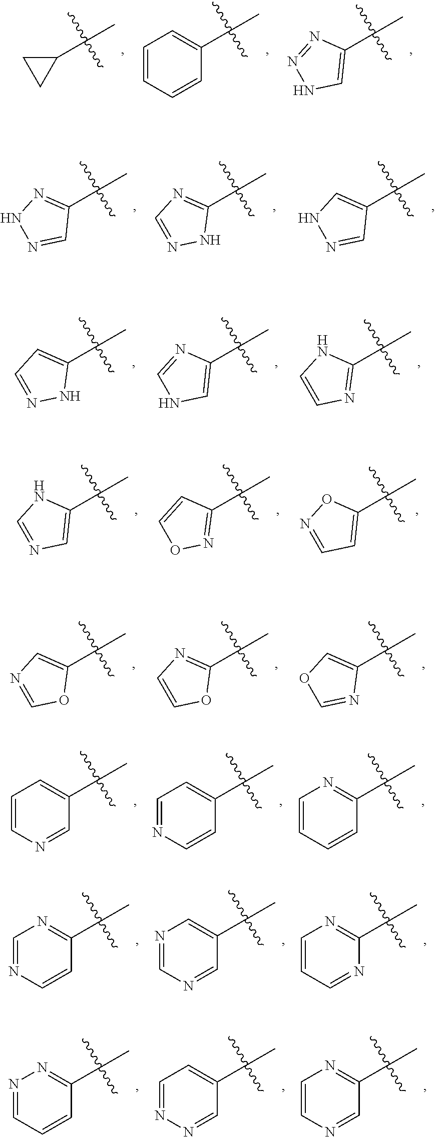

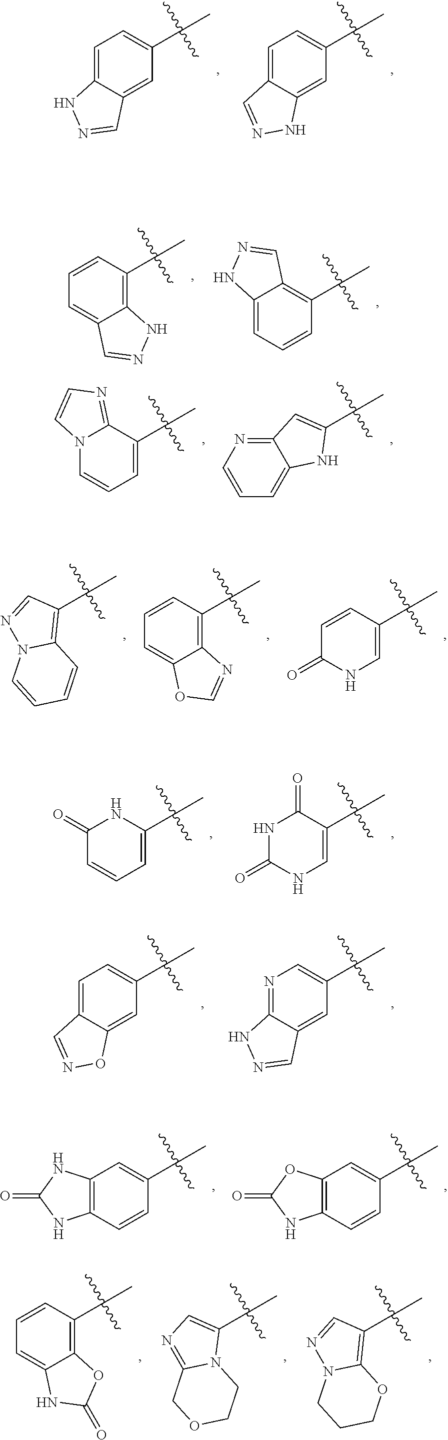

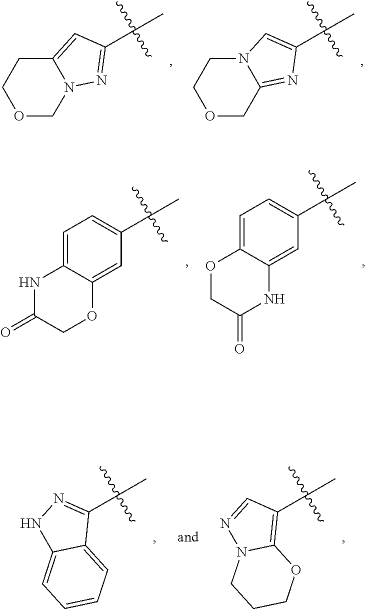 [SMILES: *c1cc2n(n1)COCC2 ; *c1cn2c(n1)COCC2 ; *c1ccc2c(c1)OCC(=O)N2 ; *c1ccc2c(c1)NC(=O)CO2 ; *c1n[nH]c2ccccc12 ; *c1cnn2c1OCCC2]

each of which is optionally substituted with 1, 2, 3, 4, or 5 R^{a} groups; and all other variables not specifically defined herein are as defined in any one of the foregoing embodiments.

In some embodiments, in a compound, tautomer, deuterated derivative, or pharmaceutically acceptable salt of the disclosure, R² is chosen from —CH₃ and Ring B, wherein Ring B is chosen from

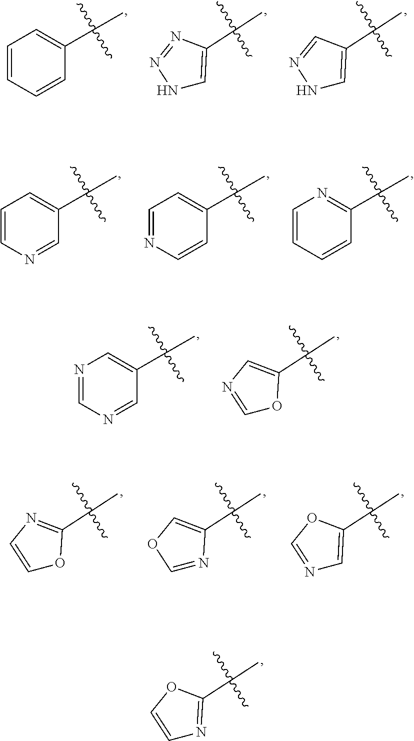

and each of which is optionally substituted with 1, 2, 3, 4, or 5 R^{a} groups; and all other variables not specifically defined herein are as defined in any one of the foregoing embodiments. In some embodiments, Ring B is

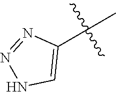 [SMILES: *c1c[nH]nn1]

which is optionally substituted with 1 R^{a} group.

In some embodiments, in a compound, tautomer, deuterated derivative, or pharmaceutically acceptable salt of the disclosure, R³ is chosen from C₁-C₄ alkyl, —C(═O)O(C₁-C₂ alkyl), C₃-C₆ cycloalkyl, and 5 to 10-membered heterocyclyl groups, wherein:
the C₁-C₄ alkyl of R³ is optionally substituted with 1 to 3 groups independently chosen from halogen, cyano, —OH, and C₁-C₂ alkoxy groups; and
the C₃-C₆ cycloalkyl and the 5- to 10-membered heterocyclyl of R³ are each optionally substituted with 1 to 3 groups independently chosen from halogen, cyano, —OH, C₁-C₂ alkyl, and C₁-C₂ alkoxy groups;
   and all other variables not specifically defined herein are as defined in any one of the foregoing embodiments.

In some embodiments, in a compound, tautomer, deuterated derivative, or pharmaceutically acceptable salt of the disclosure, R³ is chosen from C₁-C₂ alkyl, —C(═O)O(C₁-C₂ alkyl), cyclopropyl, cyclobutyl, and 5- to 6-membered heterocyclyl groups, wherein:
the C₁-C₂ alkyl of R³ is optionally substituted with 1 to 3 groups independently chosen from F, Cl, Br, cyano, —OH, and C₁-C₂ alkoxy groups; and
the cyclopropyl, the cyclobutyl, and the 5- to 6-membered heterocyclyl of R³ are each optionally substituted with 1 to 3 groups independently chosen from F, Cl, Br, cyano, —OH, C₁-C₂ alkyl, and C₁-C₂ alkoxy groups;
and all other variables not specifically defined herein are as defined in any one of the foregoing embodiments.

In some embodiments, in a compound, tautomer, deuterated derivative, or pharmaceutically acceptable salt of the disclosure, R³ is chosen from —CH₃, —CH₂CH₃, —CH₂OH, —C(═O)OCH₃, —CH₂OCH₃, —CH(CH₃)₂, cyclopropyl, difluorocyclopropyl, and tetrahydro-2H-pyranyl; and all other variables not specifically defined herein are as defined in any one of the foregoing embodiments.

In some embodiments, in a compound, tautomer, deuterated derivative, or pharmaceutically acceptable salt of the disclosure, R³ is —CH₃; and all other variables not specifically defined herein are as defined in any one of the foregoing embodiments.

In some embodiments, in a compound, tautomer, deuterated derivative, or pharmaceutically acceptable salt of the disclosure, wherein R¹, for each occurrence, is independently chosen from hydrogen, halogen, cyano, —OH, C₁-C₄ alkyl, C₁-C₄ alkoxy, —C(═O)N(R^{c})₂, and C₃-C₆ cycloalkyl groups, wherein:
R^{c}, for each occurrence, is independently chosen from hydrogen and C₁-C₂ alkyl groups;
the C₁-C₄ alkyl of R¹ is optionally substituted with 1 to 3 groups independently chosen from halogen, cyano, —OH, and C₁-C₂ alkoxy groups;
the C₁-C₄ alkoxy of R¹ is optionally substituted with 1 to 3 independently chosen from halogen groups; and
the C₃-C₆ cycloalkyl of R¹ is optionally substituted with 1 to 3 groups independently chosen from halogen, cyano, —OH, and C₁-C₂ alkoxy groups;
   and all other variables not specifically defined herein are as defined in any one of the foregoing embodiments.

In some embodiments, in a compound, tautomer, deuterated derivative, or pharmaceutically acceptable salt of the disclosure, R¹, for each occurrence, is independently chosen from hydrogen, halogen, cyano, —OH, C₁-C₄ alkyl, C₁-C₄ alkoxy, and C₃-C₆ cycloalkyl; wherein:
the C₁-C₄ alkyl of R¹ is optionally substituted with 1 to 3 groups independently chosen from halogen, cyano, —OH, and C₁-C₂ alkoxy;
the C₁-C₄ alkoxy of R¹ is optionally substituted with 1 to 3 independently chosen halogen groups; and
the C₃-C₆ cycloalkyl of R¹ is optionally substituted with 1 to 3 groups independently chosen from halogen, cyano, —OH, and C₁-C₂ alkoxy;
and all other variables not specifically defined herein are as defined in any one of the foregoing embodiments.

In some embodiments, in a compound, tautomer, deuterated derivative, or pharmaceutically acceptable salt of the disclosure, R¹, for each occurrence, is independently chosen from F, Cl, Br, C₁-C₄ alkyl, and C₃-C₆ cycloalkyl, wherein:
the C₁-C₄ alkyl of R¹ is optionally substituted with 1 to 3 groups independently chosen from halogen and —OH; and
the C₃-C₆ cycloalkyl of R¹ is optionally substituted with 1 to 3 groups independently chosen from halogen and —OH;
   and all other variables not specifically defined herein are as defined in any one of the foregoing embodiments.

In some embodiments, in a compound, tautomer, deuterated derivative, or pharmaceutically acceptable salt of the disclosure, R¹, for each occurrence, is independently chosen from F, Cl, Br, C₁-C₄ alkyl, C₁-C₄ alkoxy, —C(═O)N(R^{c})₂, and C₃-C₆ cycloalkyl groups, wherein:
R^{c}, for each occurrence, is independently chosen from hydrogen and C₁-C₂ alkyl groups;
the C₁-C₄ alkyl of R¹ is optionally substituted with 1 to 3 groups independently chosen from halogen and —OH;
the C₁-C₄ alkoxy of R¹ is optionally substituted with 1 to 3 independently chosen from halogen groups; and
the C₃-C₆ cycloalkyl of R¹ is optionally substituted with 1 to 3 groups independently chosen from halogen and —OH;
and all other variables not specifically defined herein are as defined in any one of the foregoing embodiments.

In some embodiments, in a compound, tautomer, deuterated derivative, or pharmaceutically acceptable salt of the disclosure, R¹, for each occurrence, is independently chosen from F, Cl, Br, C₁-C₄ alkyl, and C₃-C₆ cycloalkyl; wherein:
the C₁-C₄ alkyl of R¹ is optionally substituted with 1 to 3 groups independently chosen from halogen and —OH; and
the C₃-C₆ cycloalkyl of R¹ is optionally substituted with 1 to 3 groups independently chosen from halogen and —OH;
   and all other variables not specifically defined herein are as defined in any one of the foregoing embodiments.

In some embodiments, in a compound, tautomer, deuterated derivative, or pharmaceutically acceptable salt of the disclosure, R¹, for each occurrence, is independently chosen from F, Cl, Br, C₁-C₄ alkyl, C₁-C₄ alkoxy, —C(═O)N(R^{c})₂, and C₃-C₆ cycloalkyl groups, wherein:
R^{c}, for each occurrence, is independently chosen from hydrogen and C₁-C₂ alkyl groups;
the C₁-C₄ alkyl of R¹ is optionally substituted with 1 to 3 groups independently chosen from halogen and —OH; and
the C₁-C₄ alkoxy of R¹ is optionally substituted with 1 to 3 independently chosen from halogen groups;
   and all other variables not specifically defined herein are as defined in any one of the foregoing embodiments.

In some embodiments, in a compound, tautomer, deuterated derivative, or pharmaceutically acceptable salt of the disclosure, R¹, for each occurrence, is independently chosen from Cl, Br, —CH₃, —CF₃, —CH₂CH₃, —CH(CH₃)₂, —CH₂CHF₂, —CH₂CH(CH₃)₂, difluorocyclobutyl, and cyclohexyl; and all other variables not specifically defined herein are as defined in any one of the foregoing embodiments.

In some embodiments, in a compound, tautomer, deuterated derivative, or pharmaceutically acceptable salt of the disclosure, R¹, for each occurrence, is independently chosen from F, Cl, Br, —CH₃, —CH(CH₃)₂, —CF₃, —OCH₃, —OCF₃, —C(═O)N(CH₃)₂, and cyclopropyl; and all other variables not specifically defined herein are as defined in any one of the foregoing embodiments.

In some embodiments, in a compound, tautomer, deuterated derivative, or pharmaceutically acceptable salt of the disclosure, R¹, for each occurrence, is C₁; and all other variables not specifically defined herein are as defined in any one of the foregoing embodiments.

In some embodiments, in a compound, tautomer, deuterated derivative, or pharmaceutically acceptable salt of the disclosure, R¹, for each occurrence, is independently chosen from halogen, —OH, and C₁-C₄ alkyl; wherein:
the C₁-C₄ alkyl of R¹ is optionally substituted with 1 to 3 groups independently chosen from halogen and —OH;
   and all other variables not specifically defined herein are as defined in any one of the foregoing embodiments.

In some embodiments, in a compound, tautomer, deuterated derivative, or pharmaceutically acceptable salt of the disclosure, R¹, for each occurrence, is independently chosen from F, Cl, Br, —OH, and C₁-C₂ alkyl; wherein:
the C₁-C₂ alkyl of R¹ is optionally substituted with 1 to 3 groups independently chosen from F, Cl, and —OH;
   and all other variables not specifically defined herein are as defined in any one of the foregoing embodiments.

In some embodiments, in a compound, tautomer, deuterated derivative, or pharmaceutically acceptable salt of the disclosure, R¹, for each occurrence, is independently chosen from F, —OH, —CH₃, —CHF₂, and —CH₂OH; and all other variables not specifically defined herein are as defined in any one of the foregoing embodiments.

In some embodiments, in a compound, tautomer, deuterated derivative, or pharmaceutically acceptable salt of the disclosure, R^{a}, for each occurrence, is independently chosen from halogen, cyano, C₁-C₆ alkyl, C₁-C₄ alkoxy, C₁-C₆ haloalkyl, C₁-C₆ haloalkoxy, —C(═O)NR^{h}Rⁱ, —NR^{h}Rⁱ, —NR^{h}C(═O)R^{k}, OR^{k}, —[O(CH₂)_{q}]ᵣO(C₁-C₆ alkyl), —S(═O)₂R^{k}, —S(═O)₂NR^{h}Rⁱ, C₃-C₆ cycloalkyl, 5 to 10-membered heterocyclyl, phenyl, and 5- to 8-membered heteroaryl; wherein:
the C₁-C₆ alkyl of R^{a} is optionally substituted with 1 to 3 groups independently chosen from cyano, —C(═O)NR^{h}Rⁱ, —NR^{h}Rⁱ, —NR^{h}C(═O)R^{k}, —NR^{h}C(═O)OR^{k}, —NR^{h}C(═O)NRⁱR^{j}, —NR^{h}S(═O)ₚR^{k}, —OR^{k}, —S(═O)₂R^{k}, —S(═O)ₚNR^{h}Rⁱ, and C₃-C₆ cycloalkyl;
the C₃-C₆ cycloalkyl, the 5 to 10-membered heterocyclyl, the phenyl, and the 5- to 8-membered heteroaryl of R^{a} are each optionally substituted with 1 to 3 groups independently chosen from halogen, C₁-C₂ alkyl, and —OR^{k}, wherein:
   R^{h}, Rⁱ, and R^{j}, for each occurrence, are each independently chosen from hydrogen, C₁-C₂ alkyl, cyclopropyl, and cyclobutyl, wherein:
      the C₁-C₂ alkyl of any one of R^{h}, Rⁱ, and R^{j} is optionally substituted with 1 to 3 groups independently chosen from halogen and —OH;
   R^{k}, for each occurrence, is each independently chosen from hydrogen and C₁-C₄ alkyl, wherein:
      the C₁-C₄ alkyl of R^{k} is optionally substituted with 1 to 3 groups independently chosen from halogen and —OH; and
q and r are each an integer chosen from 1, 2, and 3;
   and all other variables not specifically defined herein are as defined in any one of the foregoing embodiments.

In some embodiments, in a compound, tautomer, deuterated derivative, or pharmaceutically acceptable salt of the disclosure, R^{a}, for each occurrence, is independently chosen from halogen, cyano, C₁-C₆ alkyl, C₁-C₄ alkoxy, C₁-C₄ haloalkyl, C₁-C₄ haloalkoxy, —C(═O)NR^{h}Rⁱ, —NR^{h}Rⁱ, —NR^{h}C(O)R^{k}, —OR^{k}, —[O(CH₂)_{q}]ᵣO(C₁-C₄ alkyl), —S(═O)₂R^{k}, —S(═O)₂NR^{h}Rⁱ, cyclopropyl, cyclobutyl, 5- to 6-membered heterocyclyl, phenyl, and 5- to 6-membered heteroaryl, wherein:
the C₁-C₆ alkyl of R^{a} is optionally substituted with 1 to 3 groups independently chosen from cyano, —C(═O)NR^{h}Rⁱ, —NR^{h}Rⁱ, —OR^{k}, cyclopropyl, and cyclobutyl;
the cyclopropyl, the cyclobutyl, the 5- to 6-membered heterocyclyl, the phenyl, and the 5 to 6-membered heteroaryl of R^{a} are each optionally substituted with 1 to 3 groups independently chosen from halogen, —CH₃, —OH, and —OCH₃, wherein:
   R^{h} and Rⁱ, for each occurrence, are each independently chosen from hydrogen, —CH₃, cyclopropyl, and cyclobutyl, wherein:
      the —CH₃ of any one of R^{h} and Rⁱ is optionally substituted with 1 to 3 groups independently chosen from F, Cl, and —OH;
   R^{k}, for each occurrence, is each independently chosen from hydrogen and —CH₃; wherein:
      the —CH₃ of R^{k} is optionally substituted with 1 to 3 groups independently chosen from halogen and —OH;
         and all other variables not specifically defined herein are as defined in any one of the foregoing embodiments.

In some embodiments, in a compound, tautomer, deuterated derivative, or pharmaceutically acceptable salt of the disclosure, R^{a}, for each occurrence, is independently chosen from F, Cl, Br, cyano, C₁-C₆ alkyl, C₁-C₂ alkoxy, C₁-C₂ haloalkyl, —C(═O)NR^{h}Rⁱ, —NR^{h}Rⁱ, —NR^{h}C(═O)R^{k}, OR^{k}, —[O(CH₂)_{q}]ᵣO(C₁-C₂ alkyl), —S(═O)₂R^{k}, —S(═O)₂NR^{h}Rⁱ, cyclopropyl, cyclobutyl, 5-membered heterocyclyl, phenyl, and 6-membered heteroaryl, wherein:
the C₁-C₆ alkyl of R^{a} is optionally substituted with 1 to 3 groups independently chosen from cyano, —C(═O)NR^{h}Rⁱ, —OR^{k}, and cyclopropyl;
the cyclopropyl, the cyclobutyl, the 5 to 6-membered heterocyclyl, the phenyl, and the 5 to 6-membered heteroaryl of R^{a} are each optionally substituted with 1 to 3 groups independently chosen from halogen, —CH₃, —OH, and —OCH₃, wherein:
   R^{h} and Rⁱ, for each occurrence, are each independently chosen from hydrogen, —CH₃, and cyclopropyl; wherein:
      the —CH₃ of any one of R^{h} and Rⁱ is optionally substituted with 1 to 3 groups independently chosen from F, Cl, and —OH;
   R^{k}, for each occurrence, is each independently chosen from hydrogen and —CH₃; and
q and r are each an integer independently chosen from 1 and 2;
   and all other variables not specifically defined herein are as defined in any one of the foregoing embodiments.

In some embodiments, in a compound, tautomer, deuterated derivative, or pharmaceutically acceptable salt of the disclosure, R^{a}, for each occurrence, is independently chosen from F, cyano, —OH, —CH₃, —CF₃, —CH(CH₃)₂, —(CH₂)₂OH, —(CH₂)₂OCH₃, —CH₂CH(OH)C₂H5, —CH₂C(CH₃)(CH₂OH)₂, —OCH₃, —OCH₂CH₃, —[O(CH₂)₂]₂OCH₃, —CH₂C(═O)NHCH₃, —(CH₂)₂SO₂CH₃, —CH₂C(═O)N(CH₃)₂, —CH₂(cyclopropyl), —C(═O)NH₂, —C(═O)NH(cyclopropyl), —NH₂, —NHCH₃, —N(CH₃)₂, —NHC(CH₃)₂CH₂OH, —NHC(═O)CH₃, —SO₂CH₃, —SO₂NH₂, cyclopropyl, 2-methoxyphenyl, N-methylpiperazinyl, tetrahydro-2H-pyranyl, methylpyrazolyl, pyridinyl, and tetrahydrothiophenyl 1,1-dioxide; and all other variables not specifically defined herein are as defined in any one of the foregoing embodiments.

In some embodiments, in a compound, tautomer, deuterated derivative, or pharmaceutically acceptable salt of the disclosure, R^{a}, for each occurrence, is independently chosen from —CH₃ and —(CH₂)₂SO₂CH₃; and all other variables not specifically defined herein are as defined in any one of the foregoing embodiments.

In some embodiments, a compound, tautomer, deuterated derivative, or pharmaceutically acceptable salt of the disclosure is represented by one of the following structural formula:

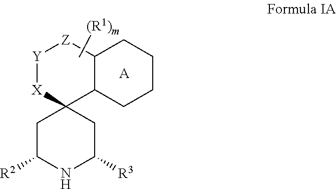

a tautomer thereof, a deuterated derivative of that compound or tautomer, or a pharmaceutically acceptable salt of any of the foregoing; and all other variables not specifically defined herein are as defined in any one of the foregoing embodiments.

In some embodiments, at least one compound, tautomer, deuterated derivative, or pharmaceutically acceptable salt of the disclosure is a compound represented by one of the following structural formulae:

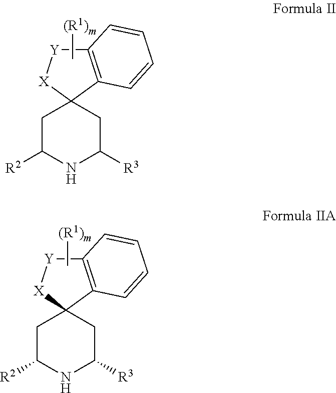

a tautomer thereof, a deuterated derivative of that compound or tautomer, or a pharmaceutically acceptable salt of any of the foregoing; and all other variables not specifically defined herein are as defined in any one of the foregoing embodiments.

In some embodiments, a compound, tautomer, deuterated derivative, or pharmaceutically acceptable salt of the disclosure is represented by one of the following structural formulae:

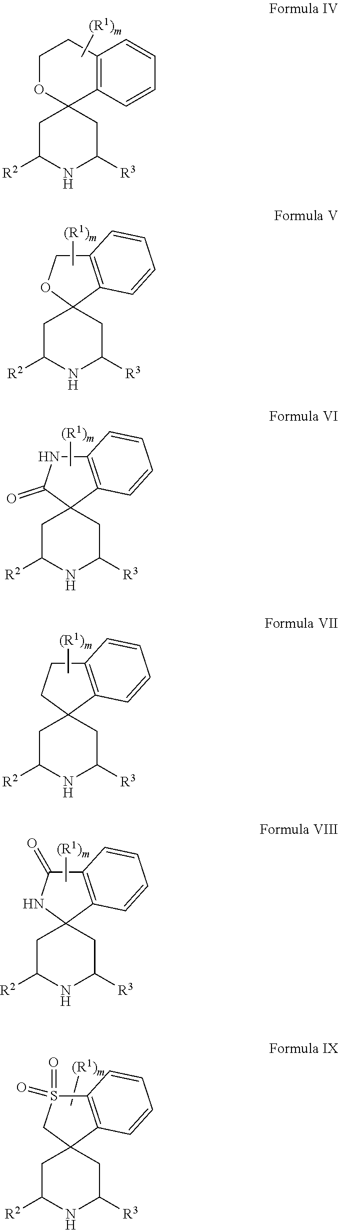

a tautomer thereof, a deuterated derivative of that compound or tautomer, or a pharmaceutically acceptable salt of any of the foregoing; and all other variables not specifically defined herein are as defined in any one of the foregoing embodiments.

In some embodiments, at least one compound, tautomer, deuterated derivative, or pharmaceutically acceptable salt of the disclosure is a compound represented by one of the following structural formulae:

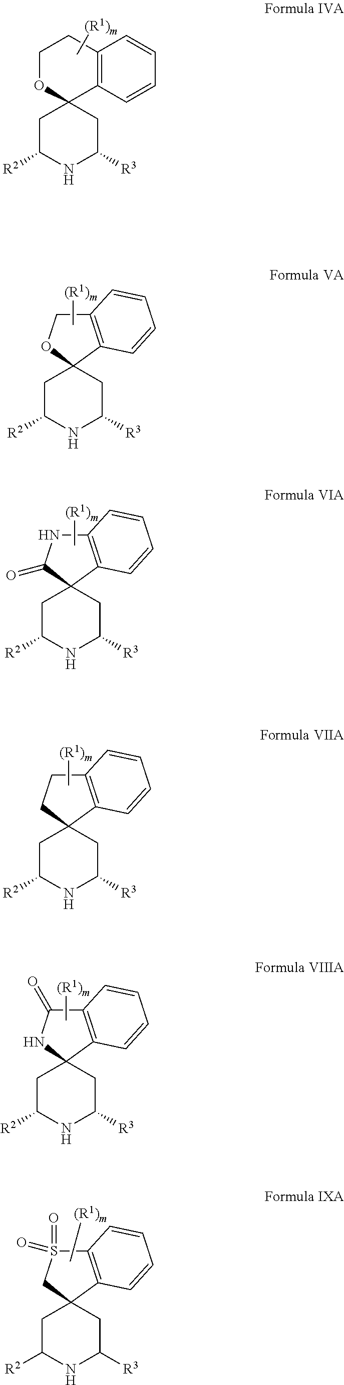

a tautomer thereof, a deuterated derivative of that compound or tautomer, or a pharmaceutically acceptable salt of any of the foregoing; and all other variables not specifically defined herein are as defined in any one of the foregoing embodiments.

In some embodiments, at least one compound, tautomer, deuterated derivative, or pharmaceutically acceptable salt of the disclosure is a compound represented by the one of the following structural formulae:

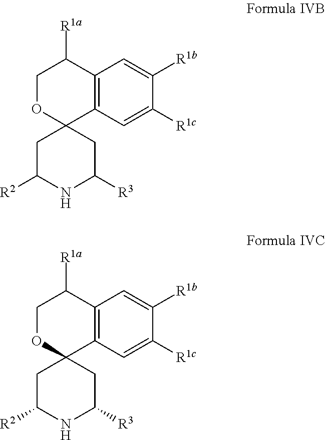

a tautomer thereof, a deuterated derivative of that compound or tautomer, or a pharmaceutically acceptable salt of any of the foregoing, wherein:
   R^{1a} is chosen from hydrogen, halogen, —OH, and phenyl groups, wherein:
   the phenyl of R^{1a} is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups;
   R^{1b} and R^{1c} are each independently chosen from hydrogen, halogen, —OH, cyano, C₁-C₄ alkyl, C₁-C₄ alkoxy, —C(═O)OR^{c}, —C(═O)N(R^{c})₂, and —OS(═O)₂R^{c} groups, wherein:
   R^{c}, for each occurrence, is independently chosen from hydrogen, C₁-C₄ alkyl, and C₁-C₄ haloalkyl groups; and
   the C₁-C₆ alkyl of R^{1b} and/or R^{1c} is optionally substituted with 1 to 3 groups independently chosen from halogen and —OH groups; and
all variables not specifically defined herein are as defined in any one of the foregoing embodiments.

In some embodiments, at least one compound, tautomer, deuterated derivative, or pharmaceutically acceptable salt of the disclosure is a compound represented by one of the following structural formulae:

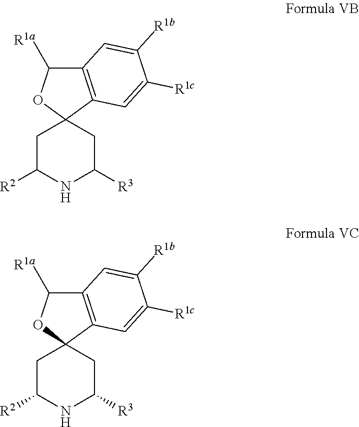

a tautomer thereof, a deuterated derivative of that compound or tautomer, or a pharmaceutically acceptable salt of any of the foregoing, wherein:
   R^{1a} is chosen from hydrogen, phenyl, and C(═O)N(RI)₂ groups, wherein:
      the phenyl of R^{1a} is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy,
   —C(═O)NH₂, —C(═O)NH(C₁-C₄ alkyl), and —C(═O)N(C₁-C₄ alkyl)₂ groups;
   R^{c1}, for each occurrence, is independently chosen from hydrogen and C₁-C₄ alkyl groups;
   R^{1b} and Rid are each independently chosen from hydrogen and halogen groups; and all variables not specifically defined herein are as defined in any one of the foregoing embodiments.

In some embodiments, at least one compound, tautomer, deuterated derivative, or pharmaceutically acceptable salt of the disclosure is a compound represented by one of the following structural formulae:

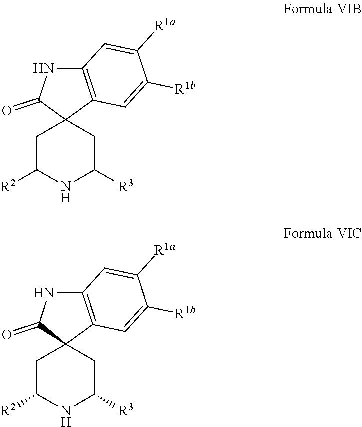

a tautomer thereof, a deuterated derivative of that compound or tautomer, or a pharmaceutically acceptable salt of any of the foregoing, wherein:
   R^{1a} and R^{1b} are each independently chosen from hydrogen, halogen, C₁-C₄ alkyl, and C₁-C₄ haloalkyl groups; and
all variables not specifically defined herein are as defined in any one of the foregoing embodiments.

In some embodiments, at least one compound, tautomer, deuterated derivative, or pharmaceutically acceptable salt of the disclosure is a compound represented by one of the following structural formulae:

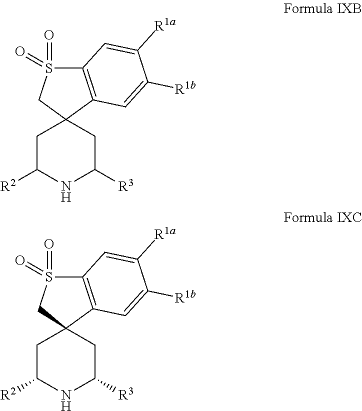

a tautomer thereof, a deuterated derivative of that compound or tautomer, or a pharmaceutically acceptable salt of any of the foregoing, wherein:
   R^{1a} and R^{1b} are each independently chosen from hydrogen, halogen, C₁-C₄ alkyl, and C₁-C₄ haloalkyl groups; and
   all variables not specifically defined herein are as defined in any one of the foregoing embodiments.

In some embodiments, at least one compound, tautomer, deuterated derivative, or pharmaceutically acceptable salt of the disclosure is a silicon derivative represented by one of the following structural formulae:

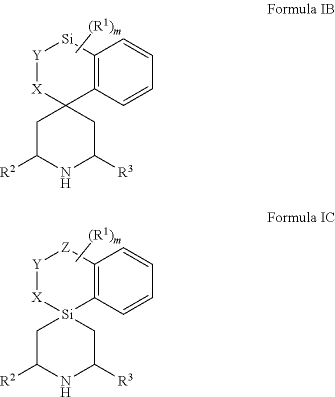

a tautomer thereof, a deuterated derivative of that silicon derivative or tautomer, or a pharmaceutically acceptable salt of any of the foregoing, wherein all variables not specifically defined herein are as defined in any one of the foregoing embodiments.

In some embodiments, at least one compound, tautomer, deuterated derivative, or pharmaceutically acceptable salt of the disclosure is a boron derivative represented by one of the following structural formula:

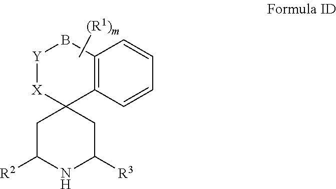

a tautomer thereof, a deuterated derivative of that boron derivative or tautomer, or a pharmaceutically acceptable salt of any of the foregoing, wherein all variables not specifically defined herein are as defined in any one of the foregoing embodiments.

In some embodiments, the at least one compound, tautomer, deuterated derivative, or pharmaceutically acceptable salt of the disclosure is chosen from Compounds 1 to 42 depicted in Table 1, a tautomer thereof, a deuterated derivative of that compound or tautomer, or a pharmaceutically acceptable salt of any of the foregoing. A wavy line in a compound in Table 1 (i.e.,

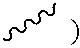

depicts a bond between two atoms and indicates a position of mixed stereochemistry for a collection of molecules, such as a racemic mixture, cis/trans isomers, or (E)/(Z) isomers. An asterisk adjacent to an atom (e.g.,

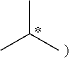

in a compound in Table 1, indicates a chiral position in the molecule.

In some embodiments, the at least one compound, tautomer, deuterated derivative, or pharmaceutically acceptable salt of the disclosure is chosen from Compounds I1 to I36 depicted in Table 2, a tautomer thereof, a deuterated derivative of that compound or tautomer, or a pharmaceutically acceptable salt of any of the foregoing. A wavy line in a compound in Table 2 (i.e.,

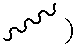

depicts a bond between two atoms and indicates a position of mixed stereochemistry for a collection of molecules, such as a racemic mixture, cis/trans isomers, or (E)/(Z) isomers. An asterisk adjacent to an atom (e.g.,

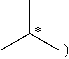

in a compound in Table 2, indicates a chiral position in the molecule.

TABLE 1
Compounds 1 to 42

| | |
|---|---|
| 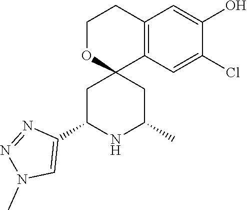 [SMILES: C[C@H]1C[C@@]2(C[C@@H](c3cn(C)nn3)N1)OCCc1cc(O)c(Cl)cc12] | Compound 1 |
| 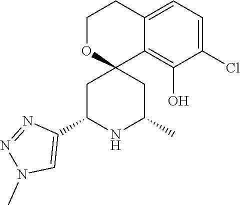 [SMILES: C[C@H]1C[C@@]2(C[C@@H](c3cn(C)nn3)N1)OCCc1ccc(Cl)c(O)c12] | Compound 2 |
| 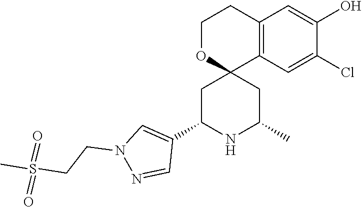 [SMILES: C[C@H]1C[C@@]2(C[C@@H](c3cnn(CCS(C)(=O)=O)c3)N1)OCCc1cc(O)c(Cl)cc12] | Compound 3 |
| 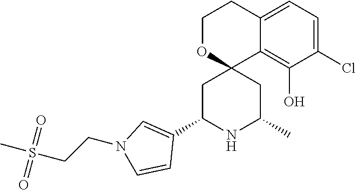 [SMILES: C[C@H]1C[C@@]2(C[C@@H](c3ccn(CCS(C)(=O)=O)c3)N1)OCCc1ccc(Cl)c(O)c12] | Compound 4 |
| 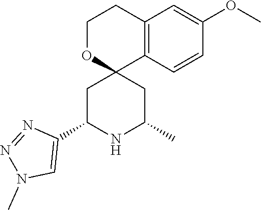 [SMILES: COc1ccc2c(c1)CCO[C@@]21C[C@@H](c2cn(C)nn2)N[C@@H](C)C1] | Compound 5 |
| 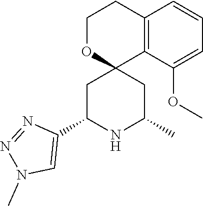 [SMILES: COc1cccc2c1[C@]1(C[C@@H](c3cn(C)nn3)N[C@@H](C)C1)OCC2] | Compound 6 |
| 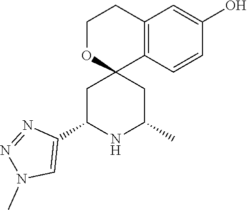 [SMILES: C[C@H]1C[C@@]2(C[C@@H](c3cn(C)nn3)N1)OCCc1cc(O)ccc12] | Compound 7 |
| 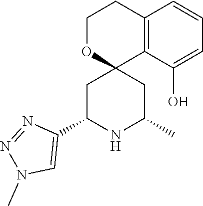 [SMILES: C[C@H]1C[C@@]2(C[C@@H](c3cn(C)nn3)N1)OCCc1cccc(O)c12] | Compound 8 |
| 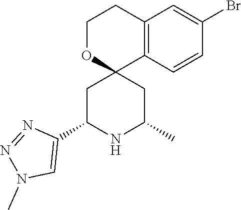 [SMILES: C[C@H]1C[C@@]2(C[C@@H](c3cn(C)nn3)N1)OCCc1cc(Br)ccc12] | Compound 9 |
| 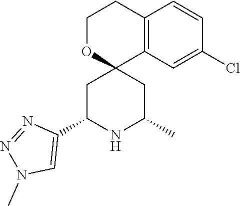 [SMILES: C[C@H]1C[C@@]2(C[C@@H](c3cn(C)nn3)N1)OCCc1ccc(Cl)cc12] | Compound 10 |
| 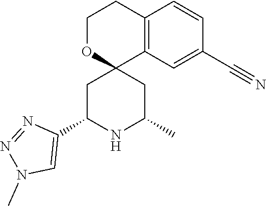 [SMILES: C[C@H]1C[C@@]2(C[C@@H](c3cn(C)nn3)N1)OCCc1ccc(C#N)cc12] | Compound 11 |
| 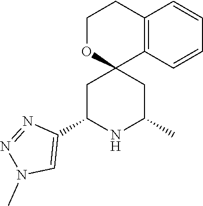 [SMILES: C[C@H]1C[C@@]2(C[C@@H](c3cn(C)nn3)N1)OCCc1ccccc12] | Compound 12 |
| 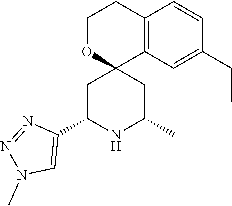 [SMILES: CCc1ccc2c(c1)[C@]1(C[C@@H](c3cn(C)nn3)N[C@@H](C)C1)OCC2] | Compound 13 |
| 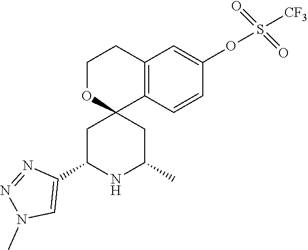 [SMILES: C[C@H]1C[C@@]2(C[C@@H](c3cn(C)nn3)N1)OCCc1cc(OS(=O)(=O)C(F)(F)F)ccc12] | Compound 14 |
| 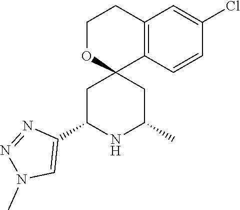 [SMILES: C[C@H]1C[C@@]2(C[C@@H](c3cn(C)nn3)N1)OCCc1cc(Cl)ccc12] | Compound 15 |
| 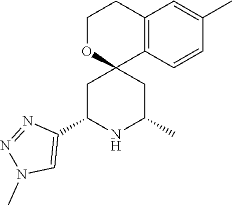 [SMILES: Cc1ccc2c(c1)CCO[C@@]21C[C@@H](c2cn(C)nn2)N[C@@H](C)C1] | Compound 16 |
| 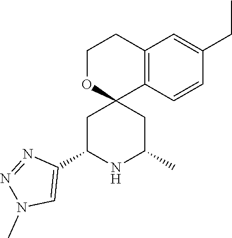 [SMILES: CCc1ccc2c(c1)CCO[C@@]21C[C@@H](c2cn(C)nn2)N[C@@H](C)C1] | Compound 17 |
| 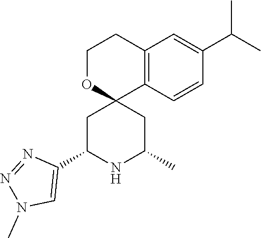 [SMILES: CC(C)c1ccc2c(c1)CCO[C@@]21C[C@@H](c2cn(C)nn2)N[C@@H](C)C1] | Compound 18 |
| 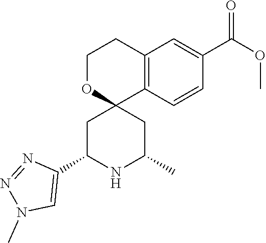 [SMILES: COC(=O)c1ccc2c(c1)CCO[C@@]21C[C@@H](c2cn(C)nn2)N[C@@H](C)C1] | Compound 19 |
| 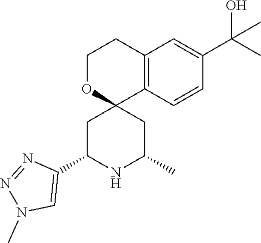 [SMILES: C[C@H]1C[C@@]2(C[C@@H](c3cn(C)nn3)N1)OCCc1cc(C(C)(C)O)ccc12] | Compound 20 |
| 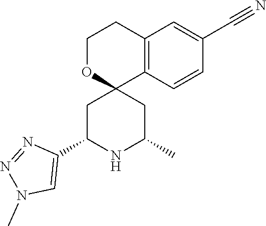 [SMILES: C[C@H]1C[C@@]2(C[C@@H](c3cn(C)nn3)N1)OCCc1cc(C#N)ccc12] | Compound 21 |
| 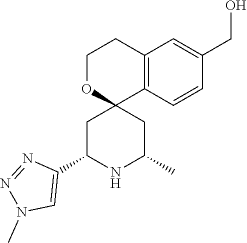 [SMILES: C[C@H]1C[C@@]2(C[C@@H](c3cn(C)nn3)N1)OCCc1cc(CO)ccc12] | Compound 22 |
| 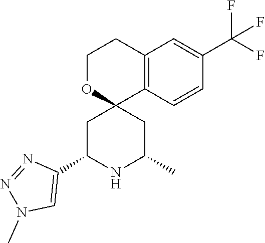 [SMILES: C[C@H]1C[C@@]2(C[C@@H](c3cn(C)nn3)N1)OCCc1cc(C(F)(F)F)ccc12] | Compound 23 |
| 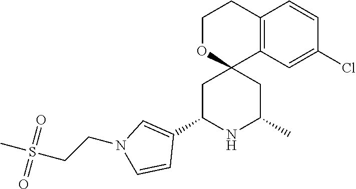 [SMILES: C[C@H]1C[C@@]2(C[C@@H](c3ccn(CCS(C)(=O)=O)c3)N1)OCCc1ccc(Cl)cc12] | Compound 24 |
| 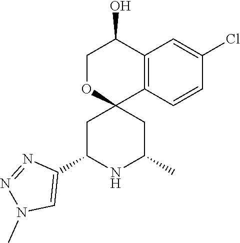 [SMILES: C[C@H]1C[C@@]2(C[C@@H](c3cn(C)nn3)N1)OC[C@@H](O)c1cc(Cl)ccc12] | Compound 25 |
| 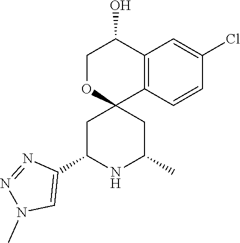 [SMILES: C[C@H]1C[C@@]2(C[C@@H](c3cn(C)nn3)N1)OC[C@H](O)c1cc(Cl)ccc12] | Compound 26 |
| 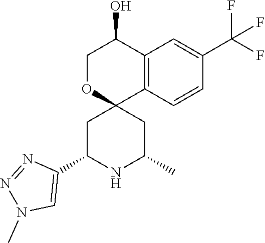 [SMILES: C[C@H]1C[C@@]2(C[C@@H](c3cn(C)nn3)N1)OC[C@@H](O)c1cc(C(F)(F)F)ccc12] | Compound 27 |
| 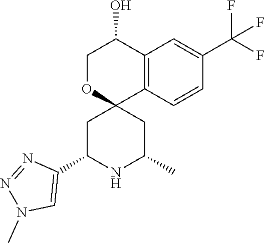 [SMILES: C[C@H]1C[C@@]2(C[C@@H](c3cn(C)nn3)N1)OC[C@H](O)c1cc(C(F)(F)F)ccc12] | Compound 28 |
| 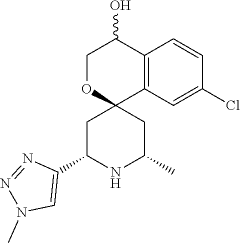 | Compound 29 |
| 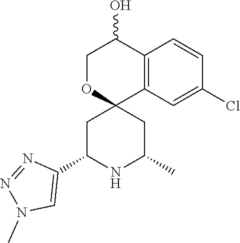 | Compound 30 |
| 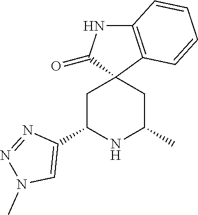 [SMILES: C[C@H]1C[C@]2(C[C@@H](c3cn(C)nn3)N1)C(=O)Nc1ccccc12] | Compound 31 |
| 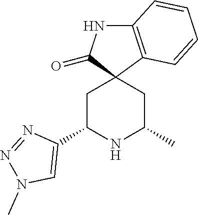 [SMILES: C[C@H]1C[C@@]2(C[C@@H](c3cn(C)nn3)N1)C(=O)Nc1ccccc12] | Compound 32 |
| 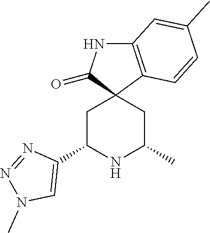 [SMILES: Cc1ccc2c(c1)NC(=O)[C@@]21C[C@@H](c2cn(C)nn2)N[C@@H](C)C1] | Compound 33 |
| 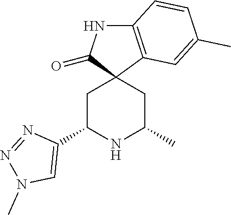 [SMILES: Cc1ccc2c(c1)[C@]1(C[C@@H](c3cn(C)nn3)N[C@@H](C)C1)C(=O)N2] | Compound 34 |
| 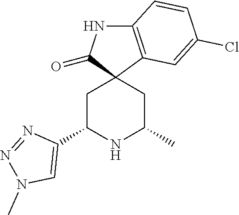 [SMILES: C[C@H]1C[C@@]2(C[C@@H](c3cn(C)nn3)N1)C(=O)Nc1ccc(Cl)cc12] | Compound 35 |
| 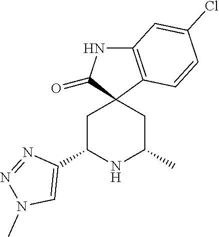 [SMILES: C[C@H]1C[C@@]2(C[C@@H](c3cn(C)nn3)N1)C(=O)Nc1cc(Cl)ccc12] | Compound 36 |
| 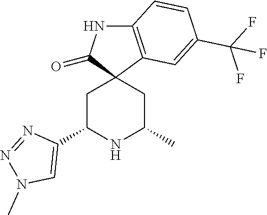 [SMILES: C[C@H]1C[C@@]2(C[C@@H](c3cn(C)nn3)N1)C(=O)Nc1ccc(C(F)(F)F)cc12] | Compound 37 |
| 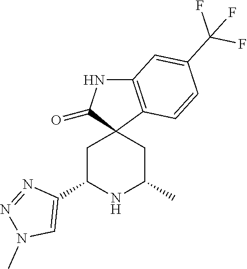 [SMILES: C[C@H]1C[C@@]2(C[C@@H](c3cn(C)nn3)N1)C(=O)Nc1cc(C(F)(F)F)ccc12] | Compound 38 |
| 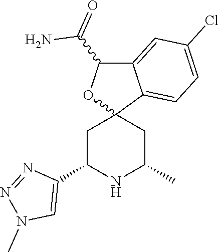 [SMILES: C[C@H]1CC2(C[C@@H](c3cn(C)nn3)N1)OC(C(N)=O)c1cc(Cl)ccc12] | Compound 39 |
| 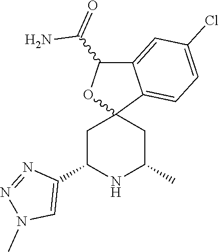 [SMILES: C[C@H]1CC2(C[C@@H](c3cn(C)nn3)N1)OC(C(N)=O)c1cc(Cl)ccc12] | Compound 40 |
| 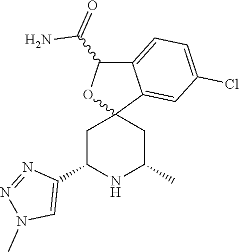 [SMILES: C[C@H]1CC2(C[C@@H](c3cn(C)nn3)N1)OC(C(N)=O)c1ccc(Cl)cc12] | Compound 41 |
| 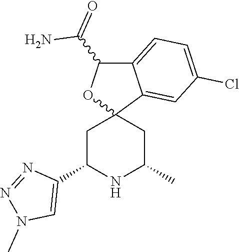 [SMILES: C[C@H]1CC2(C[C@@H](c3cn(C)nn3)N1)OC(C(N)=O)c1ccc(Cl)cc12] | Compound 42 |

TABLE 2
Compounds I1 to I36

| | |
|---|---|
| 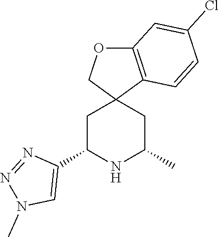 [SMILES: C[C@H]1CC2(COc3cc(Cl)ccc32)C[C@@H](c2cn(C)nn2)N1] | Compound I1 |
| 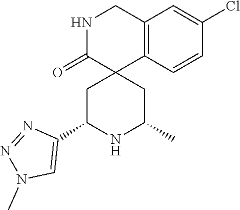 [SMILES: C[C@H]1CC2(C[C@@H](c3cn(C)nn3)N1)C(=O)NCc1cc(Cl)ccc12] | Compound I2 |
| 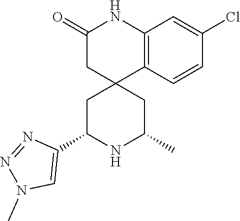 [SMILES: C[C@H]1CC2(CC(=O)Nc3cc(Cl)ccc32)C[C@@H](c2cn(C)nn2)N1] | Compound I3 |
| 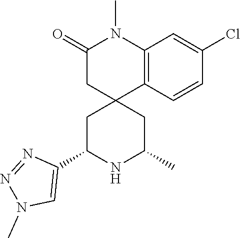 [SMILES: C[C@H]1CC2(CC(=O)N(C)c3cc(Cl)ccc32)C[C@@H](c2cn(C)nn2)N1] | Compound I4 |
| 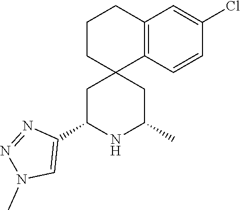 [SMILES: C[C@H]1CC2(CCCc3cc(Cl)ccc32)C[C@@H](c2cn(C)nn2)N1] | Compound I5 |
| 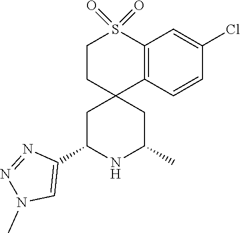 [SMILES: C[C@H]1CC2(CCS(=O)(=O)c3cc(Cl)ccc32)C[C@@H](c2cn(C)nn2)N1] | Compound I6 |
| 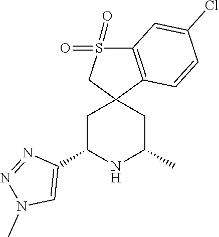 [SMILES: C[C@H]1CC2(C[C@@H](c3cn(C)nn3)N1)CS(=O)(=O)c1cc(Cl)ccc12] | Compound I7 |
| 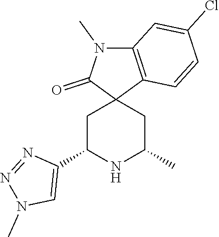 [SMILES: C[C@H]1CC2(C[C@@H](c3cn(C)nn3)N1)C(=O)N(C)c1cc(Cl)ccc12] | Compound I8 |
| 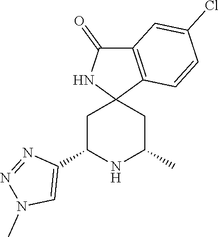 [SMILES: C[C@H]1CC2(C[C@@H](c3cn(C)nn3)N1)NC(=O)c1cc(Cl)ccc12] | Compound I9 |
| 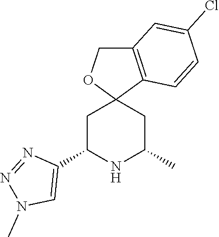 [SMILES: C[C@H]1CC2(C[C@@H](c3cn(C)nn3)N1)OCc1cc(Cl)ccc12] | Compound I10 |
| 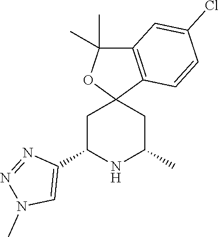 [SMILES: C[C@H]1CC2(C[C@@H](c3cn(C)nn3)N1)OC(C)(C)c1cc(Cl)ccc12] | Compound I11 |
| 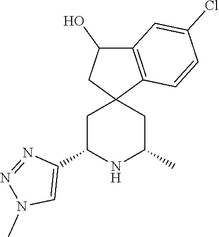 [SMILES: C[C@H]1CC2(CC(O)c3cc(Cl)ccc32)C[C@@H](c2cn(C)nn2)N1] | Compound I12 |
| 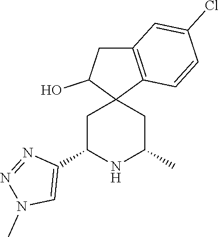 [SMILES: C[C@H]1CC2(C[C@@H](c3cn(C)nn3)N1)c1ccc(Cl)cc1CC2O] | Compound I13 |
| 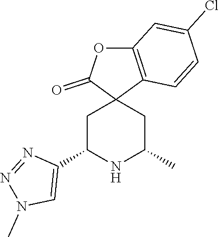 [SMILES: C[C@H]1CC2(C[C@@H](c3cn(C)nn3)N1)C(=O)Oc1cc(Cl)ccc12] | Compound I14 |
| 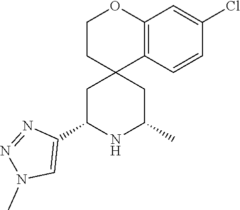 [SMILES: C[C@H]1CC2(CCOc3cc(Cl)ccc32)C[C@@H](c2cn(C)nn2)N1] | Compound I15 |
| 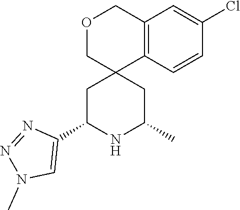 [SMILES: C[C@H]1CC2(COCc3cc(Cl)ccc32)C[C@@H](c2cn(C)nn2)N1] | Compound I16 |
| 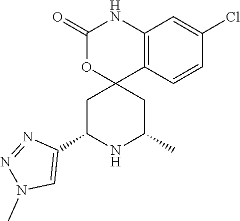 [SMILES: C[C@H]1CC2(C[C@@H](c3cn(C)nn3)N1)OC(=O)Nc1cc(Cl)ccc12] | Compound I17 |
| 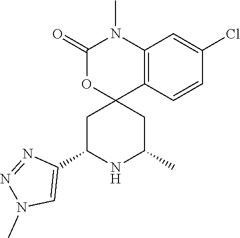 [SMILES: C[C@H]1CC2(C[C@@H](c3cn(C)nn3)N1)OC(=O)N(C)c1cc(Cl)ccc12] | Compound I18 |
| 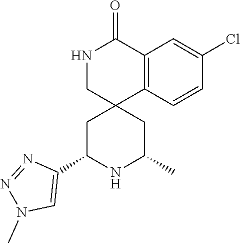 [SMILES: C[C@H]1CC2(CNC(=O)c3cc(Cl)ccc32)C[C@@H](c2cn(C)nn2)N1] | Compound I19 |
| 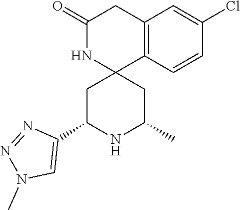 [SMILES: C[C@H]1CC2(C[C@@H](c3cn(C)nn3)N1)NC(=O)Cc1cc(Cl)ccc12] | Compound I20 |
| 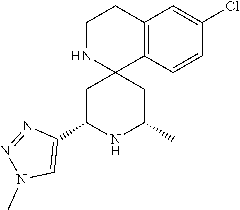 [SMILES: C[C@H]1CC2(C[C@@H](c3cn(C)nn3)N1)NCCc1cc(Cl)ccc12] | Compound I21 |
| 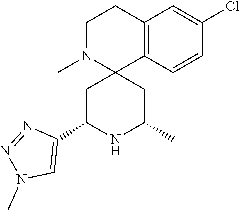 [SMILES: C[C@H]1CC2(C[C@@H](c3cn(C)nn3)N1)c1ccc(Cl)cc1CCN2C] | Compound I22 |
| 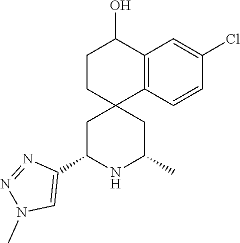 [SMILES: C[C@H]1CC2(CCC(O)c3cc(Cl)ccc32)C[C@@H](c2cn(C)nn2)N1] | Compound I23 |
| 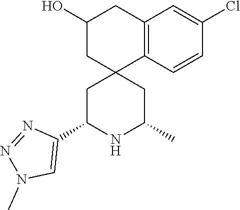 [SMILES: C[C@H]1CC2(CC(O)Cc3cc(Cl)ccc32)C[C@@H](c2cn(C)nn2)N1] | Compound I24 |
| 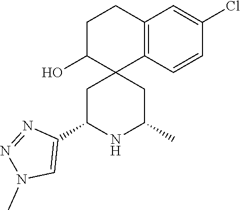 [SMILES: C[C@H]1CC2(C[C@@H](c3cn(C)nn3)N1)c1ccc(Cl)cc1CCC2O] | Compound I25 |
| 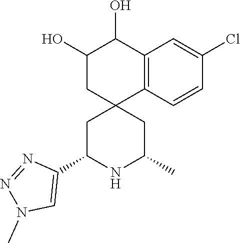 [SMILES: C[C@H]1CC2(CC(O)C(O)c3cc(Cl)ccc32)C[C@@H](c2cn(C)nn2)N1] | Compound I26 |
| 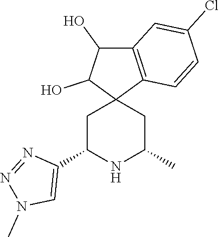 [SMILES: C[C@H]1CC2(C[C@@H](c3cn(C)nn3)N1)c1ccc(Cl)cc1C(O)C2O] | Compound I27 |
| 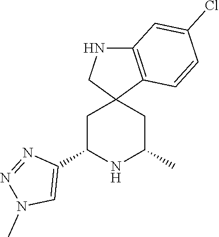 [SMILES: C[C@H]1CC2(CNc3cc(Cl)ccc32)C[C@@H](c2cn(C)nn2)N1] | Compound I28 |
| 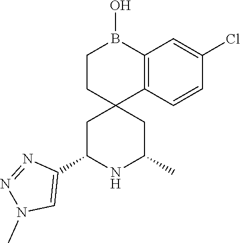 [SMILES: C[C@H]1CC2(CCB(O)c3cc(Cl)ccc32)C[C@@H](c2cn(C)nn2)N1] | Compound I29 |
| 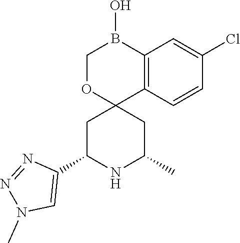 [SMILES: C[C@H]1CC2(C[C@@H](c3cn(C)nn3)N1)OCB(O)c1cc(Cl)ccc12] | Compound I30 |
| 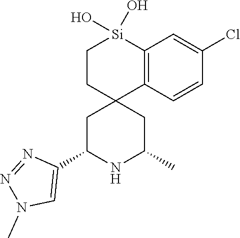 [SMILES: C[C@H]1CC2(CC[Si](O)(O)c3cc(Cl)ccc32)C[C@@H](c2cn(C)nn2)N1] | Compound I31 |
| 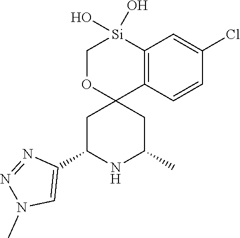 [SMILES: C[C@H]1CC2(C[C@@H](c3cn(C)nn3)N1)OC[Si](O)(O)c1cc(Cl)ccc12] | Compound I32 |
| 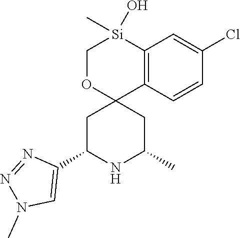 [SMILES: C[C@H]1CC2(C[C@@H](c3cn(C)nn3)N1)OC[Si](C)(O)c1cc(Cl)ccc12] | Compound I33 |
| 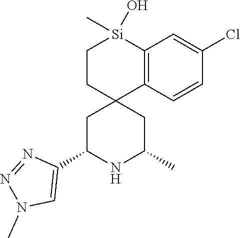 [SMILES: C[C@H]1CC2(CC[Si](C)(O)c3cc(Cl)ccc32)C[C@@H](c2cn(C)nn2)N1] | Compound I34 |
| 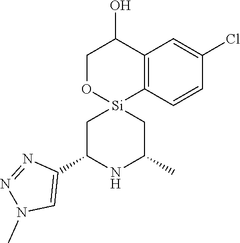 [SMILES: C[C@H]1C[Si]2(C[C@@H](c3cn(C)nn3)N1)OCC(O)c1cc(Cl)ccc12] | Compound I35 |
| 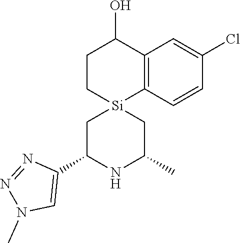 [SMILES: C[C@H]1C[Si]2(CCC(O)c3cc(Cl)ccc32)C[C@@H](c2cn(C)nn2)N1] | Compound I36 |

Some embodiments of the disclosure include derivatives of Compounds 1 to 42 and Compounds I1 to I36 or compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, tautomers thereof, deuterated derivatives of those compounds or tautomers, or pharmaceutically acceptable salts of any of the foregoing. In some embodiments, the derivatives are silicon derivatives in which at least one carbon atom in a compound, tautomer, deuterated derivative, or pharmaceutically acceptable salt chosen from Compounds 1 to 42 and Compounds I1 to I36 or compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, tautomers thereof, deuterated derivatives of those compounds or tautomers, and pharmaceutically acceptable salts of any of the foregoing, has been replaced by silicon. In some embodiments, the derivatives are boron derivatives, in which at least one carbon atom in a compound, tautomer, deuterated derivative, or pharmaceutically acceptable salt chosen from Compounds 1 to 42 and Compounds I1 to I36 or compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, tautomers thereof, deuterated derivatives of those compounds or tautomers, and pharmaceutically acceptable salts of any of the foregoing, has been replaced by boron. In other embodiments, the derivatives are phosphorus derivatives, in which at least one carbon atom in a compound, tautomer, deuterated derivative, or pharmaceutically acceptable salt chosen from Compounds 1 to 42 and Compounds I1 to I36 or compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, tautomers thereof, deuterated derivatives of those compounds or tautomers, and pharmaceutically acceptable salts of any of the foregoing, has been replaced by phosphorus.

In some embodiments, the derivative is a silicon derivative in which one carbon atom in a compound, tautomer, deuterated derivative, or pharmaceutically acceptable salt chosen from Compounds 1 to 42 and Compounds I1 to I36 or compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, tautomers thereof, deuterated derivatives of those compounds or tautomers, and pharmaceutically acceptable salts of any of the foregoing, has been replaced by silicon or a silicon derivative (e.g., —Si(CH₃)₂— or —Si(OH)₂—). The carbon replaced by silicon may be a non-aromatic carbon. In other embodiments, a fluorine has been replaced by silicon derivative (e.g., —Si(CH₃)₃). In some embodiments, the silicon derivatives of the disclosure may include one or more hydrogen atoms replaced by deuterium. In some embodiments, a silicon derivative of compound, tautomer, deuterated derivative, or pharmaceutically acceptable salt chosen from Compounds 1 to 42 and Compounds I1 to I36 or compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, a tautomer thereof, a deuterated derivative of that compound or tautomer, or a pharmaceutically acceptable salt of any of the foregoing, may have silicon incorporated into a heterocycle ring.

In some embodiments, the derivative is a boron derivative in which one carbon atom in a compound, tautomer, deuterated derivative, or pharmaceutically acceptable salt chosen from Compounds 1 to 42 and Compounds I1 to I36 or compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, tautomers thereof, deuterated derivatives of those compounds or tautomers, and pharmaceutically acceptable salts of any of the foregoing, has been replaced by boron or a boron derivative.

In some embodiments, the derivative is a phosphorus derivative in which one carbon atom in a compound, tautomer, deuterated derivative, or pharmaceutically acceptable salt chosen from Compounds 1 to 42 and Compounds I1 to I36 or compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, tautomers thereof, deuterated derivatives of those compounds or tautomers, and pharmaceutically acceptable salts of any of the foregoing, has been replaced by phosphorus or a phosphorus derivative.

Another aspect of the disclosure provides pharmaceutical compositions comprising at least one compound, tautomer, deuterated derivative, or pharmaceutically acceptable salt according to any one formula chosen from Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC and Compounds 1 to 42 and Compounds 11 to 136, tautomers thereof, deuterated derivatives of those compounds or tautomers, and pharmaceutically acceptable salts of any of the foregoing. In some embodiments, the pharmaceutical composition comprising at least one compound, tautomer, deuterated derivative, or pharmaceutically acceptable salt chosen from Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC and Compounds 1 to 42 and Compounds I1 to I36, tautomers thereof, deuterated derivatives of those compounds or tautomers, and pharmaceutically acceptable salts of any of the foregoing is administered to a patient in need thereof.

A pharmaceutical composition may further comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include at least one additional active therapeutic agent. Alternatively, a pharmaceutical composition comprising at least one compound, tautomer, deuterated derivative, or pharmaceutically acceptable salt chosen from compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, tautomers thereof, deuterated derivatives of those compounds or tautomers, and pharmaceutically acceptable salts of any of the foregoing can be administered as a separate composition concurrently with, prior to, or subsequent to, a composition comprising at least one other active therapeutic agent. In some embodiments, a pharmaceutical composition comprising at least one compound, tautomer, deuterated derivative, or pharmaceutically acceptable salt chosen from Compounds 1 to 42 and Compounds I1 to I36, tautomers thereof, deuterated derivatives of those compounds or tautomers, and pharmaceutically acceptable salts of any of the foregoing can be administered as a separate composition concurrently with, prior to, or subsequent to, a composition comprising at least one other active therapeutic agent.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as, e.g., human serum albumin), buffer substances (such as, e.g., phosphates, glycine, sorbic acid, and potassium sorbate), partial glyceride mixtures of saturated vegetable fatty acids, water, salts, and electrolytes (such as, e.g., protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as, e.g., lactose, glucose, and sucrose), starches (such as, e.g., corn starch and potato starch), cellulose and its derivatives (such as, e.g., sodium carboxymethyl cellulose, ethyl cellulose, and cellulose acetate), powdered tragacanth, malt, gelatin, talc, excipients (such as, e.g., cocoa butter and suppository waxes), oils (such as, e.g., peanut oil, cottonseed oil, safflower oil, sesame oil, olive oil, corn oil, and soybean oil), glycols (such as, e.g., propylene glycol and polyethylene glycol), esters (such as, e.g., ethyl oleate and ethyl laurate), agar, buffering agents (such as, e.g., magnesium hydroxide and aluminum hydroxide), alginic acid, pyrogen-free water, isotonic saline, Ringer's solution, ethyl alcohol, phosphate buffer solutions, non-toxic compatible lubricants (such as, e.g., sodium lauryl sulfate and magnesium stearate), coloring agents, releasing agents, coating agents, sweetening agents, flavoring agents, perfuming agents, preservatives, and antioxidants.

In some embodiments of the disclosure, the compounds and the pharmaceutical compositions described herein are used to treat FSGS and/or NDKD. In some embodiments, FSGS is mediated by APOL1. In some embodiments, NDKD is mediated by APOL1.

In some embodiments of the disclosure, the compounds and the pharmaceutical compositions described herein are used to treat cancer. In some embodiments, the cancer is mediated by APOL1.

In some embodiments of the disclosure, the compounds and the pharmaceutical compositions described herein are used to treat pancreatic cancer. In some embodiments, the pancreatic cancer is mediated by APOL1.

In some embodiments, the methods of the disclosure comprise administering to a patient in need thereof at least one compound, tautomer, deuterated derivative, or pharmaceutically acceptable salt chosen from compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, tautomers thereof, deuterated derivatives of those compounds and tautomers, and pharmaceutically acceptable salts of any of the foregoing. In some embodiments, the compound, tautomer, deuterated derivative, or pharmaceutically acceptable salt is chosen from Compounds 1 to 42 and Compounds I1 to I36, tautomer thereof, deuterated derivatives of those compounds or tautomers, and pharmaceutically acceptable salts of any of the foregoing. In some embodiments, said patient in need thereof possesses APOL1 genetic variants, i.e., GI: S342G:1384M and G2: N388del:Y389del.

Another aspect of the disclosure provides methods of inhibiting APOL1 activity comprising contacting said APOL1 with at least one compound, tautomer, deuterated derivative, or pharmaceutically acceptable salt chosen from compounds of Formulae I, IA, IB, IC, ID, TI, IIA, IV, IVA, IVB, IVC, V, VA, VB, VC, VI, VIA, VIB, VIC, VII, VIIA, VIII, VIIIA, IX, IXA, IXB, or IXC, tautomers thereof, deuterated derivatives of those compounds or tautomers, and pharmaceutically acceptable salts of any of the foregoing. In some embodiments, the methods of inhibiting APOL1 activity comprise contacting said APOL1 with at least one compound, tautomer, deuterated derivative, or pharmaceutically acceptable salt chosen from Compounds 1 to 42 and Compounds I1 to I36, tautomers thereof, deuterated derivatives of those compounds or tautomers, and pharmaceutically acceptable salts of any of the foregoing.

### Non-Limiting Example Embodiments

Without limitation, some embodiments of the present disclosure include:
1. A compound represented by the following structural formula:

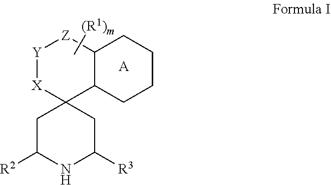

a tautomer thereof, a deuterated derivative of that compound or tautomer, or a pharmaceutically acceptable salt of any of the foregoing, wherein:
   Ring A is chosen from 6-membered aryl and 6-membered heteroaryl groups;
   X is chosen from —CH₂—, —C(O)—, —S(O)₂—, —NH—, and —O—;
   Y is chosen from —CH₂—, —C(O)—, —S(O)₂—, —NH—, and —O—;
   Z is chosen from a bond, —CH₂—, —NH—, —C(O)—, —S(O)₂—, and —O—, wherein:
      at least one of X and Y is chosen from —CH₂— and —C(O)—; and
      for each of X, Y, and Z, a hydrogen atom in each instance of —CH₂— or —NH— is optionally replaced by R¹;
   R¹, for each occurrence, is independently chosen from halogen, —OH, cyano, phenyl, C₁-C₆ alkyl, C₁-C₆ alkoxy, C₃-C₆ carbocyclyl, 4- to 6-membered heterocyclyl, —C(═O)OR^{c}, —C(═O)N(R^{c})₂, and —OS(═O)₂R^{c} groups, wherein:
      R^{c}, for each occurrence, is independently chosen from hydrogen, C₁-C₄ alkyl, and C₁-C₄ haloalkyl groups;
      the 4- to 6-membered heterocyclyl of R¹ comprises one heteroatom chosen from nitrogen and oxygen;
      the C₁-C₆ alkyl of R¹ is optionally substituted with 1 to 3 groups independently chosen from halogen, cyano, —OH, —NH₂, —NH(C₁-C₄ alkyl), —N(C₁-C₄ alkyl)₂, and C₁-C₄ alkoxy groups;
      the C₁-C₆ alkoxy of R¹ is optionally substituted with 1 to 3 groups independently chosen from —OH, cyano, and halogen groups;
      the C₃-C₆ carbocyclyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups; and
      the phenyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups;
   R² is chosen from cyano, C₁-C₆ alkyl, —C(═O)O(C₁-C₄ alkyl), C₂-C₆ alkynyl, and

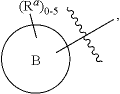

wherein:
   the C₁-C₆ alkyl of R² is optionally substituted with 1 to 3 groups independently chosen from halogen, cyano, —OH, —NH₂, —NH(C₁-C₄ alkyl), —N(C₁-C₄ alkyl)₂, C₁-C₄ alkoxy, —C(═O)NH₂, —C(═O)NH(C₁-C₄ alkyl), —C(═O)N(C₁-C₄ alkyl)₂, C₃-C₆ carbocyclyl, 5- to 10-membered heterocyclyl, C₆ aryl, and 5- to 10-membered heteroaryl groups;
   Ring B is chosen from C₃-C₁₂ carbocyclyl, 3- to 12-membered heterocyclyl, C₆ and C₁₀ aryl, and 5- to 10-membered heteroaryl groups, wherein Ring B is optionally substituted with 1, 2, 3, 4, or 5 R^{a} groups, wherein:
      R^{a}, for each occurrence, is independently chosen from halogen, cyano, C₁-C₆ alkyl, C₂-C₆ alkenyl, C₁-C₆ alkoxy, C₁-C₆ haloalkyl, C₁-C₆ haloalkenyl, C₁-C₆ haloalkoxy, —C(═O)NR^{h}Rⁱ, —NR^{h}i, —NR^{h}C(O)R^{k}, —NR^{h}C(O)OR^{k}, —NR^{h}C(═O)NRⁱR^{j}, —NR^{h}S(═O)ₚR^{k}, —OR^{k}, —OC(═O)R^{k}, —OC(═O)OR^{k}, —OC(═O)NR^{h}Rⁱ, —[O(CH₂)_{q}]ᵣO(C₁-C₆ alkyl), —S(═O)ₚR^{k}, —S(═O)ₚNR^{h}Rⁱ, —C(═O)OR^{k}, C₃-C₁₂ carbocyclyl, 3- to 12-membered heterocyclyl, C₆ and C₁₀ aryl, and 5- to 10-membered heteroaryl groups, wherein:
      the C₁-C₆ alkyl, C₁-C₆ alkoxy, and the C₂-C₆ alkenyl of R^{a} are each optionally substituted with 1 to 3 groups independently chosen from C₆ to C₁₀ aryl (optionally substituted with 1 to 3 R^{m} groups), 5- to 10-membered heterocyclyl (optionally substituted with 1 to 3 R^{m} groups), 5-to 10-membered heteroaryl (optionally substituted with 1 to 3 R^{m}groups), cyano, —C(═O)R^{k}, —C(═O)OR^{k}, —C(═O)NR^{h}Rⁱ, —NR^{h}Rⁱ, —NR^{h}C(═O)R^{k},
      —NR^{h}C(═O)OR^{k}, —NR^{h}C(═O)NRⁱR^{j}, —NR^{h}S(═O)ₚR^{k}, —OR^{k}, —OC(═O)R^{k}, —OC(═O)OR^{k}, —OC(═O)NR^{h}Rⁱ, —S(═O)ₚR^{k}, —S(═O)ₚNR^{h}Rⁱ, —O(C₆ aryl) (optionally substituted with 1 to 3 R^{m} groups), and C₃-C₆ carbocyclyl groups (optionally substituted with 1 to 3 R^{m} groups);
      the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from halogen, cyano, C₁-C₄ alkyl, —NR^{h}Rⁱ, and —OR^{k} groups, wherein:
      R^{h},Rⁱ, and R^{j}, for each occurrence, are each independently chosen from hydrogen, C₁-C₄ alkyl, C₆-C₁₀ aryl, and C₃-C₆ cycloalkyl groups, wherein:
      the C₁-C₄ alkyl of any one of R^{h}, Rⁱ, and R^{j} is optionally substituted with 1 to 3 groups independently chosen from halogen, cyano, and —OH groups;
      R^{k}, for each occurrence, is independently chosen from hydrogen, C₁-C₄ alkyl, 5- to 10-membered heterocyclyl, and C₃-C₆ carbocyclyl groups, wherein:
      the C₁-C₄ alkyl of any one of R^{k} is optionally substituted with 1 to 3 groups independently chosen from halogen, cyano, and —OH groups;
      R^{k}, for each occurrence, is independently chosen from halogen, cyano, oxo, C₁-C₆ alkyl, C₁-C₆ alkoxy, —S(═O)ₚR^{k}, and —OR^{k} groups, wherein:
      the C₁-C₆ alkyl of R^{m} is optionally substituted with 1 to 3 groups independently chosen from halogen, cyano, —OH, and —O(C₁-C₄ alkyl) groups;
R³ is chosen from C₁-C₆ alkyl, —C(═O)O(C₁-C₄ alkyl), C₃-C₁₂ carbocyclyl, 3- to 12-membered heterocyclyl, C₆ and C₁₀ aryl, and 5- to 10-membered heteroaryl groups, wherein:
   the C₁-C₆ alkyl of R³ is optionally substituted with 1 to 3 groups independently chosen from halogen, cyano, —OH, —NH₂, —NH(C₁-C₄ alkyl), —N(C₁-C₄ alkyl)₂, C₁-C₄ alkoxy, —C(═O)NH₂, —C(═O)NH(C₁-C₄ alkyl), and —C(═O)N(C₁-C₄ alkyl)₂ groups;
   the C₃-C₁₂ carbocyclyl, the 3- to 12-membered heterocyclyl, the C₆ and C₁₀ aryl, and the 5- to 10-membered heteroaryl of R³ are each optionally substituted with 1 to 3 groups independently chosen from halogen, cyano, —OH, —NH₂, —NH(C₁-C₄ alkyl) (optionally substituted with —OH), —N(C₁-C₄ alkyl)₂, C₁-C₅ alkyl (optionally substituted with —OH or —S(═O)₂(C₁-C₄ alkyl)), C₁-C₄ alkoxy, —C(═O)NH₂, —C(═O)NH(C₁-C₄ alkyl), —NHC(═O)(C₁-C₄ alkyl), —C(═O)(C₁-C₄ alkoxy), and —C(═O)N(C₁-C₄ alkyl)₂ groups;
m is an integer chosen from 0, 1, 2, 3, 4, and 5;
p, for each occurrence, is an integer independently chosen from 1 and 2; and
q and r, for each occurrence, are each an integer independently chosen from 1, 2, 3, and 4.
2. The compound, tautomer, deuterated derivative, or pharmaceutically acceptable salt according to Embodiment 1, wherein:
   Ring A is chosen from 6-membered aryl and 6-membered heteroaryl groups;
   X is chosen from —CH₂—, —C(O)—, —S(O)₂—, —NH—, and —O—;
   Y is chosen from —CH₂—, —C(O)—, —S(O)₂—, —NH—, and —O—;
   Z is chosen from a bond, —CH₂—, —NH—, —C(O)—, —S(O)₂—, and —O—, wherein:
      at least one of X and Y is chosen from —CH₂— and —C(O)—; and
      for each of X, Y, and Z, a hydrogen atom in each instance of —CH₂— or —NH— is optionally replaced by R¹;
   R¹, for each occurrence, is independently chosen from halogen, —OH, cyano, phenyl, C₁-C₆ alkyl, C₁-C₆ alkoxy, —C(═O)OR^{c}, —C(═O)N(R^{c})₂, and —OS(═O)₂R^{c} groups, wherein:
      R^{c}, for each occurrence, is independently chosen from hydrogen, C₁-C₄ alkyl, and C₁-C₄ haloalkyl groups;
      the C₁-C₆ alkyl of R¹ is optionally substituted with 1 to 3 groups independently chosen from halogen, cyano, —OH, —NH₂, —NH(C₁-C₄ alkyl), —N(C₁-C₄ alkyl)₂, and C₁-C₄ alkoxy groups;
      the C₁-C₆ alkoxy of R¹ is optionally substituted with 1 to 3 groups independently chosen from —OH, cyano, and halogen groups;
      the phenyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups;
   R² is chosen from C₁-C₆ alkyl and

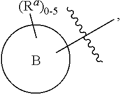

wherein:
   the C₁-C₆ alkyl of R² is optionally substituted with 1 to 3 groups independently chosen from halogen, cyano, —OH, —NH₂, —NH(C₁-C₄ alkyl), —N(C₁-C₄ alkyl)₂, C₁-C₄ alkoxy, —C(═O)NH₂, —C(═O)NH(C₁-C₄ alkyl), —C(═O)N(C₁-C₄ alkyl)₂, C₃-C₆ carbocyclyl, 5- to 10-membered heterocyclyl, C₆ aryl, and 5- to 10-membered heteroaryl groups;
   Ring B is chosen from 3- to 12-membered heterocyclyl, C₆ aryl, and 5- to 10-membered heteroaryl groups, wherein Ring B is optionally substituted with 1, 2, 3, 4, or 5 R^{a} groups, wherein:
      R^{a}, for each occurrence, is independently chosen from halogen, cyano, C₁-C₆ alkyl, C₂-C₆ alkenyl, C₁-C₆ alkoxy, C₁-C₆ haloalkyl, C₁-C₆ haloalkenyl, C₁-C₆ haloalkoxy, —C(═O)NR^{h}Rⁱ, —NR^{h}Rⁱ, —NR^{h}C(═O)R^{k}, —NR^{h}C(═O)OR^{k}, —NR^{h}C(═O)NRⁱR^{j}, —NR^{h}S(═O)ₚR^{k}, —OR^{k}, —OC(═O)R^{k}, —OC(═O)OR^{k}, —OC(═O)NR^{h}Rⁱ, —[O(CH₂)_{q}]ᵣO(C₁-C₆ alkyl), —S(═O)ₚR^{k}, —S(═O)ₚNR^{h}Rⁱ, —C(═O)OR^{k}, C₃-C₁₂ carbocyclyl, 3- to 12-membered heterocyclyl, C₆ and C₁₀ aryl, and 5- to 10-membered heteroaryl groups, wherein:
      the C₁-C₆ alkyl, C₁-C₆ alkoxy, and the C₂-C₆ alkenyl of R^{a} are each optionally substituted with 1 to 3 groups independently chosen from C₆ to C₁₀ aryl (optionally substituted with 1 to 3 R^{m} groups), 5- to 10-membered heterocyclyl (optionally substituted with 1 to 3 R^{m} groups), 5-to 10-membered heteroaryl (optionally substituted with 1 to 3 R^{m}groups), cyano, —C(═O)R^{k}, —C(═O)OR^{k}, —C(═O)NR^{h}Rⁱ, —NR^{h}Rⁱ, —NR^{h}C(═O)R^{k},
      —NR^{h}C(═O)OR^{k}, —NR^{h}C(═O)NRⁱR^{j}, —NR^{h}S(═O)ₚR^{k}, —OR^{k}, —OC(═O)R^{k}, —OC(═O)OR^{k}, —OC(═O)NR^{h}Rⁱ, —S(═O)ₚR^{k}, —S(═O)ₚNR^{h}Rⁱ, and C₃-C₆ carbocyclyl groups (optionally substituted with 1 to 3 R^{m} groups);
      the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from halogen, cyano, C₁-C₄ alkyl, —NR^{h}Rⁱ, and —OR^{k} groups, wherein:
      R^{h}, Rⁱ, and R^{j}, for each occurrence, are each independently chosen from hydrogen, C₁-C₄ alkyl, C₆-C₁₀ aryl, and C₃-C₆ cycloalkyl groups, wherein:
      the C₁-C₄ alkyl of any one of R^{h}, Rⁱ and R^{j} is optionally substituted with 1 to 3 groups independently chosen from halogen, cyano, and —OH groups;
      R^{k}, for each occurrence, is independently chosen from hydrogen, C₁-C₄ alkyl, 5- to 10-membered heterocyclyl, and C₃-C₆ carbocyclyl groups, wherein:
      the C₁-C₄ alkyl of any one of R^{k} is optionally substituted with 1 to 3 groups independently chosen from halogen, cyano, and —OH groups;
      R^{m}, for each occurrence, is independently chosen from halogen, cyano, oxo, C₁-C₆ alkyl, C₁-C₆ alkoxy, —S(═O)ₚR^{k}, and —OR^{k} groups, wherein:
      the C₁-C₆ alkyl of R^{m} is optionally substituted with 1 to 3 groups independently chosen from halogen, cyano, and —OH groups;
R³ is chosen from C₁-C₆ alkyl groups, wherein:
   the C₁-C₆ alkyl of R³ is optionally substituted with 1 to 3 groups independently chosen from halogen, cyano, —OH, —NH₂, —NH(C₁-C₄ alkyl), —N(C₁-C₄ alkyl)₂, C₁-C₄ alkoxy, —C(═O)NH₂, —C(═O)NH(C₁-C₄ alkyl), and —C(═O)N(C₁-C₄ alkyl)₂ groups;
m is an integer chosen from 0, 1, 2, and 3;
p, for each occurrence, is an integer independently chosen from 1 and 2; and
q and r, for each occurrence, are each an integer independently chosen from 1, 2, 3, and 4.
3. The compound, tautomer, deuterated derivative, or pharmaceutically acceptable salt according to Embodiment 1 or 2, wherein:
   Ring A is chosen from 6-membered aryl and 6-membered heteroaryl groups;
   X is chosen from —CH₂—, —C(O)—, —S(O)₂—, —NH—, and —O—;
   Y is chosen from —CH₂—, —C(O)—, —S(O)₂—, —NH—, and —O—;
   Z is chosen from a bond, —CH₂—, —NH—, —C(O)—, —S(O)₂—, and —O—, wherein:
      at least one of X and Y is chosen from —CH₂— and —C(O)—; and
      for each of X, Y, and Z, a hydrogen atom in each instance of —CH₂— or —NH— is optionally replaced by R¹;
   R¹, for each occurrence, is independently chosen from halogen, —OH, cyano, C₁-C₄ alkyl, C₁-C₄ alkoxy, —C(═O)OR^{c}, —C(═O)N(R^{c})₂, and —OS(═O)₂R^{c} groups, wherein:
      R^{c}, for each occurrence, is independently chosen from hydrogen, C₁-C₄ alkyl, and C₁-C₄ haloalkyl groups;
      the C₁-C₆ alkyl of R¹ is optionally substituted with 1 to 3 groups independently chosen from halogen and —OH groups;
      R² is

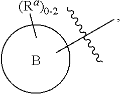

wherein:
Ring B is chosen from 5-membered heterocyclyl and 5-membered heteroaryl groups, wherein Ring B is optionally substituted with 1 or 2 R^{a} groups, wherein:
   R^{a}, for each occurrence, is independently chosen from C₁-C₆ alkyl groups optionally substituted with 1 group independently chosen from —S(═O)ₚR^{k} groups, wherein:
   R^{k}, for each occurrence, is independently chosen from C₁-C₄ alkyl groups;
R³ is chosen from C₁-C₃ alkyl groups;
m is an integer chosen from 0, 1, 2, and 3; and
p, for each occurrence, is an integer independently chosen from 1 and 2.
4. The compound, tautomer, deuterated derivative, or pharmaceutically acceptable salt according to any one of Embodiments 1-3, wherein:
   Ring A is chosen from 6-membered aryl and 6-membered heteroaryl groups;
   X is chosen from —CH₂—, —C(O)—, —S(O)₂—, —NH—, and —O—;
   Y is chosen from —CH₂—, —C(O)—, —S(O)₂—, —NH—, and —O—;
   Z is chosen from a bond, —CH₂—, —NH—, —C(O)—, —S(O)₂—, and —O—, wherein:
      at least one of X and Y is chosen from —CH₂— and —C(O)—; and
      for each of X, Y, and Z, a hydrogen atom in each instance of —CH₂— or —NH— is optionally replaced by R¹;
   R¹, for each occurrence, is independently chosen from halogen, —OH, cyano, C₁-C₄ alkyl, C₁-C₄ alkoxy, —C(═O)OR^{c}, —C(═O)N(R^{c})₂, and —OS(═O)₂R^{c} groups, wherein:
      R¹, for each occurrence, is independently chosen from hydrogen, C₁-C₄ alkyl, and C₁-C₄ haloalkyl groups;
      the C₁-C₆ alkyl of R¹ is optionally substituted with 1 to 3 groups independently chosen from halogen and —OH groups;
      R² is

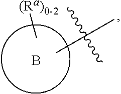

wherein:
Ring B is chosen from pyrazole and triazole groups, wherein Ring B is optionally substituted with 1 or 2 R^{a} groups, wherein:
   R^{a}, for each occurrence, is independently chosen from C₁-C₆ alkyl groups optionally substituted with 1 group independently chosen from —S(═O)ₚR^{k} groups, wherein:
   R^{k}, for each occurrence, is independently chosen from C₁-C₄ alkyl groups;
R³ is methyl;
m is an integer chosen from 0, 1, 2, and 3; and
p, for each occurrence, is an integer independently chosen from 1 and 2.
5. The compound, tautomer, deuterated derivative, or pharmaceutically acceptable salt according to any one of Embodiments 1-4, wherein Ring A is chosen from phenyl, pyrimidinyl, and pyridinyl, and all other variables not specifically defined herein are as defined in any one of Embodiments 1-4.
6. The compound, tautomer, deuterated derivative, or pharmaceutically acceptable salt according to any one of Embodiments 1-4, wherein Ring A is phenyl, and all other variables not specifically defined herein are as defined in any one of Embodiments 1-4.
7. The compound, tautomer, deuterated derivative, or pharmaceutically acceptable salt according to Embodiment 1, 5, or 6, wherein R¹, for each occurrence, is independently chosen from hydrogen, halogen, cyano, —OH, C₁-C₄ alkyl, C₁-C₄ alkoxy, —C(═O)N(R^{c})₂, and C₃-C₆ cycloalkyl groups, wherein:
   R^{c}, for each occurrence, is independently chosen from hydrogen and C₁-C₂ alkyl groups;
   the C₁-C₄ alkyl of R¹ is optionally substituted with 1 to 3 groups independently chosen from halogen, cyano, —OH, and C₁-C₂ alkoxy groups;
   the C₁-C₄ alkoxy of R¹ is optionally substituted with 1 to 3 independently chosen from halogen groups; and
   the C₃-C₆ cycloalkyl of R¹ is optionally substituted with 1 to 3 groups independently chosen from halogen, cyano, —OH, and C₁-C₂ alkoxy groups;
and all other variables not specifically defined herein are as defined in Embodiment 1, 5, or 6.
8. The compound, tautomer, deuterated derivative, or pharmaceutically acceptable salt according to Embodiment 1, 5, or 6, wherein R¹, for each occurrence, is independently chosen from F, Cl, Br, C₁-C₄ alkyl, C₁-C₄ alkoxy, —C(═O)N(R^{c})₂, and C₃-C₆ cycloalkyl groups, wherein:
   R^{c}, for each occurrence, is independently chosen from hydrogen and C₁-C₂ alkyl groups;
   the C₁-C₄ alkyl of R¹ is optionally substituted with 1 to 3 groups independently chosen from halogen and —OH;
   the C₁-C₄ alkoxy of R¹ is optionally substituted with 1 to 3 independently chosen from halogen groups; and
   the C₃-C₆ cycloalkyl of R¹ is optionally substituted with 1 to 3 groups independently chosen from halogen and —OH;
and all other variables not specifically defined herein are as defined in Embodiment 1, 5, or 6.
9. The compound, tautomer, deuterated derivative, or pharmaceutically acceptable salt according to Embodiment 1, 5, or 6, wherein R¹, for each occurrence, is independently chosen from F, Cl, Br, C₁-C₄ alkyl, C₁-C₄ alkoxy, —C(═O)N(R^{c})₂, and C₃-C₆ cycloalkyl groups, wherein:
   R^{c}, for each occurrence, is independently chosen from hydrogen and C₁-C₂ alkyl groups;
   the C₁-C₄ alkyl of R¹ is optionally substituted with 1 to 3 groups independently chosen from halogen and —OH; and
   the C₁-C₄ alkoxy of R¹ is optionally substituted with 1 to 3 independently chosen from halogen groups;
and all other variables not specifically defined herein are as defined in Embodiment 1, 5, or 6.
10. The compound, tautomer, deuterated derivative, or pharmaceutically acceptable salt according to Embodiment 1, 5, or 6, wherein R¹, for each occurrence, is independently chosen from F, Cl, Br, —CH₃, —CH(CH₃)₂, —CF₃, —OCH₃, —OCF₃, —C(═O)N(CH₃)₂, and cyclopropyl; and all other variables not specifically defined herein are as defined in Embodiment 1, 5, or 6.
11. The compound, tautomer, deuterated derivative, or pharmaceutically acceptable salt according to any one of Embodiments 1-10, wherein m is 1; and all other variables not specifically defined herein are as defined in any one of Embodiments 1-10.
12. The compound, tautomer, deuterated derivative, or pharmaceutically acceptable salt according to any one of Embodiments 1-10, wherein m is 2; and all other variables not specifically defined herein are as defined in any one of Embodiments 1-10.
13. The compound, tautomer, deuterated derivative, or pharmaceutically acceptable salt according to any one of Embodiments 1 and 5-12, wherein Ring B is chosen from cyclopropyl, 5- to 10-membered heterocyclyl, phenyl, and 5- to 9-membered heteroaryl groups; each of which is optionally substituted with 1, 2, 3, 4, or 5 R^{a} groups; and all other variables not specifically defined herein are as defined in any one of Embodiments 1 and 5-12.
14. The compound, tautomer, deuterated derivative, or pharmaceutically acceptable salt according to any one of Embodiments 1 and 5-12, wherein Ring B is chosen from cyclopropyl, 5- to 10-membered heterocyclyl comprising 1 to 3 heteroatoms chosen from N and O, phenyl, and 5- to 9-membered heteroaryl comprising 1 to 3 heteroatoms chosen from N and O; each of which is optionally substituted with 1, 2, 3, 4, or 5 R^{a} groups; and all other variables not specifically defined herein are as defined in any one Embodiments of 1 and 5-12.
15. The compound, tautomer, deuterated derivative, or pharmaceutically acceptable salt according to any one of Embodiments 1 and 5-12, wherein Ring B is chosen from cyclopropyl, 5-membered heterocyclyl comprising 1 to 3 heteroatoms chosen from N and O, 6-membered heterocyclyl comprising 1 to 3 heteroatoms chosen from N and O, 9-membered heterocyclyl comprising 1 to 3 heteroatoms chosen from N and O, 10-membered heterocyclyl comprising 1 to 3 heteroatoms chosen from N and O, phenyl, 5-membered heteroaryl comprising 1 to 3 heteroatoms chosen from N and O, 6-membered heteroaryl comprising 1 to 3 heteroatoms chosen from N and O, and 9-membered heteroaryl comprising 1 to 3 heteroatoms chosen from N and O; each of which is optionally substituted with 1, 2, 3, 4, or 5 R^{a} groups; and all other variables not specifically defined herein are as defined in any one of Embodiments 1 and 5-12.
16. The compound, tautomer, deuterated derivative, or pharmaceutically acceptable salt according to any one of Embodiments 1 and 5-12, wherein Ring B is chosen from

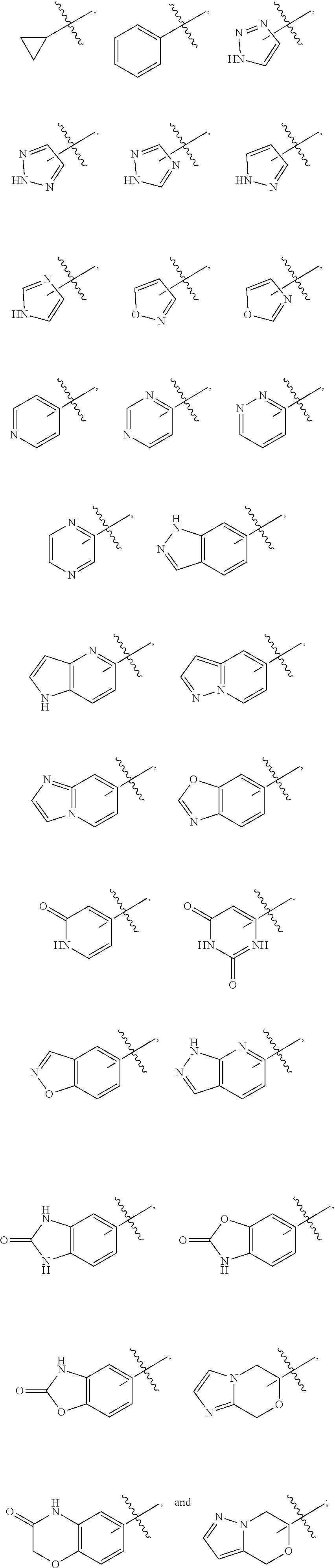

each of which is optionally substituted with 1, 2, 3, 4, or 5 R^{a} groups;
and all other variables not specifically defined herein are as defined in any one of Embodiments 1 and 5-12.
17. The compound, tautomer, deuterated derivative, or pharmaceutically acceptable salt according to any one of Embodiments 1 and 5-12, wherein Ring B is chosen from

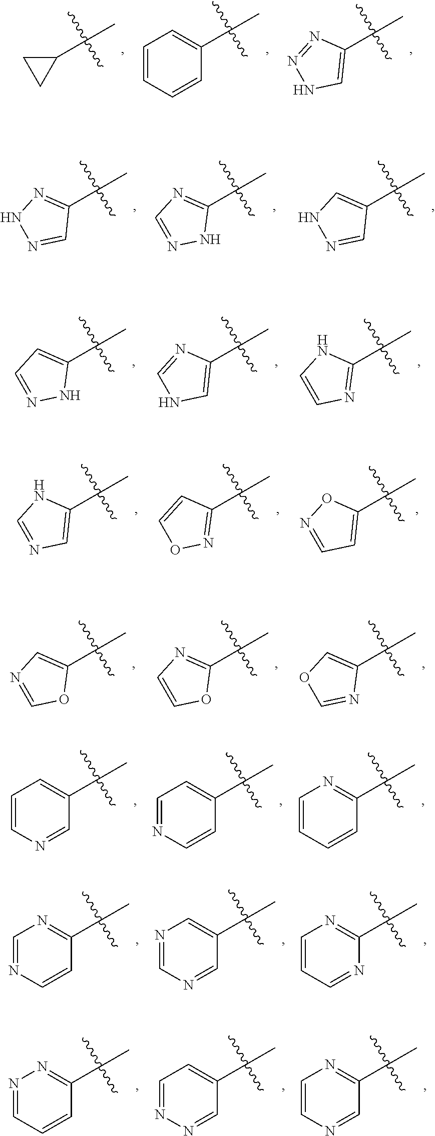

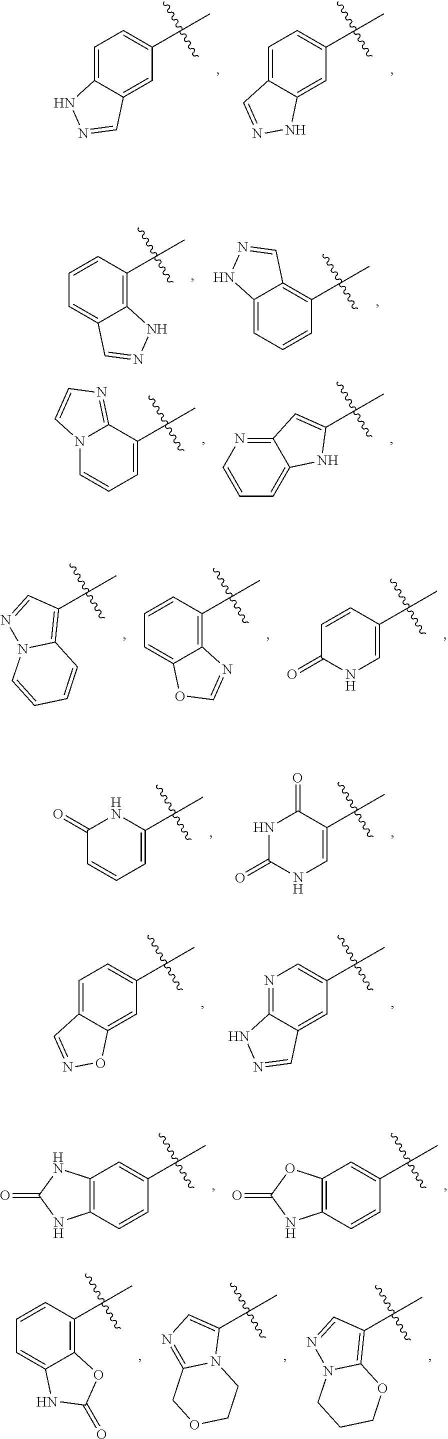

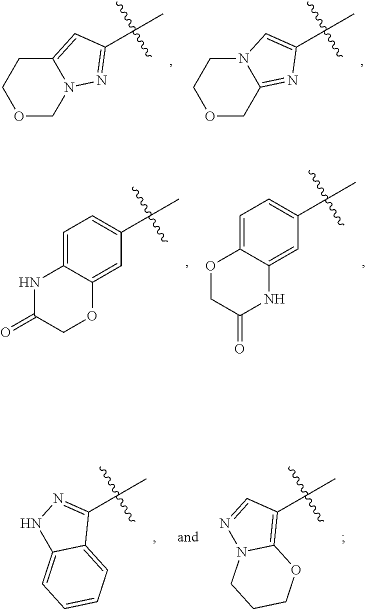 [SMILES: *c1cc2n(n1)COCC2 ; *c1cn2c(n1)COCC2 ; *c1ccc2c(c1)OCC(=O)N2 ; *c1ccc2c(c1)NC(=O)CO2 ; *c1n[nH]c2ccccc12 ; *c1cnn2c1OCCC2]

each of which is optionally substituted with 1, 2, 3, 4, or 5 R^{a} groups; and all other variables not specifically defined herein are as defined in any one of Embodiments 1 and 5-12.
18. The compound, tautomer, deuterated derivative, or pharmaceutically acceptable salt according to any one of Embodiments 1 and 5-12, wherein Ring B is

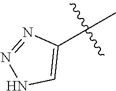 [SMILES: *c1c[nH]nn1]

which is optionally substituted with 1 R^{a} group; and all other variables not specifically defined herein are as defined in any one of Embodiments 1 and 5-12.
19. The compound, tautomer, deuterated derivative, or pharmaceutically acceptable salt according to any one of Embodiments 13-18, wherein R^{a}, for each occurrence, is independently chosen from halogen, cyano, C₁-C₆ alkyl, C₁-C₄ alkoxy, C₁-C₆ haloalkyl, C₁-C₆ haloalkoxy, —C(═O)NR^{h}Rⁱ, —NR^{h}Rⁱ, —NR^{h}C(═O)R^{k}, OR^{k}, —[O(CH₂)_{q}]ᵣO(C₁-C₆ alkyl), —S(═O)₂R^{k}, —S(═O)₂NR^{h}Rⁱ, C₃-C₆ cycloalkyl, 5 to 10-membered heterocyclyl, phenyl, and 5- to 8-membered heteroaryl groups, wherein:
   the C₁-C₆ alkyl of R^{a} is optionally substituted with 1 to 3 groups independently chosen from cyano, —C(═O)NR^{h}Rⁱ, —NR^{h}Rⁱ, —NR^{h}C(═O)R^{k}, —NR^{h}C(═O)OR^{k}, —NR^{h}C(═O)NRⁱR^{j}, —NR^{h}S(═O)ₚR^{k}, —OR^{k}, —S(═O)₂R^{k}, —S(═O)ₚNR^{h}Rⁱ, and C₃-C₆ cycloalkyl groups;
   the C₃-C₆ cycloalkyl, the 5- to 10-membered heterocyclyl, the phenyl, and the 5- to 8-membered heteroaryl of R^{a} are each optionally substituted with 1 to 3 groups independently chosen from halogen, C₁-C₂ alkyl, and —OR^{k} groups, wherein:
   R^{h}, Rⁱ, and R^{j}, for each occurrence, are each independently chosen from hydrogen, C₁-C₂ alkyl, cyclopropyl, and cyclobutyl groups, wherein:
   the C₁-C₂ alkyl of any one of R^{h}, Rⁱ, and R^{j} is optionally substituted with 1 to 3 groups independently chosen from halogen and —OH;
   R^{k}, for each occurrence, is each independently chosen from hydrogen and C₁-C₄ alkyl groups, wherein:
   the C₁-C₄ alkyl of R^{k} is optionally substituted with 1 to 3 groups independently chosen from halogen and —OH; and
   q and r are each an integer chosen from 1, 2, and 3;
and all other variables not specifically defined herein are as defined in any one of Embodiments 13-18.
20. The compound, tautomer, deuterated derivative, or pharmaceutically acceptable salt according to any one of Embodiments 13-18, wherein R^{a}, for each occurrence, is independently chosen from halogen, cyano, C₁-C₆ alkyl, C₁-C₄ alkoxy, C₁-C₄ haloalkyl, C₁-C₄ haloalkoxy, —C(═O)NR^{h}Rⁱ, —NR^{h}Rⁱ, —NR^{h}C(═O)R^{k}, OR^{k}, —[O(CH₂)_{q}]ᵣO(C₁-C₄ alkyl), —S(═O)₂R^{k}, —S(═O)₂NR^{h}Rⁱ, cyclopropyl, cyclobutyl, 5- to 6-membered heterocyclyl, phenyl, and 5- to 6-membered heteroaryl, wherein:
   the C₁-C₆ alkyl of R^{a} is optionally substituted with 1 to 3 groups independently chosen from cyano, —C(═O)NR^{h}Rⁱ, —S(═O)₂R^{k}, —NR^{h}Rⁱ, —OR^{k}, cyclopropyl, and cyclobutyl groups, wherein:
   the cyclopropyl, the cyclobutyl, the 5- to 6-membered heterocyclyl, the phenyl, and the 5 to 6-membered heteroaryl of R^{a} are each optionally substituted with 1 to 3 groups independently chosen from halogen, —CH₃, —OH, and —OCH₃; wherein:
   R^{h} and Rⁱ, for each occurrence, are each independently chosen from hydrogen, —CH₃, cyclopropyl, and cyclobutyl groups, wherein:
   the —CH₃ of any one of R^{h} and Rⁱ is optionally substituted with 1 to 3 groups independently chosen from F, Cl, and —OH;
   R^{k}, for each occurrence, is each independently chosen from hydrogen and —CH₃, wherein:
   the —CH₃ of R^{k} is optionally substituted with 1 to 3 groups independently chosen from halogen and —OH;
and all other variables not specifically defined herein are as defined in any one of Embodiments 13-18.
21. The compound, tautomer, deuterated derivative, or pharmaceutically acceptable salt according to any one of Embodiments 13-18, wherein R^{a}, for each occurrence, is independently chosen from F, Cl, Br, cyano, C₁-C₆ alkyl, C₁-C₂ alkoxy, C₁-C₂ haloalkyl, —C(═O)NR^{h}Rⁱ, —NR^{h}Rⁱ,
—NR^{h}C(═O)R^{k}, —OR^{k}, —[O(CH₂)_{q}]ᵣO(C₁-C₂ alkyl), —S(═O)₂R^{k}, —S(═O)₂NR^{h}Rⁱ, cyclopropyl, cyclobutyl, 5-membered heterocyclyl, phenyl, and 6-membered heteroaryl groups, wherein:
   the C₁-C₆ alkyl of R^{a} is optionally substituted with 1 to 3 groups independently chosen from cyano, —C(═O)NR^{h}Rⁱ, —OR^{k}, —S(═O)₂R^{k}, and cyclopropyl;
   the cyclopropyl, the cyclobutyl, the 5- to 6-membered heterocyclyl, the phenyl, and the 5- to 6-membered heteroaryl of R^{a} are each optionally substituted with 1 to 3 groups independently chosen from halogen, —CH₃, —OH, and —OCH₃, wherein:
   R^{h} and Rⁱ, for each occurrence, are each independently chosen from hydrogen, —CH₃, and cyclopropyl; wherein:
   the —CH₃ of any one of R^{h} and i is optionally substituted with 1 to 3 groups independently chosen from F, Cl, and —OH;
   R^{k}, for each occurrence, is each independently chosen from hydrogen and —CH₃; and
   q and r are each an integer independently chosen from 1 and 2;
and all other variables not specifically defined herein are as defined in any one of Embodiments 13-18.
22. The compound, tautomer, deuterated derivative, or pharmaceutically acceptable salt according to any one of Embodiments 13-18, wherein R^{a}, for each occurrence, is independently chosen from F, cyano, —OH, —CH₃, —CF₃, —CH(CH₃)₂, —(CH₂)₂OH, —(CH₂)₂OCH₃,
—CH₂CH(OH)C₂H5, —CH₂C(CH₃)(CH₂OH)₂, —OCH₃, —OCH₂CH₃, —O(CH₂)₂]₂OCH₃, —CH₂C(═O)NHCH₃, —(CH₂)₂SO₂CH₃, —CH₂C(═O)N(CH₃)₂, —CH₂(cyclopropyl), —C(═O)NH₂, —C(═O)NH(cyclopropyl), —NH₂, —NHCH₃, —N(CH₃)₂, —NHC(CH₃)₂CH₂OH, —NHC(═O)CH₃, —SO₂CH₃, —SO₂NH₂, cyclopropyl, 2-methoxyphenyl, N-methylpiperazinyl, tetrahydro-2H-pyranyl, methylpyrazolyl, pyridinyl, and tetrahydrothiophenyl 1,1-dioxide; and all other variables not specifically defined herein are as defined in any one of Embodiments 13-18.
23. The compound, tautomer, deuterated derivative, or pharmaceutically acceptable salt according to any one of Embodiments 13-18, wherein R^{a}, for each occurrence, is independently chosen from —CH₃ and —(CH₂)₂SO₂CH₃; and all other variables not specifically defined herein are as defined in any one of Embodiments 13-18.
24. The compound, tautomer, deuterated derivative, or pharmaceutically acceptable salt according to Embodiment 1, wherein the compound is represented by the following structural formula:

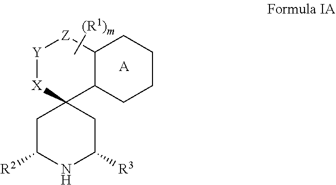

a tautomer thereof, a deuterated derivative of that compound or tautomer, or a pharmaceutically acceptable salt of any of the foregoing, wherein all variables not specifically defined herein are as defined in any one of Embodiments 1-23.
25. The compound, tautomer, deuterated derivative, or pharmaceutically acceptable salt according to Embodiment 1 or 24, wherein Z is chosen from —CH₂—, —NH—, —C(O)—, —S(O)₂—, and —O—; and all other variables not specifically defined herein are as defined in Embodiment 1 or 24.
26. The compound, tautomer, deuterated derivative, or pharmaceutically acceptable salt according to Embodiment 1, wherein the compound is represented by one of the following structural formulae:

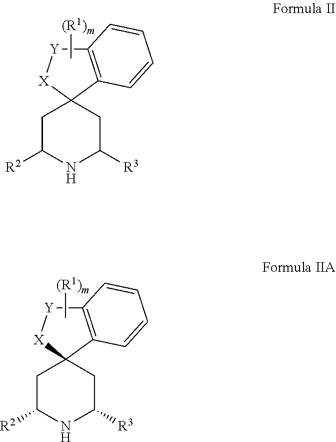

a tautomer thereof, a deuterated derivative of that compound or tautomer, or a pharmaceutically acceptable salt of any of the foregoing, wherein all variables not specifically defined herein are as defined in any one of Embodiments 1-4.
27. The compound, tautomer, deuterated derivative, or pharmaceutically acceptable salt according to Embodiment 1, wherein the compound is represented by one of the following structural formulae:

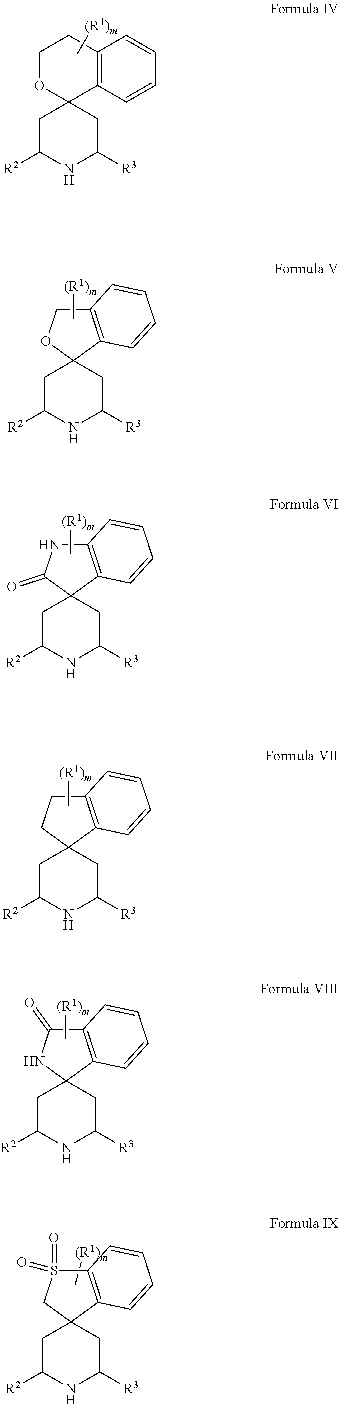

a tautomer thereof, a deuterated derivative of that compound or tautomer, or a pharmaceutically acceptable salt of any of the foregoing, wherein all variables not specifically defined herein are as defined in any one of Embodiments 1-4.
28. The compound, tautomer, deuterated derivative, or pharmaceutically acceptable salt according to Embodiment 1, wherein the compound is represented by one of the following structural formulae:

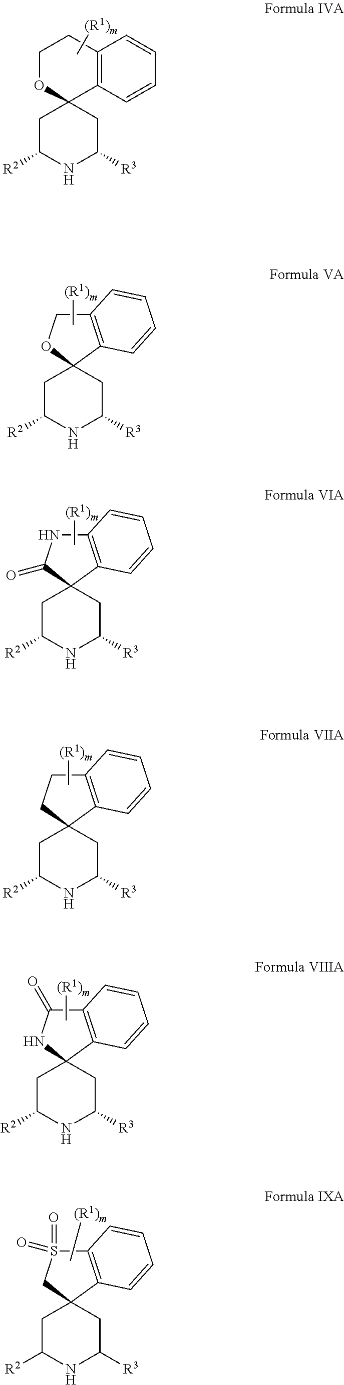

a tautomer thereof, a deuterated derivative of that compound or tautomer, or a pharmaceutically acceptable salt of any of the foregoing, wherein all variables not specifically defined herein are as defined in any one of Embodiments 1-4.
29. The compound, tautomer, deuterated derivative, or pharmaceutically acceptable salt according to Embodiment 1, wherein the compound is represented by one of the following structural formulae:

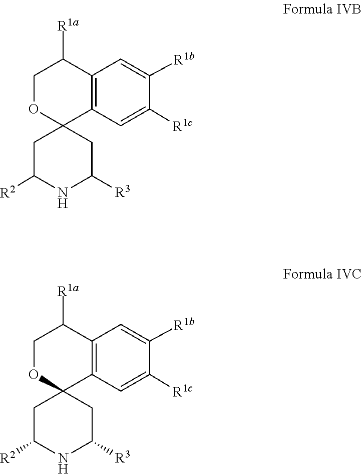

a tautomer thereof, a deuterated derivative of that compound or tautomer, or a pharmaceutically acceptable salt of any of the foregoing, wherein:
   R^{1a} is chosen from hydrogen, halogen, —OH, and phenyl groups, wherein:
   the phenyl of R^{1a} is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups;
   R^{1b} and R^{1c} are each independently chosen from hydrogen, halogen, —OH, cyano, C₁-C₄ alkyl, C₁-C₄ alkoxy, —C(═O)OR^{c}, —C(═O)N(R^{c})₂, and —OS(═O)₂R^{c} groups, wherein:
   R^{c}, for each occurrence, is independently chosen from hydrogen, C₁-C₄ alkyl, and C₁-C₄ haloalkyl groups; and
   the C₁-C₆ alkyl of R^{1b} and/or R^{1c} is optionally substituted with 1 to 3 groups independently chosen from halogen and —OH groups; and
   all variables not specifically defined herein are as defined in any one of Embodiments 1-4.
30. The compound, tautomer, deuterated derivative, or pharmaceutically acceptable salt according to Embodiment 1, wherein the compound is represented by one of the following structural formulae:

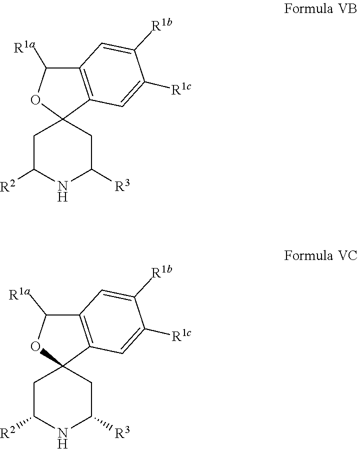

a tautomer thereof, a deuterated derivative of that compound or tautomer, or a pharmaceutically acceptable salt of any of the foregoing, wherein:
   R^{1a} is chosen from hydrogen, phenyl, and C(═O)N(R^{C}i)₂ groups, wherein:
      the phenyl of R^{a} is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy,
   —C(═O)NH₂, —C(═O)NH(C₁-C₄ alkyl), and —C(═O)N(C₁-C₄ alkyl)₂ groups;
   R^{1c}, for each occurrence, is independently chosen from hydrogen and C₁-C₄ alkyl groups;
   R^{1b} and Rid are each independently chosen from hydrogen and halogen groups; and
   all variables not specifically defined herein are as defined in any one of Embodiments 1-4.
31. The compound, tautomer, deuterated derivative, or pharmaceutically acceptable salt according to Embodiment 1, wherein the compound is represented by one of the following structural formulae:

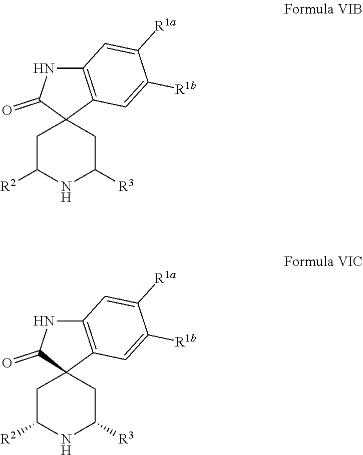

a tautomer thereof, a deuterated derivative of that compound or tautomer, or a pharmaceutically acceptable salt of any of the foregoing, wherein:
   R^{1a} and R^{1b} are each independently chosen from hydrogen, halogen, C₁-C₄ alkyl, and C₁-C₄ haloalkyl groups; and
   all variables not specifically defined herein are as defined in any one of Embodiments 1-4.
32. The compound, tautomer, deuterated derivative, or pharmaceutically acceptable salt according to Embodiment 1, wherein the compound is represented by one of the following structural formulae:

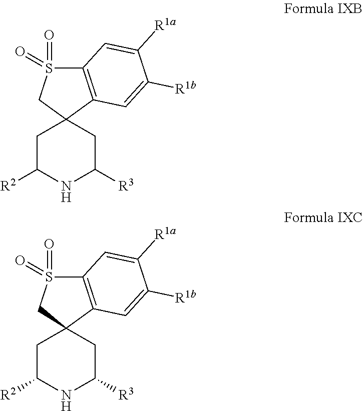

a tautomer thereof, a deuterated derivative of that compound or tautomer, or a pharmaceutically acceptable salt of any of the foregoing, wherein:
   R^{1a} and R^{1b} are each independently chosen from hydrogen, halogen, C₁-C₄ alkyl, and C₁-C₄ haloalkyl groups; and
   all variables not specifically defined herein are as defined in any one of Embodiments 1-4.
33. A compound, tautomer, deuterated derivative, or pharmaceutically acceptable salt chosen from the compounds of Table 1, tautomers thereof, deuterated derivative of those compounds and tautomers, and pharmaceutically acceptable salts of any of the foregoing.
34. A compound, tautomer, deuterated derivative, or pharmaceutically acceptable salt chosen from the compounds of Table 2, tautomers thereof, deuterated derivative of those compounds and tautomers, and pharmaceutically acceptable salts of any of the foregoing.
35. A pharmaceutical composition comprising at least one compound, tautomer, deuterated derivative, or pharmaceutically acceptable salt according to any one of Embodiments 1-34 and a pharmaceutically acceptable carrier.
36. A method of treating focal segmental glomerulosclerosis and/or non-diabetic kidney disease comprising administering to a patient in need thereof at least one compound, tautomer, deuterated derivative, or pharmaceutically acceptable salt according to any one of Embodiments 1-34 or a pharmaceutical composition according to Embodiment 35.
37. Use of at least one compound, tautomer, deuterated derivative, or pharmaceutically acceptable salt according to any one of Embodiments 1-34 or a pharmaceutical composition according to Embodiment 35 for the manufacture of a medicament for treating focal segmental glomerulosclerosis and/or non-diabetic kidney disease.
38. At least one compound, tautomer, deuterated derivative, or pharmaceutically acceptable salt according to any one of Embodiments 1-34 or a pharmaceutical composition according to Embodiment 35 for use in treating focal segmental glomerulosclerosis and/or non-diabetic kidney disease.
39. A method of inhibiting APOL1 activity comprising contacting said APOL1 with at least one compound, tautomer, deuterated derivative, or pharmaceutically acceptable salt according to any one of Embodiments 1-34 or a pharmaceutical composition according to Embodiment 35.
40. Use of at least one compound, tautomer, deuterated derivative, or pharmaceutically acceptable salt according to any one of Embodiments 1-34 or a pharmaceutical composition according to Embodiment 35 for the manufacture of a medicament for inhibiting APOL1 activity.
41. At least one compound, tautomer, deuterated derivative, or pharmaceutically acceptable salt according to any one of Embodiments 1-34 or a pharmaceutical composition according to Embodiment 35 for use in inhibiting APOL1 activity.
42. A method of treating an APOL1-mediated disease comprising administering to a patient in need thereof at least one compound, tautomer, deuterated derivative, or pharmaceutically acceptable salt according to any one of Embodiments 1-34 or a pharmaceutical composition according to Embodiment 35.
43. The method according to Embodiment 42, wherein the APOL1-mediated disease is cancer.
44. The method according to Embodiment 42 or Embodiment 43, wherein the APOL1-mediated disease is pancreatic cancer.
45. Use of at least one compound, tautomer, deuterated derivative, or pharmaceutically acceptable salt according to any one of Embodiments 1-34 or a pharmaceutical composition according to Embodiment 35 for the manufacture of a medicament for treating an APOL1-mediated disease.
46. The use according to Embodiment 45, wherein the APOL1-mediated disease is cancer.
47. The use according to Embodiment 45 or Embodiment 46, wherein the APOL1-mediated disease is pancreatic cancer.
48. At least one compound, tautomer, deuterated derivative, or pharmaceutically acceptable salt according to any one of Embodiments 1-34 or a pharmaceutical composition according to Embodiment 35 for use in treating an APOL1-mediated disease.
49. The at least one compound, tautomer, deuterated derivative, or pharmaceutically acceptable salt for use according to Embodiment 48, wherein the APOL1-mediated disease is cancer.
50. The at least one compound, tautomer, deuterated derivative, or pharmaceutically acceptable salt for use according to Embodiment 48 or Embodiment 49, wherein the APOL1-mediated disease is pancreatic cancer.
51. A method of inhibiting APOL1 activity comprising contacting said APOL1 with at least one compound, tautomer, deuterated derivative, or pharmaceutically acceptable salt according to any one of Embodiments 1-34 or a pharmaceutical composition according to Embodiment 35.
52. Use of at least one compound, tautomer, deuterated derivative, or pharmaceutically acceptable salt according to any one of Embodiments 1-34 or a pharmaceutical composition according to Embodiment 35 for the manufacture of a medicament for inhibiting APOL1 activity.
53. At least one compound, tautomer, deuterated derivative, or pharmaceutically acceptable salt according to any one of Embodiments 1-34 or a pharmaceutical composition according to Embodiment 35 for use in inhibiting APOL1 activity.
54. A silicon derivative of the at least one compound, tautomer, deuterated derivative, or pharmaceutically acceptable salt according to any one of Embodiments 1-34.
55. A pharmaceutical composition comprising a silicon derivative of Embodiment 54.
56. A method of treating focal segmental glomerulosclerosis and/or non-diabetic kidney disease comprising administering to a patient in need thereof a silicon derivative according to Embodiment 54 or a pharmaceutical composition according to Embodiment 55.
57. Use of the silicon derivative according to Embodiment 54 or a pharmaceutical composition according to Embodiment 55 for the manufacture of a medicament for treating focal segmental glomerulosclerosis and/or non-diabetic kidney disease.
58. The silicon derivative according to Embodiment 54 or a pharmaceutical composition according to Embodiment 55 for use in treating focal segmental glomerulosclerosis and/or non-diabetic kidney disease.
59. A method of treating an APOL1-mediated disease comprising administering to a patient in need thereof a silicon derivative according to Embodiment 54 or a pharmaceutical composition according to Embodiment 55.
60. The method according to Embodiment 59, wherein the APOL1-mediated disease is cancer.
61. The method according to Embodiment 59 or Embodiment 60, wherein the APOL1-mediated disease is pancreatic cancer.
62. Use of the silicon derivative according to Embodiment 54 or a pharmaceutical composition according to Embodiment 55 for the manufacture of a medicament for treating an APOL1-mediated disease.
63. The use according to Embodiment 62, wherein the APOL1-mediated disease is cancer.
64. The use according to Embodiment 62 or Embodiment 63, wherein the APOL1-mediated disease is pancreatic cancer.
65. The silicon derivative according to Embodiment 54 or a pharmaceutical composition according to Embodiment 55 for use in treating an APOL1-mediated disease.
66. The silicon derivative or pharmaceutical composition for use according to Embodiment 65, wherein the APOL1-mediated disease is cancer.
67. The silicon derivative or pharmaceutical composition for use according to Embodiment 65 or Embodiment 66, wherein the APOL1-mediated disease is pancreatic cancer.
68. A boron derivative of the at least one compound, tautomer, deuterated derivative, or pharmaceutically acceptable salt according to any one of Embodiments 1-34.
69. A pharmaceutical composition comprising a boron derivative of Embodiment 68.
70. A method of treating focal segmental glomerulosclerosis and/or non-diabetic kidney disease comprising administering to a patient in need thereof a boron derivative according to Embodiment 68 or a pharmaceutical composition according to Embodiment 69.
71. Use of the boron derivative according to Embodiment 68 or a pharmaceutical composition according to Embodiment 69 for the manufacture of a medicament for treating focal segmental glomerulosclerosis and/or non-diabetic kidney disease.
72. The boron derivative according to Embodiment 68 or a pharmaceutical composition according to Embodiment 69 for use in treating focal segmental glomerulosclerosis and/or non-diabetic kidney disease.
73. A method of treating an APOL1-mediated disease comprising administering to a patient in need thereof a boron derivative according to Embodiment 68 or a pharmaceutical composition according to Embodiment 69.
74. The method according to Embodiment 73, wherein the APOL1-mediated disease is cancer.
75. The method according to Embodiment 73 or Embodiment 74, wherein the APOL1-mediated disease is pancreatic cancer.
76. Use of the boron derivative according to Embodiment 68 or a pharmaceutical composition according to Embodiment 69 for the manufacture of a medicament for treating an APOL1-mediated disease.
77. The use according to Embodiment 76, wherein the APOL1-mediated disease is cancer.
78. The use according to Embodiment 76 or Embodiment 77, wherein the APOL1-mediated disease is pancreatic cancer.
79. The boron derivative according to Embodiment 68 or a pharmaceutical composition according to Embodiment 69 for use in treating an APOL1-mediated disease.
80. The boron derivative or pharmaceutical composition for use according to Embodiment 79, wherein the APOL1-mediated disease is cancer.
81. The boron derivative or pharmaceutical composition for use according to Embodiment 79 or Embodiment 80, wherein the APOL1-mediated disease is pancreatic cancer.
82. A phosphorus derivative of at least one compound, tautomer, deuterated derivative or pharmaceutically acceptable salt according to any one of Embodiments 1-34.
83. A pharmaceutical composition comprising a phosphorus derivative of Embodiment 82.
84. A method of treating focal segmental glomerulosclerosis and/or non-diabetic kidney disease comprising administering to a patient in need thereof a phosphorus derivative according to Embodiment 82 or a pharmaceutical composition according to Embodiment 83.
85. Use of the phosphorus derivative according to Embodiment 82 or a pharmaceutical composition according to Embodiment 83 for the manufacture of a medicament for treating focal segmental glomerulosclerosis and/or non-diabetic kidney disease.
86. The phosphorus derivative according to Embodiment 82 or a pharmaceutical composition according to Embodiment 83 for use in treating focal segmental glomerulosclerosis and/or non-diabetic kidney disease.
87. A method of treating an APOL1-mediated disease comprising administering to a patient in need thereof a phosphorus derivative according to Embodiment 82 or a pharmaceutical composition according to Embodiment 83.
88. The method according to Embodiment 87, wherein the APOL1-mediated disease is cancer.
89. The method according to Embodiment 87 or Embodiment 88, wherein the APOL1-mediated disease is pancreatic cancer.
90. Use of the phosphorus derivative according to Embodiment 82 or a pharmaceutical composition according to Embodiment 83 for the manufacture of a medicament for treating an APOL1-mediated disease.
91. The use according to Embodiment 90, wherein the APOL1-mediated disease is cancer.
92. The use according to Embodiment 90 or Embodiment 91, wherein the APOL1-mediated disease is pancreatic cancer.
93. The phosphorus derivative according to Embodiment 82 or a pharmaceutical composition according to Embodiment 83 for use in treating an APOL1-mediated disease.
94. The phosphorus derivative or pharmaceutical composition for use according to Embodiment 93, wherein the APOL1-mediated disease is cancer.
95. The phosphorus derivative or pharmaceutical composition for use according to Embodiment 93 or Embodiment 94, wherein the APOL1-mediated disease is pancreatic cancer.

### EXAMPLES

In order that the disclosure described herein may be more fully understood, the following examples are set forth. It should be understood that these examples are for illustrative purposes only and are not to be construed as limiting this disclosure in any manner.

The compounds of the disclosure may be made according to standard chemical practices or as described herein. Throughout the following synthetic schemes and in the descriptions for preparing compounds of Formulae I, IA, IB, IC, ID, II, IIA, IV, IVA, IVB, IVC, V, VA, VB, VC, VI, VIA, VIB, VIC, VII, VIIA, VIII, VIIIA, IX, IXA, IXB, or IXC, Compounds 1 to 42 and Compounds I1 to I36, a tautomer thereof, a deuterated derivative of that compound or tautomer, or a pharmaceutically acceptable salt of any of the foregoing, the following abbreviations are used:

### Abbreviations

AIBN=azobisisobutyronitrile
ARP=assay ready plate
BBBPY=4,4′-Di-tert-butyl-2,2′-dipyridyl
BF₃=boron trifluoride
BF₃·OEt₂=boro trifluoride diethyl etherate
Boc₂O=di-tert-butyl dicarbonate
CBzCl=benzyl chloroformate
CDMT=2-chloro-4,6-dimethoxy-1,3,5-triazine
DAST=diethylaminosulfur trifluoride
DBU=1,8-diazabicyclo[5.4.0]undec-7-ene
DCM=dichloromethane
DIBAL-H=diisobutylaluminum hydride
DIPEA=N,N-Diisopropylethylamine or N-ethyl-N-isopropyl-propan-2-amine
DMAP=dimethylamino pyridine
DMA=dimethyl acetamide
DME=dimethoxyethane
DMEM=Dulbecco's modified Eagle's medium
DMF=dimethylformamide
DMPU=N,N′-dimethylpropyleneurea
DMSO=dimethyl sulfoxide
DPPA=diphenylphosphoryl azide
dppb=1-4-bis[P(Ph)₂]-butane
EtOAc=ethyl acetate
EtOH=ethanol
Et₂O=diethyl ether
FBS=fetal bovine serum
FLU=fluorescent values
HATU=[dimethylamino(triazolo[4,5-b]pyridin-3-yloxy)methylene]-dimethyl-ammonium (Phosphorus Hexafluoride Ion)
HDMC=N-[(5-Chloro-3-oxido-TH-benzotriazol-1-yl)-4-morpholinylmethylene]-N-methylmethanaminium hexafluorophosphate
HEPES=4-(2-hydroxyethyl)-1-piperazineethanesulfonic acid
HBSS=Hank's balanced salt solution
IPA=isopropyl alcohol
Ir[df(CF₃)ppy]2(dtbbpy)PF₆=phosphorus hexafluoride
LDA=lithium diisopropyl amide
LED=light emitting diode
MeCN=acetonitrile
MeI=methyl iodide
MeOH=methanol
MsOH=methanesulfonic acid
MTBE or TBME=Methyl tert-butyl ether
n-BuLi=n-butyllithium
NBS=n-bromosuccinimide
NMM=N-methyl morpholine
NMP=N-methyl pyrrolidine
PBS=phosphate-buffered saline
Pd(dppf)₂Cl₂=[1,1′-Bis(diphenylphosphino)ferrocene]dichloropalladium(II)
PdCl₂(PPh₃)₂=Bis(triphenylphosphine)palladium(II) dichloride
PP=polypropylene
PTSA=p-Toluenesulfonic acid monohydrate
T3P=2,4,6-Tripropyl-1,3,5,2,4,6-trioxatriphosphorinane-2,4,6-trioxide
TBAF=tetra-n-butylammonium fluoride
TBSCl=tert-butyldimethylsilyl chloride
TEA=triethylamine
Tet=tetracycline
TFA or TFAA=trifluoroacetic acid
TfOH=triflic acid
THF=tetrahydrofuran
2-Me-THF=2=methyltetrahydrofuran
THP=tetrahydropyran
TMSCl=trimethylsilyl chloride
TMSS=Tris(trimethylsilyl)silane

### Example 1. Synthesis of Compounds

All the specific and generic compounds, and the intermediates disclosed for making those compounds, are considered to be part of the disclosure disclosed herein.

### Synthesis of Starting Materials

Preparations describe synthetic routes to intermediates used in the synthesis of Compounds 1 to 42. Similar preparations may be used to synthesize intermediates for preparing Compounds I1 to I36.

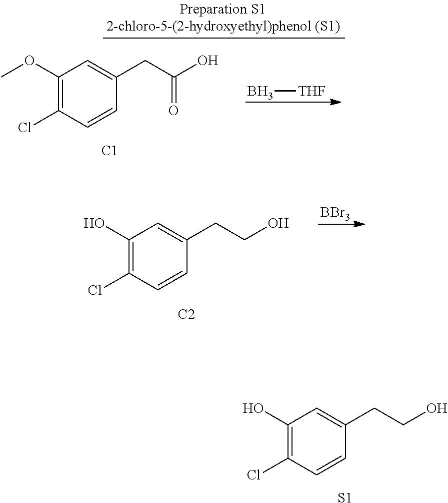

### Step 1. Synthesis of 2-chloro-5-(2-hydroxyethyl)phenol (C2)

To a solution of 2-(4-chloro-3-methoxy-phenyl)acetic acid (690 mg, 3.44 mmol) in THF (6.8 mL) at 0° C. was added dropwise BH₃-THF (6.8 mL, 6.80 mmol, 1 M in THF). The reaction was warmed to room temperature and stirred overnight. The reaction was cooled down to 0° C. and quenched slowly with MeOH (6 mL). Gas evolution was observed. The reaction was concentrated in vacuo to afford the title compound C2 as a colorless oil (641 mg, 100%). The crude was used directly without further purification. LCMS m/z 170.0 [M−OH+H]⁺.

### Step 2. Synthesis of 2-chloro-5-(2-hydroxyethyl)phenol (S1)

To a solution of 2-chloro-5-(2-hydroxyethyl)phenol (715 mg, 3.83 mmol) in DCM (12.5 mL) at 0° C. was added slowly BBr₃ (7.6 mL, 7.60 mmol, 1 M in heptane). The resulting colorless solution was slowly warmed to room temperature and stirred for 4 hours. The reaction was cooled down to 0° C. and quenched slowly with H₂O. Some white solids precipitated out and were isolated by filtration to afford the desired product. The filtrate was extracted with DCM (×3) and the combined organic extracts were washed with brine, dried over Na₂SO₄, filtered, and concentrated in vacuo to afford some additional product as a white solid. The two batches were combined to give the title compound S1 (570 mg, 86%). ¹H NMR (300 MHz, Chloroform-d) δ 7.26 (d, J=2.0 Hz, 1H), 6.91 (d, J=2.0 Hz, 1H), 6.78-6.70 (m, 1H), 5.48 (s, 1H), 3.85 (q, J=6.3 Hz, 2H), 2.81 (t, J=6.5 Hz, 2H), 1.35 (br s, 1H). LCMS m/z 154.0 [M−OH]⁺.

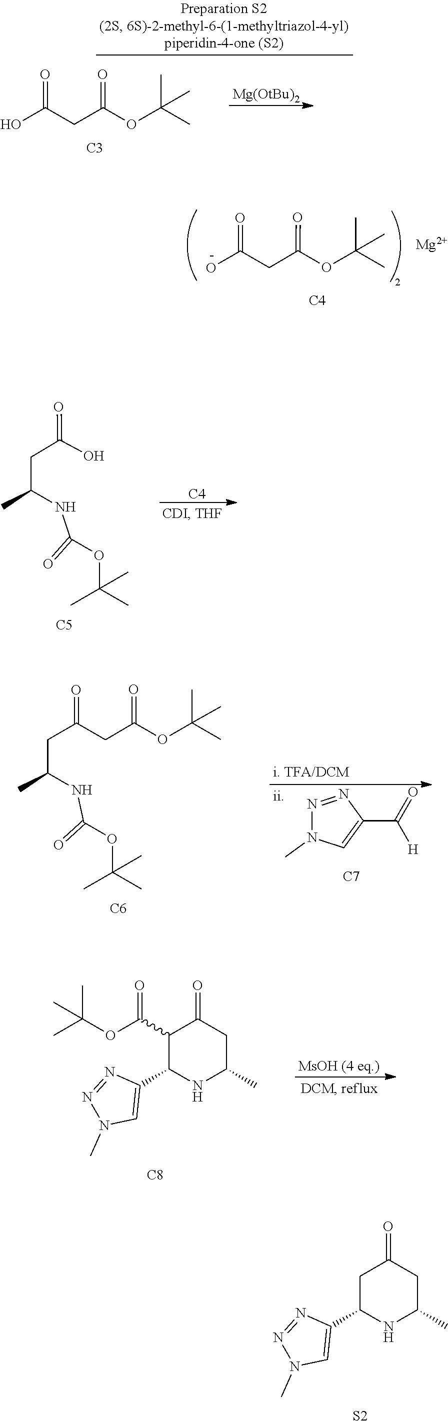

### Step 1. Synthesis of bis[(3-tert-butoxy-3-oxo-propanoyl)oxy]magnesium(C4)

A solution of 3-tert-butoxy-3-oxo-propanoic acid (321.51 g, 1.907 mol) in THF (2 L) was cooled to 5° C. in an ice-bath and Mg(OEt)₂ (111.33 g, 953.5 mmol) was added. The reaction was stirred for 30 minutes at 0° C., removed from the cooling bath, and stirred at room temperature overnight. The reaction was filtered over a plug of Celite®, and the plug was washed with additional THF. The clear, colorless filtrate was evaporated in vacuo to afford a mushy solid. The solid was triturated with 1 L of diethyl ether and filtered. The filter-cake was washed with Et₂O and dried in vacuo. The filtrate was evaporated in vacuo again and was then triturated with a small volume of Et₂O and filtered to afford a second crop of the product. The crops were combined and dried in vacuo to afford the title compound C4 (294.49 g, 90%) as a white solid. ¹H NMR (300 MHz, Methanol-d₄) δ 4.92 (s, 4H), 1.48 (s, 18H).

### Step 2. Synthesis of tert-butyl (5S)-5-(tert-butoxycarbonylamino)-3-oxo-hexanoate (C6)

To a solution of (3S)-3-(tert-butoxycarbonylamino)butanoic acid (170.15 g, 837.2 mmol) in THF (1.5 L) was added CDI (149.8 g, 923.8 mmol). The milky suspension cleared over the next few minutes. Gas evolution was observed. The reaction was stirred at room temperature for 3 hours. Bis[(3-tert-butoxy-3-oxo-propanoyl)oxy]magnesium (172.19 g, 502.6 mmol) was added. Another milky suspension was formed that cleared after stirring for 30 minutes. The reaction was stirred for 48 hours. The reaction was poured into 1.5 L of 1N HCl and extracted with MTBE (1 L). The pH was confirmed to be approximately 3. The extract was washed with saturated NaHCO₃, dried over MgSO₄, filtered, and concentrated in vacuo to afford the title compound C6 (248.5 g, 98.5%) as a colorless oil. ¹H NMR (300 MHz, Chloroform-d) δ 4.90 (d, J=18.1 Hz, 1H), 4.04 (dt, J=13.8, 6.6 Hz, 1H), 3.47-3.22 (m, 2H), 2.76 (qd, J=17.0, 5.7 Hz, 2H), 1.48 (s, 9H), 1.44 (s, 9H), 1.23 (d, J=6.8 Hz, 3H).

### Step 3. Synthesis of tert-butyl (2S,3R,6S)-6-methyl-2-(1-methyltriazol-4-yl)-4-oxo-piperidine-3-carboxylate (C8)

To a solution of tert-butyl (5S)-5-(tert-butoxycarbonylamino)-3-oxo-hexanoate (248.5 g, 824.5 mmol) in DCM (1.5 L) was added TFA (240 mL, 3.115 mol) and the reaction was stirred overnight. The reaction was evaporated in vacuo at 25° C. The solid that remained was triturated with 500 mL of pentane and filtered. The filter cake was washed with pentane and most of the solvent was pulled off of the filter-cake. The cake was transferred back to the reaction flask and dissolved in 1 L of DCM. 1-Methyltriazole-4-carbaldehyde (120.7 g, 1.086 mol) was added. The reaction was stirred at room temperature overnight. Brine (100 mL) was added, and then 6N NaOH was added until the aqueous layer remained alkaline when the funnel was shaken. The organic layer was isolated, and the aqueous layer was extracted with DCM (1 L). The organic layers were combined, dried over MgSO₄, and filtered over a plug of silica gel. The plug was eluted with 10% MeOH/EtOAc. The filtrate was evaporated in vacuo to afford a solid that was triturated with MTBE (500 mL) and filtered. The filter cake was washed with MTBE and dried in vacuo to give a crop of product. The mother liquor from the trituration was concentrated. The solid that precipitated was filtered to provide a second crop of the product. The crops were combined to give the title compound C8 (105.45 g, 43%) as a white solid. ¹H NMR (300 MHz, Chloroform-d) δ 7.48 (s, 1H), 4.52 (d, J=11.0 Hz, 1H), 4.09 (s, 3H), 3.61 (dd, J=11.0, 1.0 Hz, 1H), 3.21 (ddd, J=11.7, 6.1, 2.9 Hz, 1H), 2.55 (dd, J=13.7, 2.9 Hz, 1H), 2.37-2.13 (m, 1H), 1.98 (s, 1H), 1.39 (s, 9H), 1.29 (d, J=6.3 Hz, 3H).

### Step 4. Synthesis of (2S,6S)-2-methyl-6-(1-methyltriazol-4-yl)piperidin-4-one (S2)

To a solution of tert-butyl (2S,3R,6S)-6-methyl-2-(1-methyltriazol-4-yl)-4-oxo-piperidine-3-carboxylate (70.59 g, 239.8 mmol) in DCM (750 mL) was added MsOH (62 mL, 955.4 mmol) and the reaction was heated to reflux for 6 hours. The reaction was cooled down to room temperature, and then poured into a separatory funnel. Brine (100 mL) was added, and then 6N NaOH was added until the aqueous layer remained alkaline after shaking. The organic layer was separated and the aqueous layer was extracted with DCM (2×500 mL). The organic layers were combined, dried over MgSO₄, filtered, and concentrated in vacuo to afford the title compound S2 (43.74 g, 94%) as a pale yellow solid. ¹H NMR (300 MHz, Chloroform-d) δ 7.46 (s, 1H), 4.20 (dd, J=10.1, 5.1 Hz, 1H), 4.06 (s, 3H), 3.11 (dqd, J=12.3, 6.2, 3.0 Hz, 1H), 2.73-2.48 (m, 2H), 2.40 (ddd, J=14.1, 3.0, 1.5 Hz, 1H), 2.25-2.00 (m, 2H), 1.23 (d, J=6.2 Hz, 3H).

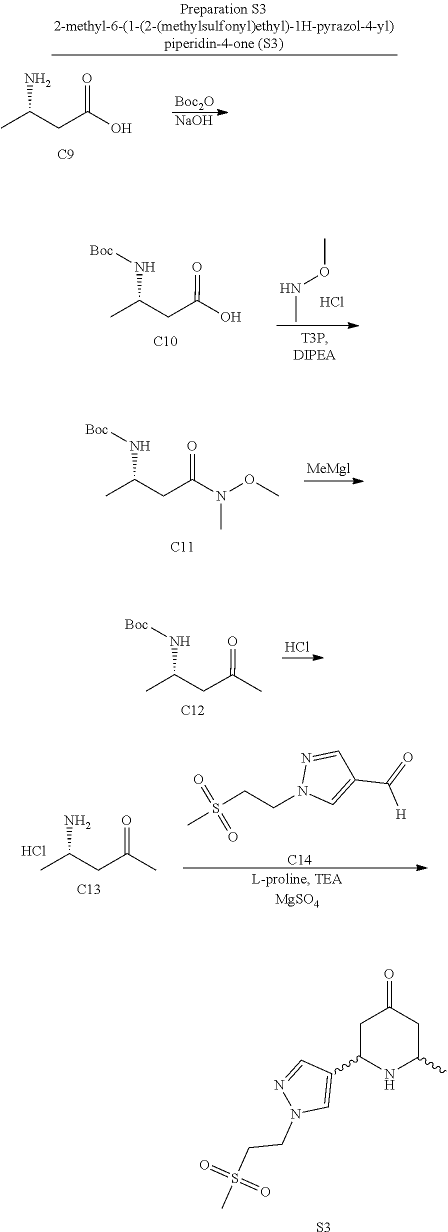

### Step 1. Synthesis of (3S)-3-(tert-butoxycarbonylamino)butanoic acid (C10)

To a solution of (3S)-3-aminobutanoic acid (100 g, 969.7 mmol) in dioxane (600 mL) was added aqueous NaOH solution (950 mL of 1 M, 950.0 mmol) over 15 minutes, followed by Boc₂O (300 g, 1.375 mol). The reaction mixture was stirred at room temperature for 12 hours. The reaction was partitioned with MTBE (1 L) and water (300 mL). The layers were separated, and the aqueous layer was extracted again with MTBE (500 mL). The aqueous layer was then acidified with 1N HCl until pH=2 and extracted with DCM (3×600 mL). The combined organic layers were washed with brine, dried over MgSO₄, filtered and concentrated in vacuo to yield the title compound C10 (176 g, 89%) as a white solid. ¹H NMR (300 MHz, Chloroform-d) δ 4.92 (s, 1H), 4.04 (s, 1H), 2.56 (dd, J=5.5, 2.9 Hz, 2H), 1.44 (s, 9H), 1.25 (d, J=6.8 Hz, 3H).

### Step 2. Synthesis of tert-butyl N-[(S)-3-[methoxy(methyl)amino]-1-methyl-3-oxo-propyl]carbamate (C11)

To a solution of (3S)-3-(tert-butoxycarbonylamino)butanoic acid (160 g, 787.3 mmol) in DCM (1.5 L) was added N-methoxymethanamine (Hydrochloride salt) (81 g, 830.4 mmol) followed by the addition of DIPEA (560 mL, 3.215 mol) over 10 minutes. The reaction mixture was cooled to 0° C. and T3P (600 g of 50% w/w in EtOAc, 942.9 mmol) was added over 45 minutes. After the addition, the cooling bath was removed and the reaction was stirred at room temperature for 1 hour. The reaction mixture was cooled to 10° C. and aqueous 1N NaOH solution (700 mL) was added and the solution stirred for 15 minutes. The organic phase was separated, washed with aqueous saturated ammonium chloride solution (200 mL) and brine (200 mL), dried, filtered through a silica gel plug, and concentrated in vacuo to afford the title compound C11 (180 g, 93%) as a clear, colorless viscous oil. ¹H NMR (300 MHz, Chloroform-d) 6 5.30 (s, 1H), 4.06 (ddd, J=14.3, 9.7, 6.0 Hz, 1H), 3.68 (s, 3H), 3.17 (s, 3H), 2.71 (dd, J=15.6, 5.2 Hz, 1H), 2.54 (dd, J=15.7, 5.7 Hz, 1H), 1.43 (s, 9H), 1.24 (d, J=6.8 Hz, 3H).

### Step 3. Synthesis of tert-butyl N-[(S)-1-methyl-3-oxo-butyl]carbamate (C12)

To a solution of tert-butyl N-[(1S)-3-[methoxy(methyl)amino]-1-methyl-3-oxo-propyl]carbamate (220 g, 893.2 mmol) in THF (4 L) at 0° C. was added iodo(methyl)magnesium (900 mL of 3M, 2.700 mol) over 40 minutes. The resulting reaction mixture was stirred at 0° C. for 4 hours. The reaction was quenched with saturated ammonium chloride solution (2 L), followed by MTBE (1 L) and water (2 L). The mixture was stirred for 30 minutes, and the organic layer was separated. The aqueous phase was extracted with MTBE (1 L) and the combined organic layers were washed with saturated ammonium chloride solution (1 L), dried over MgSO₄, filtered and concentrated in vacuo. Purification by silica gel chromatography (Gradient: 0-70% EtOAc in heptane) yielded the title compound C12 (115 g, 64%) as a white solid. ¹H NMR (300 MHz, Chloroform-d) δ 4.83 (s, 1H), 4.12-3.87 (m, 1H), 2.69 (dd, J=16.5, 5.2 Hz, 1H), 2.63-2.47 (m, 1H), 2.15 (d, J=2.3 Hz, 3H), 1.43 (d, J=2.4 Hz, 9H), 1.20 (dd, J=6.8, 2.4 Hz, 3H).

### Step 4. Synthesis of (4S)-4-aminopentan-2-one (Hydrochloride salt) (C13)

To a solution of tert-butyl N-[(1S)-1-methyl-3-oxo-butyl]carbamate (16.3 g, 80.18 mmol) in MeOH (30 mL) was added hydrogen chloride (50 mL of 4 M in dioxane, 200.0 mmol) over 3 minutes. The reaction was stirred at room temperature for 5 hours and then concentrated under reduced pressure. The residue was co-evaporated with EtOH (2×30 mL) and dried under vacuum to afford the title compound C13 (12 g, 98%) as a pink viscous oil. ¹H NMR (300 MHz, Chloroform-d) δ 8.06 (s, 3H), 3.48 (d, J=6.8 Hz, 1H), 2.88 (dd, J=18.0, 5.8 Hz, 1H), 2.75 (dd, J=18.0, 7.2 Hz, 1H), 2.13 (s, 3H), 1.17 (d, J=6.6 Hz, 3H).

### Step 5. Synthesis of 2-methyl-6-(1-(2-(methylsulfonyl)ethyl)-1H-pyrazol-4-yl)piperidin-4-one (S3)

To a mixture of (4S)-4-aminopentan-2-one (Hydrochloride salt) (580 mg, 4.088 mmol) in EtOH (13 mL) was added 1-(2-methylsulfonylethyl)pyrazole-4-carbaldehyde (760 mg, 3.758 mmol), L-Proline (94 mg, 0.8165 mmol), magnesium sulfate (600 mg, 4.985 mmol), and TEA (600 μL, 4.305 mmol). The reaction mixture was stirred at room temperature overnight. TLC indicated incomplete reaction, so additional 1-(2-methylsulfonylethyl)pyrazole-4-carbaldehyde (150 mg, 0.74 mmol) was added and the reaction was stirred overnight. The reaction mixture was filtered and concentrated under reduced pressure. The crude residue was quenched with saturated sodium bicarbonate solution and extracted with DCM (×3). The combined organic extracts were washed with brine, dried over magnesium sulfate, filtered and concentrated in vacuo. The crude was purified via silica gel chromatography (0-60% of 20% MeOH/DCM in DCM) to yield the title compound S3 (500 mg, 38%) in 7:1 cis to trans ratio. Additionally, the e.r. at the stereocenter from C13 was eroded to ˜85%. ¹H NMR (300 MHz, Chloroform-d) δ 7.58 (s, 1H), 7.53 (s, 1H), 4.60 (t, J=6.3 Hz, 2H), 4.00 (dd, J=11.6, 3.3 Hz, 1H), 3.65 (t, J=6.2 Hz, 2H), 3.10 (dqd, J=12.1, 6.0, 2.9 Hz, 1H), 2.58-2.51 (m, 4H), 2.48-2.37 (m, 2H), 2.17 (dd, J=14.1, 11.6 Hz, 1H), 1.26 (d, J=6.1 Hz, 3H) (cis isomer).

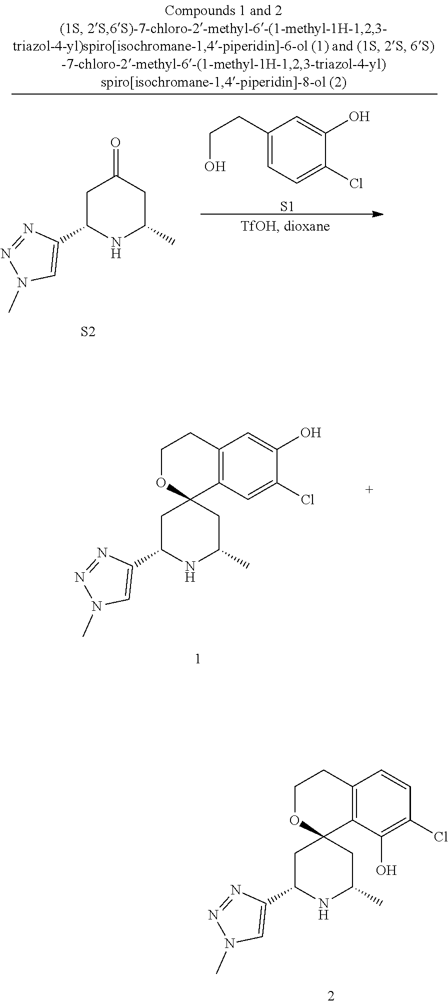

To a solution of (2S,6S)-2-methyl-6-(1-methyltriazol-4-yl)piperidin-4-one (125 mg, 0.64 mmol) and 2-chloro-5-(2-hydroxyethyl)phenol (122 mg, 0.71 mmol) in dioxane (3.2 mL) at 0° C. was added triflic acid (285 μL, 3.221 mmol). The reaction was warmed to room temperature and stirred for 5 hours. The volatile was removed, and the crude was purified by reverse-phase HPLC (Method: C18 Waters Sunfire column (30×150 mm, 5 micron), gradient: MeCN in H₂O with 0.1% trifluoroacetic acid) to afford the major regioisomer 1 (175 mg, 53%) and the minor regioisomer 2 as trifluoroacetate salts (39 mg, 12%) in ˜4:1 ratio. The absolute stereochemistry was confirmed by extensive NMR analysis.

Characterization data for compound 1: ¹H NMR (300 MHz, Methanol-d₄) δ 8.05 (s, 1H), 7.16 (s, 1H), 6.71 (s, 1H), 4.85 (s, 1H), 4.13 (s, 3H), 3.95 (t, J=5.5 Hz, 2H), 3.82 (s, 1H), 2.76 (t, J=5.5 Hz, 2H), 2.39 (d, J=8.9 Hz, 2H), 2.23 (d, J=14.6 Hz, 1H), 2.00-1.85 (m, 1H), 1.40 (d, J=6.6 Hz, 3H). LCMS m/z 349.23 [M+H]⁺.

Characterization data for compound 2: ¹H NMR (300 MHz, Methanol-d4) δ 8.04 (s, 1H), 7.21 (d, J=8.2 Hz, 1H), 6.77-6.65 (m, 1H), 4.88-4.98 (m, 1H), 4.12 (s, 3H), 3.94 (t, J=5.5 Hz, 2H), 3.86 (ddd, J=12.6, 6.5, 3.2 Hz, 1H), 3.43-3.24 (m, 1H), 2.94-2.76 (m, 3H), 2.22 (dt, J=14.5, 2.8 Hz, 1H), 2.06 (dt, J=14.5, 2.8 Hz, 1H), 1.40 (d, J=6.6 Hz, 3H). LCMS m/z 349.0 [M+H]⁺.

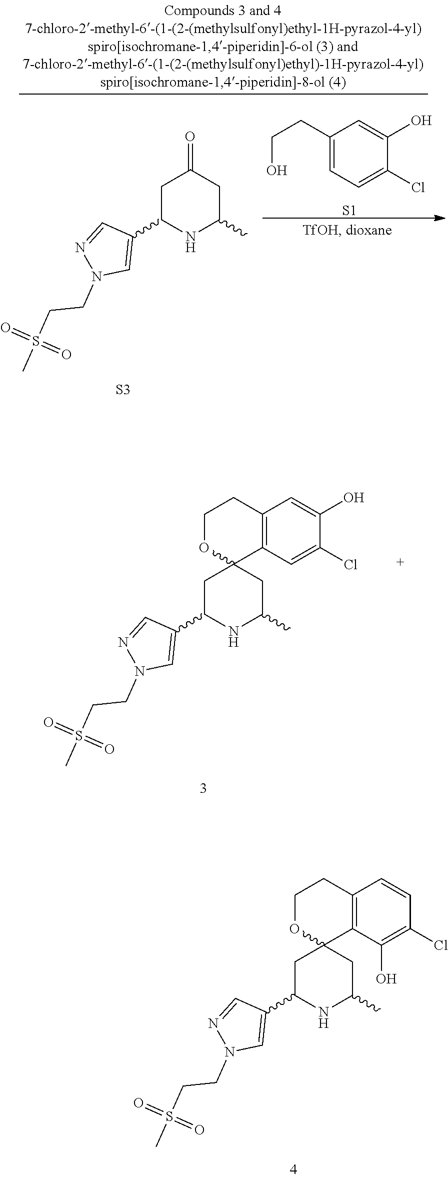

Compounds 3 and 4 were prepared from S1 and S3 following the method described for compounds 1 and 2. The reaction was purified by reverse-phase HPLC (Method: C18 Waters Sunfire column (30×150 mm, 5 micron), gradient: MeCN in H₂O with 0.1% trifluoroacetic acid) to afford the major regioisomer 3 (155 mg, 57%) and the minor regioisomer 4 as trifluoroacetate salts (25.8 mg, 9.6%) in ˜6:1 ratio.

Characterization data for compound 3: ¹H NMR (300 MHz, Methanol-d₄) δ 7.93 (s, 1H), 7.73 (s, 1H), 7.19 (s, 1H), 6.71 (s, 1H), 4.76-4.60 (m, 3H), 3.93 (t, J=5.5 Hz, 2H), 3.77 (s, 1H), 3.70 (t, J=6.3 Hz, 2H), 2.84 (s, 3H), 2.75 (t, J=5.5 Hz, 2H), 2.35-2.26 (m, 2H), 2.20 (d, J=14.7 Hz, 1H), 1.87 (dd, J=14.7, 12.2 Hz, 1H), 1.37 (d, J=6.6 Hz, 3H). LCMS m/z 440.0 [M+H]⁺.

Characterization data for compound 4: ¹H NMR (300 MHz, Methanol-d₄) δ 7.90 (s, 1H), 7.71 (s, 1H), 7.22 (d, J=8.2 Hz, 1H), 6.73 (d, J=8.3 Hz, 1H), 4.74 (dd, J=12.8, 3.0 Hz, 1H), 4.65 (t, J=6.3 Hz, 2H), 3.91 (t, J=5.4 Hz, 2H), 3.86-3.75 (m, 1H), 3.70 (t, J=6.3 Hz, 2H), 3.26-3.16 (m, 1H), 2.87-2.73 (m, 6H), 2.15 (dd, J=14.5, 2.9 Hz, 1H), 2.10-1.98 (m, 1H), 1.37 (d, J=6.6 Hz, 3H). LCMS m/z 440.0 [M+H]⁺.

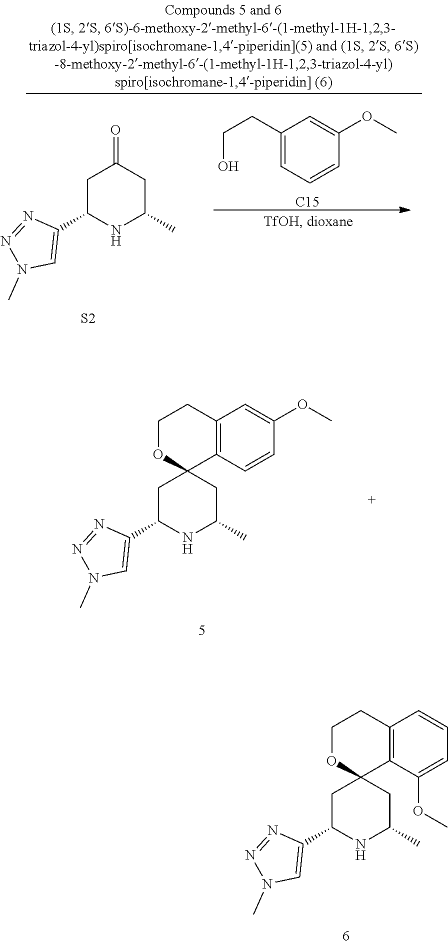

Compounds 5 and 6 were prepared from commercially available 2-(3-methoxyphenyl)ethan-1-ol (C15) and S2 following the method described for compounds 1 and 2. The reaction was purified by reverse-phase HPLC (Method: C18 Waters Sunfire column (30×150 mm, 5 micron), gradient: MeCN in H₂O with 0.2% formic acid) to afford the major regioisomer 5 (39 mg, 40%) and the minor regioisomer 6 as formate salts (8 mg, 8%) in ˜5:1 ratio. The absolute stereochemistry was assigned by analogy to compounds 1 and 2.

Characterization data for compound 5: ¹H NMR (300 MHz, Methanol-d4) δ 8.33 (s, 1H), 8.04 (s, 1H), 7.12 (d, J=8.7 Hz, 1H), 6.82 (dd, J=8.7, 2.7 Hz, 1H), 6.75-6.68 (m, 1H), 4.87 (dd, J=12.1, 3.5 Hz, 1H), 4.12 (s, 3H), 3.96 (t, J=5.5 Hz, 2H), 3.90-3.76 (m, 1H), 3.77 (s, 3H), 2.83 (t, J=5.5 Hz, 2H), 2.46 (dd, J=14.7, 12.1 Hz, 1H), 2.34 (ddd, J=14.7, 3.6, 2.2 Hz, 1H), 2.20 (dt, J=14.6, 2.8 Hz, 1H), 1.98 (dd, J=14.7, 12.1 Hz, 1H), 1.39 (d, J=6.6 Hz, 3H). LCMS m/z 328.4 [M+H]⁺.

Characterization data for compound 6: ¹H NMR (300 MHz, Methanol-d4) δ 8.49 (s, 1H), 8.02 (s, 1H), 7.20 (dd, J=8.3, 7.6 Hz, 1H), 6.89 (dd, J=8.3, 1.1 Hz, 1H), 6.79 (dd, J=7.6, 1.1 Hz, 1H), 4.90 (d, J=3.3 Hz, 1H), 4.12 (s, 3H), 3.98-3.74 (m, 6H), 3.19 (dd, J=14.5, 12.7 Hz, 1H), 2.83 (t, J=5.5 Hz, 2H), 2.79-2.62 (m, 1H), 2.19 (ddd, J=14.5, 3.3, 2.5 Hz, 1H), 2.02 (dt, J=14.5, 2.8 Hz, 1H), 1.38 (d, J=6.6 Hz, 3H). LCMS m/z 328.4 [M+H]⁺.

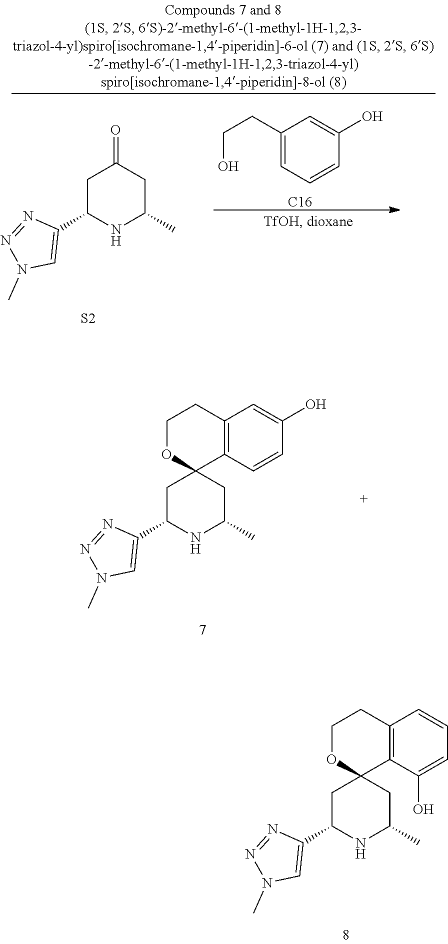

Compounds 7 and 8 were prepared from commercially available 3-(2-hydroxyethyl)phenol (C16) and S2 following the method described for compounds 1 and 2. The reaction was purified by reverse-phase HPLC (Method: C18 Waters Sunfire column (30×150 mm, 5 micron), gradient: MeCN in H₂O with 0.10% trifluoroacetic acid) to afford the major regioisomer 7 (17.7 mg, 63%) and the minor regioisomer 8 as trifluoroacetate salts (4.7 mg, 16.7%) in ˜4:1 ratio. The absolute stereochemistry was assigned by analogy to compounds 1 and 2.

Characterization data for compound 7: ¹H NMR (400 MHz, Methanol-d4) δ 8.03 (s, 1H), 7.02 (d, J=8.6 Hz, 1H), 6.72-6.65 (m, 1H), 6.57 (d, J=2.5 Hz, 1H), 4.89 (s, 1H), 4.12 (s, 3H), 3.95 (t, J=5.5 Hz, 2H), 3.81 (s, 1H), 2.77 (t, J=5.5 Hz, 2H), 2.48-2.38 (m, 1H), 2.35 (d, J=14.7 Hz, 1H), 2.19 (d, J=14.7 Hz, 1H), 2.02-1.90 (m, 1H), 1.39 (d, J=6.6 Hz, 3H). LCMS m/z 314.4 [M+H]⁺.

Characterization data for compound 8: ¹H NMR (400 MHz, Methanol-d4) δ 8.01 (s, 1H), 7.02 (t, J=7.8 Hz, 1H), 6.66 (d, J=7.9 Hz, 2H), 4.91 (s, 1H), 4.12 (s, 3H), 3.93 (t, J=5.5 Hz, 2H), 3.83 (s, 1H), 3.38 (d, J=13.7 Hz, 1H), 2.97-2.86 (m, 1H), 2.81 (t, J=5.4 Hz, 2H), 2.20 (d, J=14.7 Hz, 1H), 2.03 (d, J=14.7 Hz, 1H), 1.39 (d, J=6.6 Hz, 3H). LCMS m/z 314.4 [M+H]⁺.

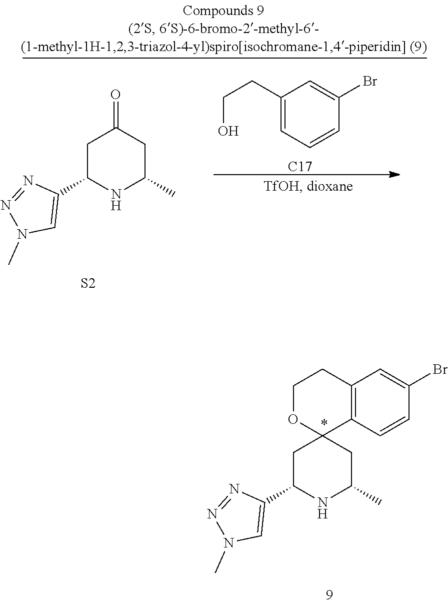

To a solution of (2S,6S)-2-methyl-6-(1-methyltriazol-4-yl)piperidin-4-one (500 mg, 2.574 mmol) and commercially available 2-(3-bromophenyl)ethanol (517.5 mg, 350.1 μL, 2.574 mmol) in dioxane (15 mL) was added dropwise triflic acid (1.93 g, 1.139 mL, 12.87 mmol). The resulting solution was heated at 100° C. overnight. The reaction was cooled down to room temperature and quenched with saturated NaHCO₃ solution. The mixture was extracted with EtOAc (×3). The combined organic extracts were washed with brine, dried over Na₂SO₄, filtered, and concentrated in vacuo. The crude was purified by silica gel chromatography (0 to 20% MeOH in DCM) to afford the title product 9 (72 mg, 28.7%), contaminated with ˜13% unknown isomer. ¹H NMR (300 MHz, Methanol-d4) δ 7.85 (s, 1H), 7.39-7.25 (m, 2H), 7.13 (d, J=8.4 Hz, 1H), 4.43 (dd, J=11.8, 2.8 Hz, 1H), 4.08 (d, J=0.7 Hz, 3H), 4.01-3.87 (m, 3H), 2.81 (t, J=5.5 Hz, 2H), 2.21 (dt, J=13.9, 2.6 Hz, 1H), 2.04-1.91 (m, 2H), 1.61 (dd, J=13.9, 11.5 Hz, 1H), 1.17 (t, J=6.2 Hz, 3H). LCMS m/z 377.3 [M+H]⁺. Preparation S4

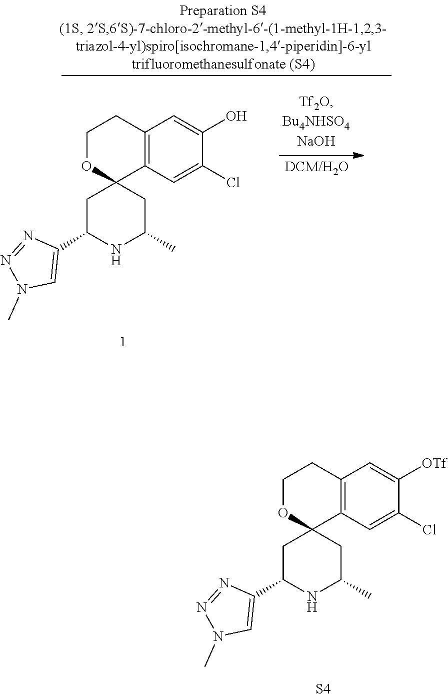 [SMILES: C[C@H]1C[C@@]2(C[C@@H](c3cn(C)nn3)N1)OCCc1cc(O)c(Cl)cc12 ; C[C@H]1C[C@@]2(C[C@@H](c3cn(C)nn3)N1)OCCc1cc(OS(=O)(=O)C(F)(F)F)c(Cl)cc12 ; "S4"]

To a suspension of (1S,2′S,6′S)-7-chloro-2′-methyl-6′-(1-methyltriazol-4-yl)spiro[isochromane-1,4′-piperidine]-6-ol (247 mg, 0.71 mmol), 1,1,1-trifluoro-N-phenyl-N-(trifluoromethylsulfonyl)methanesulfonamide (1 g, 2.80 mmol) and tetrabutylammonium hydrogen sulfate (244 mg, 0.72 mmol) in DCM (8 mL) was added an aqueous solution of NaOH (2.8 mL, 7.0 mmol, 2.5 M). The mixture was stirred vigorously at room temperature overnight. The reaction was diluted with water. The organic layer was separated, and the aqueous layer was extracted with MTBE (×2). The combined organic extracts were washed with water and brine, dried over Na₂SO₄, filtered and concentrated in vacuo. The crude was purified by silica gel chromatography (0 to 20% MeOH in DCM) to afford the title compound S4 as a white form solid (202 mg, 59%). LCMS m/z 481.0 [M+H]⁺.

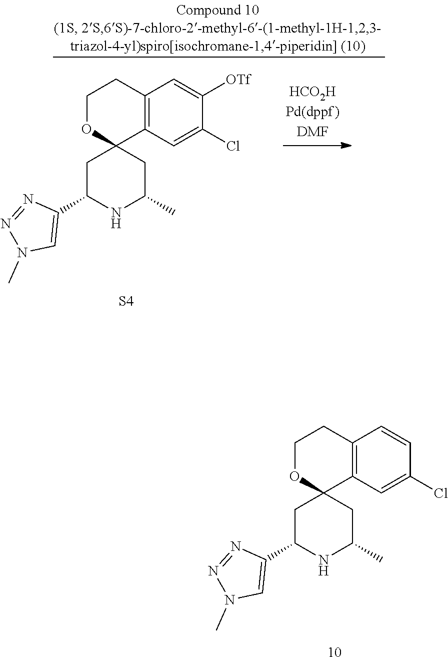 [SMILES: C[C@H]1C[C@@]2(C[C@@H](c3cn(C)nn3)N1)OCCc1cc(OS(=O)(=O)C(F)(F)F)c(Cl)cc12 ; "S4" ; C[C@H]1C[C@@]2(C[C@@H](c3cn(C)nn3)N1)OCCc1ccc(Cl)cc12 ; "10"]

To a solution of [(1S,2′S,6′S)-7-chloro-2′-methyl-6′-(1-methyltriazol-4-yl)spiro[isochromane-1,4′-piperidine]-6-yl]trifluoromethanesulfonate (28 mg, 0.058 mmol) in DMF (0.58 mL) was added Pd(dppf)Cl₂ (4.8 mg, 0.0059 mmol) and Et₃N (24 μL, 0.17 mmol) followed by formic acid (4.5 μL, 0.12 mmol). The resulting red solution was heated at 60° C. for 6 hours. The reaction was purified by reverse-phase HPLC (Method: C18 Waters Sunfire column (30×150 mm, 5 micron), gradient: MeCN in H₂O with 0.1% trifluoroacetic acid) to afford the title compound 10 as a trifluoroacetate salt (24.9 mg, 94%). ¹H NMR (300 MHz, Methanol-d4) δ 8.05 (s, 1H), 7.32-7.11 (m, 3H), 4.95-4.80 (m, 1H), 4.12 (s, 3H), 3.99 (t, J=5.5 Hz, 2H), 3.84 (s, 1H), 2.84 (t, J=5.5 Hz, 2H), 2.49-2.39 (m, 2H), 2.27 (d, J=14.5 Hz, 1H), 2.05-1.87 (m, 1H), 1.41 (d, J=6.6 Hz, 3H). LCMS m/z 332.8 [M+H]⁺.

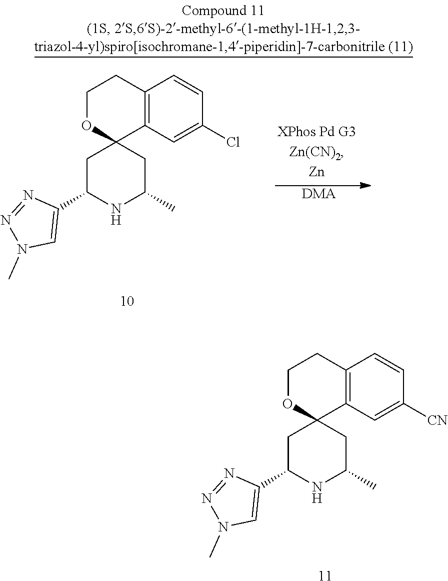

To a 1-dram vial was charged (1S,2′S,6′S)-7-chloro-2′-methyl-6′-(1-methyltriazol-4-yl)spiro[isochromane-1,4′-piperidine](15 mg, 0.045 mmol), XPhos Pd G3 (4 mg, 0.005 mmol), Zn(CN)₂ (11 mg, 0.094 mmol) and Zn powder (1 mg, 0.015 mmol). The vial was capped and purged with N₂ (×3), and then DMA (0.5 mL) was added. The resulting dark orange solution was heated at 120° C. overnight. The reaction was purified by reverse-phase HPLC (Method: C18 Waters Sunfire column (30×150 mm, 5 micron), gradient: MeCN in H₂O with 0.1% trifluoroacetic acid) to afford the title compound 11 as a trifluoroacetate salt (19.7 mg, 81%). ¹H NMR (400 MHz, DMSO-d6) δ 9.31 (d, J=10.6 Hz, 1H), 8.78 (d, J=11.0 Hz, 1H), 8.21 (s, 1H), 7.66 (dd, J=8.0, 1.6 Hz, 1H), 7.57 (s, 1H), 7.38 (d, J=8.0 Hz, 1H), 4.66 (t, J=11.1 Hz, 1H), 4.04 (s, 3H), 3.90 (t, J=5.4 Hz, 2H), 3.50-3.70 (m, 1H), 2.85 (t, J=5.4 Hz, 2H), 2.2.41-2.45 (m, 1H), 2.28 (d, J=14.2 Hz, 1H), 2.05 (dt, J=26.4, 14.1 Hz, 2H), 1.23 (d, J=14.8 Hz, 3H). LCMS m/z 323.4 [M+H]⁺.

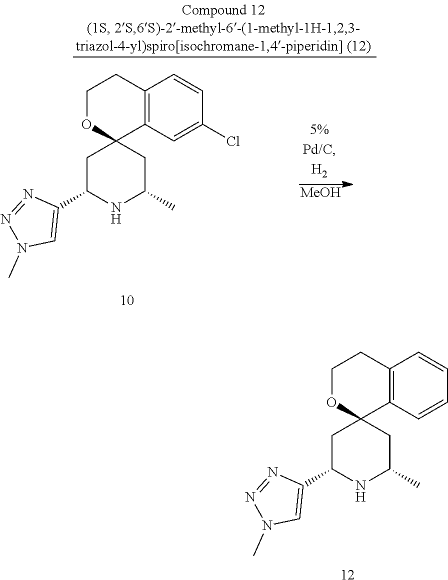 [SMILES: "MeOH" ; C[C@H]1C[C@@]2(C[C@@H](c3cn(C)nn3)N1)OCCc1ccc(Cl)cc12 ; "10" ; C[C@H]1C[C@@]2(C[C@@H](c3cn(C)nn3)N1)OCCc1ccccc12 ; "12"]

To a solution of (1S,2′S,6′S)-7-chloro-2′-methyl-6′-(1-methyltriazol-4-yl)spiro[isochromane-1,4′-piperidine](15 mg, 0.045 mmol) in MeOH (0.5 mL) was added 5% Pd/C (19 mg, 0.008927 mmol). The reaction was bubbled with H₂ for 1 minute, and then stirred under an atmosphere of H₂ (1 atm) overnight. The reaction was diluted with MeOH and filtered. The filtrate was concentrated in vacuo and the crude was purified by reverse-phase HPLC (Method: C18 Waters Sunfire column (30×150 mm, 5 micron), gradient: MeCN in H₂O with 0.1% trifluoroacetic acid) to afford the title compound 12 as a trifluoroacetate salt (19.7 mg, 81%). ¹H NMR (400 MHz, DMSO-d6) δ 9.29 (s, 1H), 8.96 (d, J=11.4 Hz, 1H), 8.24 (s, 1H), 7.35-7.06 (m, 4H), 4.72 (t, J=11.2 Hz, 1H), 4.08 (s, 3H), 3.93 (t, J=5.4 Hz, 2H), 3.63 (s, 1H), 2.80 (t, J=5.5 Hz, 2H), 2.42 (q, J=13.5 Hz, 1H), 2.28 (d, J=14.4 Hz, 1H), 2.18-1.87 (m, 2H), 1.36-1.22 (m, 3H). LCMS m/z 298.4 [M+H]⁺.

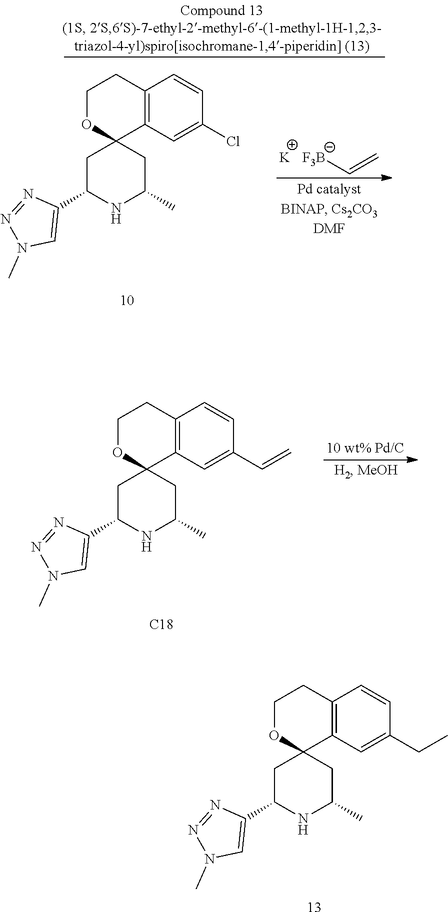

### Step 1. Synthesis of(1S,2′S,6′S)-2′-methyl-6′-(1-methyl-1H-1,2,3-triazol-4-yl)-7-vinylspiro[isochromane-1, 4′-piperidine](C18)

To a 2-dram vial was charged (1S,2′S,6′S)-7-chloro-2′-methyl-6′-(1-methyltriazol-4-yl)spiro[isochromane-1,4′-piperidine](30 mg, 0.090 mmol), potassium vinyltrifluoroborate (18 mg, 0.13 mmol), di-p-chloro-bis-[5-hydroxy-2-[1-(hydroxyimino-κN)-ethyl]-phenyl-κC]-palladium(II) dimer (N\acute{α}jera catalyst) (2.6 mg, 0.0045 mmol), 1-(2-diphenylphosphanyl-1-naphthyl)-2-naphthyl]-diphenyl-phosphane (5.6 mg, 0.0089 mmol) and cesium carbonate (88 mg, 0.27 mmol). The vial was capped and purged with N₂ (×3). DMF was added, and the mixture was heated at 120° C. overnight. The reaction was quenched with saturated NaHCO₃ and brine, and extracted with EtOAc (×3). The combined organic extracts were dried over Na₂SO₄, filtered and concentrated in vacuo. The crude was purified by reverse-phase HPLC (Method: C₁₈ Waters Sunfire column (30×150 mm, 5 micron), gradient: MeCN in H₂O with 0.1% trifluoroacetic acid) to afford the title compound C18 as a trifluoroacetate salt (14.4 mg, 32%). LCMS m/z 325.0 [M+H]⁺.

### Step 2. Synthesis of (1S,2′S,6′S)-7-ethyl-2′-methyl-6′-(1-methyl-1H-1,2,3-triazol-4-yl)spiro[isochromane-1,4′-piperidine](13)

To a solution of (1S,2′S,6′S)-2′-methyl-6′-(1-methyltriazol-4-yl)-7-vinyl-spiro[isochromane-1,4′-piperidine](trifluoroacetate salt) (14.4 mg, 0.044 mmol) in MeOH (1 mL) was added 10 wt % Pd/C (22 mg, 0.0087 mmol). The mixture was bubbled with H₂ for 2 minutes, and then stirred at room temperature under an atmosphere of H₂ (1 atm) overnight. The reaction was diluted with MeOH and filtered. The filtrate was concentrated in vacuo, and the crude was purified by reverse-phase HPLC (Method: C18 Waters Sunfire column (30×150 mm, 5 micron), gradient: MeCN in H₂O with 0.1% trifluoroacetic acid) to afford the title compound 13 as a trifluoroacetate salt (2.0 mg, 9%). ¹H NMR (400 MHz, DMSO-d6) δ 9.32 (d, J=10.2 Hz, 1H), 8.93 (s, 1H), 8.24 (s, 1H), 7.04 (d, J=28.6 Hz, 3H), 4.71 (t, J=11.2 Hz, 1H), 4.08 (s, 3H), 3.90 (t, J=5.4 Hz, 2H), 3.62 (s, 1H), 2.74 (q, J=9.1, 7.2 Hz, 2H), 2.65-2.38 (m, 3H), 2.29 (t, J=15.8 Hz, 1H), 2.13-1.94 (m, 2H), 1.29 (d, J=6.5 Hz, 3H), 1.18 (t, J=7.6 Hz, 3H). LCMS m/z 326.4 [M+H]⁺.

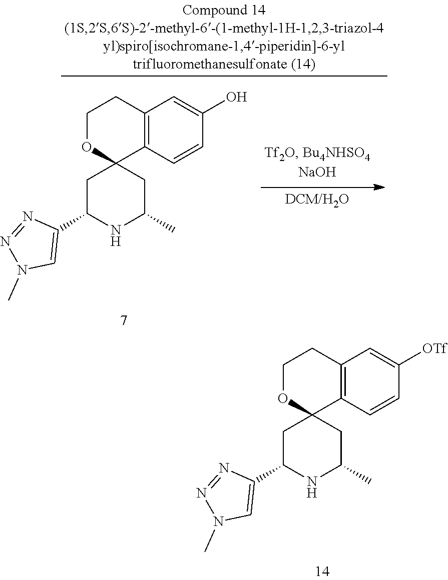 [SMILES: C[C@H]1C[C@@]2(C[C@@H](c3cn(C)nn3)N1)OCCc1cc(O)ccc12 ; C[C@H]1C[C@@]2(C[C@@H](c3cn(C)nn3)N1)OCCc1cc(OS(=O)(=O)C(F)(F)F)ccc12]

Compound 14 was prepared from compound 7 following the method described for compound S4. The reaction was purified by silica gel chromatography (0 to 20% MeOH in DCM) to afford the title compound 14 as a white form solid (338 mg, 67%). ¹H NMR (400 MHz, Methanol-d4) δ 7.84 (s, 1H), 7.39 (d, J=8.7 Hz, 1H), 7.23-7.17 (m, 1H), 7.15 (d, J=2.6 Hz, 1H), 4.40 (dd, J=11.7, 2.7 Hz, 1H), 4.08 (s, 3H), 3.97 (t, J=5.5 Hz, 2H), 3.34 (s, 1H), 2.88 (t, J=5.5 Hz, 2H), 2.27-2.19 (m, 1H), 2.02-1.91 (m, 2H), 1.61 (dd, J=13.8, 11.5 Hz, 1H), 1.17 (d, J=6.5 Hz, 3H). LCMS m/z 446.4 [M+H]⁺.

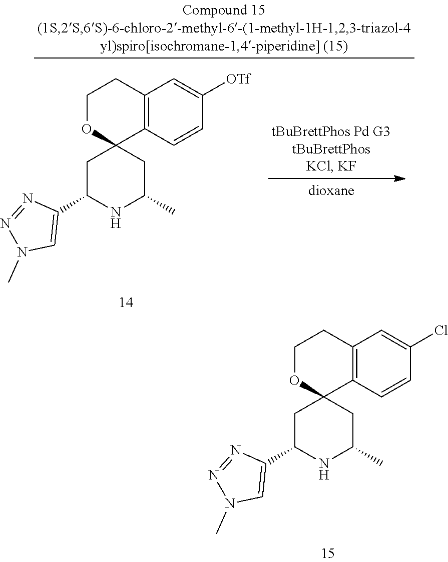 [SMILES: C[C@H]1C[C@@]2(C[C@@H](c3cn(C)nn3)N1)OCCc1cc(OS(=O)(=O)C(F)(F)F)ccc12 ; C[C@H]1C[C@@]2(C[C@@H](c3cn(C)nn3)N1)OCCc1cc(Cl)ccc12]

To a 1-dram vial was charged (1S,2′S,6′S)-2′-methyl-6′-(1-methyl-1H-1,2,3-triazol-4-yl)spiro[isochromane-1,4′-piperidin]-6-yl trifluoromethanesulfonate (25 mg, 0.056 mmol), tBuBrettPhos Pd G3 (4.8 mg, 0.0056 mmol), tBuBrettPhos (5.5 mg, 0.011 mmol), potassium chloride (8.4 mg, 0.11 mmol), and potassium fluoride (1.5 mg, 0.026 mmol). The vial was capped and purged with N₂ (×3). Dioxane (0.5 mL) was added, and the mixture was heated at 130° C. overnight. The reaction was cooled down to room temperature, filtered, and purified by reverse-phase HPLC (Method: C18 Waters Sunfire column (30×150 mm, 5 micron), gradient: MeCN in H₂O with 0.10% trifluoroacetic acid) to afford the title compound 15 as a trifluoroacetate salt (7.5 mg, 27%). ¹H NMR (400 MHz, DMSO-d6) δ 9.30 (s, 1H), 8.94 (d, J=11.2 Hz, 1H), 8.22 (s, 1H), 7.36 (dd, J=8.4, 2.3 Hz, 1H), 7.29 (d, J=2.3 Hz, 1H), 7.18 (d, J=8.4 Hz, 1H), 4.71 (t, J=11.0 Hz, 1H), 4.08 (s, 3H), 3.91 (t, J=5.4 Hz, 2H), 3.62 (s, 1H), 2.81 (t, J=5.5 Hz, 2H), 2.46-2.22 (m, 2H), 2.12 (d, J=14.2 Hz, 1H), 2.05-1.88 (m, 1H), 1.28 (d, J=6.5 Hz, 3H). LCMS m/z 332.8 [M+H]⁺.

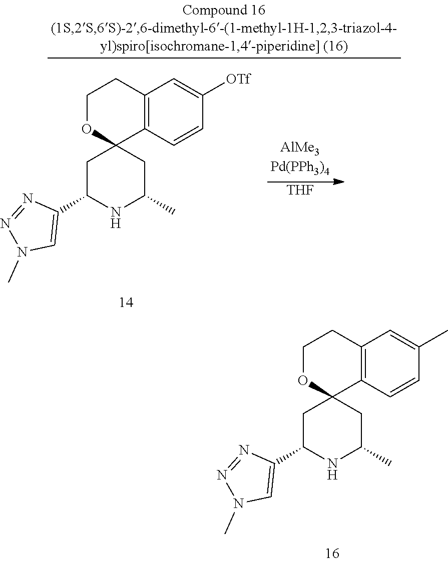 [SMILES: C[C@H]1C[C@@]2(C[C@@H](c3cn(C)nn3)N1)OCCc1cc(OS(=O)(=O)C(F)(F)F)ccc12 ; Cc1ccc2c(c1)CCO[C@@]21C[C@@H](c2cn(C)nn2)N[C@@H](C)C1 ; "THF"]

To a 1-dram vial was charged (1S,2′S,6′S)-2′-methyl-6′-(1-methyl-1H-1,2,3-triazol-4-yl)spiro[isochromane-1,4′-piperidin]-6-yl trifluoromethanesulfonate (25 mg, 0.056 mmol) and Pd(PPh₃)₄ (13.1 mg, 0.011 mmol). The vial was capped and purged with N₂ (×3). THF (0.5 mL) was added followed by AlMe₃ (70 μL, 0.14 mmol, 2M in heptane). The reaction was heated at reflux overnight. The reaction was cooled down to room temperature, and then quenched slowly with MeOH. The volatile was removed, and the crude was purified by reverse-phase HPLC (Method: C18 Waters Sunfire column (30×150 mm, 5 micron), gradient: MeCN in H₂O with 0.10% trifluoroacetic acid) to afford the title compound 16 as a trifluoroacetate salt (9.6 mg, 38%). ¹H NMR (400 MHz, DMSO-d6) δ 9.28 (d, J=10.6 Hz, 1H), 8.93 (d, J=11.2 Hz, 1H), 8.21 (s, 1H), 7.14-6.87 (m, 3H), 4.71 (s, 1H), 4.08 (s, 3H), 3.90 (t, J=5.4 Hz, 2H), 3.60 (s, 1H), 2.74 (q, J=7.5, 6.5 Hz, 2H), 2.38 (d, J=13.0 Hz, 2H), 2.26 (s, 3H), 2.03 (dd, J=37.9, 13.6 Hz, 2H), 1.28 (d, J=6.5 Hz, 3H). LCMS m/z 312.4 [M+H]⁺.

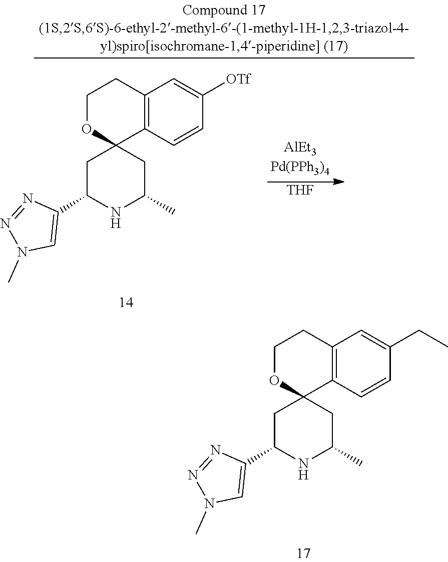 [SMILES: C[C@H]1C[C@@]2(C[C@@H](c3cn(C)nn3)N1)OCCc1cc(OS(=O)(=O)C(F)(F)F)ccc12 ; CCc1ccc2c(c1)CCO[C@@]21C[C@@H](c2cn(C)nn2)N[C@@H](C)C1 ; "THF"]

To a 1-dram vial was charged (1S,2′S,6′S)-2′-methyl-6′-(1-methyl-1H-1,2,3-triazol-4-yl)spiro[isochromane-1,4′-piperidin]-6-yl trifluoromethanesulfonate (25 mg, 0.056 mmol) and Pd(PPh₃)₄ (13.1 mg, 0.011 mmol). The vial was capped and purged with N₂ (×3). THF (0.5 mL) was added followed by AlEt₃ (75 μL, 0.5571 mmol, 25 wt. % in toluene). The reaction was heated at reflux overnight. The reaction was cooled down to room temperature, and then quenched slowly with MeOH. The volatile was removed, and the crude was purified by reverse-phase HPLC (Method: C18 Waters Sunfire column (30×150 mm, 5 micron), gradient: MeCN in H₂O with 0.10% trifluoroacetic acid) to afford the title compound 17 as a trifluoroacetate salt (17.3 mg, 69%). ¹H NMR (400 MHz, DMSO-d6) δ 9.28 (d, J=9.7 Hz, 1H), 8.92 (d, J=11.0 Hz, 1H), 8.23 (s, 1H), 7.18-6.91 (m, 3H), 4.71 (t, J=11.4 Hz, 1H), 4.09 (d, J=2.6 Hz, 3H), 3.91 (t, J=5.4 Hz, 2H), 3.55 (s, 1H), 2.83-2.62 (m, 2H), 2.55-2.45 (m, 3H), 2.30-1.89 (m, 3H), 1.28 (d, J=6.5 Hz, 3H), 1.16 (td, J=7.6, 2.0 Hz, 3H). LCMS m/z 326.4 [M+H]⁺.

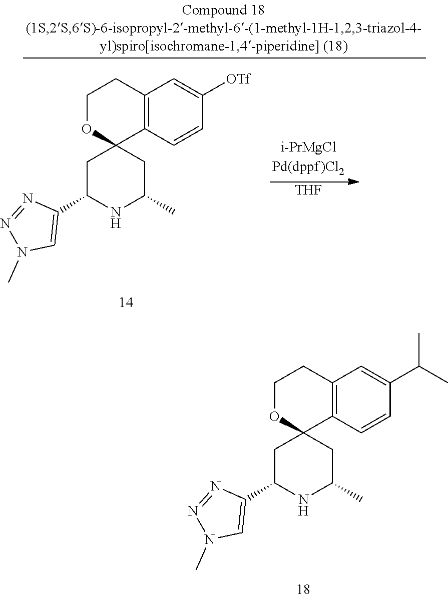 [SMILES: C[C@H]1C[C@@]2(C[C@@H](c3cn(C)nn3)N1)OCCc1cc(OS(=O)(=O)C(F)(F)F)ccc12 ; CC(C)c1ccc2c(c1)CCO[C@@]21C[C@@H](c2cn(C)nn2)N[C@@H](C)C1 ; "THF"]

To an oven-dried 1-dram vial was charged (1S,2′S,6′S)-2′-methyl-6′-(1-methyl-1H-1,2,3-triazol-4-yl)spiro[isochromane-1,4′-piperidin]-6-yl trifluoromethanesulfonate (30 mg, 0.064 mmol) and Pd(dppf)Cl₂ (5.2 mg, 0.0064 mmol). The vial was capped and purged with N₂ (×3). THF (0.5 mL) was added followed by i-PrMgCl (96 μL, 0.19 mmol, 2M in THF). The reaction was heated at 50° C. for 3.5 hours. The reaction was cooled down to room temperature, and then quenched slowly with MeOH. The volatile was removed, and the crude was purified by reverse-phase HPLC (Method: C18 Waters Sunfire column (30×150 mm, 5 micron), gradient: MeCN in H₂O with 0.10% trifluoroacetic acid) to afford the title compound 18 as a trifluoroacetate salt (15.9 mg, 49%). ¹H NMR (400 MHz, Methanol-d4) δ 8.03 (s, 1H), 7.12 (s, 2H), 7.03 (s, 1H), 4.93-4.85 (m, 1H), 4.12 (s, 3H), 3.97 (t, J=5.5 Hz, 2H), 3.83 (s, 1H), 2.92-2.80 (m, 3H), 2.46 (dd, J=14.6, 12.2 Hz, 1H), 2.41-2.33 (m, 1H), 2.26-2.18 (m, 1H), 2.05-1.93 (m, 1H), 1.40 (d, J=6.6 Hz, 3H), 1.22 (d, J=6.9 Hz, 6H). LCMS m/z 340.4 [M+H]⁺.

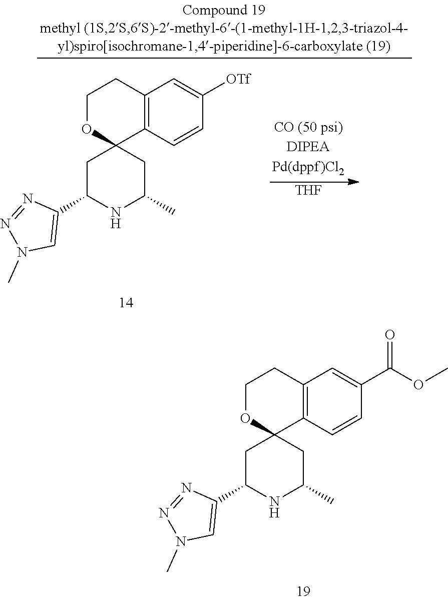 [SMILES: C[C@H]1C[C@@]2(C[C@@H](c3cn(C)nn3)N1)OCCc1cc(OS(=O)(=O)C(F)(F)F)ccc12 ; COC(=O)c1ccc2c(c1)CCO[C@@]21C[C@@H](c2cn(C)nn2)N[C@@H](C)C1 ; "THF"]

To a solution of (1S,2′S,6′S)-2′-methyl-6′-(1-methyl-1H-1,2,3-triazol-4-yl)spiro[isochromane-1,4′-piperidin]-6-yl trifluoromethanesulfonate (300 mg, 0.67 mmol) in MeOH (6 mL) in a 100-mL pressure tube was added Pd(dppf)Cl₂ (55 mg, 0.067 mmol) and DIPEA (328 μL, 1.88 mmol). The tube was purged sequentially with N2 (×3) and CO (×3), and then heated at 80° C. under an atmosphere of CO (50 psi) for 48 hours. The reaction was cooled down to room temperature, and the volatile was removed. The crude was purified by silica gel chromatography (0 to 20% MeOH in DCM) to afford the title compound 19 (200 mg, 76%). ¹H NMR (300 MHz, Methanol-d4) δ 7.93-7.73 (m, 3H), 7.35 (d, J=8.2 Hz, 1H), 4.52 (dd, J=11.8, 3.0 Hz, 1H), 4.09 (s, 3H), 3.98 (t, J=5.5 Hz, 2H), 3.89 (s, 3H), 3.45 (ddd, J=11.6, 6.5, 2.7 Hz, 1H), 2.89 (t, J=5.5 Hz, 2H), 2.33-2.20 (m, 1H), 2.18-1.97 (m, 2H), 1.72 (dd, J=14.0, 11.6 Hz, 1H), 1.23 (d, J=6.5 Hz, 3H). LCMS m/z 357.2 [M+H]⁺.

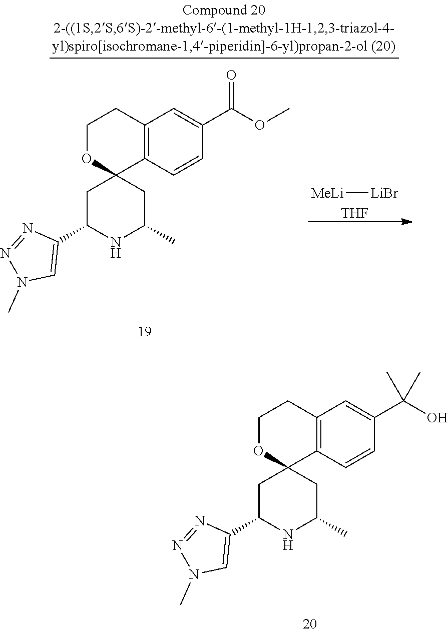

To an oven-dried 2-dram vial was charged methyl (1S,2′S,6′S)-2′-methyl-6′-(1-methyltriazol-4-yl)spiro[isochromane-1,4′-piperidine]-6-carboxylate (43 mg, 0.12 mmol) and THF (1 mL). The resulting solution was cooled down to −78° C., and MeLi-LiBr (322 μL, 0.48 mmol, 1.5M in Et₂O) was added slowly. The reaction was stirred at the same temperature for 50 minutes, and then quenched with saturated NaHCO₃, and extracted with EtOAc (×3). The combined organic extracts were washed with brine, dried over MgSO₄, filtered, and concentrated in vacuo. The crude was purified by reverse-phase HPLC (Method: C18 Waters Sunfire column (30×150 mm, 5 micron), gradient: MeCN in H₂O with 0.1% trifluoroacetic acid) to afford the title compound 20 as a trifluoroacetate salt (1.2 mg, 3%). ¹H NMR (300 MHz, Methanol-d4) δ 8.01 (s, 1H), 7.36 (d, J=8.3 Hz, 1H), 7.28 (s, 1H), 7.16 (d, J=8.3 Hz, 1H), 4.81 (s, 1H), 4.11 (s, 3H), 3.98 (t, J=5.4 Hz, 2H), 3.78 (s, 1H), 2.86 (t, J=5.4 Hz, 2H), 2.52-2.27 (m, 2H), 2.19 (d, J=14.9 Hz, 1H), 2.04-1.88 (m, 1H), 1.50 (s, 6H), 1.38 (d, J=6.6 Hz, 3H). LCMS m/z 356.5 [M+H]⁺.

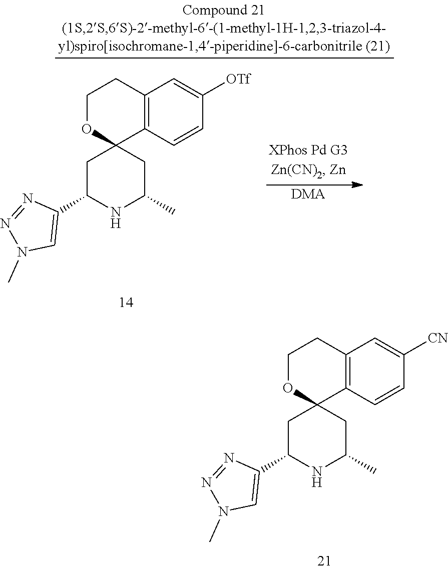

Compound 21 was prepared from compound 14 following the method described for compound 11. The reaction was purified by reverse-phase HPLC (Method: C18 Waters Sunfire column (30×150 mm, 5 micron), gradient: MeCN in H₂O with 0.1% trifluoroacetic acid) to afford the title compound 21 as a trifluoroacetate salt (23.3 mg, 26%). ¹H NMR (400 MHz, Methanol-d4) δ 8.04 (d, J=1.5 Hz, 1H), 7.66-7.57 (m, 2H), 7.41 (dd, J=8.2, 2.1 Hz, 1H), 4.91 (dd, J=12.3, 3.7 Hz, 1H), 4.12 (s, 3H), 4.02 (t, J=5.5 Hz, 2H), 3.85 (s, 1H), 2.93 (d, J=11.1 Hz, 1H), 2.93 (s, 1H), 2.57-2.46 (m, 1H), 2.42 (d, J=13.9 Hz, 1H), 2.28 (d, J=14.7 Hz, 1H), 2.02 (t, J=13.5 Hz, 1H), 1.41 (d, J=6.6 Hz, 3H). LCMS m/z 323.4 [M+H]⁺.

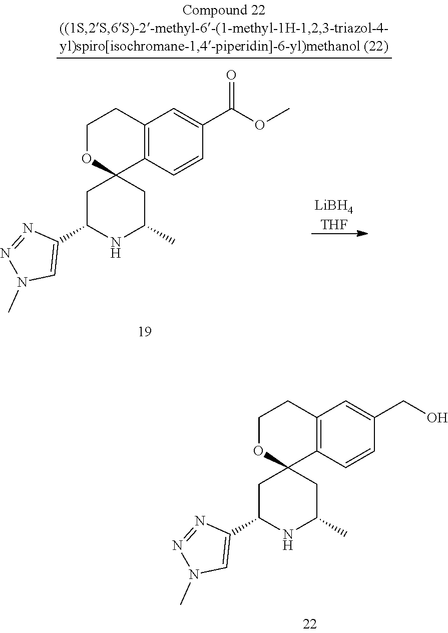 [SMILES: COC(=O)c1ccc2c(c1)CCO[C@@]21C[C@@H](c2cn(C)nn2)N[C@@H](C)C1 ; C[C@H]1C[C@@]2(C[C@@H](c3cn(C)nn3)N1)OCCc1cc(CO)ccc12 ; "LiBH4THF"]

To a solution of methyl (1S,2′S,6′S)-2′-methyl-6′-(1-methyltriazol-4-yl)spiro[isochromane-1,4′-piperidine]-6-carboxylate (20 mg, 0.056 mmol) in THF (0.5 mL) was added LiBH₄ (2.5 mg, 0.1148 mmol). The mixture was heated at reflux for 2 hours. The reaction was cooled down to room temperature and quenched with EtOAc. The volatile was removed, and the crude was purified by reverse-phase HPLC (Method: C18 Waters Sunfire column (30×150 mm, 5 micron), gradient: MeCN in H₂O with 0.1% trifluoroacetic acid) to afford the title compound 22 as a trifluoroacetate salt (14.7 mg, 75%). ¹H NMR (300 MHz, Methanol-d4) δ 8.04 (s, 1H), 7.35-7.12 (m, 3H), 4.90-4.80 (m, 1H), 4.57 (s, 2H), 4.12 (s, 3H), 3.99 (t, J=5.4 Hz, 2H), 3.87 (s, 1H), 2.86 (t, J=5.5 Hz, 2H), 2.57-2.30 (m, 2H), 2.23 (d, J=14.7 Hz, 1H), 2.01 (dd, J=14.7, 12.1 Hz, 1H), 1.40 (d, J=6.6 Hz, 3H). LCMS m/z 328.4 [M+H]⁺.

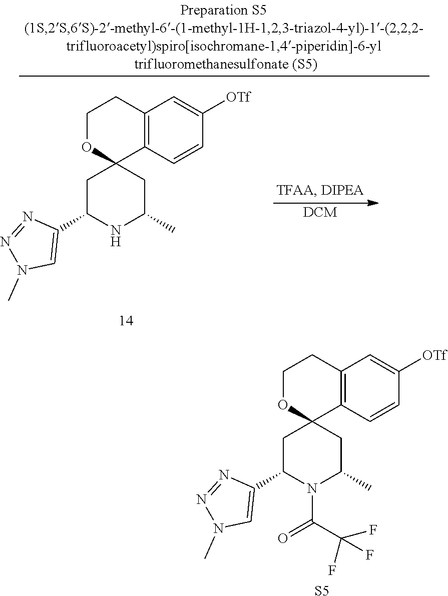

To a solution of [(1S,2′S,6′S)-2′-methyl-6′-(1-methyltriazol-4-yl)spiro[isochromane-1,4′-piperidine]-6-yl]trifluoromethanesulfonate (529 mg, 1.185 mmol) in DCM (6 mL) was added DIPEA (268 μL, 1.539 mmol). The resulting solution was cooled down to 0° C., and (2,2,2-trifluoroacetyl) 2,2,2-trifluoroacetate (181 μL, 1.302 mmol) was added dropwise over 15 minutes. The reaction was stirred at the same temperature for 2 hours. The reaction was quenched with saturated NH₄Cl solution, and then extracted with DCM (×3). The combined organic extracts were washed with brine, dried over Na₂SO₄, filtered, and concentrated in vacuo. The crude was purified by silica gel chromatography (0 to 100% EtOAc in heptane) to afford the title compound S5 as a white form solid (632 mg, 98%). LCMS m/z 543.2 [M+H]⁺.

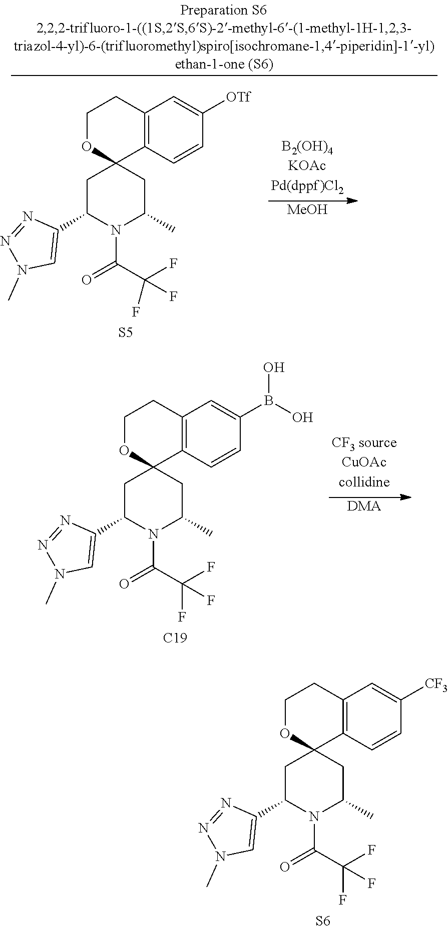

### Step 1. Synthesis of ((1S,2′S,6′S)-2′-methyl-6′-(1-methyl-1H-1, 2,3-triazol-4-yl)-1′-(2,2,2-trifluoroacetyl)spiro[isochromane-1,4′-piperidin]-6-yl)boronic acid (C19)

To a 2-dram vial was charged (1S,2′S,6′S)-2′-methyl-6′-(1-methyl-1H-1,2,3-triazol-4-yl)-1′-(2,2,2-trifluoroacetyl)spiro[isochromane-1,4′-piperidin]-6-yl]trifluoromethanesulfonate (120 mg, 0.22 mmol), tetrahydroxydiboron (40 mg, 0.45 mmol), KOAc (65 mg, 0.66 mmol), and Pd(dppf)Cl₂ (18 mg, 0.022 mmol). The vial was capped and purged with N₂ (×3), and then MeOH (1 mL) was added. The reaction was heated at 40° C. overnight. The reaction was cooled down to room temperature, quenched with water, and extracted with DCM (×3). The combined organic extracts were washed with brine, dried over Na₂SO₄, filtered, and concentrated in vacuo. The crude was purified by silica gel chromatography (0 to 20% MeOH in DCM) to afford the title compound C19 as a brown solid (90.3 mg, 93%). LCMS m/z 439.3 [M+H]⁺.

### Step 2. Synthesis of 2,2,2-trifluoro-]-((1S,2′S,6′S)-2′-methyl-6′-(1-methyl-1H-1, 2, 3-triazol-4-yl)-6-(trifluoromethyl)spiro[isochromane-1,4′-piperidin]-1′-yl)ethan-I-one (S6)

To a 1-dram vial was charged ((1S,2′S,6′S)-2′-methyl-6′-(1-methyl-1H-1,2,3-triazol-4-yl)-1′-(2,2,2-trifluoroacetyl)spiro[isochromane-1,4′-piperidin]-6-yl)boronic acid (1020 mg, 2.328 mmol), CuOAc (71 mg, 0.579 mmol), and 5-(trifluoromethyl)dibenzothiophen-5-ium trifluoromethanesulfonate (1.50 g, 3.728 mmol). The vial was capped and purged with N₂ (×3), and then DMA (6 mL) was added followed by collidine (616 μL, 4.661 mmol). The resulting dark green solution was stirred at room temperature overnight. The reaction was quenched with water, extracted with EtOAc (×3). The combined organic extracts were washed with brine, dried over Na₂SO₄, filtered, and concentrated in vacuo. The crude was purified by silica gel chromatography (0 to 100% EtOAc in heptane) to afford the title compound S6 as a white form solid (578 mg, 54%). LCMS m/z 463.2 [M+H]⁺.

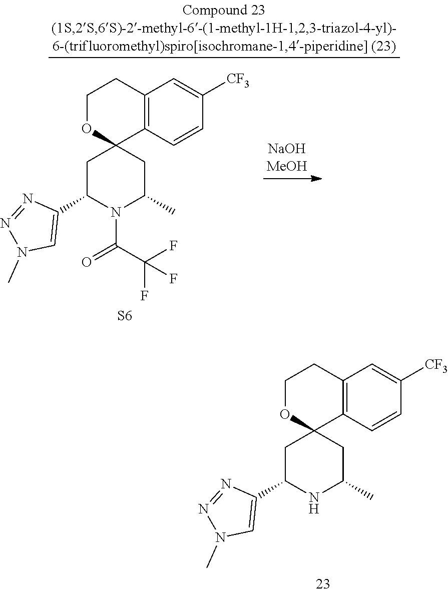

To a solution of 2,2,2-trifluoro-1-[(1S,2′S,6′S)-2′-methyl-6′-(1-methyltriazol-4-yl)-6-(trifluoromethyl)spiro[isochromane-1,4′-piperidine]-1′-yl]ethanone (52 mg, 0.11 mmol) in MeOH (1 mL) was added a 6 M aqueous solution of NaOH (225 μL, 1.35 mmol). The reaction was heated at 60° C. for 1 hour, and then cooled down to room temperature. The volatile was removed, and the crude was purified by reverse-phase HPLC (Method: C18 Waters Sunfire column (30×150 mm, 5 micron), gradient: MeCN in H₂O with 0.1% trifluoroacetic acid) to afford the title compound 23 as a trifluoroacetate salt (13.2 mg, 21%). ¹H NMR (400 MHz, Methanol-d4) δ 8.05 (s, 1H), 7.56 (d, J=8.3 Hz, 1H), 7.51 (s, 1H), 7.42 (d, J=8.2 Hz, 1H), 4.97-4.88 (m, 1H), 4.12 (d, J=1.6 Hz, 3H), 4.03 (t, J=5.5 Hz, 2H), 3.86 (s, 1H), 2.95 (t, J=5.5 Hz, 2H), 2.49 (dt, J=29.7, 14.6 Hz, 2H), 2.29 (d, J=14.8 Hz, 1H), 2.04 (dd, J=14.8, 12.2 Hz, 1H), 1.42 (d, J=6.4 Hz, 3H). LCMS m/z 366.4 [M+H]⁺.

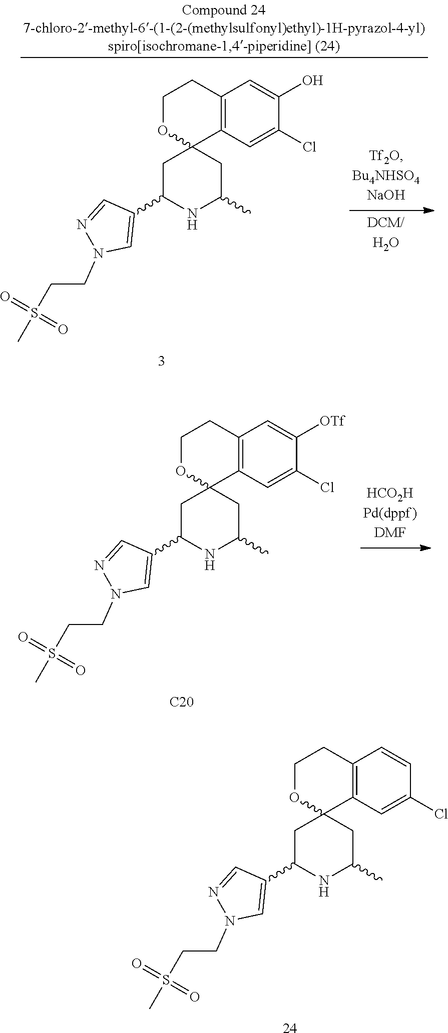

### Step 1. Synthesis of 7-chloro-2′-methyl-6′-(1-(2-(methylsulfonyl)ethyl)-]H-pyrazol-4-yl)spiro[isochromane-1,4′-piperidin]-6-yl trifluoromethanesulfonate (C20)

Compound C20 was prepared from compound 3 following the method described for compound S4. The reaction was purified by silica gel chromatography (0 to 20% MeOH in DCM) to afford the title compound C20 as a white solid (111 mg, 72%). LCMS m/z 571.1 [M+H]⁺.

### Step 2. Synthesis of 7-chloro-2′-methyl-6′-(1-(2-(methylsulfonyl)ethyl)-]H-pyrazol-4-yl)spiro[isochromane-1,4′-piperidine](24)

Compound 24 was prepared from compound C20 following the method described for compound 10. The reaction was purified by reverse-phase HPLC (Method: C18 Waters Sunfire column (30×150 mm, 5 micron), gradient: MeCN in H₂O with 0.1% trifluoroacetic acid) to afford the title compound 24 as a trifluoroacetate salt (16.6 mg, 77%). ¹H NMR (300 MHz, Methanol-d4) δ 7.94 (s, 1H), 7.73 (s, 1H), 7.31-7.15 (m, 3H), 4.78-4.69 (m, 1H), 4.66 (t, J=6.3 Hz, 2H), 3.97 (t, J=5.5 Hz, 2H), 3.81 (s, 1H), 3.71 (t, J=6.3 Hz, 2H), 2.85 (s, 5H), 2.40-2.30 (m, 2H), 2.24 (d, J=14.9 Hz, 1H), 1.99-1.83 (m, 1H), 1.38 (d, J=6.6 Hz, 3H). LCMS m/z 424.0 [M+H]⁺.

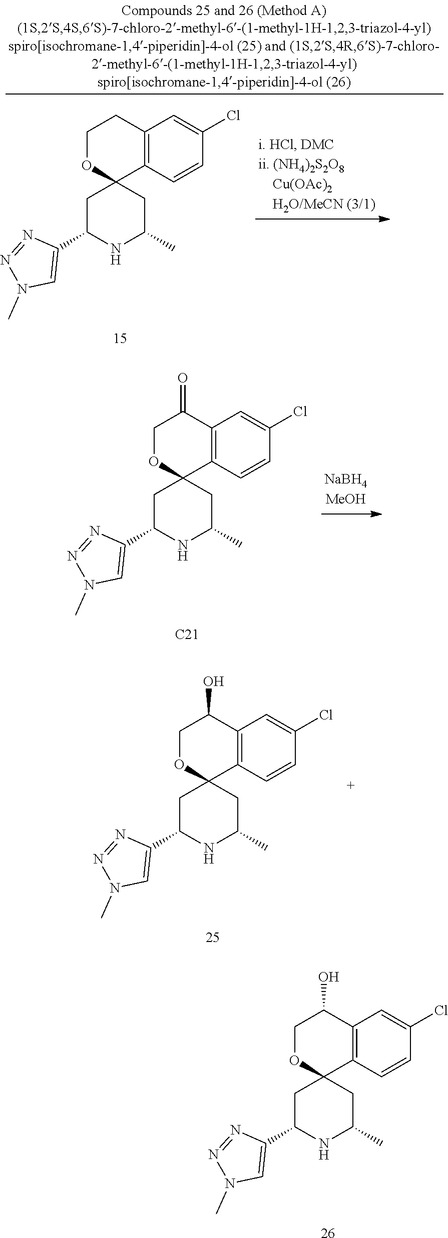

### Step 1. Synthesis of (]S,2′S,6′S)-7-chloro-2′-methyl-6′-(1-methyl-1H-1,2,3-triazol-4-yl)spiro[isochromane-1,4′-piperidin]-4-one (C21)

To a solution of (1S,2′S,6′S)-6-chloro-2′-methyl-6′-(1-methyltriazol-4-yl)spiro[isochromane-1,4′-piperidine](hydrochloride salt) (29.8 mg, 0.081 mmol) in MeCN (0.15 mL) and H₂O (0.45 mL) was added ammonium hydrogen sulfate (65 mg, 0.28 mmol) and Cu(OAc)₂ (4.5 mg, 0.025 mmol). The resulting pale blue solution was stirred at 50° C. overnight. The reaction was quenched with saturated NaHCO₃ solution and brine, and extracted with EtOAc (×3). The combined organic extracts were washed with brine, dried over Na₂SO₄, filtered, and concentrated in vacuo to afford the title compound C21. The crude was directly without further purification. LCMS m/z 349.0 [M+H]⁺.

### Step 2. Synthesis of (1S,2′S,4S,6′S)-7-chloro-2′-methyl-6′-(1-methyl-1H-1,2,3-triazol-4-yl)spiro[isochromane-1,4′-piperidin]-4-ol (25) and (]S,2′S,4R,6′S)-7-chloro-2′-methyl-6′-(1-methyl-1H-1,2,3-triazol-4-yl)spiro[isochromane-1,4′-piperidin]-4-ol (26)

To a solution of (1S,2′S,6′S)-7-chloro-2′-methyl-6′-(1-methyl-1H-1,2,3-triazol-4-yl)spiro[isochromane-1,4′-piperidin]-4-one (28 mg, 0.081 mmol) in MeOH (1.5 mL) was added NaBH₄ (3.0 mg, 0.079 mmol). The reaction was stirred at room temperature for 15 minutes. EtOAc was added and stirred for 10 minutes, and then the volatile was removed. The crude was purified by chiral SFC separation (Column: Daicel Chiralpak® AD-H, 10×250 mm; Mobile Phase: 40% ethanol (5 mM ammonia), 60% CO₂. Flow: 15 mL/min, isocratic) to afford diastereomer 25 (1.0 mg, 3%) and diastereomer 26 (1.2 mg, 4%). The absolute stereochemistry was assigned by comparison to the authentic sample obtained from asymmetric reduction of the ketone intermediate (Method B).

Characterization data for compound 25: ¹H NMR (400 MHz, Methanol-d4) δ 7.83 (s, 1H), 7.46 (d, J=2.1 Hz, 1H), 7.29 (dd, J=8.4, 2.2 Hz, 1H), 7.22 (d, J=8.4 Hz, 1H), 4.55 (t, J=4.8 Hz, 1H), 4.37 (dd, J=11.9, 2.7 Hz, 1H), 4.08 (s, 3H), 4.01 (dd, J=11.7, 4.0 Hz, 1H), 3.75 (dd, J=11.8, 5.8 Hz, 1H), 3.40-3.30 (m, 1H), 2.17 (dt, J=13.8, 2.5 Hz, 1H), 2.06 (dt, J=14.1, 2.6 Hz, 1H), 1.89 (dd, J=13.7, 11.9 Hz, 1H), 1.58 (dd, J=13.8, 11.5 Hz, 1H), 1.17 (d, J=6.4 Hz, 3H). LCMS m/z 348.8 [M+H]⁺.

Characterization data for compound 26: ¹H NMR (300 MHz, Methanol-d4) δ 7.84 (s, 1H), 7.46 (d, J=2.2 Hz, 1H), 7.33-7.17 (m, 2H), 4.56 (dd, J=5.9, 4.1 Hz, 1H), 4.40 (dd, J=11.7, 2.7 Hz, 1H), 4.08 (s, 3H), 4.01 (dd, J=11.8, 4.1 Hz, 1H), 3.75 (dd, J=11.8, 6.0 Hz, 1H), 3.60 (q, J=7.1 Hz, 1H), 2.29 (dt, J=13.7, 2.6 Hz, 1H), 2.01-1.86 (m, 2H), 1.53 (dd, J=13.8, 11.4 Hz, 1H), 1.23-1.08 (m, 3H). LCMS m/z 348.8 [M+H]⁺.

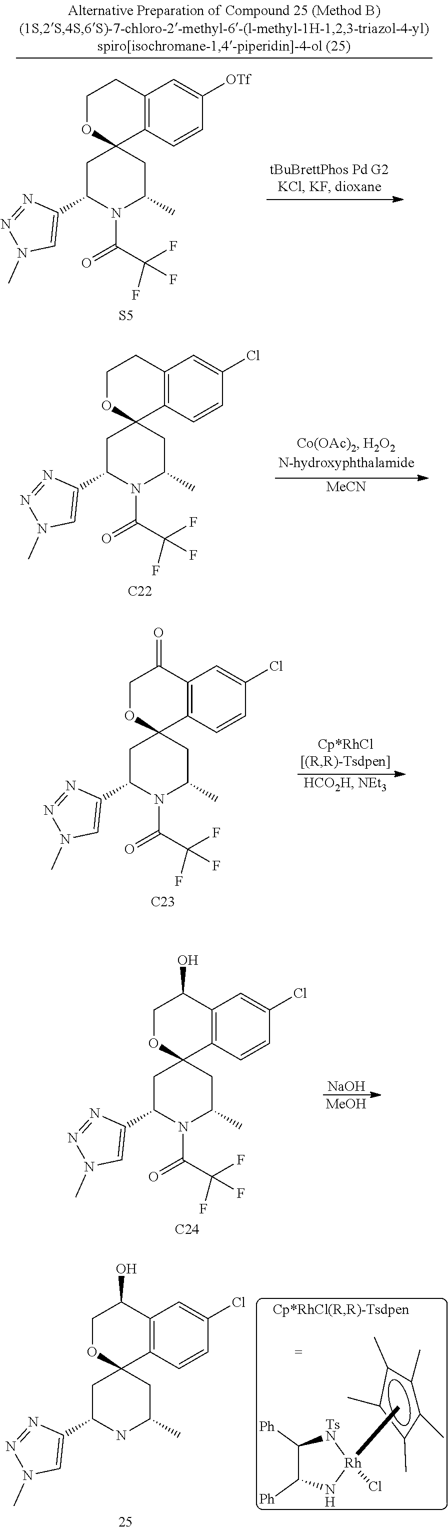

### Step 1. Synthesis of]-((1S,2′S,6′S)-6-chloro-2′-methyl-6′-(1-methyl-1H-1, 2, 3-triazol-4-yl)spiro[isochromane-1,4′-piperidin]-1′-yl)-2,2,2-trifluoroethan-1-one (C22)

To a pressure tube was charged [(1S,2′S,6′S)-2′-methyl-6′-(1-methyltriazol-4-yl)-1′-(2,2,2-trifluoroacetyl)spiro[isochromane-1,4′-piperidine]-6-yl]trifluoromethanesulfonate (4 g, 7.005 mmol), tBuBrettPhos Pd G2 (630 mg, 0.737 mmol), potassium chloride (1.35 g, 18.11 mmol) and potassium fluoride (270 mg, 4.647 mmol). The tube was capped and purged with N₂ (×3), and then dioxane (36 mL) was added. The tube was sealed and heated at 130° C. behind a blast shield for 24 hours. The reaction was cooled down to room temperature, quenched with water and brine (1/1), and then extracted with DCM (×3). The combined organic extracts were concentrated in vacuo. The crude was purified by silica gel chromatography (0 to 40% EtOAc in heptane) to afford the title compound C22 as a light brown foam solid (2.0 g, 63%). ¹H NMR (300 MHz, Chloroform-d) δ 7.53 (s, 1H), 7.23-7.11 (m, 1H), 7.01 (d, J=2.1 Hz, 1H), 5.53 (s, 1H), 4.35 (q, J=7.2 Hz, 1H), 4.04 (s, 3H), 3.78 (t, J=5.6 Hz, 2H), 3.22 (dd, J=14.6, 6.4 Hz, 1H), 2.72 (q, J=5.3 Hz, 2H), 2.44 (dd, J=14.6, 8.3 Hz, 1H), 2.22-1.95 (m, 1H), 1.34-1.10 (m, 5H). LCMS m/z 429.1 [M+H]⁺.

### Step 2. Synthesis of (1S,2′S,6′S)-6-chloro-2′-methyl-6′-(1-methyl-1H-1,2,3-triazol-4-yl)-1′-(2,2,2-trifluoroacetyl)spiro[isochromane-1, 4′-piperidin]-4-one (C23)

To a solution of 1-[(1S,2′S,6′S)-6-chloro-2′-methyl-6′-(1-methyltriazol-4-yl)spiro[isochromane-1,4′-piperidine]-1′-yl]-2,2,2-trifluoro-ethanone (2.96 g, 6.902 mmol) in MeCN (100 mL) was added cobalt acetate tetrahydrate (90 mg, 0.361 mmol) and N-hydroxy phthalimide (2.9 g, 17.78 mmol). H₂O₂ (1.6 mL, 15.66 mmol, 30% w/w) was added slowly. The reaction was heated at 50° C. for 5 hours, during which additional H₂O₂ (1.6 mL, 15.66 mmol, 30% w/w) and cobalt acetate tetrahydrate (90 mg, 0.361 mmol) were added at every hour. The reaction was cooled down to room temperature, and then quenched with saturated Na₂S203 solution and saturated NaHCO₃ solution. The reaction was extracted with DCM (×3). The combined organic extracts were concentrated in vacuo. The crude was purified by silica gel chromatography (0 to 50% EtOAc in heptane) to afford the title compound C23 as a white solid (916 mg, 29%). 1H NMR (300 MHz, Chloroform-d) δ 8.00 (s, 1H), 7.67-7.58 (m, 3H), 5.65 (s, 2H), 4.49-4.33 (m, 3H), 4.13 (s, 3H), 3.47 (dd, J=15.0, 5.3 Hz, 1H), 2.67 (dd, J=15.1, 8.7 Hz, 1H), 2.23 (s, 2H), 1.25 (s, 3H). LCMS m/z 443.1 [M+H]⁺.

### Step 3. Synthesis of]-((S,2′S,4S,6′S)-6-chloro-4-hydroxy-2′-methyl-6′-(1-methyl-1H-1,2,3-triazol-4-yl)spiro[isochromane-1,4′-piperidin]-1′-yl)-2,2,2-trifluoroethan-1-one (C24)

To a 100-mL 3-neck RBF was added 1,2,3,4,5-pentamethylcyclopentane; rhodium tetrachloride (8.2 mg, 0.013 mmol) and N-[(1R,2R)-2-amino-1,2-diphenyl-ethyl]-4-methyl-benzenesulfonamide (12 mg, 0.0327 mmol) followed by MeCN (7 mL). The mixture was stirred at room temperature for 20 minutes, and then TEA (605 μL, 4.341 mmol) and formic acid (410 μL, 10.87 mmol) (a 5:2 commercial solution from Oakwood) were added. The reaction mixture turned bright orange immediately, and some effervescence was observed. The mixture was cooled down to −15° C. in an acetone/dry ice bath. To a separated flask was added (1S,2′S,6′S)-6-chloro-2′-methyl-6′-(1-methyltriazol-4-yl)-1′-(2,2,2-trifluoroacetyl)spiro[isochromane-1,4′-piperidine]-4-one (910 mg, 1.973 mmol) and MeCN (7 mL) and DCM (3 mL). The mixture was cooled down to −15° C. to form a slurry, and then added to the first flask. The reaction was stirred while maintaining the internal temperature between −5° C. and −25° C. for 5 hours. The reaction was quenched with saturated NaHCO₃ solution, and then extracted with DCM (×2). The combined organic extracts were concentrated in vacuo. The crude was purified by silica gel chromatography (0 to 50% EtOAc in heptane) to afford the title compound C24 as an off-white foam solid (766 mg, 84%). ¹H NMR (300 MHz, Chloroform-d) δ 7.60 (s, 2H), 7.43 (s, 1H), 7.34 (d, J=7.8 Hz, 1H), 5.58 (s, 1H), 4.60-4.43 (m, 2H), 4.11 (s, 3H), 3.96 (dd, J=12.1, 3.2 Hz, 1H), 3.80 (dd, J=12.1, 4.4 Hz, 1H), 3.20 (dd, J=15.1, 6.1 Hz, 1H), 2.58 (d, J=11.7 Hz, 1H), 2.38-2.17 (m, 3H), 1.53-0.83 (m, 3H). LCMS m/z 444.1 [M+H]⁺.

Note that stereochemistry of alcohol C24 was assigned based on literature understanding of reductions using this catalyst and ligand system. (Reference: New Chiral Rhodium and Iridium Complexes with Chiral Diamine Ligands for Asymmetric Transfer Hydrogenation of Aromatic Ketones. Kunihiko Murata, Takao Ikariya, and Ryoji Noyori. The Journal of Organic Chemistry 1999 64 (7), 2186-2187).

### Step 4. Synthesis of (1S,2′S,4S,6′S)-7-chloro-2′-methyl-6′-(1-methyl-1H-1,2, 3-triazol-4-yl)spiro[isochromane-1,4′-piperidin]-4-ol (25)

To a solution of 1-[(1S,2′S,4S,6′S)-6-chloro-4-hydroxy-2′-methyl-6′-(1-methyltriazol-4-yl)spiro[isochromane-1,4′-piperidine]-1′-yl]-2,2,2-trifluoro-ethanone (760 mg, 1.642 mmol) in MeOH (9 mL) was added NaOH (3 mL, 18.00 mmol, 6M in H₂O). The reaction was heated at 60° C. for 1.5 hours. The reaction was cooled down to room temperature, and then diluted with water. The pH was adjusted to 11 by the addition of saturated NH₄Cl solution. MeOH was removed in vacuo, and the remaining aqueous solution was extracted with MTBE (×3). The combined organic extracts were washed with brine, dried over Na₂SO₄, filtered, and concentrated in vacuo to afford the title compound 25 as a white solid (560 mg, 97%). ¹H NMR (400 MHz, Methanol-d4) δ 7.83 (s, 1H), 7.46 (d, J=2.1 Hz, 1H), 7.29 (dd, J=8.4, 2.2 Hz, 1H), 7.22 (d, J=8.4 Hz, 1H), 4.55 (t, J=4.8 Hz, 1H), 4.37 (dd, J=11.9, 2.7 Hz, 1H), 4.08 (s, 3H), 4.01 (dd, J=11.7, 4.0 Hz, 1H), 3.75 (dd, J=11.8, 5.8 Hz, 1H), 3.40-3.30 (m, 1H), 2.17 (dt, J=13.8, 2.5 Hz, 1H), 2.06 (dt, J=14.1, 2.6 Hz, 1H), 1.89 (dd, J=13.7, 11.9 Hz, 1H), 1.58 (dd, J=13.8, 11.5 Hz, 1H), 1.17 (d, J=6.4 Hz, 3H). LCMS m/z 349.1 [M+H]⁺. SFC showed a de of >99%.

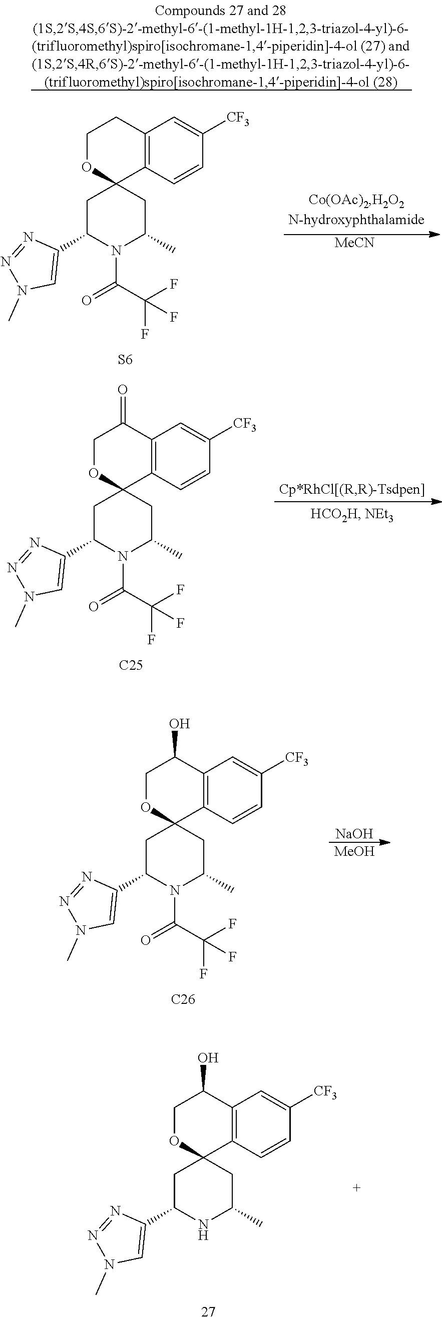

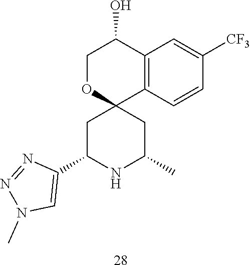 [SMILES: C[C@H]1C[C@@]2(C[C@@H](c3cn(C)nn3)N1)OC[C@H](O)c1cc(C(F)(F)F)ccc12 ; "28"]

### Step 1. Synthesis of(1S,2′S,6′S)-2′-methyl-6′-(1-methyl-1H-1,2,3-triazol-4-yl)-1′-(2,2,2-trifluoroacetyl)-6-(trifluoromethyl)spiro[isochromane-1,4′-piperidin]-4-one (C25)

Compound C25 was prepared from compound S6 following the method described for compound C23. The reaction was purified by silica gel chromatography (0 to 50% EtOAc in heptane) to afford the title compound C25 as a white solid (1032 mg, 80% purity, 34% yield). LCMS m/z 477.0 [M+H]⁺.

### Step 2. Synthesis of 2,2,2-trifluoro-]-((S,2′S,4S,6′S)-4-hydroxy-2′-methyl-6′-(1-methyl-]H-1,2, 3-triazol-4-yl)-6-(trifluoromethyl)spiro[isochromane-1,4′-piperidin]-1′-yl)ethan-1-one (C26)

Compound C26 was prepared from compound C25 following the method described for compound C24. The reaction was purified by silica gel chromatography (0 to 100% EtOAc in heptane) to afford the title compound C26 as a pale yellow oil (211 mg, 25% yield). SFC analysis showed a d.r. of 9:1. LCMS m/z 479.0 [M+H]⁺.

### Step 3. Synthesis of (1S,2′S,4S,6′S)-2′-methyl-6′-(1-methyl-1H-1, 2, 3-triazol-4-yl)-6-(trifluoromethyl)spiro[isochromane-1,4′-piperidin]-4-ol (27) and (1S,2′S,4R,6′S)-2′-methyl-6′-(1-methyl-1H-1,2, 3-triazol-4-yl)-6-(trifluoromethyl)spiro[isochromane-1,4′-piperidin]-4-ol (28)

Compounds 27 and 28 were prepared from compound C26 following the method described for compound 25. The reaction was purified by chiral SFC separation (Column: Daicel Chiralpak® AD-H, 20×250 mm; Mobile Phase: 10% MeOH (5 mM ammonia), 90% CO₂. Flow: 80 mL/min, isocratic) to afford the major diastereomer 27 (93.6 mg, 55.3%) and the minor diastereomer 28 (9.4 mg, 2.7%).

Characterization data for 27: ¹H NMR (300 MHz, Methanol-d4) δ 7.84 (s, 1H), 7.78 (s, 1H), 7.58 (d, J=8.3 Hz, 1H), 7.44 (d, J=8.3 Hz, 1H), 4.64 (t, J=4.9 Hz, 1H), 4.40 (d, J=11.1 Hz, 1H), 4.08 (s, 4H), 3.79 (dd, J=11.8, 5.8 Hz, 1H), 3.40-3.30 (m, 1H), 2.20 (d, J=13.8 Hz, 1H), 2.10 (d, J=13.9 Hz, 1H), 2.03-1.84 (m, 1H), 1.71-1.53 (m, 1H), 1.19 (d, J=6.5 Hz, 3H). ¹⁹F NMR (282 MHz, Methanol-d4) δ −64.13. LCMS m/z 382.4 [M+H]⁺.

Characterization data for 28: ¹H NMR (300 MHz, Chloroform-d) δ 7.75 (s, 1H), 7.56 (d, J=8.3 Hz, 1H), 7.47 (s, 1H), 7.35 (d, J=8.1 Hz, 1H), 4.65 (s, 1H), 4.55 (dd, J=11.3, 2.7 Hz, 1H), 4.11-4.01 (m, 4H), 3.92 (dd, J=12.2, 4.4 Hz, 1H), 3.31 (d, J=28.9 Hz, 1H), 2.12 (dt, J=39.4, 13.8 Hz, 3H), 1.60-1.38 (m, 3H), 1.17 (d, J=6.3 Hz, 3H). ¹⁹F NMR (282 MHz, Chloroform-d) δ −62.63. LCMS m/z 382.4 [M+H]⁺.

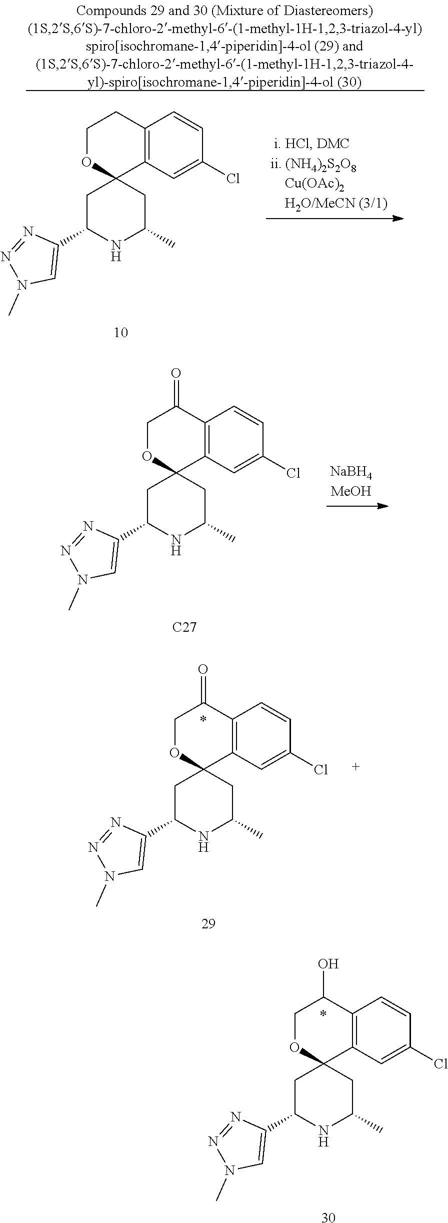

### Step 1. Synthesis of (]S,2′S,6′S)-7-chloro-2′-methyl-6′-(1-methyl-1H-1,2,3-triazol-4-yl)spiro[isochromane-1,4′-piperidin]-4-one (C27)

Compound C27 was prepared from compound 10 following the method described for compound C21. The crude was used directly without further purification. LCMS m/z 347.2 [M+H]⁺.

### Step 2. Synthesis of (]S,2′S,6′S)-7-chloro-2′-methyl-6′-(1-methyl-1H-1,2,3-triazol-4-yl)spiro[isochromane-1,4′-piperidin]-4-ol (29) and (1S,2′S,6′S)-7-chloro-2′-methyl-6′-(1-methyl-1H-1,2,3-triazol-4-yl)spiro[isochromane-1,4′-piperidin]-4-ol (30)

Compounds 29 and 30 (mixture of diastereomers) were prepared from compound C27 following the method described for compounds 25 and 26. The reaction was purified by reverse-phase HPLC (Method: C18 Waters Sunfire column (30×150 mm, 5 micron), gradient: MeCN in H₂O with 0.1% trifluoroacetic acid) to afford the title compounds as a mixture as trifluoroacetate salts (11.8 mg, 38%). The mixture was purified again by chiral SFC separation (Column: Daicel Chiralpak @AD-H, 10×250 mm; Mobile Phase: 40% Isopropanol (5 mM ammonia), 60% CO₂. Flow: 15 m/min, isocratic) to afford diastereomer 29 (2.5 mg, 10%) and diastereomer 30 (3.0 mg, 13%).

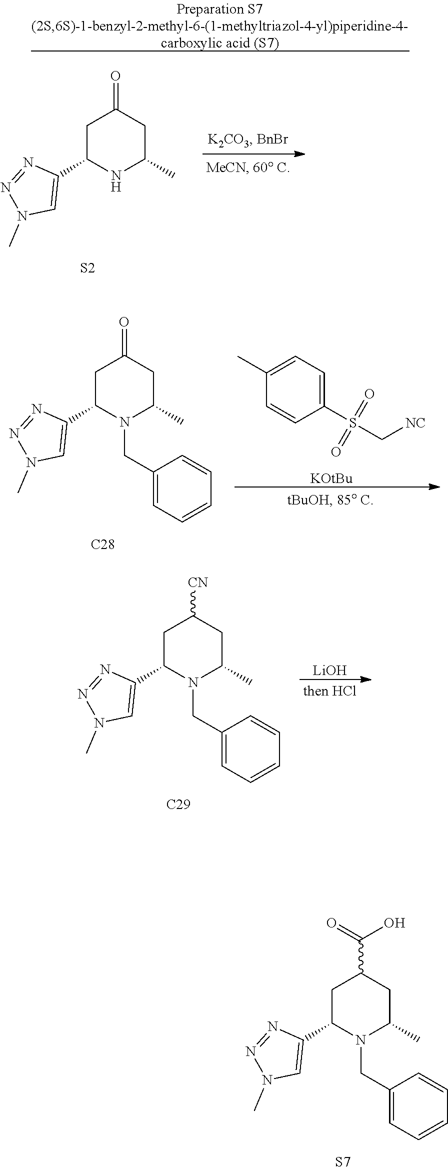

### Step 1. (2S,6S)-1-benzyl-2-methyl-6-(1-methyltriazol-4-yl)piperidin-4-one (C28)

To a 250 mL flask was added (2S,6S)-2-methyl-6-(1-methyltriazol-4-yl)piperidin-4-one S2 (2.02 g, 10.09 mmol), potassium carbonate (2.69 g, 19.46 mmol), and MeCN (20 mL). Then bromomethylbenzene (1.4 mL, 11.77 mmol) was added and the resulting mixture was heated to 60° C. After 5 hours, the reaction flask was left at room temperature overnight. The reaction was quenched with saturated NaHCO₃ solution and extracted with DCM (×4). The combined organic layer was dried over Na₂SO₄, filtered, and concentrated in vacuo. Purification by silica gel chromatography (Column: 220 g column, Gradient: 0-100% EtOAc in heptane) provided (2S,6S)-1-benzyl-2-methyl-6-(1-methyltriazol-4-yl)piperidin-4-one C₂₈ (1.67 g, 58%). ¹H NMR (300 MHz, Chloroform-d) δ 7.25-7.11 (m, 6H), 4.33 (dd, J=10.2, 3.9 Hz, 1H), 3.93 (s, 3H), 3.81 (s, 2H), 3.20 (h, J=6.5 Hz, 1H), 2.92 (dd, J=15.0, 10.2 Hz, 1H), 2.69 (dd, J=15.0, 4.0 Hz, 1H), 2.53-2.37 (m, 2H), 1.13 (d, J=6.4 Hz, 3H).

### Step 2. (2S,6S)-1-benzyl-2-methyl-6-(1-methyltriazol-4-yl)piperidine-4-carbonitrile (C29)

To an oven-dried 250-mL flask was added KOtBu (5.93 g, 52.85 mmol), purged with nitrogen for 10 minutes. tBuOH (48 mL) and DME (8 mL) were added. To another 100 mL flask was added (2S,6S)-1-benzyl-2-methyl-6-(1-methyltriazol-4-yl)piperidin-4-one C₂₈ (1.67 g, 5.873 mmol) and DME (10 mL). The C₂₈ solution was added to the KOtBu flask, and the resulting solution was stirred for 1 hour at room temperature. Then a solution of 1-(isocyanomethylsulfonyl)-4-methyl-benzene (2.22 g, 11.37 mmol) in DME (8 mL) was added. The reaction mixture was heated at 85° C. for 3 hours and then cooled to room temperature. Water was added to the brown solution. Then the reaction was extracted with DCM (×4). The combined organic layer was washed with brine, and the aqueous layer was extracted with DCM (×2). The organic layer was dried over MgSO₄, filtered, and concentrated in vacuo. Purification by silica gel chromatography (Column: 120 g column, Gradient: 0-10% MeOH in DCM) provided (2S,6S)-1-benzyl-2-methyl-6-(1-methyltriazol-4-yl)piperidine-4-carbonitrile C29 (1.47 g, 76%). LCMS m/z 296.21 [M+H]⁺. The product contained a mixture of diastereomers with a ratio of 1.6:1 based on ¹H NMR spectrum.

### Step 3. (2S,6S)-1-benzyl-2-methyl-6-(1-methyltriazol-4-yl)piperidine-4-carboxylic acid (S7)

To a stirred solution of (2S,6S)-1-benzyl-2-methyl-6-(1-methyltriazol-4-yl)piperidine-4-carbonitrile C29 (1.47 g, 4.479 mmol) in EtOH (18 mL), was added lithium hydroxide hydrate (1.895 g, 45.16 mmol) in water (18 mL) at room temperature. The reaction mixture was then heated to 100° C. After 4 hours, the reaction was cooled to room temperature and quenched with 6 M hydrogen chloride aqueous solution (7.8 mL of 6 M, 46.80 mmol) and concentrated in vacuo to remove all the solvent. Purification by silica gel chromatography (Column: 80 g column, Gradient: 0-10% MeOH in DCM) afforded (2S,6S)-1-benzyl-2-methyl-6-(1-methyltriazol-4-yl)piperidine-4-carboxylic acid S7 (874 mg, 62%). ¹H NMR (300 MHz, Methanol-d4) δ 8.08 (s, 1H), 7.45-6.94 (m, 5H), 4.98-4.76 (m, 1H), 4.51-4.25 (m, 2H), 4.13 (s, 3H), 3.79-3.47 (m, 1H), 2.95-2.66 (m, 1H), 2.50-2.12 (m, 3H), 2.10-1.79 (m, 1H), 1.56 (s, 3H). LCMS m/z 315.21 [M+H]⁺.

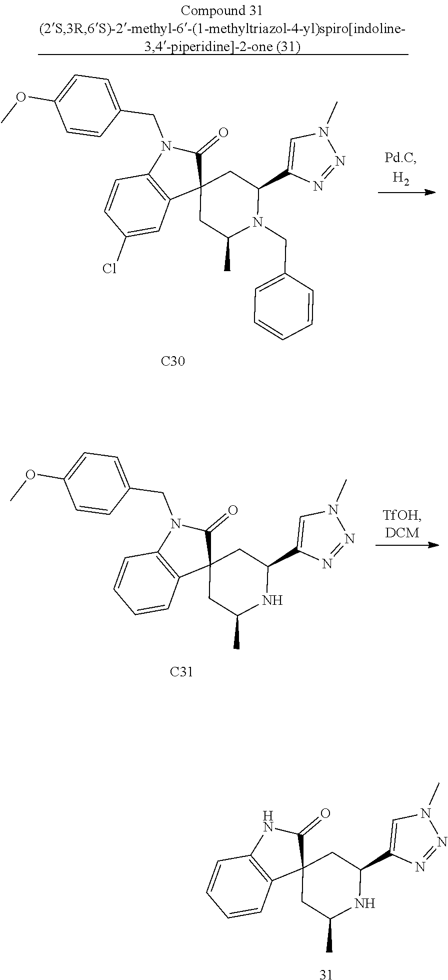

### Step 1. (2′S,3R,6′S)-5-chloro-]-[(4-methoxyphenyl)methyl]-2′-methyl-6′-(1-methyltriazol-4-yl)spiro[indoline-3,4′-piperidine]-2-one (C31)

To a stirred solution of (2′S,3R,6′S)-1′-benzyl-5-chloro-1-[(4-methoxyphenyl)methyl]-2′-methyl-6′-(1-methyltriazol-4-yl)spiro[indoline-3,4′-piperidine]-2-one C30 (17 mg, 0.03060 mmol, prepared as described below) in EtOH (500 μL) and EtOAc (500 μL) was added 5% palladium on carbon (6 mg, 0.002819 mmol). The reaction flask was evacuated and refilled with H₂ for 3 times. Then the reaction mixture was stirred under hydrogen balloon pressure. After 22 hours, the reaction flask was evacuated and refilled with H₂ for 3 times. After 11 hours, the reaction mixture was filtered through Celite® and the solids were rinsed with EtOAc. The crude material was purified with silica gel columns and eluted with 0 to 12% MeOH in DCM to provide (2′S,3R,6′S)-1-[(4-methoxyphenyl)methyl]-2′-methyl-6′-(1-methyltriazol-4-yl)spiro[indoline-3,4′-piperidine]-2-one C31 (3.8 mg, 29%). ¹H NMR (300 MHz, Chloroform-d) δ 7.77-7.70 (m, 1H), 7.51 (s, 1H), 7.24-7.13 (m, 3H), 7.03 (td, J=7.6, 1.1 Hz, 1H), 6.87-6.75 (m, 3H), 4.87 (s, 2H), 4.67 (dd, J=12.4, 2.8 Hz, 1H), 4.06 (s, 3H), 3.76 (s, 3H), 3.52 (ddd, J=11.9, 6.2, 2.7 Hz, 1H), 2.19 (t, J=12.7 Hz, 1H), 1.92-1.76 (m, 2H), 1.60-1.49 (m, 1H), 1.17 (d, J=6.2 Hz, 3H). LCMS m/z 418.3 [M+H]⁺. The chloro-product was also isolated.

### Step 2. (2′S,3R,6′S)-2′-methyl-6′-(1-methyltriazol-4-yl)spiro[indoline-3,4′-piperidine]-2-one (31)

To a 1-dram vial with (2′S,3R,6′S)-1-[(4-methoxyphenyl)methyl]-2′-methyl-6′-(1-methyltriazol-4-yl)spiro[indoline-3,4′-piperidine]-2-one C31 (3.8 mg, 0.009 mmol) was added DCM (200 μL), followed by trifluoromethanesulfonic acid (13 μL, 0.1469 mmol). After 24 hours, the reaction mixture was cooled to 0° C. and carefully quenched with saturated NaHCO₃ solution and extracted with DCM (×5). The combined organic layer was dried over Na₂SO₄, filtered, and concentrated. The combined organic layer was dried over Na₂SO₄, filtered, and concentrated. The crude material was purified with silica gel column and eluted with 0 to 20% MeOH in DCM to provide (2′S,3R,6′S)-2′-methyl-6′-(1-methyltriazol-4-yl)spiro[indoline-3,4′-piperidine]-2-one 31 (1.8 mg, 18%). ¹H NMR (300 MHz, Chloroform-d) δ 7.86 (s, 1H), 7.73 (d, J=7.6 Hz, 1H), 7.51 (s, 1H), 7.26 (dd, J=15.5, 1.2 Hz, 1H), 7.05 (td, J=7.6, 1.1 Hz, 1H), 6.93 (dt, J=7.7, 0.8 Hz, 1H), 4.67 (dd, J=12.3, 2.8 Hz, 1H), 4.05 (s, 3H), 3.61-3.48 (m, 1H), 2.14 (t, J=12.7 Hz, 1H), 1.96-1.75 (m, 2H), 1.59 (dt, J=13.1, 2.4 Hz, 1H), 1.17 (d, J=6.1 Hz, 3H). LCMS m/z 295.46 [M+H]⁺.

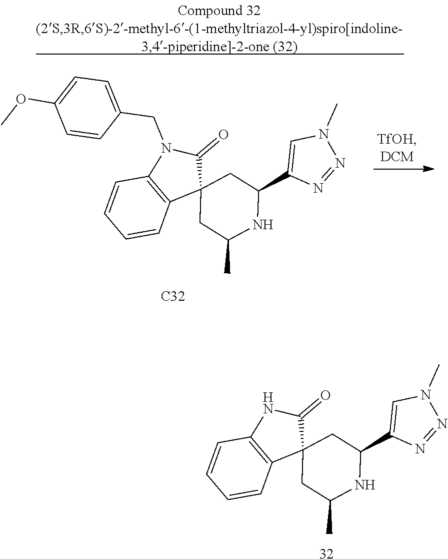

(2′S,3S,6′S)-2′-methyl-6′-(1-methyltriazol-4-yl)spiro[indoline-3,4′-piperidine]-2-one 32 was prepared from (2′S,3S,6′S)-1-[(4-methoxyphenyl)methyl]-2′-methyl-6′-(1-methyltriazol-4-yl)spiro[indoline-3,4′-piperidine]-2-one C32 (4.5 mg, 0.009269 mmol, prepared as described below), following the procedures to synthesize compound 31. Purification by reversed-phase HPLC. Method: C18 Waters Sunfire column (30×150 mm, 5 micron). Gradient: MeCN in H₂O with 0.2% formic acid. The product was isolated as (2′S,3S,6′S)-2′-methyl-6′-(1-methyltriazol-4-yl)spiro[indoline-3,4′-piperidine]-2-one (formic acid salt) (3.6 mg, 97%). ¹H NMR (300 MHz, Methanol-d4) δ 8.05 (s, 1H), 7.27 (t, J=7.7 Hz, 2H), 7.09 (t, J=7.5 Hz, 1H), 6.94 (d, J=7.7 Hz, 1H), 5.41 (dd, J=12.6, 3.2 Hz, 1H), 4.33 (h, J=6.9 Hz, 1H), 4.12 (s, 3H), 2.52 (dd, J=14.7, 12.7 Hz, 1H), 2.25 (dd, J=14.8, 3.2 Hz, 1H), 2.06 (d, J=8.2 Hz, 2H), 1.40 (d, J=6.5 Hz, 3H). LCMS m/z 298.34 [M+H]⁺.

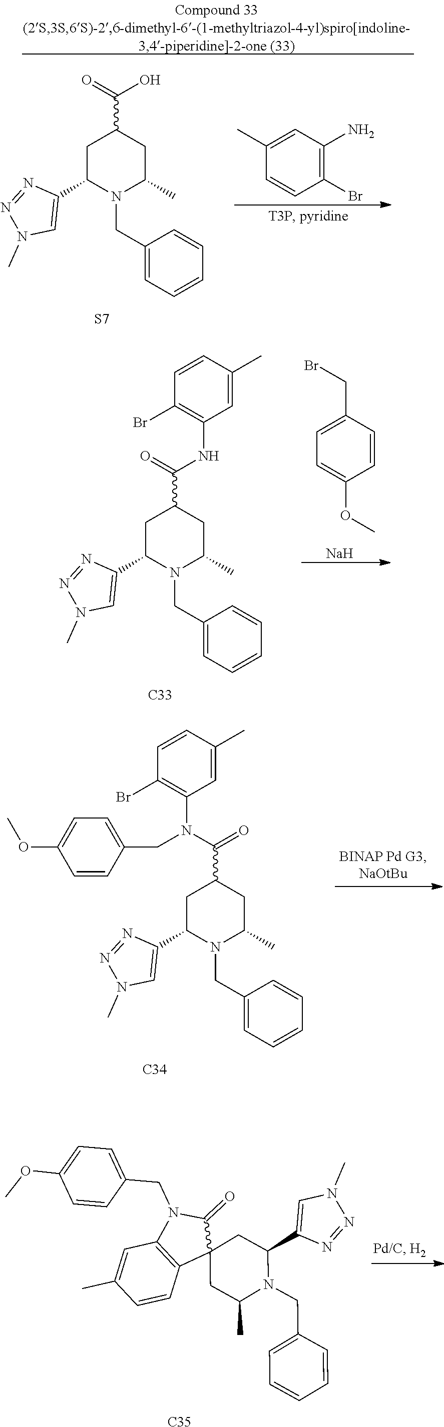

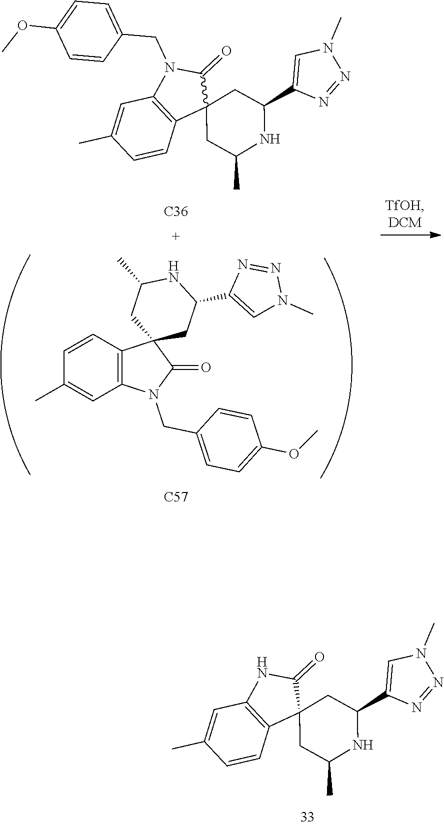

### Step]1. (2S,6S)-]-benzyl-N-(2-bromo-5-methyl-phenyl)-2-methyl-6-(1-methyltriazol-4-yl)piperidine-4-carboxamide (C33)

A 2-dram vial was charged with (2S,6S)-1-benzyl-2-methyl-6-(1-methyltriazol-4-yl)piperidine-4-carboxylic acid S7 (98 mg, 0.3117 mmol), 2-bromo-5-methyl-aniline (64 mg, 0.3440 mmol), pyridine (80 μL, 0.9891 mmol), EtOAc (1000 μL). Propylphosphonic anhydride solution (360 μL, 0.605 mmol, 50 wt % in EtOAc) was added. After 4 hours, the reaction mixture was quenched with saturated NaHCO₃ solution and extracted with EtOAc (×4). The crude mixture was purified by silica gel chromatography with 0 to 100% EA in heptane to provide (2S,6S)-1-benzyl-N-(2-bromo-5-methyl-phenyl)-2-methyl-6-(1-methyltriazol-4-yl)piperidine-4-carboxamide C33 (99 mg, 64%). ¹H NMR (300 MHz, Chloroform-d) δ 8.16 (s, 1H), 7.59 (s, 1H), 7.38 (d, J=8.2 Hz, 1H), 7.21 (q, J=5.1 Hz, 5H), 7.17-7.08 (m, 1H), 6.79 (d, J=8.5 Hz, 1H), 3.96 (dd, J=11.7, 2.9 Hz, 1H), 3.88 (s, 3H), 3.81-3.59 (m, 2H), 2.68 (s, 1H), 2.60-2.46 (m, 1H), 2.31 (s, 3H), 2.20 (s, 1H), 2.03-1.68 (m, 3H), 1.17 (d, J=6.1 Hz, 3H). LCMS m/z 482.12 [M+H]⁺.

### Step 2. (2S,6S)-]-benzyl-N-(2-bromo-5-methyl-phenyl)-N-[(4-methoxyphenyl)methyl]-2-methyl-6-(1-methyltriazol-4-yl)piperidine-4-carboxamide (C34)

To a 2-dram vial was charged (2S,6S)-1-benzyl-N-(2-bromo-5-methyl-phenyl)-2-methyl-6-(1-methyltriazol-4-yl)piperidine-4-carboxamide C33 (99 mg, 0.2008 mmol) in THF (2 mL). The vial was cooled to 0° C. and treated with sodium hydride (12.7 mg, 0.3175 mmol, 60 wt %) at 0° C. The vial was warmed to room temperature after 5 minutes. After 10 minutes, 1-(bromomethyl)-4-methoxy-benzene (35 μL, 0.2401 mmol) was added at room temperature. After 6 hours, additional NaH (4 mg, 0.1 mmol, 60 wt %) and 1-(bromomethyl)-4-methoxy-benzene (10 μL, 0.069 mmol) were added. After 2 hours, the reaction was quenched slowly with saturated NaHCO₃ solution and extracted with DCM (×3). The combined organic layer was dried over Na₂SO₄, filtered, and concentrated. The crude material was absorbed onto silica gel and purified with 0 to 100% EtOAc in heptane to provide (2S,6S)-1-benzyl-N-(2-bromo-5-methyl-phenyl)-N-[(4-methoxyphenyl)methyl]-2-methyl-6-(1-methyltriazol-4-yl)piperidine-4-carboxamide C34 (106 mg, 85%). LCMS m/z 602.07 [M+H]⁺. ¹H NMR spectrum indicated about 1.34:1 mixture of diastereomers. The material was then heated to 80° C. for 3 hours to remove residual EtOAc.

### Step 3. (2′S,6′S)-1′-benzyl-]-[(4-methoxyphenyl)methyl]-2′,6-dimethyl-6′-(1-methyltriazol-4-yl)spiro[indoline-3,4′-piperidine]-2-one (C35)

In a N2-glovebox was set up this reaction: to an oven-dried 2-dram vial was added (2S,6S)-1-benzyl-N-(2-bromo-5-methyl-phenyl)-N-[(4-methoxyphenyl)methyl]-2-methyl-6-(1-methyltriazol-4-yl)piperidine-4-carboxamide C34 (105 mg, 0.1688 mmol), followed by BINAP Pd G3 (16 mg, 0.01612 mmol) and sodium t-butoxide (35 mg, 0.3642 mmol), followed by the addition of dioxane (1.5 mL). The vial was transferred into fume hood and heated to 100° C. After 14 hours, the reaction mixture was quenched with saturated NaHCO₃ solution and extracted with EtOAc (×5). The combined organic layer was dried over Na₂SO₄, filtered, and concentrated. The crude material was purified with silica gel columns and eluted with 0 to 100% EtOAc in heptane to provide (2′S,6′S)-1′-benzyl-1-[(4-methoxyphenyl)methyl]-2′,6-dimethyl-6′-(1-methyltriazol-4-yl)spiro[indoline-3,4′-piperidine]-2-one C35 (80.9 mg, 87%). LCMS m/z 522.31 [M+H]⁺.

### Step 4. (2′S,3S,6′S)-1-[(4-methoxyphenyl)methyl]-2′, 6-dimethyl-6′-(1-methyltriazol-4-yl)spiro[indoline-3,4′-piperidine]-2-one (C36)

To a stirred solution of (2′S,6′S)-1′-benzyl-1-[(4-methoxyphenyl)methyl]-2′,6-dimethyl-6′-(1-methyltriazol-4-yl)spiro[indoline-3,4′-piperidine]-2-one C35 (80.9 mg, 0.1473 mmol) in EtOH (1.5 mL) and EtOAc (1.5 mL) was added palladium on carbon (Evonik Noblyst® P1090 5% Pd, 16 mg, 0.007517 mmol). The reaction flask was evacuated and refilled with H₂ for 4 times. Then the reaction mixture was stirred under hydrogen balloon pressure. After 25 hours, H₂ was recharged, and additional palladium (16 mg, 0.007517 mmol) was added. The reaction was left at room temperature for 24 hours, then H₂ was recharged. After another 24 hours, the reaction mixture was filtered through a plug of Celite® and washed with EtOAc. The filtrate was concentrated and purified by silica gel chromatography (Column: 12 g column, Gradient: 0-10% MeOH in DCM) afforded two fractions:

(2′S,3R,6′S)-1-[(4-methoxyphenyl)methyl]-2′,6-dimethyl-6′-(1-methyltriazol-4-yl)spiro[indoline-3,4′-piperidine]-2-one C57 (19.4 mg, 31%). ¹H NMR (300 MHz, Chloroform-d) 6 7.61 (d, J=7.6 Hz, 1H), 7.50 (s, 1H), 7.18 (d, J=8.5 Hz, 2H), 6.83 (dd, J=8.2, 3.4 Hz, 3H), 6.62 (s, 1H), 4.84 (s, 2H), 4.65 (dd, J=12.4, 2.8 Hz, 1H), 4.05 (s, 3H), 3.76 (s, 3H), 3.50 (ddd, J=12.0, 6.2, 2.9 Hz, 1H), 2.30 (s, 3H), 2.18 (t, J=12.6 Hz, 1H), 1.94-1.76 (m, 2H), 1.53 (dt, J=12.9, 2.4 Hz, 1H), 1.16 (d, J=6.1 Hz, 3H). Based on ¹H NOESY, the relative stereochemistry is assigned as cis, with key NOESY signals between oxindole C—H and the two methine protons.

(2′S,3S,6′S)-1-[(4-methoxyphenyl)methyl]-2′,6-dimethyl-6′-(1-methyltriazol-4-yl)spiro[indoline-3,4′-piperidine]-2-one C36 (34.8 mg, 55%. ¹H NMR (300 MHz, Chloroform-d) 6 7.44 (s, 1H), 7.20 (d, J=8.4 Hz, 2H), 7.08 (d, J=7.5 Hz, 1H), 6.91-6.78 (m, 3H), 6.54 (s, 1H), 4.99 (dd, J=11.5, 3.1 Hz, 1H), 4.80 (s, 2H), 4.05 (s, 3H), 3.97-3.79 (m, 1H), 3.76 (s, 3H), 2.28 (s, 3H), 2.18-1.87 (m, 2H), 1.77 (t, J=2.0 Hz, 1H), 1.60 (dd, J=13.6, 11.3 Hz, 1H), 1.14 (d, J=6.3 Hz, 3H).

This product was assigned as trans-, based on the NMR assignment of cis isomer.

### Step 5. (2′S,3S,6′S)-2′,6-dimethyl-6′-(1-methyltriazol-4-yl)spiro[indoline-3, 4′-piperidine]-2-one (33)

To a 20-mL vial with (2′S,3S,6′S)-1-[(4-methoxyphenyl)methyl]-2′,6-dimethyl-6′-(1-methyltriazol-4-yl)spiro[indoline-3,4′-piperidine]-2-one C36 (34.8 mg, 0.08064 mmol) in DCM (1.4 mL) at 0° C. was added trifluoromethanesulfonic acid (72 μL, 0.8137 mmol). The vial was warmed to room temperature after the addition of acid. After 6 hours, the reaction vial was cooled to 0° C. and carefully quenched with saturated NaHCO₃ solution and extracted with DCM (×5). The combined organic layer was dried over Na₂SO₄, filtered, and concentrated. The crude material was purified silica gel columns and eluted with 0 to 20% MeOH in DCM to provide (2′S,3S,6′S)-2′,6-dimethyl-6′-(1-methyltriazol-4-yl)spiro[indoline-3,4′-piperidine]-2-one 33 (22.1 mg, 85%). ¹H NMR (300 MHz, Chloroform-d) δ 8.68 (s, 1H), 7.43 (s, 1H), 7.05 (d, J=7.6 Hz, 1H), 6.83 (d, J=7.6 Hz, 1H), 6.72 (s, 1H), 4.96 (dd, J=8.8, 5.9 Hz, 1H), 4.03 (s, 3H), 3.96-3.72 (m, 1H), 2.32 (s, 3H), 2.06-1.96 (m, 2H), 1.77 (dd, J=13.6, 2.7 Hz, 1H), 1.58 (dd, J=13.6, 11.4 Hz, 1H), 1.13 (d, J=6.3 Hz, 3H). LCMS m/z 312.19 [M+H]⁺.

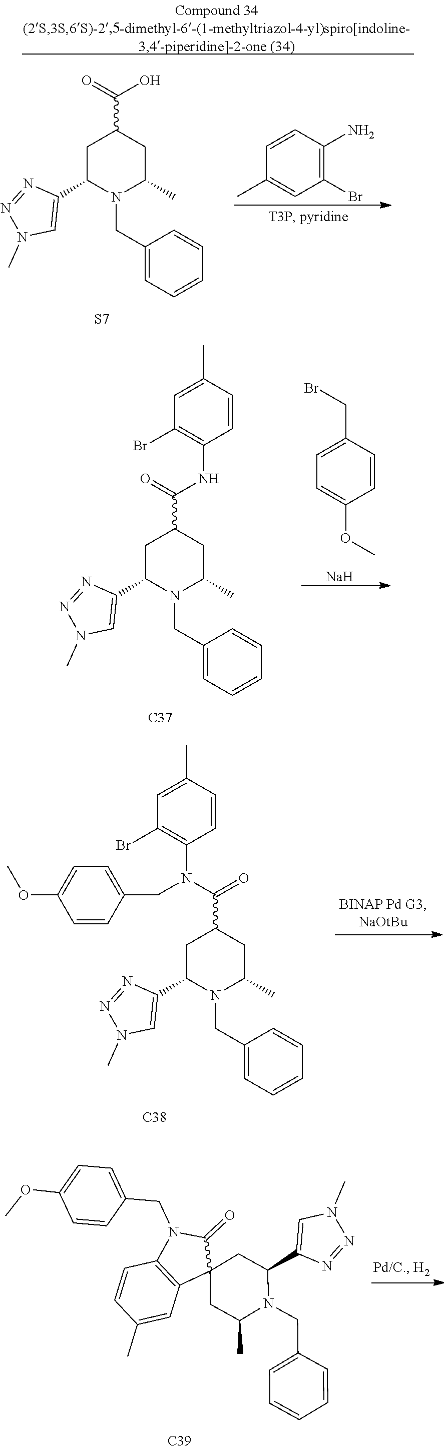

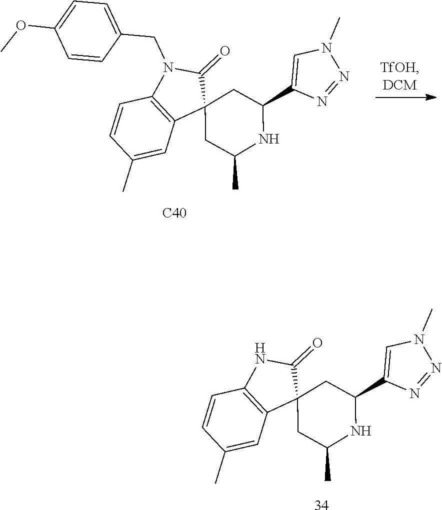

### Step 1. (2S,6S)-1-benzyl-N-(2-bromo-4-methyl-phenyl)-2-methyl-6-(1-methyltriazol-4-yl)piperidine-4-carboxamide (C37)

A 2-dram vial was charged with (2S,6S)-1-benzyl-2-methyl-6-(1-methyltriazol-4-yl)piperidine-4-carboxylic acid S7 (82 mg, 0.2608 mmol), 2-bromo-4-methyl-aniline (53 mg, 0.2849 mmol), pyridine (64 μL, 0.7913 mmol), and EtOAc (800 μL). Propylphosphonic anhydride solution (300 μL, 0.504 mmol, 50 wt % o in EtOAc) was added. After 6 hours, the reaction was quenched with saturated NaHCO₃ solution and extracted with EtOAc (×4). The crude mixture was purified by silica gel chromatography with 0 to 100% o EtOAc in heptane to provide (2S,6S)-1-benzyl-N-(2-bromo-4-methyl-phenyl)-2-methyl-6-(1-methyltriazol-4-yl)piperidine-4-carboxamide C37 (85 mg, 65%). ¹H NMR (300 MHz, Chloroform-d) δ 8.14 (d, J=8.4 Hz, 1H), 7.56 (s, 1H), 7.41-7.32 (m, 1H), 7.25-7.17 (m, 5H), 7.17-7.02 (m, 2H), 3.95 (dd, J=11.6, 2.8 Hz, 1H), 3.88 (s, 3H), 3.77-3.55 (m, 2H), 2.67 (t, J=7.5 Hz, 1H), 2.52 (tt, J=12.3, 3.7 Hz, 1H), 2.29 (s, 3H), 2.23-2.07 (m, 1H), 2.02-1.70 (m, 3H), 1.16 (d, J=6.1 Hz, 3H). LCMS m/z 482.26 [M+H]⁺.

### Step 2. (2S,6S)-]-benzyl-N-(2-bromo-4-methyl-phenyl)-N-[(4-methoxyphenyl)methyl]-2-methyl-6-(1-methyltriazol-4-yl)piperidine-4-carboxamide (C38)

To a vial was charged (2S,6S)-1-benzyl-N-(2-bromo-4-methyl-phenyl)-2-methyl-6-(1-methyltriazol-4-yl)piperidine-4-carboxamide C37 (85 mg, 0.1762 mmol) and THF (1.8 mL). The vial was cooled to 0° C. and treated with sodium hydride (14 mg, 0.3500 mmol, 60 wt %) at 0° C. The vial was warmed to room temperature after 5 minutes. After 10 minutes, 1-(bromomethyl)-4-methoxy-benzene (39 μL, 0.2675 mmol) was added at room temperature. After 7 hours, the reaction was quenched slowly with saturated NaHCO₃ solution and extracted with DCM (×3). The combined organic layer was dried over Na₂SO₄, filtered, and concentrated. The crude material was absorbed onto silica gel and purified with 0 to 100% EtOAc in heptane to provide (2S,6S)-1-benzyl-N-(2-bromo-4-methyl-phenyl)-N-[(4-methoxyphenyl)methyl]-2-methyl-6-(1-methyltriazol-4-yl)piperidine-4-carboxamide C38 (95 mg, 83%). LCMS m/z 602.25 [M+H]⁺. The material was then heated to 80° C. for 2 hours under vacuum to remove residual EtOAc. ¹H NMR indicated the product was a mixture of diastereomers with a ratio of 1.34:1.

### Step 3. (2′S,6′S)-1′-benzyl-]-[(4-methoxyphenyl)methyl]-2′,5-dimethyl-6′-(1-methyltriazol-4-yl)spiro[indoline-3,4′-piperidine]-2-one (C39)

In a N2-glovebox was set up this reaction: to a 20 ml vial was added (2S,6S)-1-benzyl-N-(2-bromo-4-methyl-phenyl)-N-[(4-methoxyphenyl)methyl]-2-methyl-6-(1-methyltriazol-4-yl)piperidine-4-carboxamide C38 (95 mg, 0.1463 mmol), followed by BINAP Pd G3 46-2153 (7.3 mg, 0.007356 mmol) and sodium t-butoxide (28 mg, 0.2914 mmol). Lastly, dioxane (1.3 mL) was added. The vial was relocated from the glovebox to the bench and heated to 100° C. After 15 hours, the reaction was quenched with saturated NaHCO₃ solution and extracted with EtOAc (×5). The combined organic layer was dried over Na₂SO₄, filtered, and concentrated. The crude material was purified with silica gel columns and eluted with 0 to 100% EtOAc in heptane to provide (2′S,6′S)-1′-benzyl-1-[(4-methoxyphenyl)methyl]-2′,5-dimethyl-6′-(1-methyltriazol-4-yl)spiro[indoline-3,4′-piperidine]-2-one C39 (83.4 mg, 0.1071 mmol, 67% purity by ¹H NMR), mixed with starting material with a ratio of 1.44:1.0:1.18 (diastereomer 1 and 2, and starting material). The mixture was carried to the next reaction.

### Step 4. (2′S, 3S,6′S)-1-[(4-methoxyphenyl)methyl]-2′, 5-dimethyl-6′-(1-methyltriazol-4-yl)spiro[indoline-3,4′-piperidine]-2-one (C40)

To a stirred solution of (2′S,6′S)-1′-benzyl-1-[(4-methoxyphenyl)methyl]-2′,5-dimethyl-6′-(1-methyltriazol-4-yl)spiro[indoline-3,4′-piperidine]-2-one C39 (83.4 mg, 0.1071 mmol, 67% purity by ¹H NMR) in EtOH (1 mL) and EtOAc (1 mL) was added palladium on carbon (Evonik Noblyst® P1090 5% Pd, 34.6 mg, 0.01626 mmol). The reaction flask was evacuated and refilled with H₂ for 4 times. Then the reaction mixture was stirred under hydrogen balloon pressure. After 18 hours, H₂ was recharged and the reaction mixture was stirred at room temperature. After another 24 hours, the reaction flask was evacuated and refilled with N2. The mixture was filtered through a plug of Celite® and washed with EtOAc. The filtrate was concentrated and purified by silica gel chromatography (Column: 24 g column, Gradient: 0-10% MeOH in DCM) to afford two diastereomers. The trans isomer was isolated as the more polar fraction. (2′S,3S,6′S)-1-[(4-methoxyphenyl)methyl]-2′,5-dimethyl-6′-(1-methyltriazol-4-yl)spiro[indoline-3,4′-piperidine]-2-one C40 (23.1 mg, 43%). ¹H NMR (300 MHz, Chloroform-d) 6 7.51 (s, 1H), 7.19 (d, J=8.5 Hz, 2H), 7.05 (s, 1H), 6.94 (d, J=7.9 Hz, 1H), 6.83 (d, J=8.4 Hz, 2H), 6.60 (d, J=7.8 Hz, 1H), 5.03 (dd, J=11.7, 2.9 Hz, 1H), 4.81 (s, 2H), 4.06 (s, 3H), 4.02-3.83 (m, 1H), 3.76 (s, 3H), 2.36 (d, J=2.2 Hz, 3H), 2.17 (t, J=12.7 Hz, 1H), 1.99 (d, J=17.5 Hz, 1H), 1.71 (dt, J=24.7, 13.1 Hz, 2H), 1.18 (d, J=6.3 Hz, 3H).

### Note:

The stereochemistry is assigned by comparing ¹H NMR spectrum from syntheses with related analogues (compound 33) and the key ¹H signal is the methine peak around 4.5-5 ppm. Trans isomer showed slight downfield shift (around 5.0 ppm), where in cis isomer it showed up around 4.6 ppm.

### Step 5. (2′S,3S,6′S)-2′,5-dimethyl-6′-(1-methyltriazol-4-yl)spiro[indoline-3, 4′-piperidine]-2-one (34)

To a 20-mL vial with (2′S,3S,6′S)-1-[(4-methoxyphenyl)methyl]-2′,5-dimethyl-6′-(1-methyltriazol-4-yl)spiro[indoline-3,4′-piperidine]-2-one C40 (23.1 mg, 0.04978 mmol) in DCM (800 μL) at 0° C. was added trifluoromethanesulfonic acid (45 μL, 0.5085 mmol). The vial was warmed to room temperature after the addition of acid. After 7 hours, the reaction was cooled to 0° C. and carefully quenched with saturated NaHCO₃ solution and extracted with DCM (×5). The combined organic layer was dried over Na₂SO₄, filtered, and concentrated. The crude material was purified with silica gel columns and eluted with 0 to 20% MeOH in DCM to provide (2′S,3S,6′S)-2′,5-dimethyl-6′-(1-methyltriazol-4-yl)spiro[indoline-3,4′-piperidine]-2-one 34 (12.8 mg, 77%). ¹H NMR (300 MHz, Chloroform-d) δ 7.85 (s, 1H), 7.44 (s, 1H), 6.99 (d, J=10.1 Hz, 2H), 6.75 (d, J=7.7 Hz, 1H), 4.95 (dd, J=9.9, 4.7 Hz, 1H), 4.05 (s, 3H), 3.82 (ddd, J=11.7, 6.4, 2.8 Hz, 1H), 2.31 (s, 3H), 2.15-1.99 (m, 2H), 1.80 (dd, J=13.7, 2.7 Hz, 1H), 1.68-1.47 (m, 1H), 1.14 (d, J=6.3 Hz, 3H). LCMS m/z 312.14 [M+H]⁺.

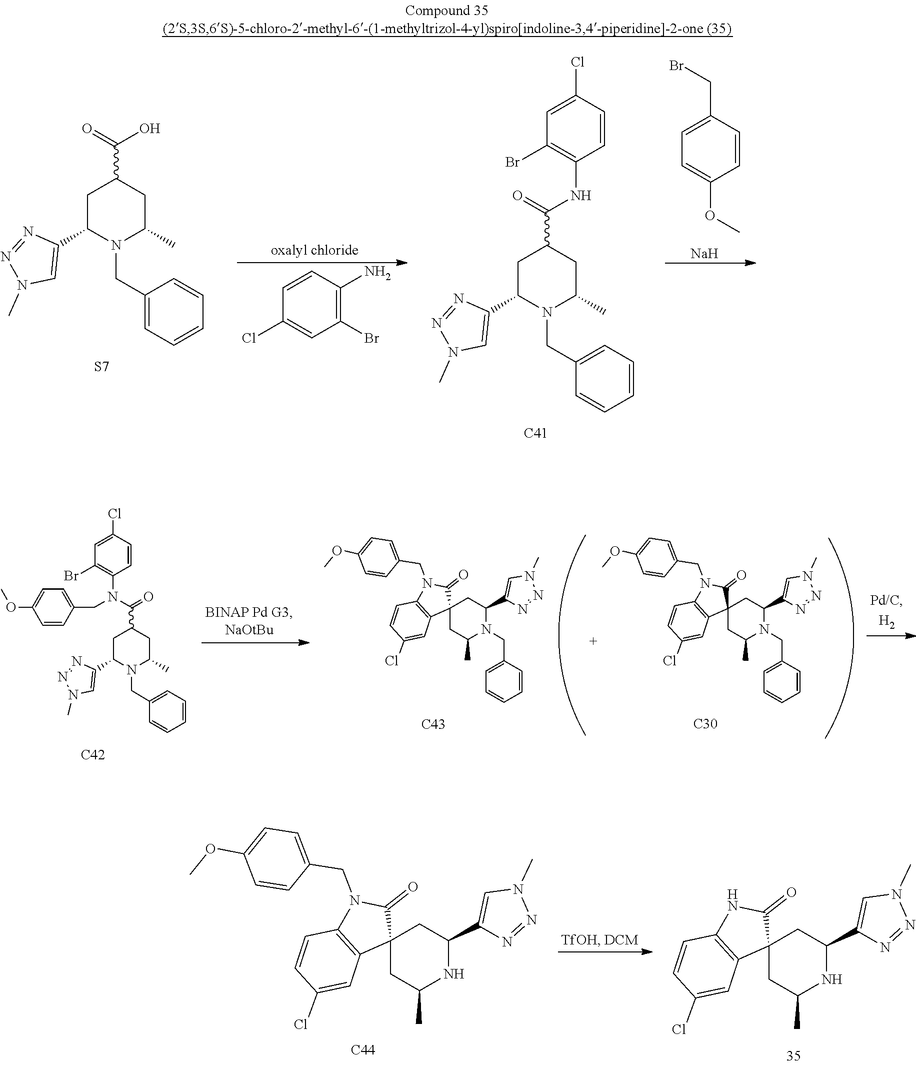

### Step 1. (2S,6S)-1-benzyl-N-(2-bromo-4-chloro-phenyl)-2-methyl-6-(1-methyltriazol-4-yl)piperidine-4-carboxamide (C41)

To a 20-mL vial was added (2S,6S)-1-benzyl-2-methyl-6-(1-methyltriazol-4-yl)piperidine-4-carboxylic acid S7 (118 mg, 0.3753 mmol), DCM (1.5 mL) and oxalyl dichloride (380 μL of 2 M, 0.75 mmol), followed by a drop of DMF. After 3 hours, the reaction mixture was concentrated in vacuo.

The crude material was dissolved in 0.5 mL pyridine and 2 mL DCM. At 0° C., 2-bromo-4-chloro-aniline (84.8 mg, 0.4107 mmol) was added. After 14 hours, the reaction was quenched with saturated NaHCO₃ solution and extracted with EtOAc (×3). The combined organic layer was dried over Na₂SO₄, filtered, and concentrated in vacuo. The crude mixture was purified by silica gel chromatography with 0 to 30% EtOAc in heptane to provide (2S,6S)-1-benzyl-N-(2-bromo-4-chloro-phenyl)-2-methyl-6-(1-methyltriazol-4-yl)piperidine-4-carboxamide C41 (101 mg, 51%). ¹H NMR (400 MHz, Chloroform-d) δ 8.29 (d, J=9.0 Hz, 1H), 7.59 (s, 1H), 7.53 (d, J=2.2 Hz, 1H), 7.31-7.27 (m, 1H), 7.25-7.17 (m, 5H), 7.15 (d, J=6.6 Hz, 1H), 3.95 (dd, J=11.5, 2.7 Hz, 1H), 3.88 (d, J=1.6 Hz, 3H), 3.76-3.60 (m, 2H), 2.67 (d, J=9.7 Hz, 1H), 2.53 (t, J=12.4 Hz, 1H), 2.17 (d, J=12.7 Hz, 1H), 2.02-1.85 (m, 2H), 1.79 (q, J=12.2 Hz, 1H), 1.17 (dd, J=6.1, 1.6 Hz, 3H). LCMS m/z 502.24 [M+H]⁺.

### Step 2. (2S,6S)-1-benzyl-N-(2-bromo-4-chloro-phenyl)-N-[(4-methoxyphenyl)methyl]-2-methyl-6-(1-methyltriazol-4-yl)piperidine-4-carboxamide (C42)

To a 2-dram vial was added (2S,6S)-1-benzyl-N-(2-bromo-4-chloro-phenyl)-2-methyl-6-(1-methyltriazol-4-yl)piperidine-4-carboxamide C41 (98 mg, 0.1949 mmol) and THF (2 mL). The vial was cooled to 0° C. and treated with sodium hydride (12 mg, 0.30 mmol, 60 wt %) at 0° C. The vial was warmed to room temperature. After 10 minutes, 1-(bromomethyl)-4-methoxy-benzene (34 μL, 0.2332 mmol) was added. After 20 hours, the reaction was quenched slowly with saturated NaHCO₃ solution and extracted with DCM (×3). The combined organic layer was dried over Na₂SO₄, filtered, and concentrated. The crude material was absorbed onto SiO2 and purified with 0 to 100% EtOAc in heptane to provide (2S,6S)-1-benzyl-N-(2-bromo-4-chloro-phenyl)-N-[(4-methoxyphenyl)methyl]-2-methyl-6-(1-methyltriazol-4-yl)piperidine-4-carboxamide C42 (102 mg, 78%). ¹H NMR (400 MHz, Chloroform-d) δ 7.70 (dd, J=2.3, 1.2 Hz, 1H), 7.23-6.94 (m, 9H), 6.88-6.69 (m, 2H), 6.60 (dd, J=26.5, 8.4 Hz, 1H), 5.53 (dd, J=14.2, 6.0 Hz, 1H), 3.93-3.86 (m, 1H), 3.84 (d, J=2.5 Hz, 3H), 3.77 (d, J=9.5 Hz, 3H), 3.71-3.47 (m, 3H), 2.46-2.23 (m, 1H), 2.12 (td, J=14.2, 13.0, 9.8 Hz, 1H), 2.02-1.63 (m, 3H), 1.03 (dd, J=28.9, 6.1 Hz, 3H). LCMS m/z 622.32 [M+H]⁺. ¹H NMR spectrum indicated about 1.3:1 mixture of diastereomers.

### Step 3. (2′S,3S,6′S) and (2′S,3R,6′S)-1′-benzyl-5-chloro-]-[(4-methoxyphenyl)methyl]-2′-methyl-6′-(1-methyltriazol-4-yl)spiro[indoline-3,4′-piperidine]-2-one (C43 and C30)

To a 20 mL vial was added (2S,6S)-1-benzyl-N-(2-bromo-4-chloro-phenyl)-N-[(4-methoxyphenyl)methyl]-2-methyl-6-(1-methyltriazol-4-yl)piperidine-4-carboxamide C42 (102 mg, 0.1354 mmol), followed by BINAP Pd G3 (17 mg, 0.01713 mmol) and sodium t-butoxide (49 mg, 0.5099 mmol). The vial was purged with N₂ for 15 minutes, followed by the addition of dioxane (1.5 mL). The vial was heated to 100° C. for 12 hours. The reaction was quenched with saturated NaHCO₃ solution and extracted with EtOAc (×3). The combined organic layer was dried over Na₂SO₄, filtered, and concentrated. The crude material was purified with silica gel column and eluted with 0 to 100% EtOAc in heptane to provide two fractions:

(2′S,3S,6′S)-1′-benzyl-5-chloro-1-[(4-methoxyphenyl)methyl]-2′-methyl-6′-(1-methyltriazol-4-yl)spiro[indoline-3,4′-piperidine]-2-one C43 (14.9 mg, 20%). ¹H NMR (300 MHz, Chloroform-d) δ 7.33-7.23 (m, 6H), 7.23-7.14 (m, 3H), 7.11 (dd, J=8.3, 2.1 Hz, 1H), 6.91-6.76 (m, 2H), 6.62 (d, J=8.3 Hz, 1H), 4.95 (dd, J=12.0, 2.9 Hz, 1H), 4.91-4.66 (m, 2H), 3.89 (s, 3H), 3.85 (d, J=8.8 Hz, 2H), 3.79 (s, 3H), 3.78-3.69 (m, 1H), 2.33 (dd, J=13.8, 12.0 Hz, 1H), 1.96 (ddd, J=13.7, 5.8, 2.6 Hz, 2H), 1.75 (dt, J=13.7, 2.8 Hz, 1H), 1.09 (d, J=6.3 Hz, 3H). LCMS m/z 542.39 [M+H]⁺.

(2′S,3R,6′S)-1′-benzyl-5-chloro-1-[(4-methoxyphenyl)methyl]-2′-methyl-6′-(1-methyltriazol-4-yl)spiro[indoline-3,4′-piperidine]-2-one C30 (17.2 mg, 14%, 59% purity). LCMS m/z 542.43 [M+H]⁺.

### Step 4. (2′S,3S,6′S)-5-chloro-]-[(4-methoxyphenyl)methyl]-2′-methyl-6′-(1-methyltriazol-4-yl)spiro[indoline-3,4′-piperidine]-2-one (C44)

To a stirred solution of (2′S,3S,6′S)-1′-benzyl-5-chloro-1-[(4-methoxyphenyl)methyl]-2′-methyl-6′-(1-methyltriazol-4-yl)spiro[indoline-3,4′-piperidine]-2-one C43 (14.9 mg, 0.02682 mmol) in EtOH (400 μL) and EtOAc (400 μL) was added 5% palladium on carbon (5.3 mg, 0.002490 mmol). The reaction flask was evacuated and refilled with H₂ for 3 times. Then the reaction mixture was stirred under hydrogen balloon pressure. After 48 hours, the reaction flask was evacuated and refilled with H₂ for 3 times. After 6 hours, the reaction mixture was filtered through Celite® and the solids were rinsed with EtOAc. The crude material was purified with silica gel columns and eluted with 0 to 10% MeOH in DCM to provide (2′S,3S,6′S)-5-chloro-1-[(4-methoxyphenyl)methyl]-2′-methyl-6′-(1-methyltriazol-4-yl)spiro[indoline-3,4′-piperidine]-2-one C44 (5.9 mg, 48%). ¹H NMR (300 MHz, Chloroform-d) δ 7.48 (s, 1H), 7.22-7.13 (m, 3H), 7.10 (dd, J=8.3, 2.1 Hz, 1H), 6.89-6.78 (m, 2H), 6.62 (d, J=8.3 Hz, 1H), 5.00 (dd, J=10.7, 3.8 Hz, 1H), 4.81 (d, J=2.3 Hz, 2H), 4.06 (s, 3H), 3.96-3.80 (m, 1H), 3.77 (s, 3H), 2.18-1.89 (m, 2H), 1.80 (dd, J=2.9, 1.4 Hz, 1H), 1.60 (dd, J=13.6, 11.3 Hz, 1H), 1.16 (d, J=6.3 Hz, 3H). LCMS m/z 452.28 [M+H]⁺. Dehalogenated product was also isolated.

Note: the stereochemistry is assigned by comparing ¹H NMR spectrum from syntheses with related analogues (compound 33), and the key ¹H signal is the methine peak around 4.5-5 ppm. Trans isomer showed slight downfield shift (around 5.0 ppm), where in cis isomer it showed up around 4.6 ppm.

### Step 5. (2′S,3S,6′S)-5-chloro-2′-methyl-6′-(1-methyltriazol-4-yl)spiro[indoline-3,4′-piperidine]-2-one (35)

To a 1-dram vial with (2′S,3S,6′S)-5-chloro-1-[(4-methoxyphenyl)methyl]-2′-methyl-6′-(1-methyltriazol-4-yl)spiro[indoline-3,4′-piperidine]-2-one C44 (5.9 mg, 0.01281 mmol) in DCM (150 μL) was added trifluoromethanesulfonic acid (12 μL, 0.1356 mmol) (caution: exothermic). After 3 hours, the reaction was cooled to 0° C. and carefully quenched with saturated NaHCO₃ solution and extracted with DCM (×4). The combined organic layer was dried over Na₂SO₄, filtered, and concentrated. The crude material was purified with silica gel columns and eluted with 0 to 20% MeOH in DCM to provide (2′S,3S,6′S)-5-chloro-2′-methyl-6′-(1-methyltriazol-4-yl)spiro[indoline-3,4′-piperidine]-2-one 35 (4.3 mg, 97%). ¹H NMR (300 MHz, Chloroform-d) δ 7.91 (s, 1H), 7.49 (s, 1H), 7.24-7.15 (m, 2H), 6.82 (d, J=8.8 Hz, 1H), 4.96 (dd, J=9.0, 5.6 Hz, 1H), 4.08 (s, 3H), 3.83 (dqd, J=12.6, 6.2, 2.7 Hz, 1H), 2.18-1.95 (m, 2H), 1.84 (dt, J=13.9, 1.7 Hz, 1H), 1.60 (dd, J=13.6, 11.4 Hz, 1H), 1.17 (d, J=6.4 Hz, 3H). LCMS m/z 332.21 [M+H]⁺.

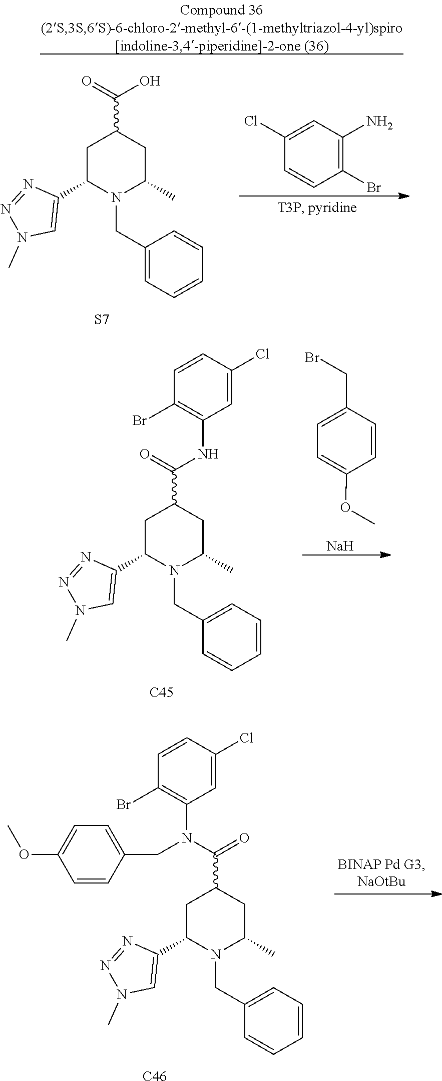

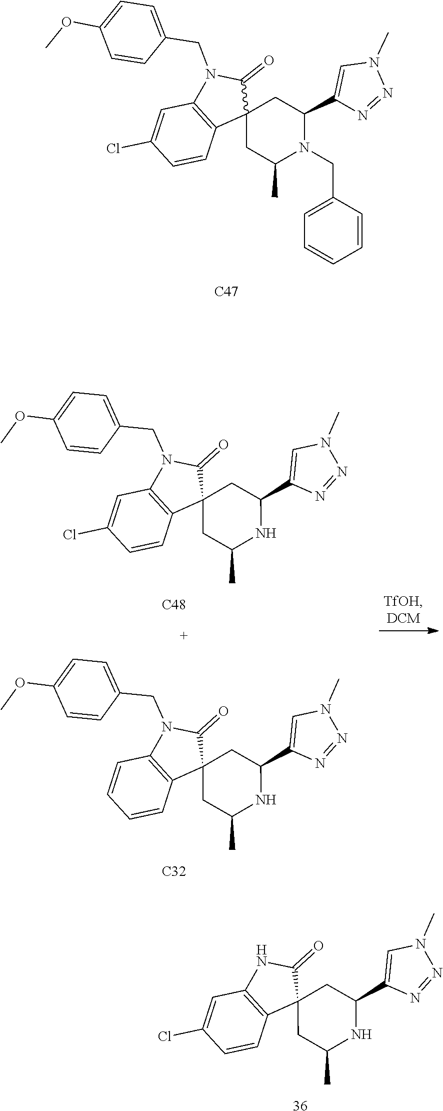

### Step 1. (2S,6S)-1-benzyl-N-(2-bromo-5-chloro-phenyl)-2-methyl-6-(1-methyltriazol-4-yl)piperidine-4-carboxamide (C45)

A 20 mL vial was charged with (2S,6S)-1-benzyl-2-methyl-6-(1-methyltriazol-4-yl)piperidine-4-carboxylic acid S7 (120 mg, 0.3817 mmol), 2-bromo-5-chloro-aniline (86.5 mg, 0.4190 mmol), pyridine (100 μL, 1.236 mmol), and EtOAc (1.5 mL). Propylphosphonic anhydride solution (480 mg, 0.7543 mmol, 50 wt % in EtOAc) was added. After 3 hours, the reaction was quenched with saturated NaHCO₃ solution and extracted with EtOAc (×3). The combined organic layer was dried over Na₂SO₄, filtered, and concentrated in vacuo. The crude mixture was purified by silica gel chromatography with 0 to 100% EtOAc in heptane to provide (2S,6S)-1-benzyl-N-(2-bromo-5-chloro-phenyl)-2-methyl-6-(1-methyltriazol-4-yl)piperidine-4-carboxamide C45 (135.5 mg, 69%). ¹H NMR (300 MHz, Chloroform-d) δ 8.44 (d, J=2.5 Hz, 1H), 7.64 (s, 1H), 7.43 (d, J=8.6 Hz, 1H), 7.24-7.10 (m, 6H), 6.96 (dd, J=8.6, 2.5 Hz, 1H), 3.96 (dd, J=11.6, 2.8 Hz, 1H), 3.89 (s, 3H), 3.81-3.63 (m, 2H), 2.68 (s, 1H), 2.61-2.44 (m, 1H), 2.16 (dd, J=13.0, 3.1 Hz, 1H), 2.08-1.71 (m, 3H), 1.18 (d, J=6.1 Hz, 3H). LCMS m/z 502.2 [M+H]⁺.

### Step 2. (2S,6S)-1-benzyl-N-(2-bromo-5-chloro-phenyl)-N-[(4-methoxyphenyl)methyl]-2-methyl-6-(1-methyltriazol-4-yl)piperidine-4-carboxamide (C46)

A solution of (2S,6S)-1-benzyl-N-(2-bromo-5-chloro-phenyl)-2-methyl-6-(1-methyltriazol-4-yl)piperidine-4-carboxamide C45 (135 mg, 0.2632 mmol) in THF (3.70 mL) was cooled in a vial to 0° C., and treated with sodium hydride (16.6 mg, 0.4150 mmol, 60 wt %). After 10 minutes, 1-(bromomethyl)-4-methoxy-benzene (46 μL, 0.3155 mmol) was added at 0° C. The reaction was warmed to room temperature after 10 minutes. After 15 hours, the reaction was quenched slowly with saturated NaHCO₃ solution and extracted with DCM (×4). The combined organic layer was dried over Na₂SO₄, filtered, and concentrated in vacuo. The crude material was absorbed onto silica gel and purified with 0 to 100% EtOAc in heptane to provide (2S,6S)-1-benzyl-N-(2-bromo-5-chloro-phenyl)-N-[(4-methoxyphenyl)methyl]-2-methyl-6-(1-methyltriazol-4-yl)piperidine-4-carboxamide C46 (151 mg, 88%). ¹H NMR (300 MHz, Chloroform-d) δ 7.63 (d, J=8.6 Hz, 1H), 7.32-6.95 (m, 9H), 6.85-6.66 (m, 3H), 5.45 (d, J=14.2 Hz, 1H), 3.99 (d, J=14.2 Hz, 1H), 3.85 (d, J=1.0 Hz, 3H), 3.79 (d, J=6.4 Hz, 3H), 3.72-3.61 (m, 1H), 3.58 (d, J=2.3 Hz, 2H), 2.36 (dddd, J=29.7, 11.1, 5.5, 2.5 Hz, 1H), 2.26-2.07 (m, 1H), 2.03-1.86 (m, 1H), 1.86-1.46 (m, 3H), 1.06 (dd, J=13.7, 6.1 Hz, 3H). LCMS m/z 622.1 [M+H]⁺. ¹H NMR spectrum indicated about 1:1 mixture of diastereomers.

### Step 3. (2′S,6′S)-1′-benzyl-6-chloro-]-[(4-methoxyphenyl)methyl]-2′-methyl-6′-(1-methyltriazol-4-yl)spiro[indoline-3,4′-piperidine]-2-one (C47)

In a N2-glovebox was set up this reaction: to an oven-dried 2-dram vial was added (2S,6S)-1-benzyl-N-(2-bromo-5-chloro-phenyl)-N-[(4-methoxyphenyl)methyl]-2-methyl-6-(1-methyltriazol-4-yl)piperidine-4-carboxamide C46 (66 mg, 0.1009 mmol), followed by BINAP Pd G3 (10 mg, 0.01008 mmol), sodium t-butoxide (29 mg, 0.3018 mmol) and dioxane (1.1 mL). The vial was transferred into fume hood and heated to 100° C. After 5 hours, the reaction was quenched with saturated NaHCO₃ solution and extracted with EtOAc (×5). The combined organic layer was dried over Na₂SO₄, filtered, and concentrated in vacuo. The crude material was purified with a 40 g silica gel column and eluted with 0 to 100% EtOAc in heptane to provide (2′S,6′S)-1′-benzyl-6-chloro-1-[(4-methoxyphenyl)methyl]-2′-methyl-6′-(1-methyltriazol-4-yl)spiro[indoline-3,4′-piperidine]-2-one C47 (35.4 mg, 29%). LCMS m/z 542.16 [M+H]⁺. ¹H NMR showed it contained a mixture of two diastereomers.

### Step 4. (2′S,3S,6′S)-6-chloro-]-[(4-methoxyphenyl)methyl]-2′-methyl-6′-(1-methyltriazol-4-yl)spiro[indoline-3,4′-piperidine]-2-one and (2′S,3S,6′S)-1-[(4-methoxyphenyl)methyl]-2′-methyl-6′-(1-methyltriazol-4-yl)spiro[indoline-3,4′-piperidine]-2-one (C48 and C32)

To a stirred solution of (2′S,6′S)-1′-benzyl-6-chloro-1-[(4-methoxyphenyl)methyl]-2′-methyl-6′-(1-methyltriazol-4-yl)spiro[indoline-3,4′-piperidine]-2-one C47 (35 mg, 0.065 mmol), palladium on carbon (Evonik Noblyst® P1090 5% Pd, 12.4 mg) in EtOH (250 μL) and EtOAc (250 μL) was added. The reaction flask was evacuated and refilled with H₂ for 4 times. Then the reaction mixture was stirred under hydrogen balloon pressure. After 48 hours, the reaction mixture was filtered through a plug of Celite® and washed with EtOAc. The filtrate was concentrated and purified by silica gel chromatography (Column: 12 g column, Gradient: 0-10% MeOH in DCM) to afford 4 fractions, including two sets of diastereomers of products and dehalogenated products. The desired trans-products were isolated as the third and fourth fraction:

(2′S,3S,6′S)-6-chloro-1-[(4-methoxyphenyl)methyl]-2′-methyl-6′-(1-methyltriazol-4-yl)spiro[indoline-3,4′-piperidine]-2-one C48 (4.9 mg, 17%). ¹H NMR (300 MHz, Chloroform-d) δ 7.48 (s, 1H), 7.19 (d, J=8.5 Hz, 2H), 7.12 (d, J=7.9 Hz, 1H), 7.00 (dd, J=7.9, 1.8 Hz, 1H), 6.86 (d, J=8.4 Hz, 2H), 6.70 (d, J=1.8 Hz, 1H), 5.00 (dd, J=11.6, 3.1 Hz, 1H), 4.80 (s, 2H), 4.06 (s, 3H), 3.94-3.80 (m, 1H), 3.78 (s, 3H), 2.17-1.95 (m, 2H), 1.82-1.56 (m, 2H), 1.17 (d, J=6.3 Hz, 3H).

(2′S,3S,6′S)-1-[(4-methoxyphenyl)methyl]-2′-methyl-6′-(1-methyltriazol-4-yl)spiro[indoline-3,4′-piperidine]-2-one C32 (4.5 mg, 12%). ¹H NMR (300 MHz, Chloroform-d) δ 7.52 (s, 1H), 7.21 (d, J=8.2 Hz, 3H), 7.15 (t, J=7.6 Hz, 1H), 7.07-6.97 (m, 1H), 6.84 (d, J=8.3 Hz, 2H), 6.72 (d, J=7.7 Hz, 1H), 5.04 (dd, J=11.8, 2.9 Hz, 1H), 4.83 (s, 2H), 4.06 (s, 3H), 4.00-3.83 (m, 1H), 3.77 (s, 3H), 2.19 (t, J=12.7 Hz, 2H), 1.84-1.67 (m, 2H), 1.19 (d, J=6.3 Hz, 3H).

### Note:

the stereochemistry is assigned by comparing ¹H NMR spectrum from syntheses with related analogues (compound 33), and the key ¹H signal is the methine peak around 4.5-5 ppm. Trans isomer showed slight downfield shift (around 5.0 ppm), where in cis isomer it showed up around 4.6 ppm.

### Step 5. (2′S,3S,6′S)-6-chloro-2′-methyl-6′-(1-methyltriazol-4-yl)spiro[indoline-3,4′-piperidine]-2-one (36)

To a 1-dram vial with (2′S,3S,6′S)-6-chloro-1-[(4-methoxyphenyl)methyl]-2′-methyl-6′-(1-methyltriazol-4-yl)spiro[indoline-3,4′-piperidine]-2-one C48 (4.9 mg, 0.01084 mmol) (E35207-226-F3) in DCM (300 μL) at 0° C. was added trifluoromethanesulfonic acid (10 μL, 0.1130 mmol) (caution: exothermic). The vial was warmed to room temperature after the addition of acid. After 5 hours, the reaction was cooled to 0° C. and carefully quenched with saturated NaHCO₃ solution and extracted with DCM (×5). The combined organic layer was dried over Na₂SO₄, filtered, and concentrated in vacuo. The crude material was purified with two 4 g silica gel columns and eluted with 0 to 20% MeOH in DCM to provide (2′S,3S,6′S)-6-chloro-2′-methyl-6′-(1-methyltriazol-4-yl)spiro[indoline-3,4′-piperidine]-2-one 36 (3.8 mg, 99%). ¹H NMR (300 MHz, Chloroform-d) δ 7.93 (s, 1H), 7.45 (s, 1H), 7.10 (d, J=8.0 Hz, 1H), 7.01 (dd, J=8.0, 1.8 Hz, 1H), 6.88 (d, J=1.8 Hz, 1H), 4.93 (dd, J=8.6, 6.0 Hz, 1H), 4.05 (s, 3H), 3.80 (ddd, J=11.4, 6.1, 2.6 Hz, 1H), 2.13-2.01 (m, 2H), 1.81 (d, J=2.7 Hz, 1H), 1.57 (dd, J=13.6, 11.4 Hz, 1H), 1.15 (d, J=6.3 Hz, 3H). LCMS m/z 332.12 [M+H]⁺.

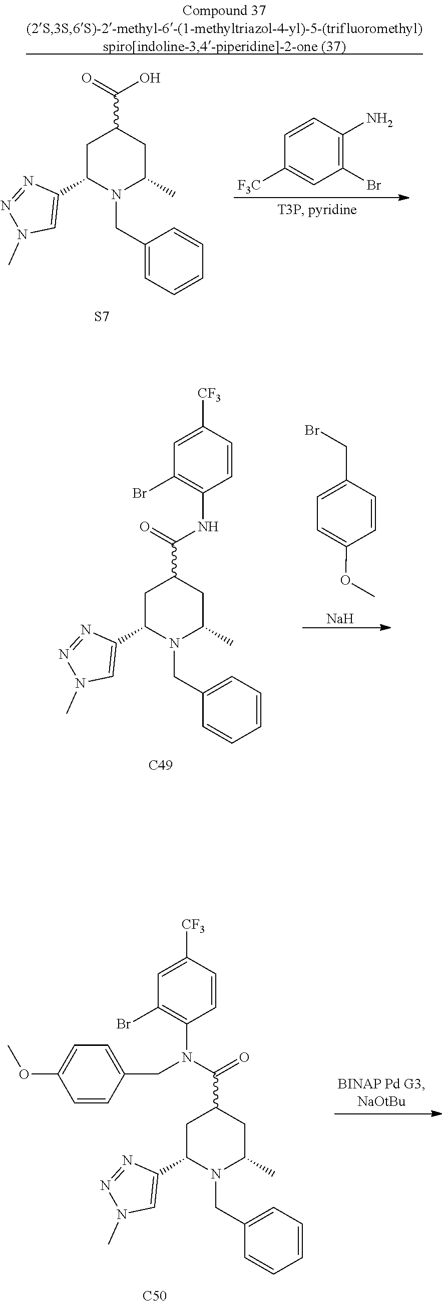

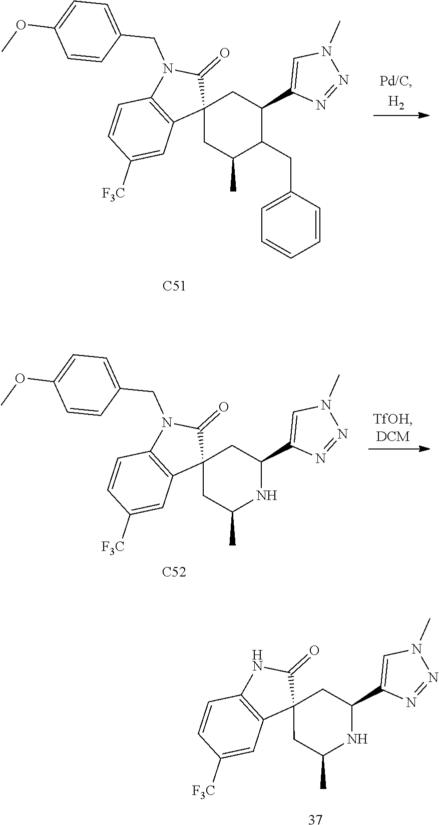

### Step 1. (2S,6S)-1-benzyl-N-[2-bromo-4-(trifluoromethyl)phenyl]-2-methyl-6-(1-methyltriazol-4-yl)piperidine-4-carboxamide (C49)

A 20-mL scintillation vial was charged with (2S,6S)-1-benzyl-2-methyl-6-(1-methyltriazol-4-yl)piperidine-4-carboxylic acid S7 (76 mg, 0.2417 mmol), 2-bromo-4-(trifluoromethyl)aniline (66 mg, 0.2750 mmol), pyridine (64 μL, 0.7913 mmol), and EtOAc (1000 μL). Propylphosphonic anhydride solution (270 μL, 0.46 mmol, 50 wt % in EtOAc) was added last. After 23 hours, the reaction was quenched with saturated NaHCO₃ solution and extracted with EtOAc (×4). The crude mixture was purified by silica gel chromatography with 0 to 100% EtOAc in heptane to provide (2S,6S)-1-benzyl-N-[2-bromo-4-(trifluoromethyl)phenyl]-2-methyl-6-(1-methyltriazol-4-yl)piperidine-4-carboxamide C49 (100 mg, 76%). ¹H NMR (300 MHz, Chloroform-d) δ 8.54 (d, J=8.7 Hz, 1H), 7.82 (d, J=2.8 Hz, 2H), 7.58 (d, J=8.7 Hz, 1H), 7.34-7.00 (m, 5H), 4.07-3.94 (m, 1H), 3.91 (s, 3H), 3.82-3.64 (m, 2H), 2.70 (d, J=7.7 Hz, 1H), 2.58 (td, J=12.4, 6.1 Hz, 1H), 2.29-2.10 (m, 1H), 2.05-1.72 (m, 3H), 1.20 (d, J=6.0 Hz, 3H). LCMS m/z 536.04 [M+H]⁺.

### Step 2. (2S,6S)-]-benzyl-N-[2-bromo-4-(trifluoromethyl)phenyl]-N-[(4-methoxyphenyl)methyl]-2-methyl-6-(1-methyltriazol-4-yl)piperidine-4-carboxamide (C50)

A solution of (2S,6S)-1-benzyl-N-[2-bromo-4-(trifluoromethyl)phenyl]-2-methyl-6-(1-methyltriazol-4-yl)piperidine-4-carboxamide C49 (100 mg, 0.1864 mmol) in THF (1.5 mL) was cooled in a vial to 0° C., and treated with sodium hydride (16 mg, 0.40 mmol, 60 wt %). The vial was warmed to room temperature after 5 minutes. After another 10 minutes, 1-(bromomethyl)-4-methoxy-benzene (41 μL, 0.2812 mmol) was added at room temperature. After 6 hours, additional 1-(bromomethyl)-4-methoxy-benzene (10 μL, 0.069 mmol) was added. After another 1 hour, the reaction was quenched slowly with saturated NaHCO₃ solution and extracted with EtOAc (×4). The combined organic layer was dried over Na₂SO₄, filtered, and concentrated. The crude material was absorbed onto silica gel and purified with 0 to 100% EtOAc in heptane to provide (2S,6S)-1-benzyl-N-[2-bromo-4-(trifluoromethyl)phenyl]-N-[(4-methoxyphenyl)methyl]-2-methyl-6-(1-methyltriazol-4-yl)piperidine-4-carboxamide C50 (37.2 mg, 27%). LCMS m/z 656.17 [M+H]⁺. The material was then heated to 80° C. for 2 hours to remove the residual EtOAc. ¹H NMR indicated the product consisted of a mixture of diastereomers with a ratio of 1.3:1.

### Step 3. (2′S,3S,6′S)-1′-benzyl-]-[(4-methoxyphenyl)methyl]-2′-methyl-6′-(]-methyltriazol-4-yl)-5-(trifluoromethyl)spiro[indoline-3,4′-piperidine]-2-one (C51)

In a N2-glovebox was set up this reaction: to a 2-dram vial was added (2S,6S)-1-benzyl-N-[2-bromo-4-(trifluoromethyl)phenyl]-N-[(4-methoxyphenyl)methyl]-2-methyl-6-(1-methyltriazol-4-yl)piperidine-4-carboxamide C50 (37.2 mg, 0.05103 mmol), followed by BINAP Pd G3 (5.1 mg, 0.005139 mmol) and sodium t-butoxide (11.2 mg, 0.1165 mmol). Lastly, dioxane (500 μL) was added. The vial was relocated from the glovebox to the bench and heated to 100° C. After 16 hours, the reaction was quenched with saturated NaHCO₃ solution and extracted with EtOAc (×5). The combined organic layer was dried over Na₂SO₄, filtered, and concentrated. The crude material was purified with silica gel columns and eluted with 0 to 100% EtOAc in heptane to provide two diastereomers. The trans isomer was isolated as the less polar fraction and also the major product: (2′S,3S,6′S)-1′-benzyl-1-[(4-methoxyphenyl)methyl]-2′-methyl-6′-(1-methyltriazol-4-yl)-5-(trifluoromethyl)spiro[indoline-3,4′-piperidine]-2-one C51 (22.5 mg, 77%). ¹H NMR (300 MHz, Chloroform-d) δ 7.48 (s, 1H), 7.41 (d, J=8.3 Hz, 1H), 7.30-7.01 (m, 8H), 6.84 (d, J=8.1 Hz, 2H), 6.76 (d, J=8.2 Hz, 1H), 5.04-4.76 (m, 3H), 3.85 (d, J=15.0 Hz, 5H), 3.77 (t, J=1.1 Hz, 4H), 2.34 (t, J=12.9 Hz, 1H), 1.99 (q, J=15.2, 14.1 Hz, 2H), 1.74 (d, J=13.7 Hz, 1H), 1.08 (d, J=6.2 Hz, 3H).

### Step 4. (2′S,3S,6′S)-1-[(4-methoxyphenyl)methyl]-2′-methyl-6′-(1-methyltriazol-4-yl)-5-(trifluoromethyl)spiro[indoline-3,4′-piperidine]-2-one (C52)

To a stirred solution of (2′S,3S,6′S)-1′-benzyl-1-[(4-methoxyphenyl)methyl]-2′-methyl-6′-(1-methyltriazol-4-yl)-5-(trifluoromethyl)spiro[indoline-3,4′-piperidine]-2-one C51 (22.5 mg, 0.0391 mmol) in EtOH (450 μL) and EtOAc (450 μL) was added palladium on carbon (Evonik Noblyst® P1090 5% Pd, 13 mg, 0.006108 mmol). The reaction flask was evacuated and refilled with H₂ for 4 times. Then the reaction mixture was stirred under hydrogen balloon pressure. After 17 h, the reaction flask was evacuated and refilled with N2, then the reaction mixture was filtered through a plug of Celite® and washed with EtOAc. The filtrate was concentrated and purified by silica gel chromatography (Column: 12 g column, Gradient: 0-12% MeOH in DCM) afforded (2′S,3S,6′S)-1-[(4-methoxyphenyl)methyl]-2′-methyl-6′-(1-methyltriazol-4-yl)-5-(trifluoromethyl)spiro[indoline-3,4′-piperidine]-2-one C52 (17.1 mg, 87%). ¹H NMR (300 MHz, Chloroform-d) δ 7.48 (s, 1H), 7.46-7.34 (m, 2H), 7.19 (d, J=8.1 Hz, 2H), 6.85 (d, J=8.2 Hz, 2H), 6.77 (d, J=8.1 Hz, 1H), 5.00 (dd, J=11.2, 3.4 Hz, 1H), 4.85 (s, 2H), 4.06 (d, J=1.4 Hz, 3H), 3.87 (t, J=8.9 Hz, 1H), 3.77 (t, J=1.3 Hz, 3H), 2.38-1.93 (m, 2H), 1.79 (dd, J=13.6, 2.3 Hz, 1H), 1.61 (d, J=12.7 Hz, 1H), 1.17 (d, J=6.3 Hz, 3H). LCMS m/z 486.17 [M+H]⁺.

### Step 5. (2′S,3S,6′S)-2′-methyl-6′-(1-methyltriazol-4-yl)-5-(trifluoromethyl)spiro[indoline-3,4′-piperidine]-2-one (37)

To a 20-mL vial with (2′S,3S,6′S)-1-[(4-methoxyphenyl)methyl]-2′-methyl-6′-(1-methyltriazol-4-yl)-5-(trifluoromethyl)spiro[indoline-3,4′-piperidine]-2-one C52 (17.1 mg, 0.03397 mmol) in DCM (500 μL) at 0° C. was added trifluoromethanesulfonic acid (31 μL, 0.3503 mmol). The vial was warmed to room temperature after the addition of acid. After 5 hours, the reaction was cooled to 0° C. and carefully quenched with saturated NaHCO₃ solution and extracted with DCM (×5). The combined organic layer was dried over Na₂SO₄, filtered, and concentrated. The crude material was purified with silica gel columns and eluted with 0 to 20% MeOH in DCM to provide (2′S,3S,6′S)-2′-methyl-6′-(1-methyltriazol-4-yl)-5-(trifluoromethyl)spiro[indoline-3,4′-piperidine]-2-one 37 (12.3 mg, 97%)¹H NMR (300 MHz, CDCl3) δ 8.83 (s, 1H), 7.47 (d, J=8.8 Hz, 3H), 6.97 (d, J=8.0 Hz, 1H), 4.96 (t, J=7.3 Hz, 1H), 4.05 (s, 3H), 3.94-3.69 (m, 1H), 2.07 (d, J=7.4 Hz, 2H), 1.82 (d, J=13.3 Hz, 1H), 1.70-1.54 (m, 1H), 1.16 (d, J=6.2 Hz, 3H). LCMS m/z 366.1 [M+H]⁺.

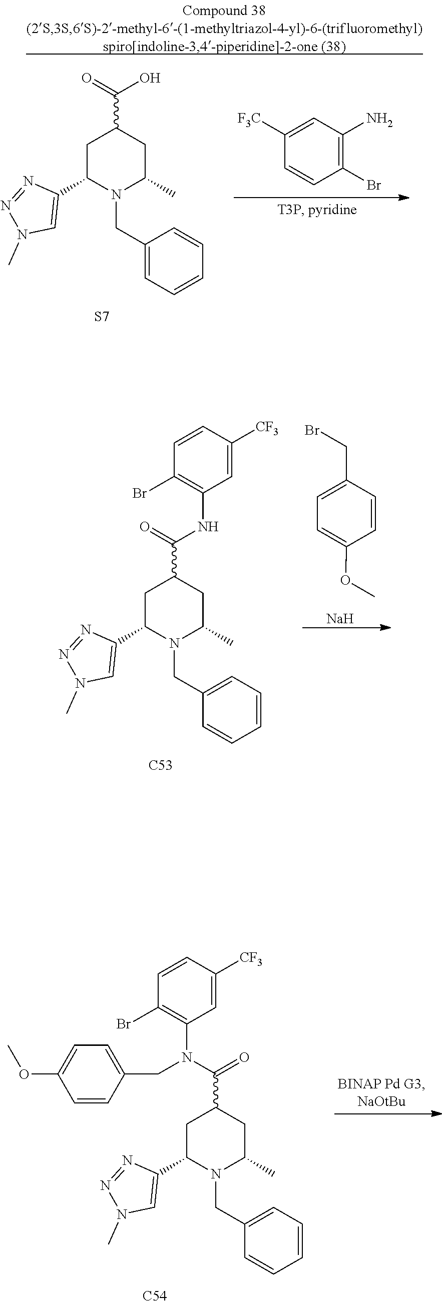

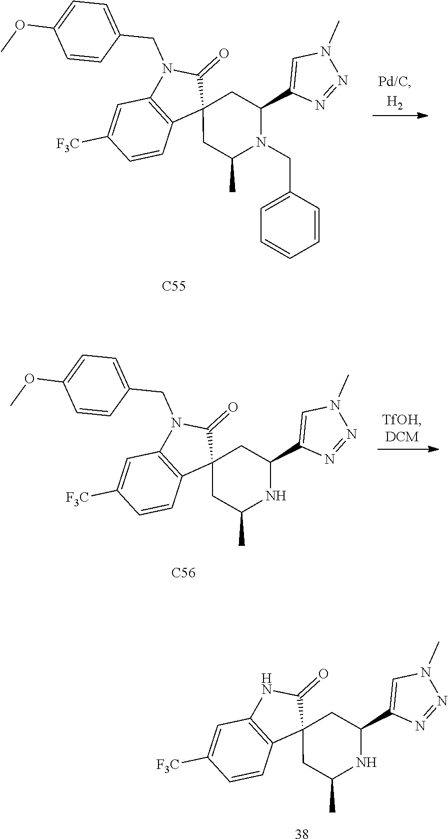

### Step 1. (2S,6S)-1-benzyl-N-[2-bromo-5-(trifluoromethyl)phenyl]-2-methyl-6-(1-methyltriazol-4-yl)piperidine-4-carboxamide (C53)

To a 2-dram vial was charged (2S,6S)-1-benzyl-2-methyl-6-(1-methyltriazol-4-yl)piperidine-4-carboxylic acid S7 (85 mg, 0.2704 mmol), 2-bromo-5-(trifluoromethyl)aniline (44 μL, 0.3071 mmol), pyridine (70 μL, 0.8655 mmol), and EtOAc (900 μL). Propylphosphonic anhydride solution (310 μL, 0.52 mmol, 50 wt % in EtOAc) was added. After 24 hours, the reaction was quenched with saturated NaHCO₃ solution and extracted with EtOAc (×4). The crude mixture was purified by silica gel chromatography with 0 to 100% EtOAc in heptane to provide (2S,6S)-1-benzyl-N-[2-bromo-5-(trifluoromethyl)phenyl]-2-methyl-6-(1-methyltriazol-4-yl)piperidine-4-carboxamide C53 (105 mg, 71%). LCMS m/z 536.04 [M+H]⁺.

### Step 2. (2S,6S)-]-benzyl-N-[2-bromo-5-(trifluoromethyl)phenyl]-N-[(4-methoxyphenyl)methyl]-2-methyl-6-(1-methyltriazol-4-yl)piperidine-4-carboxamide (C54)

To a vial was added (2S,6S)-1-benzyl-N-[2-bromo-5-(trifluoromethyl)phenyl]-2-methyl-6-(1-methyltriazol-4-yl)piperidine-4-carboxamide C53 (105 mg, 0.1958 mmol) in THF (2.2 mL). The vial was cooled to 0° C. and treated with sodium hydride (16 mg, 0.4000 mmol, 60 wt %) at 0° C. The vial was warmed to room temperature after 5 minutes. After another 10 minutes, 1-(bromomethyl)-4-methoxy-benzene (44 μL, 0.3018 mmol) was added at room temperature. After 4 hours, the reaction was quenched slowly with saturated NaHCO₃ solution and extracted with DCM (×3). The combined organic layer was dried over Na₂SO₄, filtered, and concentrated. The crude material was absorbed onto silica gel and purified with 0 to 100% EtOAc in heptane to provide (2S,6S)-1-benzyl-N-[2-bromo-5-(trifluoromethyl)phenyl]-N-[(4-methoxyphenyl)methyl]-2-methyl-6-(1-methyltriazol-4-yl)piperidine-4-carboxamide C54 (106 mg, 78%). LCMS m/z 656.12 [M+H]*. The material was then heated to 80° C. for 2 hours to remove the residual EtOAc. ¹H NMR indicated the product consisted of a mixture of diastereomers with a ratio of 1.3:1.

### Step 3. (2′S,6′S)-1′-benzyl-]-[(4-methoxyphenyl)methyl]-2′-methyl-6′-(1-methyltriazol-4-yl)-6-(trifluoromethyl)spiro[indoline-3,4′-piperidine]-2-one (C55)

In a N2-glovebox was set up this reaction: to a 20 ml vial was added (2S,6S)-1-benzyl-N-[2-bromo-5-(trifluoromethyl)phenyl]-N-[(4-methoxyphenyl)methyl]-2-methyl-6-(1-methyltriazol-4-yl)piperidine-4-carboxamide C54 (105 mg, 0.1599 mmol), followed by BINAP Pd G3 (16 mg, 0.01612 mmol) and sodium t-butoxide (35 mg, 0.3642 mmol). Lastly, dioxane (1.5 mL) was added. The vial was relocated from the glovebox to the bench and heated to 100° C. After 17 hours, the reaction was quenched with saturated NaHCO₃ solution and extracted with EtOAc (×5). The combined organic layer was dried over Na₂SO₄, filtered, and concentrated. The crude material was purified with silica gel columns and eluted with 0 to 100% EtOAc in heptane to provide (2′S,6′S)-1′-benzyl-1-[(4-methoxyphenyl)methyl]-2′-methyl-6′-(1-methyltriazol-4-yl)-6-(trifluoromethyl)spiro[indoline-3,4′-piperidine]-2-one C55 (92.3 mg, 93%). LCMS m/z 576.19 [M+H]⁺. Based on ¹⁹F NMR, the ratio of diastereomer is 2.7:1. Based on ¹H NMR, the isolated product also contained about 7% of BINAP-related impurities.

### Step 4. (2′S,3S,6′S)-1-[(4-methoxyphenyl)methyl]-2′-methyl-6′-(1-methyltriazol-4-yl)-6-(trifluoromethyl)spiro[indoline-3,4′-piperidine]-2-one (C56)

To a stirred solution of (2′S,6′S)-1′-benzyl-1-[(4-methoxyphenyl)methyl]-2′-methyl-6′-(1-methyltriazol-4-yl)-6-(trifluoromethyl)spiro[indoline-3,4′-piperidine]-2-one C55 (92.3 mg, 0.1491 mmol) in EtOH (1.6 mL) and EtOAc (1.6 mL) was added palladium on carbon (Evonik Noblyst® P1090 5% Pd, 47.5 mg, 0.02232 mmol). The reaction flask was evacuated and refilled with H₂ for 4 times. Then the reaction mixture was stirred under hydrogen balloon pressure. After 3 hours, H₂ was recharged, and the reaction was left at room temperature for 62 hours. The reaction mixture was filtered through a plug of Celite® and washed with EtOAc. The filtrate was concentrated and purified by silica gel chromatography (Column: 12 g column, Gradient: 0-12% MeOH in DCM) to afford two fractions, and the more polar fraction was isolated as (2′S,3S,6′S)-1-[(4-methoxyphenyl)methyl]-2′-methyl-6′-(1-methyltriazol-4-yl)-6-(trifluoromethyl)spiro[indoline-3,4′-piperidine]-2-one C56 (44.6 mg, 60%). ¹H NMR (300 MHz, Chloroform-d) δ 7.45 (s, 1H), 7.30 (s, 2H), 7.21 (d, J=8.2 Hz, 2H), 6.92 (s, 1H), 6.90-6.79 (m, 2H), 5.11-4.91 (m, 1H), 4.84 (s, 2H), 4.05 (d, J=1.7 Hz, 3H), 3.98-3.78 (m, 1H), 3.77 (d, J=1.5 Hz, 3H), 2.21-1.83 (m, 3H), 1.76 (dd, J=13.5, 2.5 Hz, 1H), 1.61 (t, J=12.4 Hz, 1H), 1.16 (d, J=6.3 Hz, 3H). LCMS m/z 486.13 [M+H]⁺.

### Note:

The stereochemistry is assigned by comparing ¹H NMR spectrum from syntheses with related analogues (compound 33), and the key ¹H signal is the methine peak around 4.5-5 ppm. Trans isomer showed slight downfield shift (around 5.0 ppm), while in the cis isomer it showed up around 4.6 ppm.

### Step 5. (2′S,3S,6′S)-2′-methyl-6′-(1-methyltriazol-4-yl)-6-(trifluoromethyl)spiro[indoline-3,4′-piperidine]-2-one (38)

To a 20-mL vial with (2′S,3S,6′S)-1-[(4-methoxyphenyl)methyl]-2′-methyl-6′-(1-methyltriazol-4-yl)-6-(trifluoromethyl)spiro[indoline-3,4′-piperidine]-2-one C56 (44.6 mg, 0.09186 mmol) in DCM (1.5 mL) at 0° C. was added trifluoromethanesulfonic acid (83 μL, 0.9380 mmol). The vial was warmed to room temperature after the addition of acid. After 6 hours, the reaction was cooled to 0° C. and carefully quenched with saturated NaHCO₃ solution and extracted with DCM (×5). The combined organic layer was dried over Na₂SO₄, filtered, and concentrated. The crude material was purified with silica gel columns and eluted with 0 to 20% MeOH in DCM to provide (2′S,3S,6′S)-2′-methyl-6′-(1-methyltriazol-4-yl)-6-(trifluoromethyl)spiro[indoline-3,4′-piperidine]-2-one 38 (28.0 mg, 81%). ¹H NMR (300 MHz, Chloroform-d) δ 8.99 (s, 1H), 7.46 (s, 1H), 7.30 (d, J=2.7 Hz, 2H), 7.12 (s, 1H), 4.97 (dd, J=9.1, 5.5 Hz, 1H), 4.05 (d, J=1.4 Hz, 3H), 3.93-3.71 (m, 1H), 2.22-1.99 (m, 2H), 1.81 (d, J=13.7 Hz, 1H), 1.61 (t, J=12.4 Hz, 1H), 1.15 (d, J=6.2 Hz, 3H). LCMS m/z 366.14 [M+H]⁺.

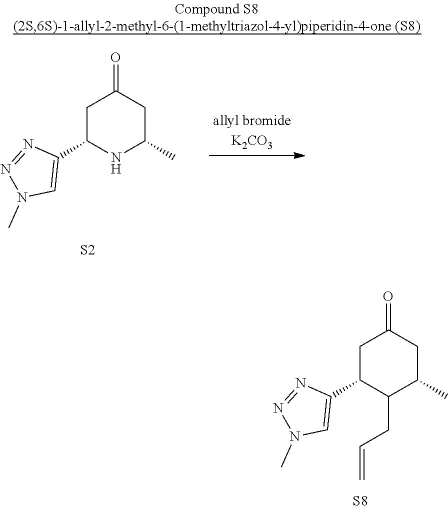 [SMILES: C[C@H]1CC(=O)C[C@@H](c2cn(C)nn2)N1 ; "S2" ; C=CCC1[C@@H](C)CC(=O)C[C@H]1c1cn(C)nn1 ; "S8"]

To a suspension of (2S,6S)-2-methyl-6-(1-methyltriazol-4-yl)piperidin-4-one (S2) (10.0 g, 50.5 mmol) and K₂CO₃ (8.0 g, 57.9 mmol) in MeCN (100 mL) was added allyl bromide (5.5 mL, 63.6 mmol), and the mixture was heated to 40° C. and stirred for 18 hours. The suspension was then filtered, rinsed with MeCN, and concentrated to about 3 volumes. The mixture was diluted with TBME/EtOAc/DCM 1:1:1 (300 mL) and water (250 mL). The aqueous layer was extracted with DCM (2×150 mL). The combined organic layer was washed with saturated brine (250 mL), dried with MgSO₄, filtered, and concentrated. The mixture was suspended in TBME (180 mL) and refluxed. Upon reflux, full dissolution to a yellow solution was observed. The mixture was removed from the bath and stirred. After about 5 minutes, significant precipitation was observed. At this time, the mixture was cooled with an ice bath for 10 minutes, filtered, and rinsed with TBME (2×15 mL). Dissolution was observed, so subsequent rinses were carried out using heptane (3×20 mL). The addition of heptane caused a significant amount of precipitation in the mother liquor, which was filtered and rinsed with heptane (3×10 mL) to yield the second crop. The crops were combined to yield the title compound S8 (2S,6S)-1-allyl-2-methyl-6-(1-methyltriazol-4-yl)piperidin-4-one (8.42 g, 71%) as an off-white solid. ¹H NMR (300 MHz, Chloroform-d) δ 7.48 (s, 1H), 5.91 (ddt, J=16.9, 11.1, 6.4 Hz, 1H), 5.13 (t, J=14.6 Hz, 2H), 4.23 (dd, J=10.9, 3.8 Hz, 1H), 4.12 (d, J=1.3 Hz, 3H), 3.44 (dd, J=16.0, 6.8 Hz, 1H), 3.17 (dd, J=16.0, 6.3 Hz, 1H), 3.06 (dt, J=10.5, 5.4 Hz, 1H), 2.88 (dd, J=14.6, 10.9 Hz, 1H), 2.59 (dd, J=14.8, 3.7 Hz, 1H), 2.53-2.34 (m, 2H), 1.27 (d, J=6.2 Hz, 3H).

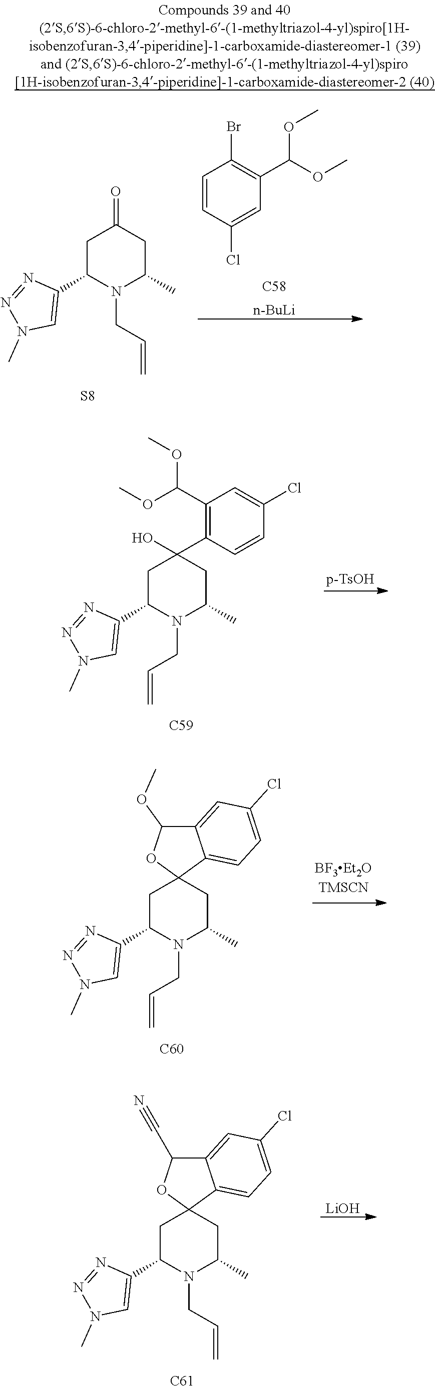

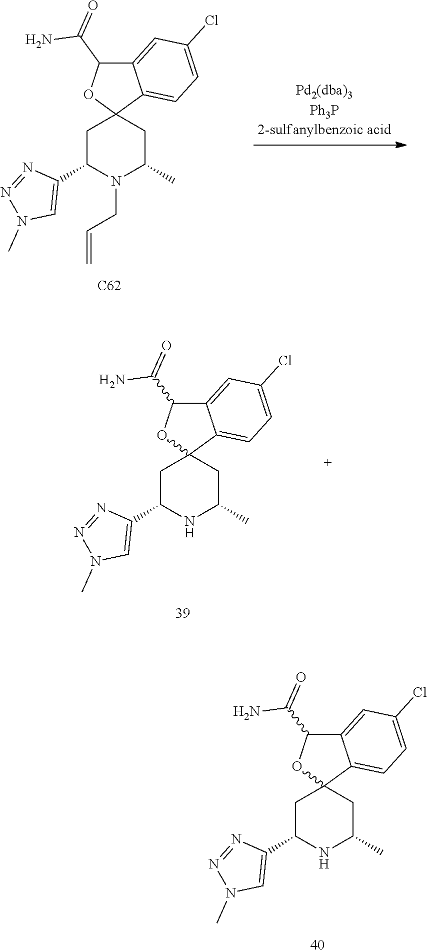 [SMILES: C=CCN1[C@@H](C)CC2(C[C@H]1c1cn(C)nn1)OC(C(N)=O)c1cc(Cl)ccc12 ; "C62" ; C[C@H]1CC2(C[C@@H](c3cn(C)nn3)N1)OC(C(N)=O)c1cc(Cl)ccc12 ; "39" ; C[C@H]1CC2(C[C@@H](c3cn(C)nn3)N1)OC(C(N)=O)c1cc(Cl)ccc12 ; "40"]

### Step 1. Synthesis of (2S,6S)-1-allyl-4-[4-chloro-2-(dimethoxymethyl)phenyl]-2-methyl-6-(1-methyltriazol-4-yl)piperidin-4-ol (C59)

To a solution of 1-bromo-4-chloro-2-(dimethoxymethyl)benzene C58 (1.38 g, 4.937 mmol) in THF (12 mL) at −78° C. under Argon was added n-BuLi (2.8 mL of 1.6 M, 4.480 mmol). The mixture was stirred for 45 minutes at −78° C., then a solution of (2S,6S)-1-allyl-2-methyl-6-(1-methyltriazol-4-yl)piperidin-4-one S8 (415 mg, 1.736 mmol) in THF (6 mL) was added. The reaction was stirred at −78° C. for 90 minutes, then warmed to 0° C. After 40 minutes at 0° C., the reaction was quenched with saturated aqueous ammonium chloride (100 mL), extracted with DCM (3×75 mL), dried over Na₂SO₄, passed through a phase separator, and then concentrated in vacuo. The resulting crude residue was used in the next step without further purification.

### Step 2. Synthesis of (2′S,6′S)-1′-allyl-6-chloro-1-methoxy-2′-methyl-6′-(1-methyltriazol-4-yl)spiro[1H-isobenzofuran-3,4′-piperidine](C60)

The crude residue of C59 from step 1 was dissolved in MeOH (40 mL) and treated with 4-methylbenzenesulfonic acid (Water (1)) (986 mg, 5.184 mmol). The mixture was stirred at room temperature for 16 hours. The reaction was then concentrated by approximately half the volume under a stream of N₂, then quenched with saturated aqueous sodium bicarbonate (50 mL), extracted with DCM (3×50 mL), dried over Na₂SO₄, passed through a phase separator, and then concentrated in vacuo to afford a crude residue which was used in the next step without further purification.

### Step 3. Synthesis of (2′S,6′S)-1′-allyl-6-chloro-2′-methyl-6′-(1-methyltriazol-4-yl)spiro[1H-isobenzofuran-3,4′-piperidine]-1-carbonitrile (C61)

The crude residue C60 from step 2 was dissolved in DCM (16 mL), cooled to −20° C., and trimethylsilylformonitrile (1.45 mL, 10.87 mmol) and diethyloxonio(trifluoro)boranuide (450 μL, 3.646 mmol) were added. The reaction was stirred for 30 minutes at −20° C., at which point it was warmed to 0° C. and stirred for 120 minutes. The reaction was quenched with DCM and MeOH, followed by 50 mL IN NaOH. The aqueous layer was extracted with DCM (×3), passed through a phase separator, and concentrated in vacuo. The crude residue was purified by silica gel chromatography (Eluent: MeOH in DCM) to provide partially purified C₆₁, which was used in the next step without further purification.

### Step 4. Synthesis of (2′S,6′S)-1′-allyl-6-chloro-2′-methyl-6′-(1-methyltriazol-4-yl)spiro[1H-isobenzofuran-3,4′-piperidine]-1-carboxamide (C62)

The residue from step 3, C61, was dissolved in THF (20 mL) and H₂O (20 mL). LiOH (84 mg, 3.508 mmol) was added. The reaction was stirred overnight at room temperature and then quenched into 1:1 saturated NH₄Cl:brine and DCM and extracted with DCM (×3). The pooled organics were passed through a phase separator and concentrated in vacuo. The crude residue was purified by silica gel chromatography (Eluent: MeOH in DCM) to provide partially purified C62, which was used in the next step without further purification.

### Step 5. Synthesis of (2′S,6′S)-6-chloro-2′-methyl-6′-(1-methyltriazol-4-yl)spiro[1H-isobenzofuran-3,4′-piperidine]-1-carboxamide-diastereomer-](39) and (2′S,6′S)-6-chloro-2′-methyl-6′-(1-methyltriazol-4-yl)spiro[1H-isobenzofuran-3,4′-piperidine]-1-carboxamide-diastereomer-2 (40)

A mixture of 4-diphenylphosphanylbutyl(diphenyl)phosphane (35 mg, 0.08207 mmol) and Pd₂(dba)₃ (34.5 mg, 0.03768 mmol) in THF (4 mL) was stirred for 30 minutes. To this mixture was added to a solution of 2-sulfanylbenzoic acid (316.4 mg, 2.052 mmol) and partially purified (2′S,6′S)-1′-allyl-6-chloro-2′-methyl-6′-(1-methyltriazol-4-yl)spiro[1H-isobenzofuran-3,4′-piperidine]-1-carboxamide (C62) from step 4 in THF (8 mL). The mixture was stirred under argon for 30 minutes. The reaction was diluted with TBME (40 mL) and 0.5 N HCl (40 mL). The layers were mixed, and the organic layer was removed and extracted with 1 N HCl (20 mL). The organic layer was removed, and the combined aqueous layer was filtered through a 0.45 micron filter and washed with additional TBME (20 mL). The pH was adjusted with 6 N NaOH until pH˜11. The hazy mixture was then extracted with DCM (3×50 mL), and the combined organic layer was passed through a phase separator and concentrated. The crude resulting residue was purified by reversed-phase HPLC. Method: Waters XBridge Prep C8 Column; 30×150 mm, 5 micron. Gradient: Acetonitrile in Water with 10 mM Ammonium Hydroxide. to yield (2′S,6′S)-6-chloro-2′-methyl-6′-(1-methyltriazol-4-yl)spiro[1H-isobenzofuran-3,4′-piperidine]-1-carboxamide-diasteromer-1 (53.3 mg, 8% over 5 steps) (39) LCMS m/z 362.19 [M+1]⁺; ¹H NMR (300 MHz, Chloroform-d) δ 7.62 (s, 1H), 7.42 (s, 1H), 7.30 (dd, J=8.1, 1.9 Hz, 1H), 7.03 (d, J=8.0 Hz, 1H), 6.68 (d, J=4.0 Hz, 1H), 5.86 (d, J=4.0 Hz, 1H), 5.47 (s, 1H), 4.46 (dd, J=11.7, 2.8 Hz, 1H), 4.05 (s, 3H), 3.44-3.32 (m, 1H), 2.19-1.63 (m, 4H), 1.20 (d, J=6.2 Hz, 3H). (2′S,6′S)-6-chloro-2′-methyl-6′-(1-methyltriazol-4-yl)spiro[1H-isobenzofuran-3,4′-piperidine]-1-carboxamide-diasteromer-2 (40) (92.7 mg, 14% over 5 steps) LCMS m/z 362.19 [M+1]⁺; ¹H NMR (300 MHz, Chloroform-d) δ 7.62 (d, J=1.7 Hz, 1H), 7.52-6.96 (m, 3H), 6.74 (d, J=4.0 Hz, 1H), 5.81-5.59 (m, 1H), 5.47 (d, J=4.5 Hz, 1H), 4.51 (dd, J=10.5, 4.3 Hz, 1H), 4.06 (d, J=5.2 Hz, 3H), 3.45-3.26 (m, 1H), 2.36-1.37 (m, 4H), 1.23-1.09 (m, 3H).

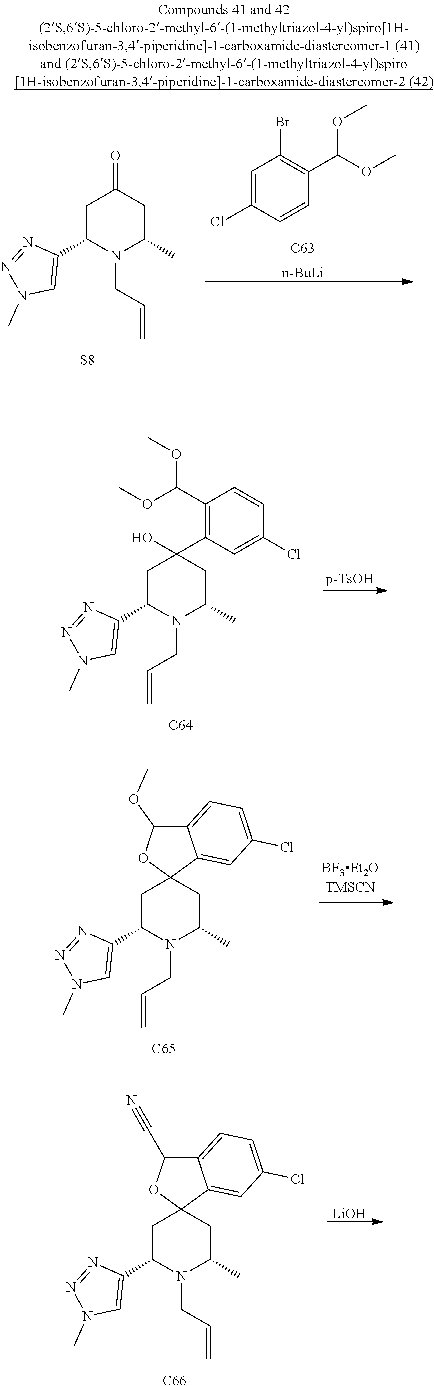

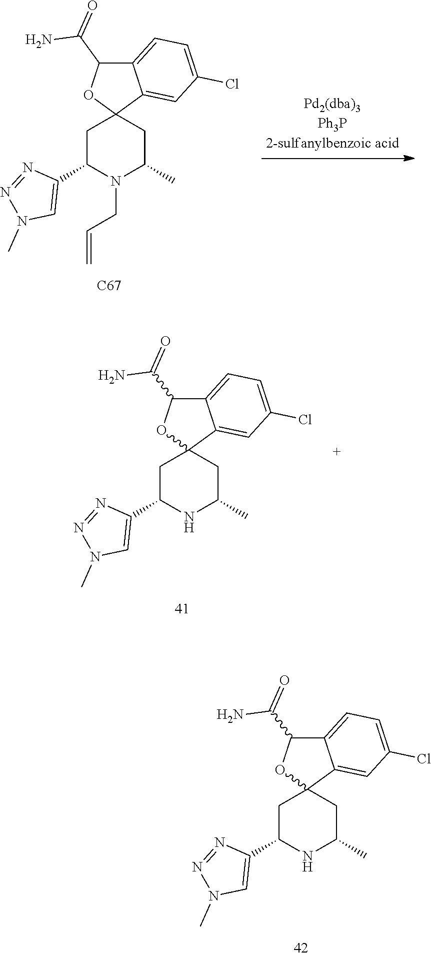 [SMILES: C=CCN1[C@@H](C)CC2(C[C@H]1c1cn(C)nn1)OC(C(N)=O)c1ccc(Cl)cc12 ; "C67" ; C[C@H]1CC2(C[C@@H](c3cn(C)nn3)N1)OC(C(N)=O)c1ccc(Cl)cc12 ; "41" ; C[C@H]1CC2(C[C@@H](c3cn(C)nn3)N1)OC(C(N)=O)c1ccc(Cl)cc12 ; "42"]

### Step 1. Synthesis of (2S,6S)-1-allyl-4-[4-chloro-2-(dimethoxymethyl)phenyl]-2-methyl-6-(1-methyltriazol-4-yl)piperidin-4-ol (C64)

To a solution of 2-bromo-4-chloro-1-(dimethoxymethyl)benzene C63 (333 mg, 1.191 mmol) in THF (3 mL) at −78° C. under Argon was added n-BuLi (700 μL of 1.6 M, 1.120 mmol). The reaction was stirred for 45 minutes at −78° C., then a solution of (2S,6S)-1-allyl-2-methyl-6-(1-methyltriazol-4-yl)piperidin-4-one S8 (101 mg, 0.4225 mmol) in THF (1.5 mL) was added. The reaction was stirred at −78° C. for 60 minutes, then warmed to 0° C. After 60 minutes at 0° C., the reaction was quenched with saturated aqueous ammonium chloride (75 mL), extracted with DCM (3×50 mL), dried over Na₂SO₄, passed through a phase separator, and then concentrated in vacuo. The resulting crude residue of (2S,6S)-1-allyl-4-[4-chloro-2-(dimethoxymethyl)phenyl]-2-methyl-6-(1-methyltriazol-4-yl)piperidin-4-ol (C64) was used in the next reaction without further purification.

### Step 2. Synthesis of(2′S,6′S)-1′-allyl-6-chloro-1-methoxy-2′-methyl-6′-(1-methyltriazol-4-yl)spiro[1H-isobenzofuran-3,4′-piperidine](C65)

The crude residue of C64 from step 1 was dissolved in MeOH (10 mL) and treated with 4-methylbenzenesulfonic acid (Water (1)) (240 mg, 1.262 mmol). The mixture was stirred at room temperature for 16 hours and then concentrated by approximately half the volume under a stream of N₂ and quenched with saturated aqueous sodium bicarbonate (50 mL), extracted with DCM (3×50 mL), dried over Na₂SO₄, passed through a phase separator, and then concentrated in vacuo to afford crude residue (2′S,6′S)-1′-allyl-6-chloro-1-methoxy-2′-methyl-6′-(1-methyltriazol-4-yl)spiro[1H-isobenzofuran-3,4′-piperidine](C65) which was used in the next reaction without further purification.

### Step 3. Synthesis of(2′S,6′S)-1′-allyl-6-chloro-2′-methyl-6′-(1-methyltriazol-4-yl)spiro[1H-isobenzofuran-3,4′-piperidine]-1-carbonitrile (C66)

The crude residue C65 from step 2 was dissolved in DCM (4 mL), cooled to −25° C., and trimethylsilylformonitrile (350 μL, 2.625 mmol) and diethyloxonio(trifluoro)boranuide (110 μL, 0.8913 mmol) were added. The reaction was stirred for 30 minutes, at which point it was warmed to 0° C. and stirred for 120 minutes. The reaction was then quenched with DCM and MeOH, followed by 50 mL 1N NaOH. The aqueous layer was extracted with DCM (×3), passed through a phase separator, and concentrated in vacuo. The crude residue was partially purified by silica gel chromatography (Eluent: MeOH in DCM) to provide impure (2′S,6′S)-1′-allyl-6-chloro-2′-methyl-6′-(1-methyltriazol-4-yl)spiro[1H-isobenzofuran-3,4′-piperidine]-1-carbonitrile (C66), which was used in the next step without further purification.

### Step 4. Synthesis of(2′S,6′S)-1′-allyl-6-chloro-2′-methyl-6′-(1-methyltriazol-4-yl)spiro[1H-isobenzofuran-3,4′-piperidine]-1-carboxamide (C67)

The partially purified residue C66 from step 3 was dissolved in THF (5 mL) and H₂O (5 mL) and LiOH (23 mg, 0.9604 mmol) was added. The reaction was stirred overnight at room temperature. The reaction was quenched into 1:1 saturated NH₄Cl:brine and DCM and extracted with DCM (×3). The pooled organics were passed through a phase separator and concentrated in vacuo. The resulting crude residue was partially purified by silica gel chromatography (Eluent: MeOH in DCM) to provide impure (2′S,6′S)-1′-allyl-5-chloro-2′-methyl-6′-(1-methyltriazol-4-yl)spiro[1H-isobenzofuran-3,4′-piperidine]-1-carboxamide (C67), which was used in the next step without further purification. Yield: 123 mg (50% purity) 36%. LCMS m/z 402.28 [M+1]⁺.

### Step 5. Synthesis of (2′S,6′S)-5-chloro-2′-methyl-6′-(1-methyltriazol-4-yl)spiro[1H-isobenzofuran-3,4′-piperidine]-1-carboxamide-diasteromer 1 (41) and (2′S,6′S)-5-chloro-2′-methyl-6′-(1-methyltriazol-4-yl)spiro[1H-isobenzofuran-3,4′-piperidine]-1-carboxamide diastereomer 2 (42)

A mixture of 4-diphenylphosphanylbutyl(diphenyl)phosphane (12.6 mg, 0.02954 mmol) and Pd₂(dba)₃ (12 mg, 0.01310 mmol) in THF (1 mL) was aged for 30 minutes and then added to a solution of 2-sulfanylbenzoic acid (77 mg, 0.4994 mmol) and partially purified (2′S,6′S)-1′-allyl-5-chloro-2′-methyl-6′-(1-methyltriazol-4-yl)spiro[1H-isobenzofuran-3,4′-piperidine]-1-carboxamide (C67) from step 4 in THF (2 mL). The mixture was stirred under argon for 30 minutes. The reaction was diluted with TBME (10 mL) and 0.5 N HCl (10 mL). The layers were mixed, and the organic layer was removed and extracted with 1 N HCl (5 mL). The organic layer was removed, and the combined aqueous layer was filtered through a 0.45 micron filter and washed with additional TBME (5 mL). The pH was adjusted with 6 N NaOH until pH˜11. The hazy mixture was then extracted with DCM (3×5 mL), and the combined organic layer was passed over a phase separator and concentrated in vacuo. The crude resulting residue was purified by reversed-phase HPLC. Method: Waters XBridge Prep C8 Column; 30×150 mm, 5 micron. Gradient: Acetonitrile in Water with 10 mM Ammonium Hydroxide. to yield (2′S,6′S)-5-chloro-2′-methyl-6′-(1-methyltriazol-4-yl)spiro[1H-isobenzofuran-3,4′-piperidine]-1-carboxamide (24 mg, 15% over 5 steps) (41) LCMS m/z 362.32 [M+1]⁺; ¹H NMR (300 MHz, Chloroform-d) δ 7.61-7.49 (m, 2H), 7.30 (dd, J=8.1, 1.9 Hz, 1H), 7.10 (d, J=1.8 Hz, 1H), 6.71 (s, 1H), 5.50 (s, 1H), 5.39 (s, 1H), 4.60-4.45 (m, 1H), 4.08 (s, 3H), 3.49-3.30 (m, 1H), 2.16-2.07 (m, 2H), 1.83 (d, J=13.5 Hz, 1H), 1.50 (d, J=12.9 Hz, 1H), 1.18 (d, J=6.3 Hz, 3H). and (2′S,6′S)-5-chloro-2′-methyl-6′-(1-methyltriazol-4-yl)spiro[1H-isobenzofuran-3,4′-piperidine]-1-carboxamide (20.6 mg, 13% over 5 steps) (42) LCMS m/z 362.32 [M+1]⁺; ¹H NMR (300 MHz, Chloroform-d) δ 7.57 (d, J=8.2 Hz, 1H), 7.48 (s, 1H), 7.30 (dd, J=8.2, 1.9 Hz, 1H), 7.11 (d, J=1.8 Hz, 1H), 6.67 (s, 1H), 5.48 (s, 1H), 5.44 (s, 1H), 4.51 (d, J=11.4 Hz, 1H), 4.06 (s, 3H), 3.53-3.34 (m, 1H), 2.13-2.01 (m, 1H), 1.94 (t, J=14.0 Hz, 1H), 1.88-1.75 (m, 2H), 1.23 (d, J=6.5 Hz, 3H).

### Example 2. Assays for Detecting and Measuring APOL1 Inhibitor Properties of Compounds MultiTox-Fluor Multiplex Cytotoxicity Assay

The MultiTox-Fluor Multiplex Cytotoxicity Assay is a single-reagent-addition, homogeneous, fluorescence assay that measures the number of live and dead cells simultaneously in culture wells. The assay measures cell viability and cytotoxicity by detecting two distinct protease activities. The live-cell protease activity is restricted to intact viable cells and is measured using a fluorogenic, cell-permeant peptide glycyl-phenylalanylamino fluorocoumarin (GF-AFC) substrate. The substrate enters intact cells, where it is cleaved to generate a fluorescent signal proportional to the number of living cells. This live-cell protease activity marker becomes inactive upon loss of membrane integrity and leakage into the surrounding culture medium. A second, cell-impermeant, fluorogenic peptide substrate (bis-AAF-R110 Substrate) is used to measure dead-cell protease that has been released from cells that have lost membrane integrity. A ratio of dead to live cells is used to normalize data.

Briefly, the tet-inducible transgenic APOL1 T-REx-HEK293 cell lines were incubated with 50 ng/mL tet to induce APOL1 in the presence of 3-(2-(4-fluorophenyl)-1H-indol-3-yl)-N-((3S,4R)-4-hydroxy-2-oxopyrrolidin-3-yl)propenamide at 10.03, 3.24, 1.13, 0.356, 0.129, 0.042, 0.129, 0.0045, 0.0015, 0.0005 μM in duplicate for 24 hours in a humidified 37° C. incubator. The MultiTox reagent was added to each well and placed back in the incubator for an additional 30 minutes. The plate was read on the EnVision plate reader. A ratio of dead to live cells was used to normalize, and data was imported, analyzed, and fit using Genedata Screener (Basel, Switzerland) software. Data was normalized using percent of control, no tet (100% viability), and 50 ng/mL tet treated (0% viability),and fit using Smart Fit. The reagents, methods, and complete protocol for the MultiTox assay are described below.

TABLE 3
Reagents Used in the Multi-Tox Assay

| Reagent | Catalog Number | Vendor |
|---|---|---|
| 384 well, transparent, | 356663 | Corning (Corning, |
| flat bottom tissue | | NY) |
| culture treated, Poly-D | | |
| lysine coated | | |
| 384 well round bottom | 3656 | CoStar (Corning, |
| polypropylene plates | | NY) |
| Universal plate lids | 250002 | Thermo Fisher |
| | | (Waltham) |
| Axygen 30 μL tips for | VT-384-31UL-R-S | Corning (Corning, |
| Bravo 384 well | | NY) |
| MultiTox-Fluor Multiplex | G9202 | Promega (Madison, |
| Cytotoxicity Assay | | WI) |
| 225 cm² flask, angled neck, | 431082 | Corning (Corning, |
| treated, vented cap | | NY) |
| Dulbecco's Phosphate- | 14190-136 | Thermo Fisher |
| Buffered Saline (DPBS), | | (Waltham) |
| calcium and magnesium- | | |
| free | | |
| Dulbecco's Modified Eagle | 11960-077 | Thermo Fisher |
| Medium (DMEM), high | | (Waltham) |
| glucose, no glutamine, no | | |
| sodium pyruvate | | |
| Fetal Bovine Serum (FBS), | 631368 | Takara (Kusatsu, |
| tetracycline-free, US- | | Japan) |
| Sourced | | |
| L-Glutamine, 200 mM | 25030-081 | Thermo Fisher |
| | | (Waltham) |
| Penicillin-Streptomycin, | 15140-122 | Thermo Fisher |
| 10,000 Units/mL | | (Waltham) |
| Blasticidin S HCl, 10 | A11139-03 | Thermo Fisher |
| mg/mL | | (Waltham) |
| Tetracycline | T7660-5G | Sigma (St. Louis, |
| hydrochloride | | MO) |
| Puromycin dihydrochloride, | A11138-03 | Thermo Fisher |
| 10 mg/mL | | (Waltham) |
| Trypsin-EDTA | 25300-054 | Thermo Fisher |
| | | (Waltham) |

TABLE 4
Equipment Used in the Multi-Tox Assay

| Instrument | Model | Supplier | Location |
|---|---|---|---|
| Bravo | 16050-101 | Agilent Technologies | Santa Clara, CA |
| Multidrop Combi | N/A | Thermo Scientific | Waltham, MA |
| EnVision | N/A | PerkinElmer | Waltham, MA |

### Multi-Tox Assay Protocol

Human embryonic kidney (HEK293) cell lines containing a tet-inducible expression system (T-REx™; Invitrogen, Carlsbad, CA) and Adeno-associated virus site 1 pAAVS1-Puro-APOL1 G0 or pAAVS1-Puro-APOL1 G1 or pAAVS1-Puro-APOL1 G2 Clones G0 DC2.13, Gi DC3.25, and G2 DC4.44 were grown in a T-225 flask at ˜90% confluency in cell growth media (DMEM, 10% Tet-free FBS, 2 mM L-glutamine, 100 Units/mL penicillin-streptomycin, 5 μg/mL blasticidin S HCl, 1 μg/mL puromycin dihydrochloride). Cells were washed with DPBS and then trypsinized to dissociate from the flask. Media was used to quench the trypsin, cells were then pelleted at 200 g and resuspended in fresh cell assay media (DMEM, 2% Tet-free FBS, 2 mM L-glutamine, 100 Units/mL penicillin-streptomycin). Cells were counted and diluted to 1.17×10⁶ cells/mL. 20 μL of cells (23,400/well) were dispensed in every well of a 384-well Poly-D-Lysine coated plate using the Multidrop dispenser. The plates were then incubated at room temperature for one hour.

Tetracycline is needed to induce APOL1 expression. 1 mg/mL tet stock in water was diluted to 250 ng/mL (5×) in cell assay media. 60 μL of cell assay media (no tet control) was dispensed in columns 1 and 24, and 60 μL of 5× tet in 384-PP-round bottom plate was dispensed in columns 2 to 23 with the Multidrop dispenser.

Assay ready plates from the Global Compound Archive were ordered using template 384_APOLICell_DR10n2_50 uM_v3. Compounds were dispensed at 200 nL in DMSO. The final top concentration was 10 μM with a 10 point 3-fold dilution in duplicate in the MultiTox assay.

20 μL was transferred from the 5× tet plate to the ARP and mixed, then 5 μL of 5× tet and the compounds were transferred to the cell plate and mixed using the Bravo. The cell plate was placed in the humidified 37° C. 5% CO₂ incubator for 24 hours.

The MultiTox-Fluor Multiplex Cytotoxicity Assay was performed in accordance with the manufacturer's protocol. After cells were incubated with tet and compound for 24 hours, 25 μL of 1x MultiTox reagent was added to each well using the Multidrop dispenser; the plates were placed on a plate shaker (600 rpm) for 2 minutes, then centrifuged briefly and placed back in the 37° C. incubator for 30 minutes. The cell viability (excitation: 400 nm, emission: 486 nm) and cytotoxicity (excitation: 485 nm, emission: 535 nm) were read using the EnVision plate reader. A ratio of dead (cytotoxicity) to live (viability) cells was reported. Data was exported and analyzed in Genedata. Data was normalized using percent of control, no tet (100% viability), and 50 ng/mL tet treated (0% viability), and fit using Smart Fit settings in Genedata.

### Potency Data for Compounds 1 to 42

The compounds of Formula I are useful as inhibitors of APOL1 activity. Table 5 below illustrates the IC₅₀ of Compounds 1 to 42 using procedures described above. The procedures above may also be used to determine the potency of Compounds I1 to I36. In Table 5 below, the following meanings apply. For IP₅₀ (i.e., IC₅₀ for cell proliferation), “+++” means <50 nM; “++” means between 50 nM and 500 nM; “+” means >500 nM. N.D.=Not determined.

TABLE 5
Potency Data for Compounds 1 to 42

| Compound No. | IP₅₀ (nM) |
|---|---|
| 1 | + |
| 2 | +++ |
| 3 | + |
| 4 | ++ |
| 5 | +++ |
| 6 | + |
| 7 | ++ |
| 8 | ++ |
| 9 | +++ |
| 10 | +++ |
| 11 | ++ |
| 12 | +++ |
| 13 | +++ |
| 14 | ++ |
| 15 | +++ |
| 16 | +++ |
| 17 | +++ |
| 18 | +++ |
| 19 | + |
| 20 | ++ |
| 21 | ++ |
| 22 | + |
| 23 | +++ |
| 24 | ++ |
| 25 | +++ |
| 26 | +++ |
| 27 | +++ |
| 28 | +++ |
| 29 | ++ |
| 30 | ++ |
| 31 | + |
| 32 | +++ |
| 33 | +++ |
| 34 | +++ |
| 35 | +++ |
| 36 | +++ |
| 37 | +++ |
| 38 | +++ |
| 39 | +++ |
| 40 | ++ |
| 41 | +++ |
| 42 | ++ |

### OTHER EMBODIMENTS

This disclosure provides merely non-limiting example embodiments of the disclosed subject matter. One skilled in the art will readily recognize from the disclosure and claims, that various changes, modifications and variations can be made therein without departing from the spirit and scope of the disclosure as defined in the following claims.

1. A compound represented by the following structural formula: 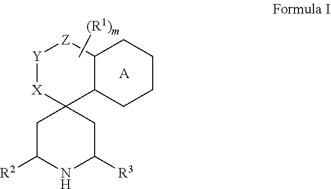
a tautomer thereof, a deuterated derivative of that compound or tautomer, or a pharmaceutically acceptable salt of any of the foregoing, wherein:
Ring A is chosen from 6-membered aryl and 6-membered heteroaryl groups;
X is chosen from —CH₂—, —C(O)—, —S(O)₂—, —NH—, and —O—;
Y is chosen from —CH₂—, —C(O)—, —S(O)₂—, —NH—, and —O—;
Z is chosen from a bond, —CH₂—, —NH—, —C(O)—, —S(O)₂—, and —O—, wherein:
at least one of X and Y is chosen from —CH₂— and —C(O)—; and
for each of X, Y, and Z, a hydrogen atom in each instance of —CH₂— or —NH— is optionally replaced by R¹;
R¹, for each occurrence, is independently chosen from halogen, —OH, cyano, phenyl, C₁-C₆ alkyl, C₁-C₆ alkoxy, C₃-C₆ carbocyclyl, 4- to 6-membered heterocyclyl, —C(═O)OR^{c}, —C(═O)N(R^{c})₂, and —OS(═O)₂R^{c} groups, wherein:
R^{c}, for each occurrence, is independently chosen from hydrogen, C₁-C₄ alkyl, and C₁-C₄ haloalkyl groups;
the 4- to 6-membered heterocyclyl of R¹ comprises one heteroatom chosen from nitrogen and oxygen;
the C₁-C₆ alkyl of R¹ is optionally substituted with 1 to 3 groups independently chosen from halogen, cyano, —OH, —NH₂, —NH(C₁-C₄ alkyl), —N(C₁-C₄ alkyl)₂, and C₁-C₄ alkoxy groups;
the C₁-C₆ alkoxy of R¹ is optionally substituted with 1 to 3 groups independently chosen from —OH, cyano, and halogen groups;
the C₃-C₆ carbocyclyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups; and
the phenyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups;
R² is chosen from cyano, C₁-C₆ alkyl, —C(═O)O(C₁-C₄ alkyl), C₂-C₆ alkynyl, and 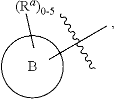
wherein:
the C₁-C₆ alkyl of R² is optionally substituted with 1 to 3 groups independently chosen from halogen, cyano, —OH, —NH₂, —NH(C₁-C₄ alkyl), —N(C₁-C₄ alkyl)₂, C₁-C₄ alkoxy, —C(═O)NH₂, —C(═O)NH(C₁-C₄ alkyl), —C(═O)N(C₁-C₄ alkyl)₂, C3-C₆ carbocyclyl, 5- to 10-membered heterocyclyl, C₆ aryl, and 5- to 10-membered heteroaryl groups;
Ring B is chosen from C₃-C₁₂ carbocyclyl, 3- to 12-membered heterocyclyl, C₆ and C₁₀ aryl, and 5- to 10-membered heteroaryl groups, wherein Ring B is optionally substituted with 1, 2, 3, 4, or 5 R^{a} groups, wherein:
R^{a}, for each occurrence, is independently chosen from halogen, cyano, C₁-C₆ alkyl, C₂-C₆ alkenyl, C₁-C₆ alkoxy, C₁-C₆ haloalkyl, C₁-C₆ haloalkenyl, C₁-C₆ haloalkoxy, —C(═O)NR^{h}Rⁱ, —NR^{h}i, —NR^{h}C(O)R^{k}, —NR^{h}C(O)OR^{k}, —NR^{h}C(═O)NRⁱR^{j}, —NR^{h}S(═O)ₚR^{k}, —OR^{k}, —OC(═O)R^{k}, —OC(═O)OR^{k}, —OC(═O)NR^{h}Rⁱ, —[O(CH₂)_{q}]ᵣO(C₁-C₆ alkyl), —S(═O)ₚR^{k}, —S(═O)ₚNR^{h}Rⁱ, —C(═O)OR^{k}, C3-C₁₂ carbocyclyl, 3- to 12-membered heterocyclyl, C₆ and C₁₀ aryl, and 5- to 10-membered heteroaryl groups, wherein:
the C₁-C₆ alkyl, C₁-C₆ alkoxy, and the C₂-C₆ alkenyl of R^{a} are each optionally substituted with 1 to 3 groups independently chosen from C₆ to C₁₀ aryl (optionally substituted with 1 to 3 R^{m} groups), 5- to 10-membered heterocyclyl (optionally substituted with 1 to 3 R^{m} groups), 5-to 10-membered heteroaryl (optionally substituted with 1 to 3 R^{m} groups), cyano, —C(═O)R^{k}, —C(═O)OR^{k}, —C(═O)NR^{h}Rⁱ, —NR^{h}Rⁱ, —NR^{h}C(═O)R^{k}, —NR^{h}C(═O)OR^{k}, —NR^{h}C(═O)NRⁱR^{j}, —NR^{h}S(═O)ₚR^{k}, —OR^{k}, —OC(═O)R^{k}, —OC(═O)OR^{k}, —OC(═O)NR^{h}Rⁱ, —S(═O)ₚR^{k}, —S(═O)ₚNR^{h}Rⁱ, —O(C6 aryl) (optionally substituted with 1 to 3 R^{m} groups), and C₃-C₆ carbocyclyl groups (optionally substituted with 1 to 3 R^{m} groups);
the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from halogen, cyano, C₁-C₄ alkyl, —NR^{h}Rⁱ, and —OR^{k} groups, wherein:
R^{h},Rⁱ, and R^{j}, for each occurrence, are each independently chosen from hydrogen, C₁-C₄ alkyl, C₆-C₁₀ aryl, and C₃-C₆ cycloalkyl groups, wherein:
the C₁-C₄ alkyl of any one of R^{h}, Rⁱ, and R^{j} is optionally substituted with 1 to 3 groups independently chosen from halogen, cyano, and —OH groups;
R^{k}, for each occurrence, is independently chosen from hydrogen, C₁-C₄ alkyl, 5- to 10-membered heterocyclyl, and C₃-C₆ carbocyclyl groups, wherein:
the C₁-C₄ alkyl of any one of R^{k} is optionally substituted with 1 to 3 groups independently chosen from halogen, cyano, and —OH groups;
R^{m}, for each occurrence, is independently chosen from halogen, cyano, oxo, C₁-C₆ alkyl, C₁-C₆ alkoxy, —S(═O)ₚR^{k}, and —OR^{k} groups, wherein:
the C₁-C₆ alkyl of R^{m} is optionally substituted with 1 to 3 groups independently chosen from halogen, cyano, —OH, and —O(C₁-C₄ alkyl) groups;
R³ is chosen from C₁-C₆ alkyl, —C(═O)O(C₁-C₄ alkyl), C₃-C₁₂ carbocyclyl, 3- to 12-membered heterocyclyl, C₆ and C₁₀ aryl, and 5- to 10-membered heteroaryl groups, wherein:
the C₁-C₆ alkyl of R³ is optionally substituted with 1 to 3 groups independently chosen from halogen, cyano, —OH, —NH₂, —NH(C₁-C₄ alkyl), —N(C₁-C₄ alkyl)₂, C₁-C₄ alkoxy, —C(═O)NH₂, —C(═O)NH(C₁-C₄ alkyl), and —C(═O)N(C₁-C₄ alkyl)₂ groups;
the C₃-C₁₂ carbocyclyl, the 3- to 12-membered heterocyclyl, the C₆ and C₁₀ aryl, and the 5- to 10-membered heteroaryl of R³ are each optionally substituted with 1 to 3 groups independently chosen from halogen, cyano, —OH, —NH₂, —NH(C₁-C₄ alkyl) (optionally substituted with —OH), —N(C₁-C₄ alkyl)₂, C₁-C₅ alkyl (optionally substituted with —OH or —S(═O)₂(C₁-C₄ alkyl)), C₁-C₄ alkoxy, —C(═O)NH₂, —C(═O)NH(C₁-C₄ alkyl), —NHC(═O)(C₁-C₄ alkyl), —C(═O)(C₁-C₄ alkoxy), and —C(═O)N(C₁-C₄ alkyl)₂ groups;
m is an integer chosen from 0, 1, 2, 3, 4, and 5;
p, for each occurrence, is an integer independently chosen from 1 and 2; and
q and r, for each occurrence, are each an integer independently chosen from 1, 2, 3, and 4.

2. The compound, tautomer, deuterated derivative, or pharmaceutically acceptable salt according to claim 1, wherein:
Ring A is chosen from 6-membered aryl and 6-membered heteroaryl groups;
X is chosen from —CH₂—, —C(O)—, —S(O)₂—, —NH—, and —O—;
Y is chosen from —CH₂—, —C(O)—, —S(O)₂—, —NH—, and —O—;
Z is chosen from a bond, —CH₂—, —NH—, —C(O)—, —S(O)₂—, and —O—, wherein:
at least one of X and Y is chosen from —CH₂— and —C(O)—; and
for each of X, Y, and Z, a hydrogen atom in each instance of —CH₂— or —NH— is optionally replaced by R¹;
R¹, for each occurrence, is independently chosen from halogen, —OH, cyano, phenyl, C₁-C₆ alkyl, C₁-C₆ alkoxy, —C(═O)OR^{c}, —C(═O)N(R^{c})₂, and —OS(═O)₂R^{c} groups, wherein:
R^{c}, for each occurrence, is independently chosen from hydrogen, C₁-C₄ alkyl, and C₁-C₄ haloalkyl groups;
the C₁-C₆ alkyl of R¹ is optionally substituted with 1 to 3 groups independently chosen from halogen, cyano, —OH, —NH₂, —NH(C₁-C₄ alkyl), —N(C₁-C₄ alkyl)₂, and C₁-C₄ alkoxy groups;
the C₁-C₆ alkoxy of R¹ is optionally substituted with 1 to 3 groups independently chosen from —OH, cyano, and halogen groups;
the phenyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups;
R² is chosen from C₁-C₆ alkyl and 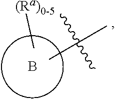
wherein:
the C₁-C₆ alkyl of R² is optionally substituted with 1 to 3 groups independently chosen from halogen, cyano, —OH, —NH₂, —NH(C₁-C₄ alkyl), —N(C₁-C₄ alkyl)₂, C₁-C₄ alkoxy, —C(═O)NH₂, —C(═O)NH(C₁-C₄ alkyl), —C(═O)N(C₁-C₄ alkyl)₂, C3-C₆ carbocyclyl, 5- to 10-membered heterocyclyl, C₆ aryl, and 5- to 10-membered heteroaryl groups;
Ring B is chosen from 3- to 12-membered heterocyclyl, C₆ aryl, and 5- to 10-membered heteroaryl groups, wherein Ring B is optionally substituted with 1, 2, 3, 4, or 5 R^{a} groups, wherein:
R^{a}, for each occurrence, is independently chosen from halogen, cyano, C₁-C₆ alkyl, C₂-C₆ alkenyl, C₁-C₆ alkoxy, C₁-C₆ haloalkyl, C₁-C₆ haloalkenyl, C₁-C₆ haloalkoxy, —C(═O)NR^{h}Rⁱ, —NR^{h}Rⁱ, —NR^{h}C(═O)R^{k}, —NR^{h}C(═O)OR^{k}, —NR^{h}C(═O)NRⁱR^{j}, —NR^{h}S(═O)ₚR^{k}, —OR^{k}, —OC(═O)R^{k}, —OC(═O)OR^{k}, —OC(═O)NR^{h}Rⁱ, —[O(CH₂)_{q}]ᵣO(C₁-C₆ alkyl), —S(═O)ₚR^{k}, —S(═O)ₚNR^{h}Rⁱ, —C(═O)OR^{k}, C₃-C₁₂ carbocyclyl, 3- to 12-membered heterocyclyl, C₆ and C₁₀ aryl, and 5- to 10-membered heteroaryl groups, wherein:
the C₁-C₆ alkyl, C₁-C₆ alkoxy, and the C₂-C₆ alkenyl of R^{a} are each optionally substituted with 1 to 3 groups independently chosen from C₆ to C₁₀ aryl (optionally substituted with 1 to 3 R^{m} groups), 5- to 10-membered heterocyclyl (optionally substituted with 1 to 3 R^{m} groups), 5-to 10-membered heteroaryl (optionally substituted with 1 to 3 R^{m}groups), cyano, —C(═O)R^{k}, —C(═O)OR^{k}, —C(═O)NR^{h}Rⁱ, —NR^{h}i, —NR^{h}C(═O)R^{k},
—NR^{h}C(═O)OR^{k}, —NR^{h}C(═O)NRⁱR^{j}, —NR^{h}S(═O)ₚR^{k}, —OR^{k}, —OC(═O)R^{k}, —OC(═O)OR^{k}, —OC(═O)NR^{h}Rⁱ, —S(═O)ₚR^{k}, —S(═O)ₚNR^{h}Rⁱ, and C₃-C₆ carbocyclyl groups (optionally substituted with 1 to 3 R^{m} groups);
the C₃-C₁₂ carbocyclyl, the 3- to 12-membered heterocyclyl, the C₆ and C₁₀ aryl, and the 5- to 10-membered heteroaryl of R^{a} are each optionally substituted with 1 to 3 groups independently chosen from halogen, cyano, C₁-C₄ alkyl, —NR^{h}Rⁱ, and —OR^{k} groups, wherein:
R^{h}, Rⁱ, and R^{j}, for each occurrence, are each independently chosen from hydrogen, C₁-C₄ alkyl, C₆-C₁₀ aryl, and C₃-C₆ cycloalkyl groups, wherein:
the C₁-C₄ alkyl of any one of R^{h}, Rⁱ, and R^{j} is optionally substituted with 1 to 3 groups independently chosen from halogen, cyano, and —OH groups;
R^{k}, for each occurrence, is independently chosen from hydrogen, C₁-C₄ alkyl, 5- to 10-membered heterocyclyl, and C₃-C₆ carbocyclyl groups, wherein:
the C₁-C₄ alkyl of any one of R^{k} is optionally substituted with 1 to 3 groups independently chosen from halogen, cyano, and —OH groups;
R^{m}, for each occurrence, is independently chosen from halogen, cyano, oxo, C₁-C₆ alkyl, C₁-C₆ alkoxy, —S(═O)ₚR^{k}, and —OR^{k} groups, wherein:
the C₁-C₆ alkyl of R^{m} is optionally substituted with 1 to 3 groups independently chosen from halogen, cyano, and —OH groups;
R³ is chosen from C₁-C₆ alkyl groups, wherein:
the C₁-C₆ alkyl of R³ is optionally substituted with 1 to 3 groups independently chosen from halogen, cyano, —OH, —NH₂, —NH(C₁-C₄ alkyl), —N(C₁-C₄ alkyl)₂, C₁-C₄ alkoxy, —C(═O)NH₂, —C(═O)NH(C₁-C₄ alkyl), and —C(═O)N(C₁-C₄ alkyl)₂ groups;
m is an integer chosen from 0, 1, 2, and 3;
p, for each occurrence, is an integer independently chosen from 1 and 2; and
q and r, for each occurrence, are each an integer independently chosen from 1, 2, 3, and 4.

3. The compound, tautomer, deuterated derivative, or pharmaceutically acceptable salt according to claim 1 or 2, wherein:
Ring A is chosen from 6-membered aryl and 6-membered heteroaryl groups;
X is chosen from —CH₂—, —C(O)—, —S(O)₂—, —NH—, and —O—;
Y is chosen from —CH₂—, —C(O)—, —S(O)₂—, —NH—, and —O—;
Z is chosen from a bond, —CH₂—, —NH—, —C(O)—, —S(O)₂—, and —O—, wherein:
at least one of X and Y is chosen from —CH₂— and —C(O)—; and
for each of X, Y, and Z, a hydrogen atom in each instance of —CH₂— or —NH— is optionally replaced by R¹;
R¹, for each occurrence, is independently chosen from halogen, —OH, cyano, C₁-C₄ alkyl, C₁-C₄ alkoxy, —C(═O)OR^{c}, —C(═O)N(R^{c})₂, and —OS(═O)₂R^{c} groups, wherein:
R^{c}, for each occurrence, is independently chosen from hydrogen, C₁-C₄ alkyl, and C₁-C₄ haloalkyl groups;
the C₁-C₆ alkyl of R¹ is optionally substituted with 1 to 3 groups independently chosen from halogen and —OH groups;
R² is 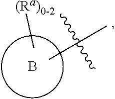
wherein:
Ring B is chosen from 5-membered heterocyclyl and 5-membered heteroaryl groups, wherein Ring B is optionally substituted with 1 or 2 R^{a} groups, wherein:
R^{a}, for each occurrence, is independently chosen from C₁-C₆ alkyl groups optionally substituted with 1 group independently chosen from —S(═O)ₚR^{k} groups, wherein:
R^{k}, for each occurrence, is independently chosen from C₁-C₄ alkyl groups;
R³ is chosen from C₁-C₃ alkyl groups;
m is an integer chosen from 0, 1, 2, and 3; and
p, for each occurrence, is an integer independently chosen from 1 and 2.

4. The compound, tautomer, deuterated derivative, or pharmaceutically acceptable salt according to any one of claims 1-3, wherein:
Ring A is chosen from 6-membered aryl and 6-membered heteroaryl groups;
X is chosen from —CH₂—, —C(O)—, —S(O)₂—, —NH—, and —O—;
Y is chosen from —CH₂—, —C(O)—, —S(O)₂—, —NH—, and —O—;
Z is chosen from a bond, —CH₂—, —NH—, —C(O)—, —S(O)₂—, and —O—, wherein:
at least one of X and Y is chosen from —CH₂— and —C(O)—; and
for each of X, Y, and Z, a hydrogen atom in each instance of —CH₂— or —NH— is optionally replaced by R¹;
R¹, for each occurrence, is independently chosen from halogen, —OH, cyano, C₁-C₄ alkyl, C₁-C₄ alkoxy, —C(═O)OR^{c}, —C(═O)N(R^{c})₂, and —OS(═O)₂R^{c} groups, wherein:
R^{c}, for each occurrence, is independently chosen from hydrogen, C₁-C₄ alkyl, and C₁-C₄ haloalkyl groups;
the C₁-C₆ alkyl of R¹ is optionally substituted with 1 to 3 groups independently chosen from halogen and —OH groups;
R² is 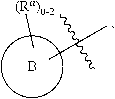
wherein:
Ring B is chosen from pyrazole and triazole groups, wherein Ring B is optionally substituted with 1 or 2 R^{a} groups, wherein:
R^{a}, for each occurrence, is independently chosen from C₁-C₆ alkyl groups optionally substituted with 1 group independently chosen from —S(═O)ₚR^{k} groups, wherein:
R^{k}, for each occurrence, is independently chosen from C₁-C₄ alkyl groups;
R³ is methyl;
m is an integer chosen from 0, 1, 2, and 3; and
p, for each occurrence, is an integer independently chosen from 1 and 2.

5. The compound, tautomer, deuterated derivative, or pharmaceutically acceptable salt according to any one of claims 1-4, wherein Ring A is chosen from phenyl, pyrimidinyl, and pyridinyl, and all other variables not specifically defined herein are as defined in any one of claims 1-4.

6. The compound, tautomer, deuterated derivative, or pharmaceutically acceptable salt according to any one of claims 1-4, wherein Ring A is phenyl, and all other variables not specifically defined herein are as defined in any one of claims 1-4.

7. The compound, tautomer, deuterated derivative, or pharmaceutically acceptable salt according to claim 1, 5, or 6, wherein R¹, for each occurrence, is independently chosen from hydrogen, halogen, cyano, —OH, C₁-C₄ alkyl, C₁-C₄ alkoxy, —C(═O)N(R^{c})₂, and C₃-C₆ cycloalkyl groups, wherein:
R^{c}, for each occurrence, is independently chosen from hydrogen and C₁-C₂ alkyl groups;
the C₁-C₄ alkyl of R¹ is optionally substituted with 1 to 3 groups independently chosen from halogen, cyano, —OH, and C₁-C₂ alkoxy groups;
the C₁-C₄ alkoxy of R¹ is optionally substituted with 1 to 3 independently chosen from halogen groups; and
the C₃-C₆ cycloalkyl of R¹ is optionally substituted with 1 to 3 groups independently chosen from halogen, cyano, —OH, and C₁-C₂ alkoxy groups;
and all other variables not specifically defined herein are as defined in claim 1, 5, or 6.

8. The compound, tautomer, deuterated derivative, or pharmaceutically acceptable salt according to claim 1, 5, or 6, wherein R¹, for each occurrence, is independently chosen from F, Cl, Br, C₁-C₄ alkyl, C₁-C₄ alkoxy, —C(═O)N(R^{c})₂, and C₃-C₆ cycloalkyl groups, wherein:
R^{c}, for each occurrence, is independently chosen from hydrogen and C₁-C₂ alkyl groups;
the C₁-C₄ alkyl of R¹ is optionally substituted with 1 to 3 groups independently chosen from halogen and —OH;
the C₁-C₄ alkoxy of R¹ is optionally substituted with 1 to 3 independently chosen from halogen groups; and
the C₃-C₆ cycloalkyl of R¹ is optionally substituted with 1 to 3 groups independently chosen from halogen and —OH;
and all other variables not specifically defined herein are as defined in claim 1, 5, or 6.

9. The compound, tautomer, deuterated derivative, or pharmaceutically acceptable salt according to claim 1, 5, or 6, wherein R¹, for each occurrence, is independently chosen from F, Cl, Br, C₁-C₄ alkyl, C₁-C₄ alkoxy, —C(═O)N(R^{c})₂, and C₃-C₆ cycloalkyl groups, wherein:
R^{c}, for each occurrence, is independently chosen from hydrogen and C₁-C₂ alkyl groups;
the C₁-C₄ alkyl of R¹ is optionally substituted with 1 to 3 groups independently chosen from halogen and —OH; and
the C₁-C₄ alkoxy of R¹ is optionally substituted with 1 to 3 independently chosen from halogen groups;
and all other variables not specifically defined herein are as defined in claim 1, 5, or 6.

10. The compound, tautomer, deuterated derivative, or pharmaceutically acceptable salt according to claim 1, 5, or 6, wherein R¹, for each occurrence, is independently chosen from F, Cl, Br, —CH₃, —CH(CH₃)₂, —CF₃, —OCH₃, —OCF₃, —C(═O)N(CH₃)₂, and cyclopropyl;
and all other variables not specifically defined herein are as defined in claim 1, 5, or 6.

11. The compound, tautomer, deuterated derivative, or pharmaceutically acceptable salt according to any one of claims 1-10, wherein m is 1; and all other variables not specifically defined herein are as defined in any one of claims 1-10.

12. The compound, tautomer, deuterated derivative, or pharmaceutically acceptable salt according to any one of claims 1-10, wherein m is 2; and all other variables not specifically defined herein are as defined in any one of claims 1-10.

13. The compound, tautomer, deuterated derivative, or pharmaceutically acceptable salt according to any one of claims 1 and 5-12, wherein Ring B is chosen from cyclopropyl, 5- to 10-membered heterocyclyl, phenyl, and 5- to 9-membered heteroaryl groups; each of which is optionally substituted with 1, 2, 3, 4, or 5 R^{a} groups; and all other variables not specifically defined herein are as defined in any one of claims 1 and 5-12.

14. The compound, tautomer, deuterated derivative, or pharmaceutically acceptable salt according to any one of claims 1 and 5-12, wherein Ring B is chosen from cyclopropyl, 5- to 10-membered heterocyclyl comprising 1 to 3 heteroatoms chosen from N and O, phenyl, and 5-to 9-membered heteroaryl comprising 1 to 3 heteroatoms chosen from N and O; each of which is optionally substituted with 1, 2, 3, 4, or 5 R^{a} groups; and all other variables not specifically defined herein are as defined in any one claims of 1 and 5-12.

15. The compound, tautomer, deuterated derivative, or pharmaceutically acceptable salt according to any one of claims 1 and 5-12, wherein Ring B is chosen from cyclopropyl, 5-membered heterocyclyl comprising 1 to 3 heteroatoms chosen from N and O, 6-membered heterocyclyl comprising 1 to 3 heteroatoms chosen from N and O, 9-membered heterocyclyl comprising 1 to 3 heteroatoms chosen from N and O, 10-membered heterocyclyl comprising 1 to 3 heteroatoms chosen from N and O, phenyl, 5-membered heteroaryl comprising 1 to 3 heteroatoms chosen from N and O, 6-membered heteroaryl comprising 1 to 3 heteroatoms chosen from N and O, and 9-membered heteroaryl comprising 1 to 3 heteroatoms chosen from N and O; each of which is optionally substituted with 1, 2, 3, 4, or 5 R^{a} groups; and all other variables not specifically defined herein are as defined in any one of claims 1 and 5-12.

16. The compound, tautomer, deuterated derivative, or pharmaceutically acceptable salt according to any one of claims 1 and 5-12, wherein Ring B is chosen from 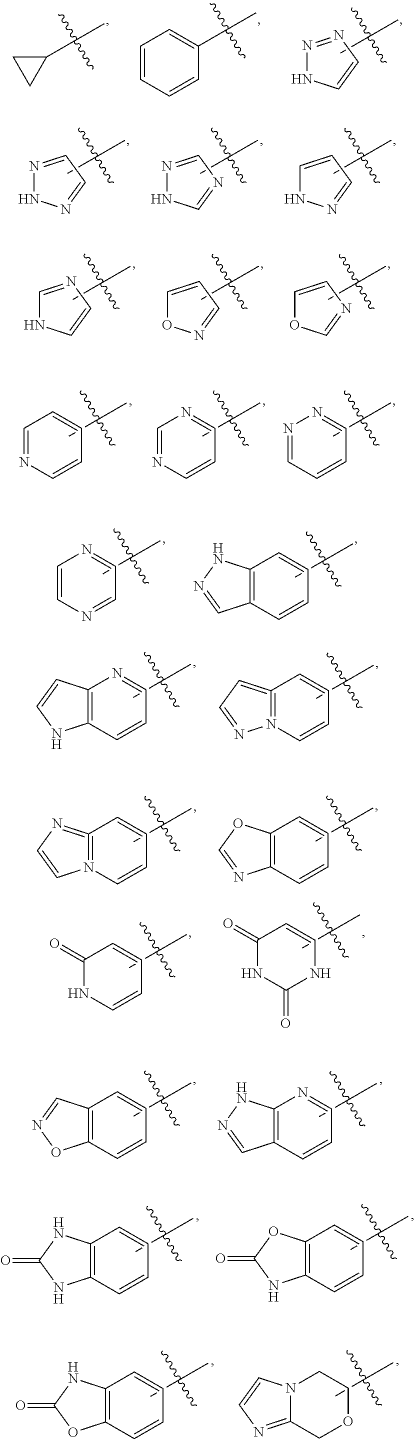 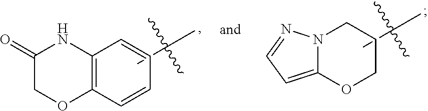
each of which is optionally substituted with 1, 2, 3, 4, or 5 R^{a} groups; and all other variables not specifically defined herein are as defined in any one of claims 1 and 5-12.

17. The compound, tautomer, deuterated derivative, or pharmaceutically acceptable salt according to any one of claims 1 and 5-12, wherein Ring B is chosen from 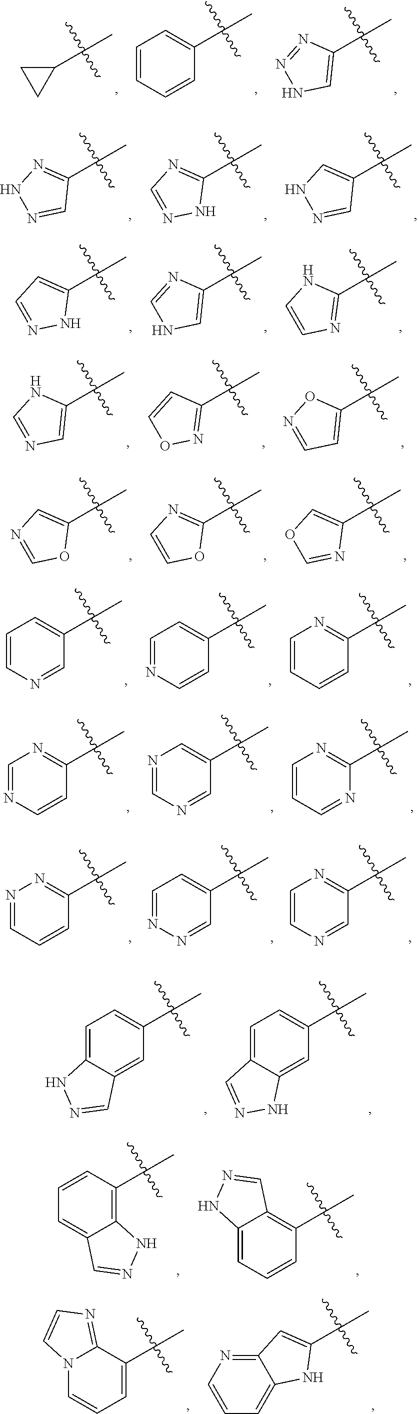 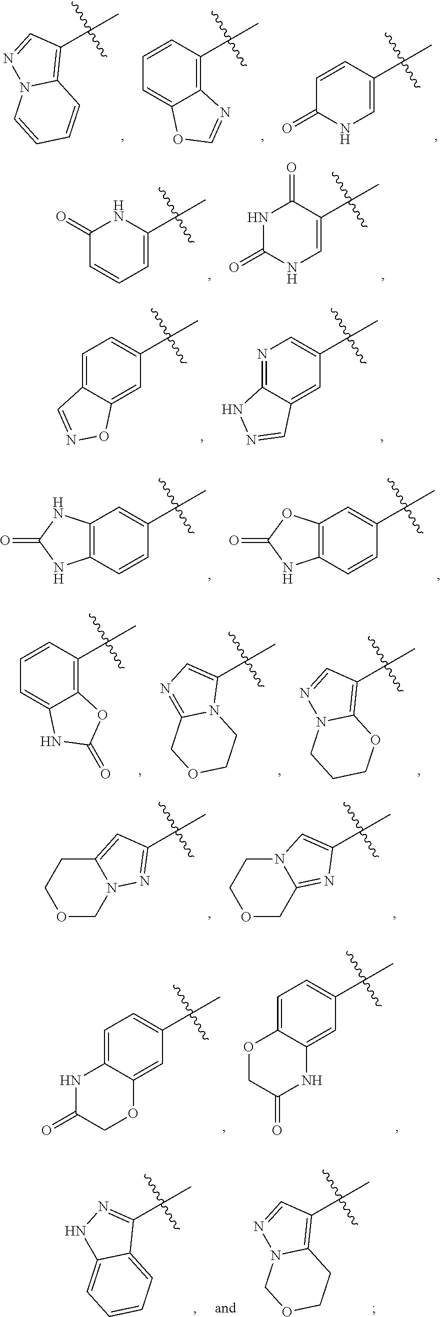
each of which is optionally substituted with 1, 2, 3, 4, or 5 R^{a} groups; and all other variables not specifically defined herein are as defined in any one of claims 1 and 5-12.

18. The compound, tautomer, deuterated derivative, or pharmaceutically acceptable salt according to any one of claims 1 and 5-12, wherein Ring B is 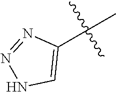 [SMILES: *c1c[nH]nn1]
which is optionally substituted with 1 R^{a} group; and all other variables not specifically defined herein are as defined in any one of claims 1 and 5-12.

19. The compound, tautomer, deuterated derivative, or pharmaceutically acceptable salt according to any one of claims 13-18, wherein R^{a}, for each occurrence, is independently chosen from halogen, cyano, C₁-C₆ alkyl, C₁-C₄ alkoxy, C₁-C₆ haloalkyl, C₁-C₆ haloalkoxy, —C(═O)NR^{h}Rⁱ, —NR^{h}Rⁱ, —NR^{h}C(O)R^{k}, —OR^{k}, —[O(CH₂)_{q}]ᵣO(C₁-C₆ alkyl), —S(═O)₂R^{k}, —S(═O)₂NR^{h}Rⁱ, C3-C₆ cycloalkyl, 5 to 10-membered heterocyclyl, phenyl, and 5- to 8-membered heteroaryl groups, wherein:
the C₁-C₆ alkyl of R^{a} is optionally substituted with 1 to 3 groups independently chosen from cyano, —C(═O)NR^{h}Rⁱ, —NR^{h}Rⁱ, —NR^{h}C(═O)R^{k}, —NR^{h}C(═O)OR^{k}, —NR^{h}C(═O)NRⁱR^{j}, —NR^{h}S(═O)ₚR^{k}, —OR^{k}, —S(═O)₂R^{k}, —S(═O)ₚNR^{h}Rⁱ, and C₃-C₆ cycloalkyl groups;
the C₃-C₆ cycloalkyl, the 5- to 10-membered heterocyclyl, the phenyl, and the 5- to 8-membered heteroaryl of R^{a} are each optionally substituted with 1 to 3 groups independently chosen from halogen, C₁-C₂ alkyl, and —OR^{k} groups, wherein:
R^{h}, Rⁱ, and R^{j}, for each occurrence, are each independently chosen from hydrogen, C₁-C₂ alkyl, cyclopropyl, and cyclobutyl groups, wherein:
the C₁-C₂ alkyl of any one of R^{h}, Rⁱ, and R^{j} is optionally substituted with 1 to 3 groups independently chosen from halogen and —OH;
R^{k}, for each occurrence, is each independently chosen from hydrogen and C₁-C₄ alkyl groups, wherein:
the C₁-C₄ alkyl of R^{k} is optionally substituted with 1 to 3 groups independently chosen from halogen and —OH; and
q and r are each an integer chosen from 1, 2, and 3;
and all other variables not specifically defined herein are as defined in any one of claims 13-18.

20. The compound, tautomer, deuterated derivative, or pharmaceutically acceptable salt according to any one of claims 13-18, wherein R^{a}, for each occurrence, is independently chosen from halogen, cyano, C₁-C₆ alkyl, C₁-C₄ alkoxy, C₁-C₄ haloalkyl, C₁-C₄ haloalkoxy, —C(═O)NR^{h}Rⁱ, —NR^{h}Rⁱ, —NR^{h}C(═O)R^{k}, OR^{k}, —[O(CH₂)_{q}]ᵣO(C₁-C₄ alkyl), —S(═O)₂R^{k}, —S(═O)₂NR^{h}Rⁱ, cyclopropyl, cyclobutyl, 5- to 6-membered heterocyclyl, phenyl, and 5- to 6-membered heteroaryl, wherein:
the C₁-C₆ alkyl of R^{a} is optionally substituted with 1 to 3 groups independently chosen from cyano, —C(═O)NR^{h}Rⁱ, —S(═O)₂R^{k}, —NR^{h}Rⁱ, —OR^{k}, cyclopropyl, and cyclobutyl groups, wherein:
the cyclopropyl, the cyclobutyl, the 5- to 6-membered heterocyclyl, the phenyl, and the 5 to 6-membered heteroaryl of R^{a} are each optionally substituted with 1 to 3 groups independently chosen from halogen, —CH₃, —OH, and —OCH₃; wherein:
R^{h} and Rⁱ, for each occurrence, are each independently chosen from hydrogen, —CH₃, cyclopropyl, and cyclobutyl groups, wherein:
the —CH₃ of any one of R^{h} and Rⁱ is optionally substituted with 1 to 3 groups independently chosen from F, Cl, and —OH;
R^{k}, for each occurrence, is each independently chosen from hydrogen and —CH₃, wherein:
the —CH₃ of R^{k} is optionally substituted with 1 to 3 groups independently chosen from halogen and —OH;
and all other variables not specifically defined herein are as defined in any one of claims 13-18.

21. The compound, tautomer, deuterated derivative, or pharmaceutically acceptable salt according to any one of claims 13-18, wherein R^{a}, for each occurrence, is independently chosen from F, Cl, Br, cyano, C₁-C₆ alkyl, C₁-C₂ alkoxy, C₁-C₂ haloalkyl, —C(═O)NR^{h}Rⁱ, —NR^{h}Rⁱ, —NR^{h}C(═O)R^{k}, OR^{k}, —[O(CH₂)_{q}]ᵣO(C₁-C₂ alkyl), —S(═O)₂R^{k}, —S(═O)₂NR^{h}Rⁱ, cyclopropyl, cyclobutyl, 5-membered heterocyclyl, phenyl, and 6-membered heteroaryl groups, wherein:
the C₁-C₆ alkyl of R^{a} is optionally substituted with 1 to 3 groups independently chosen from cyano, —C(═O)NR^{h}Rⁱ, —OR^{k}, —S(═O)₂R^{k}, and cyclopropyl;
the cyclopropyl, the cyclobutyl, the 5- to 6-membered heterocyclyl, the phenyl, and the 5- to 6-membered heteroaryl of R^{a} are each optionally substituted with 1 to 3 groups independently chosen from halogen, —CH₃, —OH, and —OCH₃, wherein:
R^{h} and Rⁱ, for each occurrence, are each independently chosen from hydrogen, —CH₃, and cyclopropyl; wherein:
the —CH₃ of any one of R^{h} and Riis optionally substituted with 1 to 3 groups independently chosen from F, Cl, and —OH;
R^{k}, for each occurrence, is each independently chosen from hydrogen and —CH₃; and
q and r are each an integer independently chosen from 1 and 2;
and all other variables not specifically defined herein are as defined in any one of claims 13-18.

22. The compound, tautomer, deuterated derivative, or pharmaceutically acceptable salt according to any one of claims 13-18, wherein R^{a}, for each occurrence, is independently chosen from F, cyano, —OH, —CH₃, —CF₃, —CH(CH₃)₂, —(CH₂)₂OH, —(CH₂)₂OCH₃, —CH₂CH(OH)C₂H₅,
—CH₂C(CH₃)(CH₂OH)₂, —OCH₃, —OCH₂CH₃, —O(CH₂)₂]₂OCH₃, —CH₂C(═O)NHCH₃, —(CH₂)₂SO₂CH₃, —CH₂C(═O)N(CH₃)₂, —CH₂(cyclopropyl), —C(═O)NH₂, —C(═O)NH(cyclopropyl), —NH₂, —NHCH₃, —N(CH₃)₂, —NHC(CH₃)₂CH₂OH, —NHC(═O)CH₃, —SO₂CH₃, —SO₂NH₂, cyclopropyl, 2-methoxyphenyl, N-methylpiperazinyl, tetrahydro-2H-pyranyl, methylpyrazolyl, pyridinyl, and tetrahydrothiophenyl 1,1-dioxide; and all other variables not specifically defined herein are as defined in any one of claims 13-18.

23. The compound, tautomer, deuterated derivative, or pharmaceutically acceptable salt according to any one of claims 13-18, wherein R^{a}, for each occurrence, is independently chosen from —CH₃ and —(CH₂)₂SO₂CH₃; and all other variables not specifically defined herein are as defined in any one of claims 13-18.

24. The compound, tautomer, deuterated derivative, or pharmaceutically acceptable salt according to claim 1, wherein the compound is represented by the following structural formula: 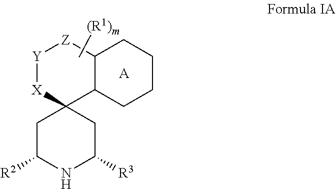
a tautomer thereof, a deuterated derivative of that compound or tautomer, or a pharmaceutically acceptable salt of any of the foregoing, wherein all variables not specifically defined herein are as defined in any one of claims 1-23.

25. The compound, tautomer, deuterated derivative, or pharmaceutically acceptable salt according to claim 1 or 24, wherein Z is chosen from —CH₂—, —NH—, —C(O)—, —S(O)₂—, and —O—; and all other variables not specifically defined herein are as defined in claim 1 or 24.

26. The compound, tautomer, deuterated derivative, or pharmaceutically acceptable salt according to claim 1, wherein the compound is represented by one of the following structural formulae: 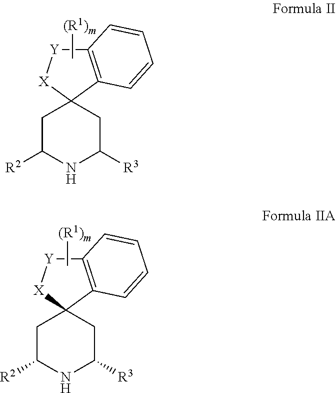
a tautomer thereof, a deuterated derivative of that compound or tautomer, or a pharmaceutically acceptable salt of any of the foregoing, wherein all variables not specifically defined herein are as defined in any one of claims 1-4.

27. The compound, tautomer, deuterated derivative, or pharmaceutically acceptable salt according to claim 1, wherein the compound is represented by one of the following structural formulae: 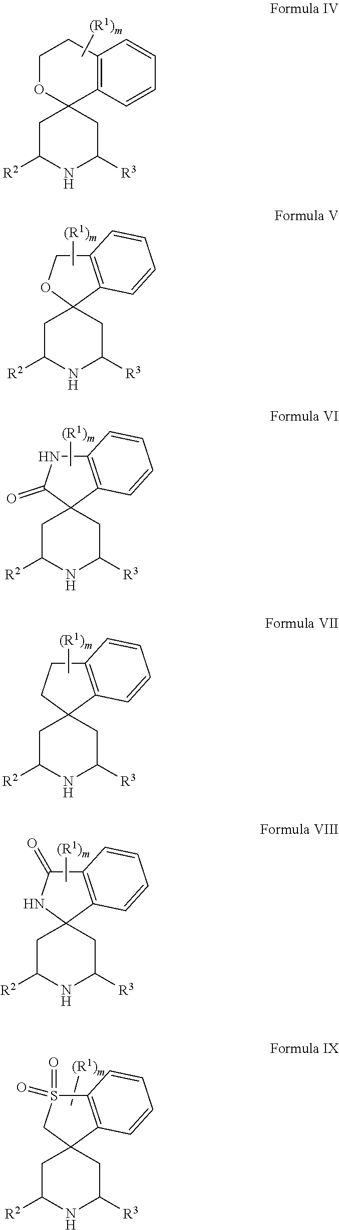
a tautomer thereof, a deuterated derivative of that compound or tautomer, or a pharmaceutically acceptable salt of any of the foregoing, wherein all variables not specifically defined herein are as defined in any one of claims 1-4.

28. The compound, tautomer, deuterated derivative, or pharmaceutically acceptable salt according to claim 1, wherein the compound is represented by one of the following structural formulae: 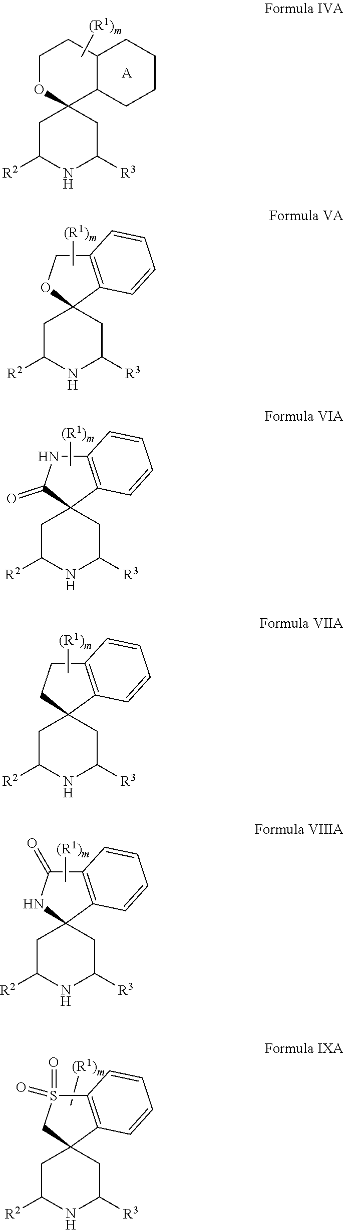
a tautomer thereof, a deuterated derivative of that compound or tautomer, or a pharmaceutically acceptable salt of any of the foregoing, wherein all variables not specifically defined herein are as defined in any one of claims 1-4.

29. The compound, tautomer, deuterated derivative, or pharmaceutically acceptable salt according to claim 1, wherein the compound is represented by one of the following structural formulae: 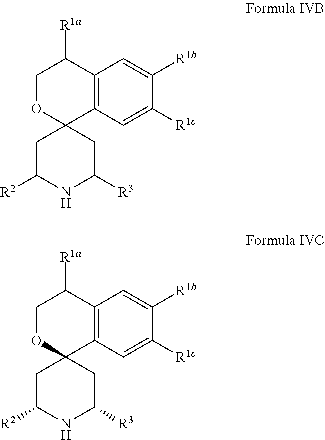
a tautomer thereof, a deuterated derivative of that compound or tautomer, or a pharmaceutically acceptable salt of any of the foregoing, wherein:
R^{1a} is chosen from hydrogen, halogen, —OH, and phenyl groups, wherein:
the phenyl of R^{1a} is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂ groups;
R^{1b} and R^{1c} are each independently chosen from hydrogen, halogen, —OH, cyano, C₁-C₄ alkyl, C₁-C₄ alkoxy, —C(═O)OR^{c}, —C(═O)N(R^{c})₂, and —OS(═O)₂R^{c} groups, wherein:
R^{c}, for each occurrence, is independently chosen from hydrogen, C₁-C₄ alkyl, and C₁-C₄ haloalkyl groups; and
the C₁-C₆ alkyl of R^{1b} and/or R^{1c} is optionally substituted with 1 to 3 groups independently chosen from halogen and —OH groups; and
all variables not specifically defined herein are as defined in any one of claims 1-4.

30. The compound, tautomer, deuterated derivative, or pharmaceutically acceptable salt according to claim 1, wherein the compound is represented by one of the following structural formulae: 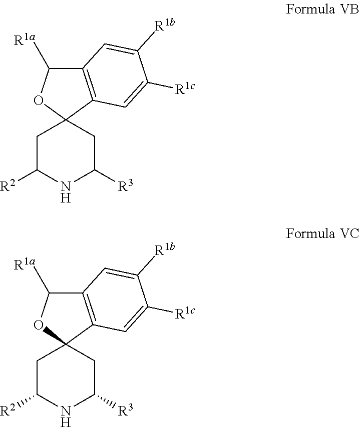
a tautomer thereof, a deuterated derivative of that compound or tautomer, or a pharmaceutically acceptable salt of any of the foregoing, wherein:
R^{1a} is chosen from hydrogen, phenyl, and C(═O)N(R^{c1})₂ groups, wherein:
the phenyl of R^{1a} is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy,
—C(═O)NH₂, —C(═O)NH(C₁-C₄ alkyl), and —C(═O)N(C₁-C₄ alkyl)₂ groups;
R^{c1}, for each occurrence, is independently chosen from hydrogen and C₁-C₄ alkyl groups;
R^{1b} and R^{1c} are each independently chosen from hydrogen and halogen groups; and
all variables not specifically defined herein are as defined in any one of claims 1-4.

31. The compound, tautomer, deuterated derivative, or pharmaceutically acceptable salt according to claim 1, wherein the compound is represented by one of the following structural formulae: 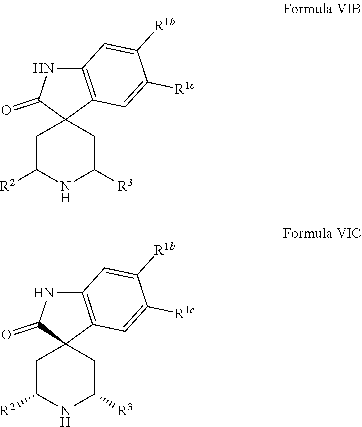
a tautomer thereof, a deuterated derivative of that compound or tautomer, or a pharmaceutically acceptable salt of any of the foregoing, wherein:
R^{1a} and R^{1b} are each independently chosen from hydrogen, halogen, C₁-C₄ alkyl, and C₁-C₄ haloalkyl groups; and
all variables not specifically defined herein are as defined in any one of claims 1-4.

32. The compound, tautomer, deuterated derivative, or pharmaceutically acceptable salt according to claim 1, wherein the compound is represented by one of the following structural formulae: 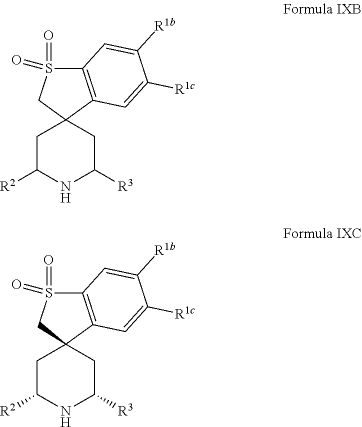
a tautomer thereof, a deuterated derivative of that compound or tautomer, or a pharmaceutically acceptable salt of any of the foregoing, wherein:
R^{1a} and R^{1b} are each independently chosen from hydrogen, halogen, C₁-C₄ alkyl, and C₁-C₄ haloalkyl groups; and
all variables not specifically defined herein are as defined in any one of claims 1-4.

33. A compound, tautomer, deuterated derivative, or pharmaceutically acceptable salt chosen from the compounds of Table 1, tautomers thereof, deuterated derivatives of those compounds and tautomers, and pharmaceutically acceptable salts of any of the foregoing.

34. A compound, tautomer, deuterated derivative, or pharmaceutically acceptable salt chosen from the compounds of Table 2, tautomers thereof, deuterated derivatives of those compounds and tautomers, and pharmaceutically acceptable salts of any of the foregoing.

35. A pharmaceutical composition comprising at least one compound, tautomer, deuterated derivative, or pharmaceutically acceptable salt according to any one of claims 1-34 and a pharmaceutically acceptable carrier.

36. A method of treating focal segmental glomerulosclerosis and/or non-diabetic kidney disease comprising administering to a patient in need thereof at least one compound, tautomer, deuterated derivative, or pharmaceutically acceptable salt according to any one of claims 1-34 or a pharmaceutical composition according to claim 35.

37. A method of inhibiting APOL1 activity comprising contacting said APOL1 with at least one compound, tautomer, deuterated derivative, or pharmaceutically acceptable salt according to any one of claims 1-34 or a pharmaceutical composition according to claim 35.

38. A silicon derivative of the at least one compound, tautomer, deuterated derivative, or pharmaceutically acceptable salt according to any one of claims 1-34.

39. A pharmaceutical composition comprising a silicon derivative according to claim 38.

40. A method of treating focal segmental glomerulosclerosis and/or non-diabetic kidney disease comprising administering to a patient in need thereof a silicon derivative according to claim 38 or a pharmaceutical composition according to claim 39.

41. A boron derivative of the at least one compound, tautomer, deuterated derivative, or pharmaceutically acceptable salt according to any one of claims 1-34.

42. A pharmaceutical composition comprising a boron derivative according to claim 41.

43. A method of treating focal segmental glomerulosclerosis and/or non-diabetic kidney disease comprising administering to a patient in need thereof a boron derivative according to claim 41 or a pharmaceutical composition according to claim 42.

44. A phosphorus derivative of at least one compound, tautomer, deuterated derivative, or pharmaceutically acceptable salt according to any one of claims 1-34.

45. A pharmaceutical composition comprising a phosphorus derivative according to claim 44.

46. A method of treating focal segmental glomerulosclerosis and/or non-diabetic kidney disease comprising administering to a patient in need thereof a phosphorus derivative according to claim 44 or a pharmaceutical composition according to claim 45.

47. A method of treating an APOL1-mediated disease comprising administering to a patient in need thereof at least one compound, tautomer, deuterated derivative, or pharmaceutically acceptable salt according to any one of claims 1-34 or a pharmaceutical composition according to claim 35.

48. The method according to claim 47, wherein the APOL1-mediated disease is cancer.

49. The method according to claim 47 or claim 48, wherein the APOL1-mediated disease is pancreatic cancer.
